(19) United States
(12) Patent Application Publication
(10) Pub. No.: US 2026/0363240 A1
(43) Pub. Date: Nov. 19, 2026

(54) 3-ALKOXYBENZAMIDE DERIVATIVE, AND PEST CONTROL AGENT

(71) Applicant: Kumiai Chemical Industry Co., Ltd., Tokyo (JP)

(72) Inventors: Kosuke TANAKA, Tokyo (JP); Sakae SAKURAI, Tokyo (JP); Yukihiro ENOMOTO, Tokyo (JP); Keiji TORIYABE, Tokyo (JP); Hirokazu ARAI, Tokyo (JP); Yuhsuke NAKANE, Tokyo (JP); Takeshi MATSUDA, Tokyo (JP); Akira TAKANEZAWA, Tokyo (JP); Shusuke KOIKE, Tokyo (JP); Shunsuke KITAHARA, Tokyo (JP); Shigeyuki MUKAWA, Tokyo (JP)

(73) Assignee: Kumiai Chemical Industry Co., Ltd., Tokyo (JP)

(21) Appl. No.: 19/676,161

(22) Filed: May 13, 2026

Related U.S. Application Data
(63) Continuation of application No. 17/796,592, filed on Jan. 29, 2021, now Pat. No. 12,677,830.

(30) Foreign Application Priority Data
Jan. 31, 2020 (JP) 2020-014653

(51) Int. Cl. A01N 37/18; A01N 25/30; A01P 5/00; A01P 7/02; A01P 7/04

(52) U.S. Cl. CPC A01N 37/18; A01N 25/30; A01P 5/00; A01P 7/02; A01P 7/04

(57) ABSTRACT

The present invention provides: a 3-halobenzamide derivative having an excellent pest control effect, or a salt thereof; and a pest control agent and a production intermediate which contain the same as an active ingredient.

Provided are: a 3-alkoxybenzamide derivative represented by general formula [I]

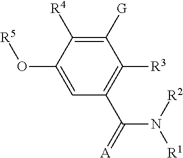

[In the formula, A represents an oxygen atom or a sulfur atom, G represents a hydrogen atom, a halogen atom, or a C₁-C₆ alkyl group, R¹ represents a C₁-C₆ alkyl group, a C₃-C₆ cycloalkyl group, etc., R² represents a hydrogen atom, etc., R³ represents a halogen atom, C₁-C₆ alkyl group, etc., R⁴ represents a halogen atom, C₁-C₆ alkyl group, etc., and R⁵ represents a C₁-C₁₂ alkyl group, etc.], or a salt thereof; and a pest control agent characterized by containing said derivative or salt thereof as an active ingredient.

### TECHNICAL FIELD

The present invention relates to a novel 3-alkoxybenzamide derivative or an agriculturally acceptable salt thereof, a pest control agent comprising the derivative as an active ingredient, and a production intermediate of the derivative.

### BACKGROUND ART

Patent Documents 1 and 2 describe 3-alkoxybenzamide derivatives, but in the compound described in Patent Document 1, the amine moiety of the amide bond is limited to an adamantylalkyl group. In addition, in the compound described in Patent Document 2, the substituent on the alkoxy group is limited to a pyridopyrimidinone ring. Patent Document 3 describes a 3-alkoxybenzamide derivative having herbicidal effect, but does not describe a pest control effect.

### CITATION LIST

### Patent Document

Patent Document 1: WO 01/042194 A1
Patent Document 2: WO 2016/068580 A2
Patent Document 3: WO 94/05153 A1

### SUMMARY OF THE INVENTION

### Problems to be Solved by the Invention

It is desired that the pest control agent to be used for useful crops is a chemical agent which is applied to soil or foliage and capable of exhibiting a sufficient pest control effect at a low dosage. In addition, due to an increasing demand for safety and influence on the environment of chemical substances, and development of a safer pest control agent is desired. Furthermore, in recent years, pests having acquired resistance to pest control agents have appeared due to long use of pest control agents such as insecticides and acaricides for many years, and therefore it is difficult to completely control pests. In addition, the use of pest control agents having high human and animal toxicity is also a problem in terms of safety for workers and the like.

Under such circumstances, an object of the present invention is to solve the above-described problems of conventional pest control agents, and to provide a pest control agent excellent in safety, control effect, residual effect, and the like.

### Means for Solving the Problems

In order to develop a pest control agent having the above-described preferable properties, the present inventors synthesized various 3-alkoxybenzamide derivatives, and conducted intensive studies on their physiological activity. As a result, the present inventors have found that 3-alkoxybenzamide derivatives (hereinafter, referred to as compounds of the present invention) of formula [I] below exhibit excellent effects on various pests, and have further continued the research to complete the present invention.

That is, the present invention has the following features.

(1) A 3-alkoxybenzamide derivative of formula [I], or an agriculturally acceptable salt thereof,

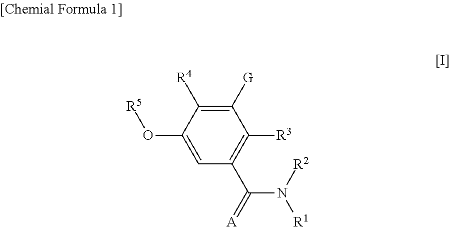

wherein in the formula [I],
A represents an oxygen atom or a sulfur atom,
G represents a hydrogen atom; a halogen atom; or a (C1-C6)alkyl,
R¹ represents a (C1-C6)alkyl; a (C1-C6)haloalkyl; a (C3-C6)cycloalkyl; a (C3-C6)halocycloalkyl; a (C2-C6)alkenyl; a (C2-C6)haloalkenyl; a (C2-C6)alkynyl; a (C2-C6)haloalkynyl; a (C1-C6)alkoxy; a (C1-C6)haloalkoxy; a (C1-C6)alkoxy (C1-C6)alkyl; a (C3-C6)cycloalkyl (C1-C6)alkyl; a (C3-C6)halocycloalkyl (C1-C6)alkyl; a cyano (C1-C6)alkyl; a cyano (C3-C6)cycloalkyl; a cyano (C3-C6)cycloalkyl (C1-C6)alkyl; a cyano heterocycloalkyl; a hydroxy (C3-C6)cycloalkyl; a (C1-C6)alkylcarbonyloxy (C3-C6)cycloalkyl; a (C1-C6)alkylsulfonyloxy (C3-C6)cycloalkyl; a (C1-C6)alkoxy (C3-C6)cycloalkyl; a (C1-C6)haloalkoxy (C3-C6)cycloalkyl; a (C1-C6)alkylthio (C3-C6)cycloalkyl; a (C1-C6)haloalkylthio (C3-C6)cycloalkyl; a (C1-C6)alkylsulfinyl (C3-C6)cycloalkyl; a (C1-C6)haloalkylsulfinyl (C3-C6)cycloalkyl; a (C1-C6)alkylsulfonyl (C3-C6)cycloalkyl; a (C1-C6)haloalkylsulfonyl (C3-C6)cycloalkyl; a (C2-C6)alkynyl (C3-C6)cycloalkyl; a (C1-C6)alkoxycarbonyl (C3-C6)cycloalkyl; a carboxy (C3-C6)cycloalkyl; a carbamoyl (C3-C6)cycloalkyl; a (C1-C6)alkylaminocarbonyl (C3-C6)cycloalkyl (the amino moiety is optionally substituted with R¹⁰); a (C4-C8)bicycloalkyl; a (C1-C6)alkoxyimino; a phenyl (the phenyl is optionally mono-substituted or poly-substituted with R⁷); a phenyl (C1-C6)alkyl (the phenyl moiety is optionally mono-substituted or poly-substituted with R⁷); a heteroaryl (the heteroaryl is optionally mono-substituted or poly-substituted with R⁷); a heteroaryl (C1-C3)alkyl (the heteroaryl moiety is optionally mono-substituted or poly-substituted with R⁷); a heteroaryl (C3-C6)cycloalkyl (the heteroaryl moiety is optionally mono-substituted or poly-substituted with R⁷); a heterocycloalkyl (the heterocycloalkyl is optionally substituted with R¹⁰); a heterocycloalkyl (C1-C6)alkyl (the heterocycloalkyl moiety is optionally substituted with R¹⁰); or a heterocycloalkyl (C3-C6)cycloalkyl (the heterocycloalkyl moiety is optionally substituted with R¹⁰),
R² represents a hydrogen atom; a (C1-C6)alkyl; a (C1-C6)haloalkyl; a (C3-C6)cycloalkyl; a (C3-C6)halocycloalkyl; a (C2-C6)alkenyl; a (C2-C6)haloalkenyl; a (C2-C6)alkynyl; a (C2-C6)haloalkynyl; a (C1-C6)alkoxy; a (C1-C6)haloalkoxy; a (C3-C6)cycloalkyl (C1-C6)alkyl; a (C3-C6)halocycloalkyl (C1-C6)alkyl; a phenyl (the phenyl is optionally mono-substituted or poly-substituted with R⁷); a phenyl (C1-C6)alkyl (the phenyl moiety is optionally mono-substituted or poly-substituted with R⁷); a heteroaryl (the heteroaryl is optionally mono-substituted or poly-substituted with R⁷); a heteroaryl (C1-C3)alkyl (the heteroaryl moiety is optionally mono-substituted or poly-substituted with R⁷); a cyano; a cyano (C1-C6)alkyl; a cyano (C3-C6)cycloalkyl; a hydroxy; a hydroxy (C1-C6)alkyl; a (C1-C6)alkoxy (C1-C6)alkyl; a formyl; an amino; a (C1-C6)alkylamino (the amino moiety is optionally substituted with R¹⁰); a (C1-C6)alkylcarbonyl; a (C1-C6)alkoxy (C1-C6)alkylcarbonyl; a (C3-C6)cycloalkylcarbonyl; a (C3-C6)cycloalkyl (C1-C6)alkylcarbonyl; a benzoyl (the benzoyl is optionally mono-substituted or poly-substituted with R⁷); a (C1-C6)alkoxycarbonyl; a (C1-C6)haloalkoxycarbonyl; a (C2-C6)alkenyloxycarbonyl; a carbamoyl; a (C1-C6)alkylaminocarbonyl (the amino moiety is optionally substituted with R¹⁰); a (C1-C6)alkoxycarbonyl (C1-C6)alkylamino (the amino moiety is optionally substituted with R¹⁰); a (C1-C6)alkoxycarbonylamino (the amino moiety is optionally substituted with R¹⁰); a ((C1-C6)alkylthio)carbonyl; a ((C1-C6)alkyl)thiocarbonyl; a ((C1-C6)alkoxy)thiocarbonyl; a thiocarbamoyl; a (C1-C6)alkylaminothiocarbonyl (the amino moiety is optionally substituted with R¹⁰); a (C1-C6)alkylsulfonyl; a (C1-C6)haloalkylsulfonyl; a sulfamoyl; a (C1-C6)alkylaminosulfonyl (the amino moiety is optionally substituted with R¹⁰); a (C1-C6)alkoxycarbonyl (C1-C6)alkyl; an aminocarbonyl (C1-C6)alkyl; a (C1-C6)alkylaminocarbonyl (C1-C6)alkyl (the amino moiety is optionally substituted with R¹¹); a (C3-C6)cycloalkylaminocarbonyl (C1-C6)alkyl (the amino moiety is optionally substituted with R¹⁰); a (C1-C6)haloalkylaminocarbonyl (C1-C6)alkyl (the amino moiety is optionally substituted with R¹¹); a (C1-C6)alkylcarbonyl (C1-C6)alkyl; a (C1-C6)haloalkylcarbonyl (C1-C6)alkyl; a (C1-C6)alkylcarbonyloxy (C1-C6)alkyl; a (C1-C6)alkoxycarbonyloxy (C1-C6)alkyl; a (C1-C6)alkoxycarbonylamino (C1-C6)alkyl (the amino moiety is optionally substituted with a (C1-C6)alkyl); a hydroxyimino; a hydroxyimino (C1-C6)alkyl; a (C1-C6)alkoxyimino; a (C1-C6)alkoxyimino (C1-C6)alkyl; or a (C1-C6)haloalkoxyimino (C1-C6)alkyl, or R¹ and R², together with the nitrogen atom to which R¹ and R² are attached form a 3- to 8-membered heterocyclic ring or a 3- to 8-membered heterocyclic ring having 1 to 4 heteroatoms selected from an oxygen atom, a sulfur atom, and a nitrogen atom, wherein the formed heterocyclic ring is optionally substituted with a halogen atom, a cyano, a nitro, a (C1-C6)alkyl, a (C1-C6)alkoxy, a (C1-C6)haloalkyl, or an oxo,
R³ represents a halogen atom; a cyano; a (C1-C6)alkyl; a (C1-C6)haloalkyl; a (C3-C6)cycloalkyl; a (C1-C6)alkoxy; or a (C1-C6)haloalkoxy,
R⁴ represents a hydrogen atom; a halogen atom; a (C1-C6)alkyl; or a (C1-C6)haloalkyl,
R⁵ represents a (C1-C12)alkyl (the (C1-C12)alkyl is optionally mono-substituted or poly-substituted with R⁶); a (C3-C6)cycloalkyl (the (C3-C6)cycloalkyl is optionally mono-substituted or poly-substituted with R⁶); a (C2-C6)alkenyl (the (C2-C6)alkenyl is optionally mono-substituted or poly-substituted with R⁶); or a (C2-C6)alkynyl (the (C2-C6)alkynyl is optionally mono-substituted or poly-substituted with R⁶),
R⁶ represents a halogen atom; a (C1-C6)alkyl; a (C1-C6)haloalkyl; a (C3-C6)cycloalkyl; a (C3-C6)halocycloalkyl; a hydroxy; a (C1-C6)alkoxy; a (C1-C6)haloalkoxy; a (C3-C6)cycloalkoxy; a (C3-C6)halocycloalkoxy; a (C1-C6)alkoxy (C1-C6)alkoxy; a (C1-C6)haloalkoxy (C1-C6)alkoxy; a (C1-C6)haloalkoxy (C1-C6)haloalkoxy; a thiol; a (C1-C6)alkylthio; a (C1-C6)haloalkylthio; a (C2-C6)alkenylthio; a (C2-C6)haloalkenylthio; a (C3-C6)cycloalkylthio; a (C3-C6)halocycloalkylthio; a (C3-C6)cycloalkyl (C1-C6)alkylthio; a (C3-C6)halocycloalkyl (C1-C6)alkylthio; a tri((C1-C6)alkyl)silyl (C1-C6)alkylthio; a (C1-C6)alkylthio (C1-C6)alkoxy; a (C1-C6)haloalkylthio (C1-C6)alkoxy; a (C1-C6)alkylsulfinyl; a (C1-C6)haloalkylsulfinyl; a (C3-C6)cycloalkylsulfinyl; a (C3-C6)halocycloalkylsulfinyl; a (C1-C6)alkylsulfonyl; a (C1-C6)haloalkylsulfonyl; a (C3-C6)cycloalkylsulfonyl; a (C3-C6)halocycloalkylsulfonyl; a formyl; a (C1-C6)alkylcarbonyl; a (C1-C6)haloalkylcarbonyl; a benzoyl (the benzoyl is optionally mono-substituted or poly-substituted with R⁷); a (C1-C6)alkoxycarbonyl; a (C1-C6)haloalkoxycarbonyl; a (C1-C6)alkylaminocarbonyl (the amino moiety is optionally substituted with R¹⁰); a (C1-C6)haloalkylaminocarbonyl (the amino moiety is optionally substituted with R¹⁰); a (C1-C6)alkylaminocarbonylthio (the amino moiety is optionally substituted with R¹⁰); a (C1-C6)haloalkylaminocarbonylthio (the amino moiety is optionally substituted with R¹⁰); a formyloxy; a (C1-C6)alkylcarbonyloxy; a (C1-C6)haloalkylcarbonyloxy; a benzoyloxy (the benzoyloxy is optionally mono-substituted or poly-substituted with R⁷); a (C1-C6)alkylsulfinyloxy; a (C1-C6)haloalkylsulfinyloxy; a (C3-C6)cycloalkylsulfinyloxy; a (C3-C6)halocycloalkylsulfinyloxy; a phenylsulfinyloxy (the phenylsulfinyloxy is optionally mono-substituted or poly-substituted with R⁷); a (C1-C6)alkylsulfonyloxy; a (C1-C6)haloalkylsulfonyloxy; a (C3-C6)cycloalkylsulfonyloxy; a (C3-C6)halocycloalkylsulfonyloxy; a phenylsulfonyloxy (the phenyl moiety is optionally mono-substituted or poly-substituted with R⁷); an amino; a (C1-C6)alkylamino (the amino moiety is optionally substituted with R¹⁰); a (C1-C6)haloalkylamino (the amino moiety is optionally substituted with R¹⁰); a (C1-C6)alkylcarbonylamino (the amino moiety is optionally substituted with R¹⁰); a (C1-C6)haloalkylcarbonylamino (the amino moiety is optionally substituted with R¹⁰); a phenylcarbonylamino (the phenyl moiety is optionally mono-substituted or poly-substituted with R⁷, and the amino moiety is optionally substituted with R¹⁰); a (C1-C6)alkoxycarbonylamino (the amino moiety is optionally substituted with R¹⁰); a (C1-C6)haloalkoxycarbonylamino (the amino moiety is optionally substituted with R¹⁰); a (C1-C6)alkylaminocarbonylamino (the amino moiety is optionally substituted with R¹⁰); a (C1-C6)haloalkylaminocarbonylamino (the amino moiety is optionally substituted with R¹⁰); a (C1-C6)alkylsulfinylamino (the amino moiety is optionally substituted with R¹⁰); a (C1-C6)haloalkylsulfinylamino (the amino moiety is optionally substituted with R¹⁰); a phenylsulfinylamino (the phenyl moiety is optionally mono-substituted or poly-substituted with R⁷, and the amino moiety is optionally substituted with R¹⁰); a (C1-C6)alkylsulfonylamino (the amino moiety is optionally substituted with R¹⁰); a (C1-C6)haloalkylsulfonylamino (the amino moiety is optionally substituted with R¹⁰); a phenylsulfonylamino (the phenyl moiety is optionally mono-substituted or poly-substituted with R⁷, and the amino moiety is optionally substituted with R¹⁰); a (C1-C6)alkoxyimino; a tri((C1-C6)alkyl)silyl; a phenyl (the phenyl is optionally mono-substituted or poly-substituted with R⁷); a phenoxy (the phenoxy is optionally mono-substituted or poly-substituted with R⁷); a phenyl (C1-C6)alkoxy (the phenyl moiety is optionally mono-substituted or poly-substituted with R⁷); a phenylthio (the phenylthio is optionally mono-substituted or poly-substituted with R⁷); a phenylsulfinyl (the phenylsulfinyl is optionally mono-substituted or poly-substituted with R⁷); a phenylsulfonyl (the phenylsulfonyl is optionally mono-substituted or poly-substituted with R⁷); a phenyl (C1-C6)alkylthio (the phenyl moiety is optionally mono-substituted or poly-substituted with R⁷); a phenyl (C1-C6)alkylsulfinyl (the phenyl moiety is optionally mono-substituted or poly-substituted with R⁷); a phenyl (C1-C6)alkylsulfonyl (the phenyl moiety is optionally mono-substituted or poly-substituted with R⁷); a —O—N═C(R⁸)(R⁹) group; an adamantyl; an azetidinyl (a nitrogen atom of the azetidinyl is optionally substituted with R¹⁰); a pyrrolidinyl (a nitrogen atom of the pyrrolidinyl is optionally substituted with R¹⁰); a piperidinyl (a nitrogen atom of the piperidinyl is optionally substituted with R¹); a 1,3-dioxolanyl; a 1,3-dioxanyl; a pyrrolyl (the pyrrolyl is optionally mono-substituted or poly-substituted with R⁷); a pyrazolyl (the pyrazolyl is optionally mono-substituted or poly-substituted with R⁷); an imidazolyl (the imidazolyl is optionally mono-substituted or poly-substituted with R⁷); a triazolyl (the triazolyl is optionally mono-substituted or poly-substituted with R⁷); an oxazolyl (the oxazolyl is optionally mono-substituted or poly-substituted with R⁷); an isoxazolyl (the isoxazolyl is optionally mono-substituted or poly-substituted with R⁷); a thiazolyl (the thiazolyl is optionally mono-substituted or poly-substituted with R⁷); an isothiazolyl (the isothiazolyl is optionally mono-substituted or poly-substituted with R⁷); a pyridyl (the pyridyl is optionally mono-substituted or poly-substituted with R⁷, and further a nitrogen atom of the pyridyl is optionally oxidized to form a N-oxide); a pyrimidinyl (the pyrimidinyl is optionally mono-substituted or poly-substituted with R⁷); a pyridyloxy (the pyridyloxy is optionally mono-substituted or poly-substituted with R⁷); a tetrahydrofuranyl (the tetrahydrofuranyl is optionally mono-substituted or poly-substituted with R⁷); a 1,3-dioxoisoindolinyl (the 1,3-dioxoisoindolinyl is optionally mono-substituted or poly-substituted with R⁷); a cyano; a nitro; a carboxy; a thiocyanato; or an aminooxy,
R⁷ represents a halogen atom; a (C1-C6)alkyl; a (C1-C6)haloalkyl; a (C3-C6)cycloalkyl; a (C3-C6)halocycloalkyl; a (C3-C6)cycloalkyl (C1-C6)alkyl; a (C3-C6)halocycloalkyl (C1-C6)alkyl; a (C1-C6)alkoxy; a (C1-C6)haloalkoxy; a (C1-C6)alkylthio; a (C1-C6)haloalkylthio; a (C1-C6)alkylsulfinyl; a (C1-C6)haloalkylsulfinyl; a (C1-C6)alkylsulfonyl; a (C1-C6)haloalkylsulfonyl; a (C1-C6)alkylthio (C1-C6)alkyl; a (C1-C6)haloalkylthio (C1-C6)alkyl; a (C1-C6)alkylsulfonyloxy; a (C1-C6)haloalkylsulfonyloxy; a phenyl (the phenyl is optionally mono-substituted or poly-substituted with a halogen atom, an alkyl, or a haloalkyl); a phenyl (C1-C6)alkyl; a phenyl (C1-C6)alkoxy; a cyano; or a nitro,
R⁸ and R⁹ are identical to or different from each other, and represent a hydrogen atom; a (C1-C6)alkyl; a (C1-C6)haloalkyl; a (C3-C6)cycloalkyl; a (C3-C6)halocycloalkyl; or a phenyl (the phenyl is optionally mono-substituted or poly-substituted with R⁷); or R⁸ and R⁹, together with the carbon atom to which R⁸ and R⁹ are attached form a 3- to 6-membered ring, and
R¹⁰ represents a (C1-C6)alkyl; a (C1-C6)haloalkyl; a (C3-C6)cycloalkyl; a (C3-C6)halocycloalkyl; a (C1-C6)alkoxy; a (C1-C6)alkoxy (C1-C6)alkyl; a (C1-C6)haloalkoxy (C1-C6)alkyl; a (C1-C6)alkoxy (C1-C6)alkoxy (C1-C6)alkyl; a (C1-C6)alkylthio (C1-C6)alkyl; a (C1-C6)alkylsulfinyl (C1-C6)alkyl; a (C1-C6)alkylsulfonyl (C1-C6)alkyl; a (C1-C6)alkylthio (C1-C6)alkoxy (C1-C6)alkyl; a (C1-C6)alkylsulfinyl (C1-C6)alkoxy (C1-C6)alkyl; a (C1-C6)alkylsulfonyl (C1-C6)alkoxy (C1-C6)alkyl; a cyano (C1-C6)alkyl; a cyano (C1-C6)alkoxy (C1-C6)alkyl; a (C1-C6)alkylcarbonyl; a (C1-C6)haloalkylcarbonyl; a (C1-C6)alkoxycarbonyl; a (C1-C6)haloalkoxycarbonyl; a (C1-C6)alkylaminocarbonyl; a (C1-C6)haloalkylaminocarbonyl; a benzoyl (the benzoyl is optionally mono-substituted or poly-substituted with R⁷); a (C1-C6)alkylsulfonyl; a (C1-C6)haloalkylsulfonyl; a (C1-C6)alkylcarbonyl (C1-C6)alkyl; a (C1-C6)alkoxycarbonyl (C1-C6)alkyl; a (C1-C6)alkylaminocarbonyl (C1-C6)alkyl; a (C1-C6)alkylcarbonyloxy (C1-C6)alkyl; a (C1-C6)alkoxycarbonyloxy (C1-C6)alkyl; a (C1-C6)alkoxycarbonylamino (C1-C6)alkyl (the amino moiety is optionally substituted with a (C1-C6)alkyl); or a 5-methyl-1,3-dioxole-2-one-4-ylmethyl.

(2) An agrochemical composition comprising the 3-alkoxybenzamide derivative or the agriculturally acceptable salt thereof according to (1) as an active ingredient.

(3) The agrochemical composition according to (2), which further comprises a surfactant.

(4) A pest control agent comprising the 3-alkoxybenzamide derivative or the agriculturally acceptable salt thereof according to (1) as an active ingredient.

(5) The pest control agent according to (4), wherein the pest control agent is an insecticide, a nematicide, and an acaricide.

(6) The pest control agent according to (5), wherein the pest control agent is capable of controlling pests in a paddy field, a dry field, a lawn, an orchard, a non-crop land, a greenhouse, a raising seeding facility, and a plant factory where an agricultural or horticultural plant is cultivated.

(7) The pest control agent according to (6), wherein the agricultural or horticultural plant is a plant to which resistance is imparted by a breeding or genetic modification techniques.

(8) A process for controlling a pest, comprising a step of applying an effective amount of the 3-alkoxybenzamide derivative or the agriculturally acceptable salt thereof according to (1).

(9) A process for controlling a pest, comprising a step of applying an agrochemical composition comprising the 3-alkoxybenzamide derivative or the agriculturally acceptable salt thereof according to (1) as an active ingredient to an agricultural or horticultural crop or a place where the agricultural or horticultural crop is to be grown or being grown, in a single or divided dose.

(10) The process for controlling a pest according to (8) or (9), wherein a place where a pest control agent is applied is a paddy field, a dry field, a lawn, an orchard, a non-crop land, a greenhouse, a raising seeding facility, and a plant factory.

(11) The process for controlling a pest according to any one of (8) to (10), wherein the 3-alkoxybenzamide derivative or agriculturally acceptable salt thereof according to (1) is used as an insecticide, a nematicide, and an acaricide.

(12) A process for using a pest control agent, characterized in that, in the process, the pest control agent according to any one of (4) to (7) is applied for controlling a pest harmful to an agricultural or horticultural crop.

(13) A hydroxybenzamide derivative of formula [II], or a salt thereof,

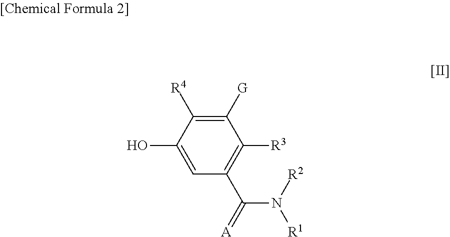

wherein in the formula [II],
A represents an oxygen atom or a sulfur atom,
G represents a hydrogen atom; a halogen atom; or a (C1-C6)alkyl,
R¹ represents a (C1-C6)alkyl; a (C1-C6)haloalkyl; a (C3-C6)cycloalkyl; a (C3-C6)halocycloalkyl; a (C2-C6)alkenyl; a (C2-C6)haloalkenyl; a (C2-C6)alkynyl; a (C2-C6)haloalkynyl; a (C1-C6)alkoxy; a (C1-C6)haloalkoxy; a (C1-C6)alkoxy (C1-C6)alkyl; a (C3-C6)cycloalkyl (C1-C6)alkyl; a (C3-C6)halocycloalkyl (C1-C6)alkyl; a cyano (C1-C6)alkyl; a cyano (C3-C6)cycloalkyl; a cyano (C3-C6)cycloalkyl (C1-C6)alkyl; a cyano heterocycloalkyl; a hydroxy (C3-C6)cycloalkyl; a (C1-C6)alkylcarbonyloxy (C3-C6)cycloalkyl; a (C1-C6)alkylsulfonyloxy (C3-C6)cycloalkyl; a (C1-C6)alkoxy (C3-C6)cycloalkyl; a (C1-C6)haloalkoxy (C3-C6)cycloalkyl; a (C1-C6)alkylthio (C3-C6)cycloalkyl; a (C1-C6)haloalkylthio (C3-C6)cycloalkyl; a (C1-C6)alkylsulfinyl (C3-C6)cycloalkyl; a (C1-C6)haloalkylsulfinyl (C3-C6)cycloalkyl; a (C1-C6)alkylsulfonyl (C3-C6)cycloalkyl; a (C1-C6)haloalkylsulfonyl (C3-C6)cycloalkyl; a (C2-C6)alkynyl (C3-C6)cycloalkyl; a (C1-C6)alkoxycarbonyl (C3-C6)cycloalkyl; a carboxy (C3-C6)cycloalkyl; a carbamoyl (C3-C6)cycloalkyl; a (C1-C6)alkylaminocarbonyl (C3-C6)cycloalkyl (the amino moiety is optionally substituted with R¹⁰); a (C4-C8)bicycloalkyl; a (C1-C6)alkoxyimino; a phenyl (the phenyl is optionally mono-substituted or poly-substituted with R⁷); a phenyl (C1-C6)alkyl (the phenyl moiety is optionally mono-substituted or poly-substituted with R⁷); a heteroaryl (the heteroaryl is optionally mono-substituted or poly-substituted with R⁷); a heteroaryl (C1-C3)alkyl (the heteroaryl moiety is optionally mono-substituted or poly-substituted with R⁷); a heteroaryl (C3-C6)cycloalkyl (the heteroaryl moiety is optionally mono-substituted or poly-substituted with R⁷); a heterocycloalkyl (the heterocycloalkyl is optionally substituted with R¹⁰); a heterocycloalkyl (C1-C6)alkyl (the heterocycloalkyl moiety is optionally substituted with R¹⁰); or a heterocycloalkyl (C3-C6)cycloalkyl (the heterocycloalkyl moiety is optionally substituted with R¹⁰),
R² represents a hydrogen atom; a (C1-C6)alkyl; a (C1-C6)haloalkyl; a (C3-C6)cycloalkyl; a (C3-C6)halocycloalkyl; a (C2-C6)alkenyl; a (C2-C6)haloalkenyl; a (C2-C6)alkynyl; a (C2-C6)haloalkynyl; a (C1-C6)alkoxy; a (C1-C6)haloalkoxy; a (C3-C6)cycloalkyl (C1-C6)alkyl; a (C3-C6)halocycloalkyl (C1-C6)alkyl; a phenyl (the phenyl is optionally mono-substituted or poly-substituted with R⁷); a phenyl (C1-C6)alkyl (the phenyl moiety is optionally mono-substituted or poly-substituted with R⁷); a heteroaryl (the heteroaryl is optionally mono-substituted or poly-substituted with R⁷); a heteroaryl (C1-C3)alkyl (the heteroaryl moiety is optionally mono-substituted or poly-substituted with R⁷); a cyano; a cyano (C1-C6)alkyl; a cyano (C3-C6)cycloalkyl; a hydroxy; a hydroxy (C1-C6)alkyl; a (C1-C6)alkoxy (C1-C6)alkyl; a formyl; an amino; a (C1-C6)alkylamino (the amino moiety is optionally substituted with R¹⁰); a (C1-C6)alkylcarbonyl; a (C1-C6)alkoxy (C1-C6)alkylcarbonyl; a (C3-C6)cycloalkylcarbonyl; a (C3-C6)cycloalkyl (C1-C6)alkylcarbonyl; a benzoyl (the benzoyl is optionally mono-substituted or poly-substituted with R⁷); a (C1-C6)alkoxycarbonyl; a (C1-C6)haloalkoxycarbonyl; a (C2-C6)alkenyloxycarbonyl; a carbamoyl; a (C1-C6)alkylaminocarbonyl (the amino moiety is optionally substituted with R¹⁰); a (C1-C6)alkoxycarbonyl (C1-C6)alkylamino (the amino moiety is optionally substituted with R¹⁰); a (C1-C6)alkoxycarbonylamino (the amino moiety is optionally substituted with R¹⁰); a ((C1-C6)alkylthio)carbonyl; a ((C1-C6)alkyl)thiocarbonyl; a ((C1-C6)alkoxy)thiocarbonyl; a thiocarbamoyl; a (C1-C6)alkylaminothiocarbonyl (the amino moiety is optionally substituted with R¹⁰); a (C1-C6)alkylsulfonyl; a (C1-C6)haloalkylsulfonyl; a sulfamoyl; a (C1-C6)alkylaminosulfonyl (the amino moiety is optionally substituted with R¹⁰); a (C1-C6)alkoxycarbonyl (C1-C6)alkyl; an aminocarbonyl (C1-C6)alkyl; a (C1-C6)alkylaminocarbonyl (C1-C6)alkyl (the amino moiety is optionally substituted with R¹¹); a (C3-C6)cycloalkylaminocarbonyl (C1-C6)alkyl (the amino moiety is optionally substituted with R¹⁰); a (C1-C6)haloalkylaminocarbonyl (C1-C6)alkyl (the amino moiety is optionally substituted with R¹¹); a (C1-C6)alkylcarbonyl (C1-C6)alkyl; a (C1-C6)haloalkylcarbonyl (C1-C6)alkyl; a (C1-C6)alkylcarbonyloxy (C1-C6)alkyl; a (C1-C6)alkoxycarbonyloxy (C1-C6)alkyl; a (C1-C6)alkoxycarbonylamino (C1-C6)alkyl (the amino moiety is optionally substituted with a (C1-C6)alkyl); a hydroxyimino; a hydroxyimino (C1-C6)alkyl; a (C1-C6)alkoxyimino; a (C1-C6)alkoxyimino (C1-C6)alkyl; or a (C1-C6)haloalkoxyimino (C1-C6)alkyl, or
R¹ and R², together with the nitrogen atom to which R¹ and R² are attached form a 3- to 8-membered heterocyclic ring or a 3- to 8-membered heterocyclic ring having 1 to 4 heteroatoms selected from an oxygen atom, a sulfur atom, and a nitrogen atom, and the heterocyclic ring is optionally substituted with a halogen atom, a cyano, a nitro, a (C1-C6)alkyl, a (C1-C6)alkoxy, a (C1-C6)haloalkyl, or an oxo,
R³ represents a halogen atom; a cyano; a (C1-C6)alkyl; a (C1-C6)haloalkyl; a (C3-C6)cycloalkyl; a (C1-C6)alkoxy; or a (C1-C6)haloalkoxy,
R⁴ represents a hydrogen atom; a halogen atom; a (C1-C6)alkyl; or a (C1-C6)haloalkyl,
R⁷ represents a halogen atom; a (C1-C6)alkyl; a (C1-C6)haloalkyl; a (C3-C6)cycloalkyl; a (C3-C6)halocycloalkyl; a (C3-C6)cycloalkyl (C1-C6)alkyl; a (C3-C6)halocycloalkyl (C1-C6)alkyl; a (C1-C6)alkoxy; a (C1-C6)haloalkoxy; a (C1-C6)alkylthio; a (C1-C6)haloalkylthio; a (C1-C6)alkylsulfinyl; a (C1-C6)haloalkylsulfinyl; a (C1-C6)alkylsulfonyl; a (C1-C6)haloalkylsulfonyl; a (C1-C6)alkylthio (C1-C6)alkyl; a (C1-C6)haloalkylthio (C1-C6)alkyl; a (C1-C6)alkylsulfonyloxy; a (C1-C6)haloalkylsulfonyloxy; a phenyl (the phenyl is optionally mono-substituted or poly-substituted with a halogen atom, an alkyl, or a haloalkyl); a phenyl (C1-C6)alkyl; a phenyl (C1-C6)alkoxy; a cyano; or a nitro, and
R¹⁰ represents a (C1-C6)alkyl; a (C1-C6)haloalkyl; a (C3-C6)cycloalkyl; a (C3-C6)halocycloalkyl; a (C1-C6)alkoxy; a (C1-C6)alkoxy (C1-C6)alkyl; a (C1-C6)haloalkoxy (C1-C6)alkyl; a (C1-C6)alkoxy (C1-C6)alkoxy (C1-C6)alkyl; a (C1-C6)alkylthio (C1-C6)alkyl; a (C1-C6)alkylsulfinyl (C1-C6)alkyl; a (C1-C6)alkylsulfonyl (C1-C6)alkyl; a (C1-C6)alkylthio (C1-C6)alkoxy (C1-C6)alkyl; a (C1-C6)alkylsulfinyl (C1-C6)alkoxy (C1-C6)alkyl; a (C1-C6)alkylsulfonyl (C1-C6)alkoxy (C1-C6)alkyl; a cyano (C1-C6)alkyl; a cyano (C1-C6)alkoxy (C1-C6)alkyl; a (C1-C6)alkylcarbonyl; a (C1-C6)haloalkylcarbonyl; a (C1-C6)alkoxycarbonyl; a (C1-C6)haloalkoxycarbonyl; a (C1-C6)alkylaminocarbonyl; a (C1-C6)haloalkylaminocarbonyl; a benzoyl (the benzoyl is optionally mono-substituted or poly-substituted with R⁷); a (C1-C6)alkylsulfonyl; a (C1-C6)haloalkylsulfonyl; a (C1-C6)alkylcarbonyl (C1-C6)alkyl; a (C1-C6)alkoxycarbonyl (C1-C6)alkyl; a (C1-C6)alkylaminocarbonyl (C1-C6)alkyl; a (C1-C6)alkylcarbonyloxy (C1-C6)alkyl; a (C1-C6)alkoxycarbonyloxy (C1-C6)alkyl; a (C1-C6)alkoxycarbonylamino (C1-C6)alkyl (the amino moiety is optionally substituted with a (C1-C6)alkyl); or a 5-methyl-1,3-dioxole-2-one-4-ylmethyl.

(14) An alkoxybenzoic acid derivative of formula [III] or a salt thereof,

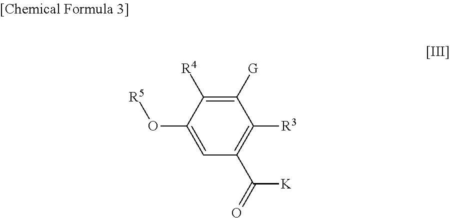

wherein in the formula [III],
G represents a hydrogen atom; a halogen atom; or a (C1-C6)alkyl,
K represents a hydroxy; a (C1-C6)alkoxy; or a phenoxy (the phenoxy is optionally mono-substituted or poly-substituted with R⁷),
R³ represents a halogen atom; a cyano; a (C1-C6)alkyl; a (C1-C6)haloalkyl; a (C3-C6)cycloalkyl; a (C1-C6)alkoxy; or a (C1-C6)haloalkoxy,
R⁴ represents a hydrogen atom; a halogen atom; a (C1-C6)alkyl; or a (C1-C6)haloalkyl,
R⁵ represents a (C1-C12)alkyl (the (C1-C12)alkyl is optionally mono-substituted or poly-substituted with R⁶); a (C3-C6)cycloalkyl (the (C3-C6)cycloalkyl is optionally mono-substituted or poly-substituted with R⁶); a (C2-C6)alkenyl (the (C2-C6)alkenyl is optionally mono-substituted or poly-substituted with R⁶); or a (C2-C6)alkynyl (the (C2-C6)alkynyl is optionally mono-substituted or poly-substituted with R⁶),
R⁶ represents a halogen atom; a (C1-C6)alkyl; a (C1-C6)haloalkyl; a (C3-C6)cycloalkyl; a (C3-C6)halocycloalkyl; a hydroxy; a (C1-C6)alkoxy; a (C1-C6)haloalkoxy; a (C3-C6)cycloalkoxy; a (C3-C6)halocycloalkoxy; a (C1-C6)alkoxy (C1-C6)alkoxy; a (C1-C6)haloalkoxy (C1-C6)alkoxy; a (C1-C6)haloalkoxy (C1-C6)haloalkoxy; a thiol; a (C1-C6)alkylthio; a (C1-C6)haloalkylthio; a (C2-C6)alkenylthio; a (C2-C6)haloalkenylthio; a (C3-C6)cycloalkylthio; a (C3-C6)halocycloalkylthio; a (C3-C6)cycloalkyl (C1-C6)alkylthio; a (C3-C6)halocycloalkyl (C1-C6)alkylthio; a tri((C1-C6)alkyl)silyl (C1-C6)alkylthio; a (C1-C6)alkylthio (C1-C6)alkoxy; a (C1-C6)haloalkylthio (C1-C6)alkoxy; a (C1-C6)alkylsulfinyl; a (C1-C6)haloalkylsulfinyl; a (C3-C6)cycloalkylsulfinyl; a (C3-C6)halocycloalkylsulfinyl; a (C1-C6)alkylsulfonyl; a (C1-C6)haloalkylsulfonyl; a (C3-C6)cycloalkylsulfonyl; a (C3-C6)halocycloalkylsulfonyl; a formyl; a (C1-C6)alkylcarbonyl; a (C1-C6)haloalkylcarbonyl; a benzoyl (the benzoyl is optionally mono-substituted or poly-substituted with R⁷); a (C1-C6)alkoxycarbonyl; a (C1-C6)haloalkoxycarbonyl; a (C1-C6)alkylaminocarbonyl (the amino moiety is optionally substituted with R¹⁰); a (C1-C6)haloalkylaminocarbonyl (the amino moiety is optionally substituted with R¹⁰); a (C1-C6)alkylaminocarbonylthio (the amino moiety is optionally substituted with R¹⁰); a (C1-C6)haloalkylaminocarbonylthio (the amino moiety is optionally substituted with R¹⁰); a formyloxy; a (C1-C6)alkylcarbonyloxy; a (C1-C6)haloalkylcarbonyloxy; a benzoyloxy (the benzoyloxy is optionally mono-substituted or poly-substituted with R⁷); a (C1-C6)alkylsulfinyloxy; a (C1-C6)haloalkylsulfinyloxy; a (C3-C6)cycloalkylsulfinyloxy; a (C3-C6)halocycloalkylsulfinyloxy; a phenylsulfinyloxy (the phenylsulfinyloxy is optionally mono-substituted or poly-substituted with R⁷); a (C1-C6)alkylsulfonyloxy; a (C1-C6)haloalkylsulfonyloxy; a (C3-C6)cycloalkylsulfonyloxy; a (C3-C6)halocycloalkylsulfonyloxy; a phenylsulfonyloxy (the phenyl moiety is optionally mono-substituted or poly-substituted with R⁷); an amino; a (C1-C6)alkylamino (the amino moiety is optionally substituted with R¹⁰); a (C1-C6)haloalkylamino (the amino moiety is optionally substituted with R¹⁰); a (C1-C6)alkylcarbonylamino (the amino moiety is optionally substituted with R¹⁰); a (C1-C6)haloalkylcarbonylamino (the amino moiety is optionally substituted with R¹⁰); a phenylcarbonylamino (the phenyl moiety is optionally mono-substituted or poly-substituted with R⁷, and the amino moiety is optionally substituted with R¹⁰); a (C1-C6)alkoxycarbonylamino (the amino moiety is optionally substituted with R¹⁰); a (C1-C6)haloalkoxycarbonylamino (the amino moiety is optionally substituted with R¹⁰); a (C1-C6)alkylaminocarbonylamino (the amino moiety is optionally substituted with R¹⁰); a (C1-C6)haloalkylaminocarbonylamino (the amino moiety is optionally substituted with R¹⁰); a (C1-C6)alkylsulfinylamino (the amino moiety is optionally substituted with R¹⁰); a (C1-C6)haloalkylsulfinylamino (the amino moiety is optionally substituted with R¹⁰); a phenylsulfinylamino (the phenyl moiety is optionally mono-substituted or poly-substituted with R⁷, and the amino moiety is optionally substituted with R¹⁰); a (C1-C6)alkylsulfonylamino (the amino moiety is optionally substituted with R¹⁰); a (C1-C6)haloalkylsulfonylamino (the amino moiety is optionally substituted with R¹⁰); a phenylsulfonylamino (the phenyl moiety is optionally mono-substituted or poly-substituted with R⁷, and the amino moiety is optionally substituted with R¹⁰); a (C1-C6)alkoxyimino; a tri((C1-C6)alkyl)silyl; a phenyl (the phenyl is optionally mono-substituted or poly-substituted with R⁷); a phenoxy (the phenoxy is optionally mono-substituted or poly-substituted with R⁷); a phenyl (C1-C6)alkoxy (the phenyl moiety is optionally mono-substituted or poly-substituted with R⁷); a phenylthio (the phenylthio is optionally mono-substituted or poly-substituted with R⁷); a phenylsulfinyl (the phenylsulfinyl is optionally mono-substituted or poly-substituted with R⁷); a phenylsulfonyl (the phenylsulfonyl is optionally mono-substituted or poly-substituted with R⁷); a phenyl (C1-C6)alkylthio (the phenyl moiety is optionally mono-substituted or poly-substituted with R⁷); a phenyl (C1-C6)alkylsulfinyl (the phenyl moiety is optionally mono-substituted or poly-substituted with R⁷); a phenyl (C1-C6)alkylsulfonyl (the phenyl moiety is optionally mono-substituted or poly-substituted with R⁷); a —O—N═C(R⁸)(R⁹) group; an adamantyl; an azetidinyl (a nitrogen atom of the azetidinyl is optionally substituted with R¹⁰); a pyrrolidinyl (a nitrogen atom of the pyrrolidinyl is optionally substituted with R¹⁰); a piperidinyl (a nitrogen atom of the piperidinyl is optionally substituted with R¹⁰); a 1,3-dioxolanyl; a 1,3-dioxanyl; a pyrrolyl (the pyrrolyl is optionally mono-substituted or poly-substituted with R⁷); a pyrazolyl (the pyrazolyl is optionally mono-substituted or poly-substituted with R⁷); an imidazolyl (the imidazolyl is optionally mono-substituted or poly-substituted with R⁷); a triazolyl (the triazolyl is optionally mono-substituted or poly-substituted with R⁷); an oxazolyl (the oxazolyl is optionally mono-substituted or poly-substituted with R⁷); an isoxazolyl (the isoxazolyl is optionally mono-substituted or poly-substituted with R⁷); a thiazolyl (the thiazolyl is optionally mono-substituted or poly-substituted with R⁷); an isothiazolyl (the isothiazolyl is optionally mono-substituted or poly-substituted with R⁷); a pyridyl (the pyridyl is optionally mono-substituted or poly-substituted with R⁷, and further a nitrogen atom of the pyridyl is optionally oxidized to form a N-oxide); a pyrimidinyl (the pyrimidinyl is optionally mono-substituted or poly-substituted with R⁷); a pyridyloxy (the pyridyloxy is optionally mono-substituted or poly-substituted with R⁷); a tetrahydrofuranyl (the tetrahydrofuranyl is optionally mono-substituted or poly-substituted with R⁷); a 1,3-dioxoisoindolinyl (the 1,3-dioxoisoindolinyl is optionally mono-substituted or poly-substituted with R⁷); a cyano; a nitro; a carboxy; a thiocyanato; or an aminooxy,
R⁷ represents a halogen atom; a (C1-C6)alkyl; a (C1-C6)haloalkyl; a (C3-C6)cycloalkyl; a (C3-C6)halocycloalkyl; a (C3-C6)cycloalkyl (C1-C6)alkyl; a (C3-C6)halocycloalkyl (C1-C6)alkyl; a (C1-C6)alkoxy; a (C1-C6)haloalkoxy; a (C1-C6)alkylthio; a (C1-C6)haloalkylthio; a (C1-C6)alkylsulfinyl; a (C1-C6)haloalkylsulfinyl; a (C1-C6)alkylsulfonyl; a (C1-C6)haloalkylsulfonyl; a (C1-C6)alkylthio (C1-C6)alkyl; a (C1-C6)haloalkylthio (C1-C6)alkyl; a (C1-C6)alkylsulfonyloxy; a (C1-C6)haloalkylsulfonyloxy; a phenyl (the phenyl is optionally mono-substituted or poly-substituted with a halogen atom, an alkyl, or a haloalkyl); a phenyl (C1-C6)alkyl; a phenyl (C1-C6)alkoxy; a cyano; or a nitro,
R⁸ and R⁹ are identical to or different from each other, and represent a hydrogen atom; a (C1-C6)alkyl; a (C1-C6)haloalkyl; a (C3-C6)cycloalkyl; a (C3-C6)halocycloalkyl; or a phenyl (the phenyl is optionally mono-substituted or poly-substituted with R⁷), or R⁸ and R⁹, together with the carbon atom to which R⁸ and R⁹ are attached form a 3- to 6-membered ring, and
R¹⁰ represents a (C1-C6)alkyl; a (C1-C6)haloalkyl; a (C3-C6)cycloalkyl; a (C3-C6)halocycloalkyl; a (C1-C6)alkoxy; a (C1-C6)alkoxy (C1-C6)alkyl; a (C1-C6)haloalkoxy (C1-C6)alkyl; a (C1-C6)alkoxy (C1-C6)alkoxy (C1-C6)alkyl; a (C1-C6)alkylthio (C1-C6)alkyl; a (C1-C6)alkylsulfinyl (C1-C6)alkyl; a (C1-C6)alkylsulfonyl (C1-C6)alkyl; a (C1-C6)alkylthio (C1-C6)alkoxy (C1-C6)alkyl; a (C1-C6)alkylsulfinyl (C1-C6)alkoxy (C1-C6)alkyl; a (C1-C6)alkylsulfonyl (C1-C6)alkoxy (C1-C6)alkyl; a cyano (C1-C6)alkyl; a cyano (C1-C6)alkoxy (C1-C6)alkyl; a (C1-C6)alkylcarbonyl; a (C1-C6)haloalkylcarbonyl; a (C1-C6)alkoxycarbonyl; a (C1-C6)haloalkoxycarbonyl; a (C1-C6)alkylaminocarbonyl; a (C1-C6)haloalkylaminocarbonyl; a benzoyl (the benzoyl is optionally mono-substituted or poly-substituted with R⁷); a (C1-C6)alkylsulfonyl; a (C1-C6)haloalkylsulfonyl; a (C1-C6)alkylcarbonyl (C1-C6)alkyl; a (C1-C6)alkoxycarbonyl (C1-C6)alkyl; a (C1-C6)alkylaminocarbonyl (C1-C6)alkyl; a (C1-C6)alkylcarbonyloxy (C1-C6)alkyl; a (C1-C6)alkoxycarbonyloxy (C1-C6)alkyl; a (C1-C6)alkoxycarbonylamino (C1-C6)alkyl (the amino moiety is optionally substituted with a (C1-C6)alkyl); or a 5-methyl-1,3-dioxole-2-one-4-ylmethyl.

### Effects of the Invention

The pest control agent comprising the compound of the present invention exhibits an excellent control effect on a wide range of pests such as Hemiptera pests, Lepidoptera pests, Coleoptera pests, Diptera pests, Hymenoptera pests, Orthoptera pests, Isoptera pests, Thysanoptera pests, Acari pests, and plant parasitic nematodes, and can also control pests having acquired chemical resistance.

### DESCRIPTION OF EMBODIMENTS

Symbols and terms described in the present specification will be described.

In the present invention, the “pest control agent” means insecticides, acaricides, nematicides, and the like for agricultural or horticultural fields, animals such as domestic animals and pets, household use, or prevention of epidemics.

In the present invention, the “halogen atom” represents a fluorine atom; a chlorine atom; a bromine atom; or an iodine atom.

In the present invention, the description of “(C1-C6)” or the like indicates that the number of carbon atoms of the substituent following this description is 1 to 6 in this case.

In the present invention, unless otherwise specified, the “(C1-C6)alkyl” refers to a linear or branched alkyl group having 1 to 6 carbon atoms. Examples thereof include methyl, ethyl, n-propyl, isopropyl, n-butyl, sec-butyl, isobutyl, tert-butyl, n-pentyl, 1-methylbutyl, 2-methylbutyl, 3-methylbutyl, 1-ethylpropyl, 1,1-dimethylpropyl, 1,2-dimethylpropyl, neopentyl, n-hexyl, 1-methylpentyl, 2-methylpentyl, 3-methylpentyl, 4-methylpentyl, 1-ethylbutyl, 2-ethylbutyl, 1,1-dimethylbutyl, 1,2-dimethylbutyl, 1,3-dimethylbutyl, 2,2-dimethylbutyl, 2,3-dimethylbutyl, 3,3-dimethylbutyl, 1,1,2-trimethylpropyl, 1,2,2-trimethylpropyl, 1-ethyl-1-methylpropyl, and 1-ethyl-2-methylpropyl groups.

In the present invention, unless otherwise specified, the “(C1-C6)haloalkyl” refers to a linear or branched alkyl group having 1 to 6 carbon atoms and being substituted with 1 to 13 halogen atoms and which are identical or different. Examples thereof include fluoromethyl, difluoromethyl, trifluoromethyl, chloromethyl, dichloromethyl, trichloromethyl, bromomethyl, dibromomethyl, tribromomethyl, iodomethyl, chlorodifluoromethyl, dichlorofluoromethyl, 1-fluoroethyl, 2-fluoroethyl, 1,1-difluoroethyl, 2,2-difluoroethyl, 2,2,2-trifluoroethyl, 1,1,2,2-tetrafluoroethyl, pentafluoroethyl, 1-chloroethyl, 2-chloroethyl, 1,1-dichloroethyl, 2,2-dichloroethyl, 2,2,2-trichloroethyl, 1,1,2,2-tetrachloroethyl, pentachloroethyl, 1-bromoethyl, 2-bromoethyl, 2,2,2-tribromoethyl, 1-iodoethyl, 2-iodoethyl, 2-chloro-2,2-difluoroethyl, 2,2-dichloro-2-fluoroethyl, 1-fluoropropyl, 2-fluoropropyl, 3-fluoropropyl, 1,1-difluoropropyl, 2,2-difluoropropyl, 3,3-difluoropropyl, 3,3,3-trifluoropropyl, 2,2,3,3,3-pentafluoropropyl, 1,1,2,3,3,3-hexafluoropropyl, heptafluoropropyl, 1-fluoro-2-propyl, 2-fluoro-2-propyl, 1,1-difluoro-2-propyl, 1,2-difluoro-2-propyl, 1,3-difluoro-2-propyl, 1,2,3-trifluoro-2-propyl, 1,1,3,3-tetrafluoro-2-propyl, 1,1,1,3,3,3-hexafluoro-2-propyl, heptafluoro-2-propyl, 1-chloropropyl, 2-chloropropyl, 3-chloropropyl, 1,1-dichloropropyl, 2,2-dichloropropyl, 3,3-dichloropropyl, 3,3,3-trichloropropyl, 2,2,3,3,3-pentachloropropyl, heptachloropropyl, 1-chloro-2-propyl, 2-chloro-2-propyl, 1,1-dichloro-2-propyl, 1,2-dichloro-2-propyl, 1,3-dichloro-2-propyl, 1,2,3-trichloro-2-propyl, 1,1,3,3-tetrachloro-2-propyl, 1,1,1,3,3,3-hexachloro-2-propyl, heptachloro-2-propyl, 1-bromopropyl, 2-bromopropyl, 3-bromopropyl, 1-bromo-2-propyl, 2-bromo-2-propyl, 1-iodopropyl, 2-iodopropyl, 3-iodopropyl, 1-iodo-2-propyl, 2-iodo-2-propyl, 1-fluorobutyl, 2-fluorobutyl, 3-fluorobutyl, 4-fluorobutyl, 4,4-difluorobutyl, 4,4,4-trifluorobutyl, 4,4,4-trifluoro-3-methylbutyl, 3,3,4,4,4-pentafluorobutyl, 2,2,3,4,4,4-hexafluorobutyl, 2,2,3,3,4,4,4-heptafluorobutyl, nonafluorobutyl, 1,1,1-trifluoro-2-butyl, 4,4,4-trifluoro-2-butyl, 3,3,4,4,4-pentafluoro-2-butyl, nonafluoro-2-butyl, 1,1,1,3,3,3-hexafluoro-2-(trifluoromethyl)-2-propyl, 1-chlorobutyl, 2-chlorobutyl, 3-chlorobutyl, 4-chlorobutyl, 4,4-dichlorobutyl, 4,4,4-trichlorobutyl, nonachlorobutyl, 1,1,1-trichloro-2-butyl, 4,4,4-trichloro-2-butyl, nonachloro-2-butyl, 1-bromobutyl, 2-bromobutyl, 3-bromobutyl, 4-bromobutyl, 1-iodobutyl, 2-iodobutyl, 3-iodobutyl, 4-iodobutyl, 4-chloro-1,1,2,2,3,3,4,4-octafluorobutyl, 4-bromo-1,1,2,2,3,3,4,4-octafluorobutyl, 1-fluoropentyl, 2-fluoropentyl, 3-fluoropentyl, 4-fluoropentyl, 5-fluoropentyl, 5,5,5-trifluoropentyl, 4,4,5,5,5-pentafluoropentyl, 3,3,4,4,5,5,5-heptafluoropentyl, 2,2,3,3,4,4,5,5-octafluoropentyl, 2,2,3,3,4,4,5,5,5-nonafluoropentyl, undecafluoropentyl, 1-chloropentyl, 2-chloropentyl, 3-chloropentyl, 4-chloropentyl, 5-chloropentyl, 5,5,5-trichloropentyl, 4,4,5,5,5-pentachloropentyl, 3,3,4,4,5,5,5-heptachloropentyl, 2,2,3,3,4,4,5,5,5-nonachloropentyl, undecachloropentyl, 1-bromopentyl, 2-bromopentyl, 3-bromopentyl, 4-bromopentyl, 5-bromopentyl, 5-iodopentyl, 1-fluorohexyl, 2-fluorohexyl, 3-fluorohexyl, 4-fluorohexyl, 5-fluorohexyl, 6-fluorohexyl, 6,6,6-trifluorohexyl, 5,5,6,6,6-pentafluorohexyl, 4,4,5,5,6,6,6-heptafluorohexyl, 3,3,4,4,5,5,6,6,6-nonafluorohexyl, 2,2,3,3,4,4,5,5,6,6,6-undecafluorohexyl, 2,2,3,3,4,4,5,5,6,6-decafluorohexyl, tridecafluorohexyl, 1-chlorohexyl, 2-chlorohexyl, 3-chlorohexyl, 4-chlorohexyl, 5-chlorohexyl, 6-chlorohexyl, 5-bromohexyl, 6-bromohexyl, 5-iodohexyl, and 6-iodohexyl groups.

In the present invention, unless otherwise specified, the “(C3-C6)cycloalkyl” refers to a cycloalkyl group having 3 to 6 carbon atoms, and the ring may be arbitrarily substituted with alkyl groups in the range of the designated number of carbon atoms. Examples thereof include cyclopropyl, 1-methylcyclopropyl, 2-methylcyclopropyl, 2,2-dimethylcyclopropyl, cyclobutyl, 1-methylcyclobutyl, cyclopentyl, 1-methylcyclopentyl, and cyclohexyl groups.

In the present invention, unless otherwise specified, the “(C3-C6)halocycloalkyl” refers to a cycloalkyl group having 3 to 6 carbon atoms and being substituted with 1 to 11 halogen atoms and which are identical or different, and the ring may be arbitrarily substituted with alkyl groups or haloalkyl groups within the specified range of carbon atoms. Examples thereof include 1-fluorocyclopropyl, 2-fluorocyclopropyl, 2,2-difluorocyclopropyl, 2,2,3,3-tetrafluorocyclopropyl, 1-chlorocyclopropyl, 2-chlorocyclopropyl, 2,2-dichlorocyclopropyl, 2,2,3,3-tetrachlorocyclopropyl, 1-bromocyclopropyl, 2,2-dibromocyclopropyl, 1-iodocyclopropyl, 1-fluoromethylcyclopropyl, 1-difluoromethylcyclopropyl, 1-trifluoromethylcyclopropyl, 1-chloromethylcyclopropyl, 1-bromomethylcyclopropyl, 2,2-diiodocyclopropyl, 1-fluorocyclobutyl, 2-fluorocyclobutyl, 3-fluorocyclobutyl, 3,3-difluorocyclobutyl, heptafluorocyclobutyl, 1-difluoromethylcyclobutyl, 1-trifluoromethylcyclobutyl, 2-chlorocyclobutyl, 3-chlorocyclobutyl, 3,3-dichlorocyclobutyl, 3,3-dibromocyclobutyl, 3,3-diiodocyclobutyl, 1-fluorocyclopentyl, 2-fluorocyclopentyl, 3-fluorocyclopentyl, 2,2-difluorocyclopentyl, 3,3-difluorocyclopentyl, nonafluorocyclopentyl, 2,2-dichlorocyclopentyl, 3,3-dichlorocyclopentyl, 2,2-dibromocyclopentyl, 3,3-dibromocyclopentyl, 2,2-diiodocyclopentyl, 3,3-diiodocyclopentyl, 1-fluorocyclohexyl, 2-fluorocyclohexyl, 3-fluorocyclohexyl, 4-fluorocyclohexyl, 2,2-difluorocyclohexyl, 3,3-difluorocyclohexyl, 4,4-difluorocyclohexyl, 1-chlorocyclohexyl, 2-chlorocyclohexyl, 3-chlorocyclohexyl, 4-chlorocyclohexyl, 2,2-dichlorocyclohexyl, 3,3-dichlorocyclohexyl, 4,4-dichlorocyclohexyl, 3,3-dibromocyclohexyl, 4,4-dibromocyclohexyl, 3,3-diiodocyclohexyl, and 4,4-diiodocyclohexyl groups.

In the present invention, unless otherwise specified, the “(C2-C6)alkenyl” refers to a linear or branched alkenyl group having 2 to 6 carbon atoms. Examples thereof include vinyl, 1-propenyl, isopropenyl, 2-propenyl, 1-butenyl, 1-methyl-1-propenyl, 2-butenyl, 1-methyl-2-propenyl, 3-butenyl, 2-methyl-1-propenyl, 2-methyl-2-propenyl, 1,3-butadienyl, 1-pentenyl, 1-ethyl-2-propenyl, 2-pentenyl, 1-methyl-1-butenyl, 3-pentenyl, 1-methyl-2-butenyl, 4-pentenyl, 1-methyl-3-butenyl, 3-methyl-1-butenyl, 1,2-dimethyl-2-propenyl, 1,1-dimethyl-2-propenyl, 2-methyl-2-butenyl, 3-methyl-2-butenyl, 1,2-dimethyl-1-propenyl, 2-methyl-3-butenyl, 3-methyl-3-butenyl, 1,3-pentadienyl, 1-vinyl-2-propenyl, 1-hexenyl, 1-propyl-2-propenyl, 2-hexenyl, 1-methyl-1-pentenyl, 1-ethyl-2-butenyl, 3-hexenyl, 4-hexenyl, 5-hexenyl, 1-methyl-4-pentenyl, 1-ethyl-3-butenyl, 1-(isobutyl)vinyl, 1-ethyl-1-methyl-2-propenyl, 1-ethyl-2-methyl-2-propenyl, 1-(isopropyl)-2-propenyl, 2-methyl-2-pentenyl, 3-methyl-3-pentenyl, 4-methyl-3-pentenyl, 1,3-dimethyl-2-butenyl, 1,1-dimethyl-3-butenyl, 3-methyl-4-pentenyl, 4-methyl-4-pentenyl, 1,2-dimethyl-3-butenyl, 1,3-dimethyl-3-butenyl, 1,1,2-trimethyl-2-propenyl, 1,5-hexadienyl, 1-vinyl-3-butenyl, and 2,4-hexadienyl groups.

In the present invention, unless otherwise specified, the “(C2-C6)haloalkenyl” refers to a linear or branched alkenyl group having 2 to 6 carbon atoms and being substituted with 1 to 11 halogen atoms and which are identical or different. Examples thereof include 1-fluorovinyl, 2-fluorovinyl, 1,2-difluorovinyl, 2,2-difluorovinyl, trifluorovinyl, 1-chlorovinyl, 2-chlorovinyl, 1,2-dichlorovinyl, 2,2-dichlorovinyl, trichlorovinyl, 1,2-dibromovinyl, 2,2-dibromovinyl, tribromovinyl, 1,2-diiodovinyl, 2,2-diiodovinyl, triiodovinyl, 1-fluoro-2-propenyl, 2-fluoro-2-propenyl, 3-fluoro-2-propenyl, 2,3-difluoro-2-propenyl, 3,3-difluoro-2-propenyl, 3,3-difluoro-1-propenyl, 2,3,3-trifluoro-2-propenyl, 3,3,3-trifluoro-1-propenyl, 2-chloro-3,3,3-trifluoro-1-propenyl, 1,2,3,3,3-pentafluoro-1-propenyl, 1-chloro-2-propenyl, 2-chloro-2-propenyl, 3-chloro-2-propenyl, 2,3-dichloro-2-propenyl, 3,3-dichloro-2-propenyl, 3,3-dichloro-1-propenyl, 2,3,3-trichloro-2-propenyl, 3,3,3-trichloro-1-propenyl, 3-bromo-2-propenyl, 3,3-dibromo-2-propenyl, 3,3-diiodo-2-propenyl, 2,2-difluoro-1-propene-2-yl, 3,3,3-trifluoro-1-propene-2-yl, 3,3,3-trichloro-1-propene-2-yl, 4-fluoro-3-butenyl, 4,4-difluoro-3-butenyl, 4,4-difluoro-3-butene-2-yl, 4,4,4-trifluoro-2-butenyl, 3,4,4-trifluoro-3-butenyl, 2-trifluoromethyl-2-propenyl, 2-trifluoromethyl-3,3-difluoro-2-propenyl, 4,4,4-trifluoro-3-chloro-2-butenyl, 4,4-dichloro-3-butenyl, 4,4,4-trichloro-2-butenyl, 2-trichloromethyl-2-propenyl, 5,5-difluoro-4-pentenyl, 4,5,5-trifluoro-4-pentenyl, 5,5,5-trifluoro-3-pentenyl, 4,4,4-trifluoro-3-methyl-2-butenyl, 4,4,4-trifluoro-3-trifluoromethyl-2-butenyl, 5,5-dichloro-4-pentenyl, 4,4,4-trichloro-3-methyl-2-butenyl, 6,6-difluoro-5-hexenyl, 5,6,6-trifluoro-5-pentenyl, 6,6,6-trifluoro-4-pentenyl, 5,5,5-trifluoro-4-methyl-3-pentenyl, 5,5,5-trifluoro-4-trifluoromethyl-3-pentenyl, 6,6-dichloro-5-hexenyl, and 5,5,5-trichloro-4-methyl-3-pentenyl groups.

In the present invention, unless otherwise specified, the “(C2-C6)alkynyl” refers to a linear or branched alkynyl group having 2 to 6 carbon atoms. Examples thereof include ethynyl, 1-propynyl, 2-propynyl, 1-butynyl, 1-methyl-2-propynyl, 2-butynyl, 3-butynyl, 1-pentynyl, 1-ethyl-2-propynyl, 2-pentynyl, 3-pentynyl, 1-methyl-2-butynyl, 4-pentynyl, 1-methyl-3-butynyl, 2-methyl-3-butynyl, 1-hexynyl, 1-(n-propyl)-2-propynyl, 2-hexynyl, 1-ethyl-2-butynyl, 3-hexynyl, 1-methyl-2-pentynyl, 1-methyl-3-pentynyl, 4-methyl-1-pentynyl, 3-methyl-1-pentynyl, 5-hexynyl, 1-ethyl-3-butynyl, 1-ethyl-1-methyl-2-propynyl, 1-(isopropyl)-2-propynyl, 1,1-dimethyl-2-butynyl, and 2,2-dimethyl-3-butynyl groups.

In the present invention, unless otherwise specified, the “(C2-C6)haloalkynyl” refers to a linear or branched alkynyl group having 2 to 6 carbon atoms and being substituted with 1 to 9 halogen atoms and which are identical or different. Examples thereof include fluoroethynyl, chloroethynyl, bromoethynyl, iodoethynyl, 3-fluoro-2-propynyl, 3-chloro-2-propynyl, 3-bromo-2-propynyl, 3-iodo-2-propynyl, 4-fluoro-3-butynyl, 4-chloro-3-butynyl, 4-bromo-3-butynyl, 4-iodo-3-butynyl, 4,4-difluoro-2-butynyl, 4,4-dichloro-2-butynyl, 4,4,4-trifluoro-2-butynyl, 4,4,4-trichloro-2-butynyl, 3-fluoro-1-methyl-2-propynyl, 3-chloro-1-methyl-2-propynyl, 5-fluoro-4-pentynyl, 5-chloro-4-pentynyl, 5,5,5-trifluoro-3-pentynyl, 5,5,5-trichloro-3-pentynyl, 4-fluoro-2-methyl-3-butynyl, 4-chloro-2-methyl-3-butynyl, 6-fluoro-5-hexynyl, 6-chloro-5-hexynyl, 6,6,6-trifluoro-4-hexynyl, 6,6,6-trichloro-4-hexynyl, 5-fluoro-3-methyl-4-pentynyl, and 5-chloro-3-methyl-4-pentynyl groups.

In the present invention, unless otherwise specified, the “(C1-C6)alkoxy” refers to a ((C₁-C₆)alkyl)-O— group in which the alkyl moiety is as defined above. Examples thereof include methoxy, ethoxy, n-propoxy, isopropoxy, n-butoxy, isobutoxy, sec-butoxy, tert-butoxy, n-pentoxy, 1-methylbutoxy, 2-methylbutoxy, 3-methylbutoxy, 1-ethylpropoxy, 1,1-dimethylpropoxy, 1,2-dimethylpropoxy, and n-hexyloxy groups.

In the present invention, unless otherwise specified, the “(C1-C6)haloalkoxy” refers to a ((C₁-C₆)haloalkyl)-O— group in which the haloalkyl moiety is as defined above. Examples thereof include difluoromethoxy, dichloromethoxy, trifluoromethoxy, trichloromethoxy, tribromomethoxy, chlorodifluoromethoxy, bromodifluoromethoxy, 2-fluoroethoxy, 1-chloroethoxy, 2-chloroethoxy, 1-bromoethoxy, 2-bromoethoxy, 2,2-difluoroethoxy, 1,2-dichloroethoxy, 2,2-dichloroethoxy, 2,2,2-trifluoroethoxy, 2,2,2-trichloroethoxy, 1,1,2,2-tetrafluoroethoxy, pentafluoroethoxy, 2-bromo-2-chloroethoxy, 2-chloro-1,1,2,2-tetrafluoroethoxy, 1-chloro-1,2,2,2-tetrafluoroethoxy, 1-chloropropoxy, 2-chloropropoxy, 3-chloropropoxy, 2-bromopropoxy, 3-bromopropoxy, 2-bromo-1-methylethoxy, 3-iodopropoxy, 2,3-dichloropropoxy, 2,3-dibromopropoxy, 3,3,3-trifluoropropoxy, 3,3,3-trifluoro-2-propoxy, 3,3,3-trichloropropoxy, 3-bromo-3,3-difluoropropoxy, 2,2-difluoropropoxy, 3,3-dichloro-3-fluoropropoxy, 2,2,3,3-tetrafluoropropoxy, 1-bromo-3,3,3-trifluoropropoxy, 2,2,3,3,3-pentafluoropropoxy, 2,2,2-trifluoro-1-trifluoromethylethoxy, heptafluoropropoxy, heptafluoro-2-propoxy, 1,2,2,2-tetrafluoro-1-trifluoromethylethoxy, 1,1,2,3,3,3-hexafluoropropoxy, 2-chlorobutoxy, 3-chlorobutoxy, 4-chlorobutoxy, 2-chloro-1,1-dimethylethoxy, 4-bromobutoxy, 3-bromo-2-methylpropoxy, 2-bromo-1,1-dimethylethoxy, 2,2-dichloro-1,1-dimethylethoxy, 2-chloro-1-chloromethyl-2-methylethoxy, 4,4,4-trifluorobutoxy, 3,3,3-trifluoro-1-methylpropoxy, 3,3,3-trifluoro-2-methylpropoxy, 2,3,4-trichlorobutoxy, 2,2,2-trichloro-1,1-dimethylethoxy, 4-chloro-4,4-difluorobutoxy, 4,4-dichloro-4-fluorobutoxy, 4-bromo-4,4-difluorobutoxy, 2,4-dibromo-4,4-difluorobutoxy, 3,4-dichloro-3,4,4-trifluorobutoxy, 3,3-dichloro-4,4,4-trifluorobutoxy, 4-bromo-3,3,4,4-tetrafluorobutoxy, 4-bromo-3-chloro-3,4,4-trifluorobutoxy, 2,2,3,3,4,4-hexafluorobutoxy, 2,2,3,4,4,4-hexafluorobutoxy, 2,2,2-trifluoro-1-methyl-1-trifluoromethylethoxy, 3,3,3-trifluoro-2-trifluoromethylpropoxy, 2,2,3,3,4,4,4-heptafluorobutoxy, 3,3,4,4,4-pentafluoro-2-butoxy, 2,3,3,3-tetrafluoro-2-trifluoromethylpropoxy, 1,1,2,2,3,3,4,4-octafluorobutoxy, nonafluorobutoxy, perfluoro-tert-butoxy, 4-chloro-1,1,2,2,3,3,4,4-octafluorobutoxy, 5,5,5-trifluoropentoxy, 4,4,5,5,5-pentafluoropentoxy, 3,3,4,4,5,5,5-heptafluoropentoxy, 3,3,4,4,5,5,5-heptafluoro-2-pentoxy, 2,2,3,3,4,4,5,5,5-nonafluoropentoxy, 2,2,3,3,4,4,5,5-octafluoropentoxy, perfluoropentoxy, 4,4,5,5,5-pentafluoro-2-butoxy, 2,2-bis(trifluoromethyl)propoxy, 2,2,3,3,4,4,5,5,6,6,6-undecafluorohexyloxy, 3,3,4,4,5,5,6,6,6-nonafluorohexyloxy, 4,4,5,5,6,6,6-heptafluorohexyloxy, 2,2,3,3,4,4,5,5,6,6-decafluorohexyloxy, 4,4,4-trifluoro-3,3-bis(trifluoromethyl)butyloxy, and perfluorohexyloxy groups.

In the present invention, unless otherwise specified, the “(C3-C6)cycloalkyl C1-C6 alkyl” refers to a ((C3-C6)cycloalkyl)-((C1-C6)alkyl)- group in which the cycloalkyl moiety and the alkyl moiety are as defined above. Examples thereof include cyclopropylmethyl, cyclobutylmethyl, cyclopentylmethyl, cyclohexylmethyl, 1-(cyclopropyl)ethyl, 2-(cyclopropyl)ethyl, and 1-(cyclopropyl)propyl groups.

In the present invention, unless otherwise specified, the “(C3-C6)halocycloalkyl (C1-C6)alkyl” refers to a ((C3-C6)halocycloalkyl)-((C1-C6)alkyl)- group in which the halocycloalkyl moiety and the alkyl moiety are as defined above. Examples thereof include 2,2-difluorocyclopropylmethyl, 2,2-dichlorocyclopropylmethyl, 1-(2,2-difluorocyclopropyl)ethyl, 2-(2,2-difluorocyclopropyl)ethyl, 1-(2,2-dichlorocyclopropyl)ethyl, 2-(2,2-dichlorocyclopropyl)ethyl, 2-(2,2-difluorocyclopropyl)propyl, 3,3-difluorocyclobutylmethyl, 2,2,3,3-tetrafluorocyclobutylmethyl, 2-(2,2,3,3-tetrafluorocyclobutyl)ethyl, 2-(3,3-difluorocyclobutyl)ethyl, 2-(3,3-difluorocyclobutyl)propyl, 3,3-difluorocyclopentylmethyl, and 2-(3,3-difluorocyclopentyl)propyl groups.

In the present invention, unless otherwise specified, the “phenyl (C1-C6)alkyl” refers to a (phenyl)-((C1-C6)alkyl)-group. Examples thereof include benzyl, 1-phenylethyl, 2-phenylethyl, 2-phenylpropyl, 2-phenylbutyl, and 1-phenylpentyl groups.

In the present invention, unless otherwise specified, the “heteroaryl” refers to a 5- or 6-membered monocyclic heterocyclic ring having 1 to 6 heteroatoms selected from a nitrogen atom, an oxygen atom, and a sulfur atom, in addition to a carbon atom. Examples thereof include thiophene-2-yl, thiophene-3-yl, furan-2-yl, furan-3-yl, pyrrole-1-yl, pyrrole-2-yl, pyrrole-3-yl, oxazole-2-yl, oxazole-4-yl, oxazole-5-yl, isoxazole-3-yl, isoxazole-4-yl, isoxazole-5-yl, thiazole-2-yl, thiazole-4-yl, thiazole-5-yl, isothiazole-3-yl, isothiazole-4-yl, isothiazole-5-yl, imidazole-1-yl, imidazole-2-yl, imidazole-4-yl, pyrazole-1-yl, pyrazole-3-yl, pyrazole-4-yl, pyrazole-5-yl, 1,3,4-oxadiazole-2-yl, 1,2,3-oxadiazole-4-yl, 1,2,3-oxadiazole-5-yl, 1,2,4-oxadiazole-3-yl, 1,2,4-oxadiazole-5-yl, 1,2,5-oxadiazole-3-yl, 1,3,4-thiadiazole-2-yl, 1,2,3-thiadiazole-4-yl, 1,2,3-thiadiazole-5-yl, 1,2,4-thiadiazole-3-yl, 1,2,4-thiadiazole-5-yl, 1,2,5-thiadiazole-3-yl, 1,2,4-triazole-3-yl, 1,2,4-triazole-1-yl, 1,2,3-triazole-1-yl, 1,2,3-triazole-2-yl, 1,3,4-triazole-1-yl, tetrazole-1-yl, tetrazole-2-yl, tetrazole-5-yl, pyridine-2-yl, pyridine-3-yl, pyridine-4-yl, pyrimidine-2-yl, pyrimidine-4-yl, pyrimidine-5-yl, pyridazine-3-yl, pyridazine-4-yl, pyrazine-2-yl, and 1,3,5-triazine-2-yl groups.

In the present invention, unless otherwise specified, the “heteroaryl (C1-C3)alkyl” refers to a (heteroaryl)-((C1-C3)alkyl)- group in which the heteroaryl moiety is as defined above. Examples thereof include (pyridine-2-yl)methyl, (pyridine-3-yl)methyl, (pyridine-4-yl)methyl, (pyrimidine-2-yl)methyl, (pyrimidine-4-yl)methyl, (thiazole-2-yl)methyl, (thiazole-4-yl)methyl, (thiazole-5-yl)methyl, (pyrazole-1-yl)methyl, (pyrazole-3-yl)methyl, (pyrazole-4-yl)methyl, and (pyrazole-5-yl)methyl groups.

In the present invention, unless otherwise specified, the “heteroaryl (C3-C6)cycloalkyl” refers to a (heteroaryl)-((C3-C6)cycloalkyl)- group in which the heteroaryl moiety and the cycloalkyl moiety are as defined above. Examples thereof include 1-(pyridine-2-yl)cyclopropyl, 1-(pyridine-3-yl)cyclopropyl, 1-(pyridine-4-yl)cyclopropyl, 1-(pyrimidine-2-yl)cyclopropyl, 1-(pyrimidine-4-yl)cyclopropyl, 1-(thiazole-2-yl)cyclopropyl, 1-(thiazole-4-yl)cyclopropyl, 1-(thiazole-5-yl)cyclopropyl, 1-(pyrazole-1-yl)cyclopropyl, 1-(pyrazole-3-yl)cyclopropyl, 1-(pyrazole-4-yl)cyclopropyl, and 1-(pyrazole-5-yl)cyclopropyl groups.

In the present invention, unless otherwise specified, the “heterocycloalkyl” refers to a 3- to 6-membered saturated heterocyclic ring containing one or more atoms selected from an oxygen atom, a nitrogen atom, and a sulfur atom, in addition to a carbon atom. Examples thereof include oxetane-2-yl, oxetane-3-yl, thietane-3-yl, tetrahydrofuran-2-yl, tetrahydrofuran-3-yl, tetrahydrothiophene-3-yl, tetrahydropyran-2-yl, tetrahydropyran-3-yl, tetrahydropyran-4-yl, tetrahydrothiopyran-4-yl, morpholine-4-yl, azetidine-1-yl, azetidine-3-yl, pyrrolidine-3-yl, and piperidine-4-yl groups.

In the present invention, unless otherwise specified, the “cyano heterocycloalkyl” refers to a (cyano)-(heterocycloalkyl)- group in which the heterocycloalkyl moiety is as defined above. Examples thereof include 3-cyanooxetane-3-yl, 3-cyanothietane-3-yl, 3-cyanotetrahydrofuran-3-yl, 3-cyanotetrahydrothiophene-3-yl, 3-cyanotetrahydropyran-3-yl, 4-cyanotetrahydrothiopyran-4-yl, 3-cyanoazetidine-3-yl, 3-cyanopyrrolidine-3-yl, and 4-cyanopiperidine-4-yl groups.

In the present invention, unless otherwise specified, the “heterocycloalkyl (C1-C6)alkyl” refers to a (3- to 6-membered heterocycloalkyl)-((C1-C6)alkyl)- group in which the heterocycloalkyl moiety and the alkyl moiety are as defined above. Examples thereof include (oxetane-3-yl)methyl, (tetrahydrofuran-2-yl)methyl, (tetrahydrofuran-3-yl)methyl, (tetrahydropyran-2-yl)methyl, (tetrahydropyran-3-yl)methyl, and (tetrahydropyran-4-yl)methyl groups.

In the present invention, unless otherwise specified, the “cyano (C1-C6)alkyl” refers to a (cyano)-((C1-C6)alkyl)- group in which the alkyl moiety is as defined above. Examples thereof include cyanomethyl, 1-cyanoethyl, 2-cyanoethyl, 1-cyanopropyl, 3-cyanopropyl, 2-cyano-2-propyl, 1-cyanobutyl, 4-cyanobutyl, 5-cyanopentyl, and 6-cyanohexyl groups.

In the present invention, unless otherwise specified, the “cyano (C3-C6)cycloalkyl” refers to a (cyano)-((C3-C6)cycloalkyl)- group in which the cycloalkyl moiety is as defined above. Examples thereof include 1-cyanocyclopropyl, 2-cyanocyclopropyl, 1-cyanomethylcyclopropyl, 1-cyanocyclobutyl, 3-cyanocyclobutyl, 1-cyanocyclopentyl, and 1-cyanocyclohexyl groups.

In the present invention, unless otherwise specified, the “hydroxy (C1-C6)alkyl” refers to a (hydroxy)-((C1-C6)alkyl)- group in which the alkyl moiety is as defined above. Examples thereof include hydroxymethyl, 1-hydroxyethyl, 2-hydroxyethyl, 1-hydroxypropyl, 2-hydroxypropyl, 3-hydroxypropyl, 1-hydroxybutyl, and 4-hydroxybutyl groups.

In the present invention, unless otherwise specified, the “(C1-C6)alkoxy (C1-C6)alkyl” refers to a ((C1-C6)alkoxy)-((C1-C6)alkyl)- group in which the alkoxy moiety and the alkyl moiety are as defined above. Examples thereof include methoxymethyl, 1-methoxyethyl, 2-methoxyethyl, 2-ethoxyethyl, 2-methoxypropyl, 3-methoxypropyl, 2-ethoxypropyl, 3-ethoxypropyl, 1-methyl-3-methoxybutyl, and 3-butoxybutyl groups.

In the present invention, unless otherwise specified, the “(C1-C6)alkylamino” refers to a ((C1-C6)alkyl)-NH— group in which the alkyl moiety is as defined above. Examples thereof include methylamino, ethylamino, n-propylamino, and isopropylamino groups.

In the present invention, unless otherwise specified, the “(C1-C6)alkylcarbonyl” refers to a ((C1-C6)alkyl)-C(═O)— group in which the alkyl moiety is as defined above. Examples thereof include acetyl, propionyl, 2-methylpropionyl, 2,2-dimethylpropionyl, butanoyl, pivaloyl, 2-methylbutanoyl, 3-methylbutanoyl, 2-ethylbutanoyl, 2,2-dimethylbutanoyl, 2,3-dimethylbutanoyl, 3,3-dimethylbutanoyl, pentanoyl, 2-methylpentanoyl, 3-methylpentanoyl, 4-methylpentanoyl, and hexanoyl groups.

In the present invention, unless otherwise specified, the “(C1-C6)alkoxy (C1-C6)alkylcarbonyl” refers to a ((C1-C6)alkyl)-O—((C1-C6)alkyl)-C(═O)— group in which the alkyl moiety is as defined above. Examples thereof include methoxyacetyl, ethoxyacetyl, propoxyacetyl, isopropoxyacetyl, butoxyacetyl, 2-methoxypropionyl, 3-methoxypropionyl, 2-ethoxypropionyl, 3-ethoxypropionyl, 2-methoxybutanoyl, 4-methoxybutanoyl, 2-methoxypentanoyl, and 5-methoxypentanoyl groups.

In the present invention, unless otherwise specified, the “(C3-C6)cycloalkylcarbonyl” refers to a ((C3-C6)cycloalkyl)-C(═O)— group in which the cycloalkyl moiety is as defined above. Examples thereof include cyclopropanecarbonyl, cyclobutanecarbonyl, cyclopentanecarbonyl, and cyclohexanecarbonyl groups.

In the present invention, unless otherwise specified, the “(C3-C6)cycloalkyl (C1-C6)alkylcarbonyl” refers to a ((C3-C6)cycloalkyl)-((C1-C6)alkyl)-C(═O)— group in which the cycloalkyl and alkyl moieties are as defined above. Examples thereof include 2-cyclopropylacetyl, 2-cyclobutylacetyl, 2-cyclopentylacetyl, 2-cyclohexylacetyl, 2-cyclopropylpropanol, and 2-cyclopropylbutanoyl groups.

In the present invention, unless otherwise specified, the “(C1-C6)alkoxycarbonyl” refers to a ((C1-C6)alkoxy)-C(═O)— group in which the alkoxy moiety is as defined above. Examples thereof include methoxycarbonyl, ethoxycarbonyl, n-propoxycarbonyl, isopropoxycarbonyl, n-butoxycarbonyl, isobutoxycarbonyl, sec-butoxycarbonyl, tert-butoxycarbonyl, n-pentoxycarbonyl, 1-methylbutoxycarbonyl, 2-methylbutoxycarbonyl, 3-methylbutoxycarbonyl, 1-ethylpropoxycarbonyl, 1,1-dimethylpropoxycarbonyl, 1,2-dimethylpropoxycarbonyl, and 2,2-dimethylpropoxycarbonyl groups.

In the present invention, unless otherwise specified, the “(C1-C6)alkylaminocarbonyl” refers to a ((C1-C6)alkyl)-NH—C(═O)— group in which the alkyl moiety is as defined above. Examples thereof include methylaminocarbonyl, ethylaminocarbonyl, n-propylaminocarbonyl, isopropylaminocarbonyl, n-butylaminocarbonyl, isobutylaminocarbonyl, sec-butylaminocarbonyl, tert-butylaminocarbonyl, n-pentylaminocarbonyl, 1-methylbutylaminocarbonyl, 2-methylbutylaminocarbonyl, 3-methylbutylaminocarbonyl, 1-ethylpropylaminocarbonyl, 1,1-dimethylpropylaminocarbonyl, 1,2-dimethylpropylaminocarbonyl, 2,2-dimethylpropylaminocarbonyl, and n-hexylaminocarbonyl groups.

In the present invention, unless otherwise specified, the “(C1-C6)alkoxycarbonyl (C1-C6)alkylamino” refers to a ((C1-C6)alkyl)-O—C(═O)—((C1-C6)alkyl)-NH— group in which the alkyl moiety is as defined above. Examples thereof include methoxycarbonylmethylamino, ethoxycarbonylmethylamino, tert-butoxycarbonylmethylamino, and 1-(tert-butoxycarbonyl)ethylamino groups.

In the present invention, unless otherwise specified, the “(C1-C6)alkoxycarbonylamino” refers to a ((C1-C6)alkyl)-O—C(═O)—NH— group in which the alkyl moiety is as defined above. Examples thereof include methoxycarbonylamino, ethoxycarbonylamino, isopropoxycarbonylamino, tert-butoxycarbonylamino, and isobutoxycarbonylamino groups.

In the present invention, unless otherwise specified, the “(C1-C6)alkylthio” refers to a ((C1-C6)alkyl)-S— group in which the alkyl moiety is as defined above. Examples thereof include methylthio, ethylthio, n-propylthio, isopropylthio, n-butylthio, isobutylthio, sec-butylthio, and tert-butylthio groups.

In the present invention, unless otherwise specified, the “((C1-C)alkylthio)carbonyl” refers to a ((C1-C)alkylthio)-C(═O)— group in which the alkylthio moiety is as defined above. Examples thereof include methylthiocarbonyl, ethylthiocarbonyl, and isopropylthiocarbonyl groups.

In the present invention, unless otherwise specified, the “((C1-C6)alkyl) thiocarbonyl” refers to a ((C1-C6)alkyl)-C(═S)— group in which the alkyl moiety is as defined above. Examples thereof include methyl(thiocarbonyl), ethyl(thiocarbonyl), n-propyl(thiocarbonyl), isopropyl(thiocarbonyl), n-butyl(thiocarbonyl), isobutyl(thiocarbonyl), sec-butyl(thiocarbonyl), tert-butyl(thiocarbonyl), and n-pentyl(thiocarbonyl) groups.

In the present invention, unless otherwise specified, the “((C1-C6)alkoxy) thiocarbonyl” refers to a ((C1-C6)alkyl)-O—C(═S)— group in which the alkyl moiety is as defined above. Examples thereof include methoxy(thiocarbonyl), ethoxy(thiocarbonyl), n-propoxy(thiocarbonyl), isopropoxy(thiocarbonyl), n-butoxy(thiocarbonyl), isobutoxy(thiocarbonyl), sec-butoxy(thiocarbonyl), tert-butoxy(thiocarbonyl), n-pentoxy(thiocarbonyl), 1-methylbutoxy(thiocarbonyl), 2-methylbutoxy(thiocarbonyl), 3-methylbutoxy(thiocarbonyl), 1-ethylpropoxy(thiocarbonyl), 1,1-dimethylpropoxy(thiocarbonyl), 1,2-dimethylpropoxy(thiocarbonyl), and 2,2-dimethylpropoxy(thiocarbonyl) groups.

In the present invention, unless otherwise specified, the “(C1-C6)alkylaminothiocarbonyl” refers to a ((C1-C6)alkyl)-NH—C(═S)— group in which the alkyl moiety is as defined above. Examples thereof include methylamino(thiocarbonyl), ethylamino(thiocarbonyl), n-propylamino(thiocarbonyl), isopropylamino(thiocarbonyl), n-butylamino(thiocarbonyl), isobutylamino(thiocarbonyl), sec-butylamino(thiocarbonyl), tert-butylamino(thiocarbonyl), n-pentylamino(thiocarbonyl), 1-methylbutylamino(thiocarbonyl), 2-methylbutylamino(thiocarbonyl), 3-methylbutylamino(thiocarbonyl), 1-ethylpropylamino(thiocarbonyl), 1,1-dimethylpropylamino(thiocarbonyl), 1,2-dimethylpropylamino(thiocarbonyl), 2,2-dimethylpropylamino(thiocarbonyl), and n-hexylamino(thiocarbonyl) groups.

In the present invention, unless otherwise specified, the “(C1-C6)alkylsulfonyl” refers to a ((C1-C6)alkyl)-S(═O)₂— group in which the alkyl moiety is as defined above. Examples thereof include methylsulfonyl, ethylsulfonyl, n-propylsulfonyl, isopropylsulfonyl, n-butylsulfonyl, isobutylsulfonyl, sec-butylsulfonyl, and tert-butylsulfonyl groups.

In the present invention, unless otherwise specified, the “(C1-C6)haloalkylsulfonyl” refers to a ((C1-C6)haloalkyl)-S(═O)₂— group in which the haloalkyl moiety is as defined above. Examples thereof include difluoromethylsulfonyl, trifluoromethylsulfonyl, trichloromethylsulfonyl, 2,2,2-trifluoroethylsulfonyl, pentafluoroethylsulfonyl, 3,3,3-trifluoropropylsulfonyl, heptafluoropropylsulfonyl, heptafluoro-2-propylsulfonyl, and nonafluorobutylsulfonyl groups.

In the present invention, unless otherwise specified, the “(C1-C6)alkylaminosulfonyl” refers to a ((C1-C6)alkyl)-NH—S(═O)₂— group in which the alkyl moiety is as defined above. Examples thereof include methylaminosulfonyl, ethylaminosulfonyl, n-propylaminosulfonyl, isopropylaminosulfonyl, n-butylaminosulfonyl, isobutylaminosulfonyl, sec-butylaminosulfonyl, tert-butylaminosulfonyl, n-pentylaminosulfonyl, 1-methylbutylaminosulfonyl, 2-methylbutylaminosulfonyl, 3-methylbutylaminosulfonyl, 1-ethylpropylaminosulfonyl, 1,1-dimethylpropylaminosulfonyl, 1,2-dimethylpropylaminosulfonyl, 2,2-dimethylpropylaminosulfonyl, and n-hexylaminosulfonyl groups.

In the present invention, unless otherwise specified, the “(C1-C6)alkoxycarbonyl (C1-C6)alkyl” refers to a ((C1-C6)alkoxy)-C(═O)—((C1-C6)alkyl)- group in which the alkoxy moiety and the alkyl moiety are as defined above. Examples thereof include methoxycarbonylmethyl, ethoxycarbonylmethyl, n-propoxycarbonylmethyl, isopropoxycarbonylmethyl, tert-butoxycarbonylmethyl, 1-(methoxycarbonyl)ethyl, 2-(methoxycarbonyl)ethyl, 1-(ethoxycarbonyl)ethyl, 2-(ethoxycarbonyl)ethyl, 1-(methoxycarbonyl)-1-methylethyl, 1-(ethoxycarbonyl)-1-methylethyl, and 2-(tert-butoxycarbonyl)ethyl groups.

In the present invention, unless otherwise specified, the “aminocarbonyl (C1-C6)alkyl” refers to a H₂N—C(═O)—((C1-C6)alkyl)- group in which the alkyl moiety is as defined above. Examples thereof include carbamoylmethyl, 1-carbamoylethyl, and 2-carbamoylethyl groups.

In the present invention, unless otherwise specified, the “(C1-C6)alkylaminocarbonyl (C1-C6)alkyl” refers to a ((C1-C6)alkyl)-NH—C(═O)—((C1-C6)alkyl)- group in which the alkyl moiety is as defined above. Examples thereof include N-methylcarbamoylmethyl, N-ethylcarbamoylmethyl, and N-(tert-butyl)carbamoylmethyl groups.

In the present invention, unless otherwise specified, the “(C3-C6)cycloalkylaminocarbonyl (C1-C6)alkyl” refers to a ((C3-C6)cycloalkyl)-NH—C(═O)—((C1-C6)alkyl)- group in which the cycloalkyl moiety and the alkyl moiety are as defined above. Examples thereof include N-cyclopropylcarbamoylmethyl, N-cyclobutylcarbamoylmethyl, N-cyclopentylcarbamoylmethyl, and N-cyclohexylcarbamoylmethyl groups.

In the present invention, unless otherwise specified, the “(C1-C6)haloalkylaminocarbonyl (C1-C6)alkyl” refers to a ((C1-C6)haloalkyl)-NH—C(═O)—((C1-C6)alkyl)- group in which the haloalkyl moiety and the alkyl moiety are as defined above. Examples thereof include N-(2,2-difluoroethyl)carbamoylmethyl, N-(2,2,2-trifluoroethyl)carbamoylmethyl, 1-[N-(2,2-difluoroethyl)carbamoyl]ethyl, 1-[N-(2,2,2-trifluoroethyl)carbamoyl]ethyl, 1-[N-(2,2-difluoroethyl)carbamoyl]-1-methylethyl, and 1-[N-(2,2,2-trifluoroethyl)carbamoyl]-1-methylethyl groups.

In the present invention, unless otherwise specified, the “(C1-C6)alkylcarbonyl (C1-C6)alkyl” refers to a ((C1-C6)alkyl)-C(═O)—((C1-C6)alkyl)- group in which the alkyl moiety is as defined above. Examples thereof include acetonyl, propionylmethyl, 2-methylpropionylmethyl, pivaloylmethyl, 2-acetylethyl, 2-propionylethyl, 2-(2-methylpropionyl)ethyl, 2-pivaloylethyl, 3-acetylpropyl, 3-propionylpropyl, 3-(2-methylpropionyl)propyl, and 3-pivaloylpropyl groups.

In the present invention, unless otherwise specified, the “(C1-C6)haloalkylcarbonyl (C1-C6)alkyl group” refers to a ((C1-C6)haloalkyl)-C(═O)—(C1-C6)alkyl)- group in which the haloalkyl moiety and the alkyl moiety are as defined above. Examples thereof include 2-oxo-3,3-difluoropropyl, 3-chloro-2-oxo-3,3-difluoropropyl, 2-oxo-3,3,3-trifluoropropyl, 3-oxo-4,4-difluorobutyl, 4-chloro-3-oxo-4,4-difluorobutyl, 3-oxo-4,4,4-trifluorobutyl, 3-oxo-4,4,5,5,5-pentafluoropentyl, 4-oxo-5,5,5-trifluoropentyl, and 4-oxo-5,5,6,6,6-pentafluorohexyl groups.

In the present invention, unless otherwise specified, the “hydroxyimino (C1-C6)alkyl” refers to a HO—N═((C1-C6)alkyl)- group in which the alkyl moiety is as defined above. Examples thereof include 1-(hydroxyimino)ethyl, 2-(hydroxyimino)ethyl, 1-(hydroxyimino)propyl, 2-(hydroxyimino)propyl, and 3-(hydroxyimino)propyl groups.

In the present invention, unless otherwise specified, the “(C1-C6)alkoxyimino” refers to a ((C1-C6)alkyl)-O—N═C— group in which the alkyl moiety is as defined above. Examples thereof include methoxyimino, ethoxyimino, and isopropoxyimino groups.

In the present invention, unless otherwise specified, the “(C1-C6)alkoxyimino (C1-C6)alkyl” refers to a ((C1-C6)alkyl)-O—N═C—((C1-C6)alkyl)- group in which the alkyl moiety is as defined above. Examples thereof include 1-(methoxyimino)ethyl, 2-(methoxyimino)ethyl, 1-(methoxyimino)propyl, 2-(methoxyimino)propyl, 3-(methoxyimino)propyl, 1-(ethoxyimino)ethyl, 2-(ethoxyimino)ethyl, 1-(ethoxyimino)propyl, 2-(ethoxyimino)propyl, 3-(ethoxyimino)propyl, 1-(isopropoxyimino)ethyl, and 2-(isopropoxyimino)ethyl groups.

In the present invention, unless otherwise specified, the “(C1-C6)haloalkoxyimino (C1-C6)alkyl” refers to a ((C1-C6)haloalkyl)-O—N═((C1-C6)alkyl)- group in which the haloalkyl moiety and the alkyl moiety are as defined above. Examples thereof include (2,2,2-trifluoroethoxyimino)methyl, 2-(2,2-difluoroethoxyimino)ethyl, 2-(2,2,2-trifluoroethoxyimino)ethyl, 2-(2,2,2-trifluoroethoxyimino)propyl, and 3-(2,2,2-trifluoroethoxyimino)propyl groups.

In the present invention, unless otherwise specified, the “(C1-C12)alkyl” refers to a linear or branched alkyl group having 1 to 12 carbon atoms. Examples thereof include, in addition to the examples of the (C1-C6)alkyl group, n-heptyl, n-octyl, n-nonyl, n-decyl, n-undecyl, n-dodecyl, 4,4-dimethylpentyl, 5-methylhexyl, 5,5-dimethylhexyl, 3,5,5-trimethylhexyl, 6-methylheptyl, 6,6-dimethylheptyl, 3,6,6-trimethylheptyl, 7-methyloctyl, 7,7-dimethyloctyl, 8-methylnonyl, 8,8-dimethylnonyl, 9-methyldecyl, 9,9-dimethyldecyl, and 10-methylundecyl groups.

In the present invention, unless otherwise specified, the “(C3-C6)cycloalkoxy” refers to a ((C3-C6)cycloalkyl)-O— group in which the cycloalkyl moiety is as defined above. Examples thereof include cyclopropoxy, cyclobutoxy, cyclopentyloxy, and cyclohexyloxy groups.

In the present invention, unless otherwise specified, the “(C3-C6)halocycloalkoxy” refers to a ((C3-C6)halocycloalkyl)-O— group in which the halocycloalkyl moiety is as defined above. Examples thereof include 2,2-difluorocyclopropoxy, 2,2-dichlorocyclopropoxy, 3,3-difluorocyclobutoxy, 3,3-dichlorocyclobutoxy, 3-fluorocyclopentyloxy, 3,3-difluorocyclopentyloxy, nonafluorocyclopentyloxy, 3,3-dichlorocyclopentyloxy, 4,4-difluorocyclohexyloxy, and 4,4-dichlorocyclohexyloxy groups.

In the present invention, unless otherwise specified, the “(C1-C6)alkoxy (C1-C6)alkoxy” refers to a ((C1-C6)alkoxy)-((C1-C6)alkoxy)- group in which the alkoxy moiety is as defined above. Examples thereof include 2-methoxyethoxy, 3-methoxypropoxy, 2-ethoxyisopropoxy, 2-isopropoxybutoxy, 5-ethoxypentyloxy, 6-ethoxyhexyloxy, 2-(tert-butoxy)ethoxy, 2-methoxyisopentyloxy, and 2-isopropoxyisobutoxy groups.

In the present invention, unless otherwise specified, the “(C1-C6)haloalkoxy (C1-C6)alkoxy” refers to a ((C1-C6)haloalkoxy)-((C1-C6)alkoxy)- group in which the haloalkoxy and alkoxy moieties are as defined above. Examples thereof include 2-difluoromethoxyethoxy, 2-trifluoromethoxyethoxy, 3-trifluoromethoxypropoxy, and 2-(2,2,2-trifluoroethoxy)ethoxy groups.

In the present invention, unless otherwise specified, the “(C1-C6)haloalkoxy (C1-C6)haloalkoxy” refers to a ((C1-C6)haloalkoxy)-((C1-C6)haloalkoxy)- group in which the haloalkoxy moiety is as defined above. Examples thereof include 2-(difluoromethoxy)-1,1,2,2-tetrafluoroethoxy, 2-(trifluoromethoxy)-1,1,2,2-tetrafluoroethoxy, 1,1,2,3,3,3-hexafluoro-2-(heptafluoropropoxy)propoxy, and 2-(2,2,2-trifluoroethoxy)-1,1,2,2-tetrafluoroethoxy groups.

In the present invention, unless otherwise specified, the “(C1-C6)haloalkylthio” refers to a ((C1-C6)haloalkyl)-S— group in which the haloalkyl moiety is as defined above. Examples thereof include fluoromethylthio, difluoromethylthio, trifluoromethylthio, trichloromethylthio, 2,2,2-trifluoroethylthio, pentafluoroethylthio, 2,2,2-trichloroethylthio, 3,3,3-trifluoropropylthio, heptafluoropropylthio, 1,1,1,3,3,3-hexafluoropropan-2-ylthio, heptafluoropropan-2-ylthio, and 4,4,4-trifluorobutylthio groups.

In the present invention, unless otherwise specified, the “(C2-C6)alkenylthio” refers to a ((C2-C6)alkenyl)-S— group in which the alkenyl moiety is as defined above. Examples thereof include vinylthio, 1-propenylthio, isopropenylthio, 2-propenylthio, 2-butenylthio, 3-butenylthio, 2-pentenylthio, 3-pentenylthio, 4-pentenylthio, 2-methyl-2-butenylthio, 2,4-pentadienylthio, 2-hexenylthio, 3-hexenylthio, 4-hexenylthio, 5-hexenylthio, and 2,4-hexadienylthio groups.

In the present invention, unless otherwise specified, the “(C2-C6)haloalkenylthio” refers to a ((C2-C6)haloalkenyl)-S— group in which the haloalkenyl moiety is as defined above. Examples of thereof include 2,2-difluorovinylthio, 2,2-dichlorovinylthio, 3,3-difluoro-2-propenylthio, 2,3,3-trifluoro-2-propenylthio, 3-chloro-2-propenylthio, 3,3-dichloro-2-propenylthio, 3-bromo-2-propenylthio, 4,4-difluoro-3-butenylthio, 4,4-difluoro-3-butene-2-ylthio, 3,4,4-trifluoro-3-butenylthio, 4,4,4-trifluoro-3-chloro-2-butenylthio, 4,4-dichloro-3-butenylthio, 4,5,5-trifluoro-4-pentenylthio, 5,5,5-trifluoro-3-pentenylthio, 4,4,4-trifluoro-3-trifluoromethyl-2-butenylthio, 6,6-difluoro-5-hexenylthio, 5,6,6-trifluoro-5-hexenylthio, and 6,6-dichloro-5-hexenylthio groups.

In the present invention, unless otherwise specified, the “(C3-C6)cycloalkylthio” refers to a ((C3-C6)cycloalkyl)-S— group in which the cycloalkyl moiety is as defined above. Examples thereof include cyclopropylthio, cyclobutylthio, cyclopentylthio, and cyclohexylthio groups.

In the present invention, unless otherwise specified, the “(C3-C6)halocycloalkylthio” refers to a ((C3-C6)halocycloalkyl)-S— group in which the halocycloalkyl moiety is as defined above. Examples thereof include 2,2-difluorocyclopropylthio, 2,2-dichlorocyclopropylthio, 3,3-difluorocyclobutylthio, 3,3-difluorocyclopentylthio, and 4,4-difluorocyclohexylthio groups.

In the present invention, unless otherwise specified, the “(C3-C6)cycloalkyl (C1-C6)alkylthio” refers to a ((C3-C6)cycloalkyl)-((C1-C6)alkyl)-S— group in which the cycloalkyl and alkyl moieties are as defined above. Examples thereof include cyclopropylmethylthio, 2-cyclopropylethylthio, 3-cyclopropylpropylthio, 4-cyclopropylbutylthio, 5-cyclopropylpentylthio, cyclobutylmethylthio, cyclopentylmethylthio, and cyclohexylmethylthio groups.

In the present invention, unless otherwise specified, the “(C3-C6)halocycloalkyl (C1-C6)alkylthio” refers to a ((C3-C6)halocycloalkyl)-((C1-C6)alkyl)-S— group in which the halocycloalkyl and alkyl moieties are as defined above. Examples thereof include 2,2-difluorocyclopropylmethylthio, 2,2-dichlorocyclopropylmethylthio, 2-(2,2-difluorocyclopropyl)ethylthio, 2-(2,2-dichlorocyclopropyl)ethylthio, 2,2-difluorocyclobutylmethylthio, and 4,4-difluorocyclohexylmethylthio groups.

In the present invention, unless otherwise specified, the “tri((C1-C6)alkyl)silyl (C1-C6)alkylthio” refers to a ((C1-C6)alkyl)₃Si—((C1-C6)alkyl)-S— group in which the alkyl moiety is as defined above, and the three alkyl groups of the ((C1-C6)alkyl)₃ moiety are identical to or different from each other. Examples thereof include trimethylsilylmethylthio, triethylsilylmethylthio, trimethylsilylethylthio, tert-butyldimethylsilylmethylthio, and trimethylsilylpropylthio groups.

In the present invention, unless otherwise specified, the “(C1-C6)alkylthio (C1-C6)alkoxy” refers to a ((C1-C6)alkyl)-S—((C1-C6)alkyl)-O— group in which the alkyl moiety is as defined above. Examples thereof include methylthiomethoxy, 2-methylthioethoxy, ethylthiomethoxy, 2-ethylthioethoxy, tert-butylthiomethoxy, and 2-(tert-butylthio)ethoxy groups.

In the present invention, unless otherwise specified, the “(C1-C6)haloalkylthio (C1-C6)alkoxy” refers to a ((C1-C6)haloalkyl)-S—((C1-C6)alkyl)-O— group in which the haloalkyl and alkyl moieties are as defined above. Examples thereof include fluoromethylthiomethoxy, difluoromethylthiomethoxy, trifluoromethylthiomethoxy, 1-(fluoromethylthio)ethoxy, 1-(difluoromethylthio)ethoxy, 1-(trifluoromethylthio)ethoxy, 2-(fluoromethylthio)ethoxy, 2-(difluoromethylthio)ethoxy, and 2-(trifluoromethylthio)ethoxy groups.

In the present invention, unless otherwise specified, the “(C1-C6)alkylsulfinyl” refers to a ((C1-C6)alkyl)-S(═O)— group in which the alkyl moiety is as defined above. Examples thereof include methylsulfinyl, ethylsulfinyl, n-propylsulfinyl, isopropylsulfinyl, n-butylsulfinyl, isobutylsulfinyl, sec-butylsulfinyl, and tert-butylsulfinyl groups.

In the present invention, unless otherwise specified, the “(C1-C6)haloalkylsulfinyl” refers to a ((C1-C6)haloalkyl)-S(═O)— group in which the haloalkyl moiety is as defined above. Examples thereof include difluoromethylsulfinyl, trifluoromethylsulfinyl, 2,2,2-trifluoroethylsulfinyl, 2,2,2-trichloroethylsulfinyl, pentafluoroethylsulfinyl, heptafluoropropylsulfinyl, trichloromethylsulfinyl, and heptafluoro-2-propylsulfinyl groups.

In the present invention, unless otherwise specified, the “(C3-C6)cycloalkylsulfinyl” refers to a ((C3-C6)cycloalkyl)-S(═O)— group in which the cycloalkyl moiety is as defined above. Examples thereof include cyclopropylsulfinyl, cyclobutylsulfinyl, cyclopentylsulfinyl, and cyclohexylsulfinyl groups.

In the present invention, unless otherwise specified, the “(C3-C6)halocycloalkylsulfinyl” refers to a ((C3-C6)halocycloalkyl)-S(═O)— group in which the halocycloalkyl moiety is as defined above. Examples thereof include 2,2-difluorocyclopropylsulfinyl, 2,2-dichlorocyclopropylsulfinyl, 3,3-difluorocyclobutylsulfinyl, 3,3-difluorocyclopentylsulfinyl, and 4,4-difluorocyclohexylsulfinyl groups.

In the present invention, unless otherwise specified, the “(C3-C6)cycloalkylsulfonyl” refers to a ((C3-C6)cycloalkyl)-S(═O)₂— group in which the cycloalkyl moiety is as defined above. Examples thereof include cyclopropylsulfonyl, cyclobutylsulfonyl, cyclopentylsulfonyl, and cyclohexylsulfonyl groups.

In the present invention, unless otherwise specified, the “(C3-C6)halocycloalkylsulfonyl” refers to a ((C3-C6)halocycloalkyl)-S(═O)₂— group in which the halocycloalkyl moiety is as defined above. Examples thereof include 2,2-difluorocyclopropylsulfonyl, 2,2-dichlorocyclopropylsulfonyl, 3,3-difluorocyclobutylsulfonyl, 3,3-difluorocyclopentylsulfonyl, and 4,4-difluorocyclohexylsulfonyl groups.

In the present invention, unless otherwise specified, the “(C1-C6)haloalkylcarbonyl” refers to a ((C1-C6)haloalkyl)-C(═O)— group in which the haloalkyl moiety is as defined above. Examples thereof include fluoroacetyl, difluoroacetyl, trifluoroacetyl, chloroacetyl, trichloroacetyl, tribromoacetyl, 3,3,3-trifluoropropionyl, 3,3-difluoropropionyl, and 4,4,4-trifluorobutyryl groups.

In the present invention, unless otherwise specified, the “(C1-C6)haloalkoxycarbonyl” refers to a ((C1-C6)haloalkyl)-O—C(═O)— group in which the haloalkyl moiety is as defined above. Examples thereof include 2-fluoroethoxycarbonyl, 2,2,2-trifluoroethoxycarbonyl, 2,2,2-trichloroethoxycarbonyl, pentafluoroethoxycarbonyl, 3,3,3-trifluoropropoxycarbonyl, and heptafluoro-2-propoxycarbonyl groups.

In the present invention, unless otherwise specified, the “(C1-C6)haloalkylaminocarbonyl” refers to a ((C1-C6)haloalkyl)-NH—C(═O)— group in which the haloalkyl moiety is as defined above. Examples thereof include 2-fluoroethylaminocarbonyl, 2,2,2-trifluoroethylaminocarbonyl, 2,2,2-trichloroethylaminocarbonyl, pentafluoroethylaminocarbonyl, and 1,1,1,3,3,3-hexafluoro-2-propylaminocarbonyl groups.

In the present invention, unless otherwise specified, the “(C1-C6)alkylaminocarbonylthio” refers to a ((C1-C6)alkyl)-NH—C(═O)—S— group in which the alkyl moiety is as defined above. Examples thereof include methylaminocarbonylthio, ethylaminocarbonylthio, propylaminocarbonylthio, and isopropylaminocarbonylthio groups.

In the present invention, unless otherwise specified, the “(C1-C6)haloalkylaminocarbonylthio” refers to a ((C1-C6)haloalkyl)-NH—C(═O)—S— group in which the haloalkyl moiety is as defined above. Examples thereof include 2-fluoroethylaminocarbonylthio, 2,2,2-trifluoroethylaminocarbonylthio, 2,2,2-trichloroethylaminocarbonylthio, pentafluoroethylaminocarbonylthio, and 1,1,1,3,3,3-hexafluoro-2-propylaminocarbonylthio groups.

In the present invention, unless otherwise specified, the “(C1-C6)alkylcarbonyloxy” refers to a ((C1-C6)alkyl)-C(═O)O— group in which the alkyl moiety is as defined above. Examples thereof include acetoxy and propionyloxy groups.

In the present invention, unless otherwise specified, the “(C1-C6)haloalkylcarbonyloxy” refers to a ((C1-C6)haloalkyl)-C(═O)O— group in which the haloalkyl moiety is as defined above. Examples thereof include fluoroacetoxy, difluoroacetoxy, trifluoroacetoxy, chloroacetoxy, trichloroacetoxy, tribromoacetoxy, 3,3,3-trifluoropropionyloxy, 3,3-difluoropropionyloxy, and 4,4,4-trifluorobutyryloxy groups.

In the present invention, unless otherwise specified, the “(C1-C6)alkylsulfinyloxy” refers to a ((C1-C6)alkyl)-S(═O)—O— group in which the alkyl moiety is as defined above. Examples thereof include methylsulfinyloxy, ethylsulfinyloxy, n-propylsulfinyloxy, and isopropylsulfinyloxy groups.

In the present invention, unless otherwise specified, the “(C1-C6)haloalkylsulfinyloxy” refers to a ((C1-C6)haloalkyl)-S(═O)—O— group in which the haloalkyl moiety is as defined above. Examples thereof include difluoromethylsulfinyloxy, trifluoromethylsulfinyloxy, 2,2,2-trifluoroethylsulfinyloxy, pentafluoroethylsulfinyloxy, heptafluoropropylsulfinyloxy, trichloromethylsulfinyloxy, and heptafluoro-2-propylsulfinyloxy groups.

In the present invention, unless otherwise specified, the “(C3-C6)cycloalkylsulfinyloxy” refers to a ((C3-C6)cycloalkyl)-S(═O)—O— group in which the cycloalkyl moiety is as defined above. Examples thereof include cyclopropylfinyloxy, cyclobutylsulfinyloxy, cyclopentylsulfinyloxy, and cyclohexylsulfinyloxy groups.

In the present invention, unless otherwise specified, the “(C3-C6)halocycloalkylsulfinyloxy” refers to a ((C3-C6)halocycloalkyl)-S(═O)—O— group in which the halocycloalkyl moiety is as defined above. Examples thereof include 2,2-difluorocyclopropylsulfinyloxy, 2,2-dichlorocyclopropylsulfinyloxy, 3,3-difluorocyclobutylsulfinyloxy, 3,3-difluorocyclopentylsulfinyloxy, and 4,4-difluorocyclohexylsulfinyloxy groups.

In the present invention, unless otherwise specified, the “(C1-C6)alkylsulfonyloxy” refers to a ((C1-C6)alkyl)-S(═O)₂—O— group in which the alkyl moiety is as defined above. Examples thereof include methylsulfonyloxy, ethylsulfonyloxy, n-propylsulfonyloxy, and isopropylsulfonyloxy groups.

In the present invention, unless otherwise specified, the “(C1-C6)haloalkylsulfonyloxy” refers to a ((C1-C6)haloalkyl)-S(═O)₂—O— group in which the haloalkyl moiety is as defined above. Examples thereof include difluoromethylsulfonyloxy, trifluoromethylsulfonyloxy, trichloromethylsulfonyloxy, 2,2,2-trifluoroethylsulfonyloxy, 2,2,2-trichloroethylsulfonyloxy, 3,3,3-trifluoropropylsulfonyloxy, and heptafluoro-2-propylsulfonyloxy groups.

In the present invention, unless otherwise specified, the “(C3-C6)cycloalkylsulfonyloxy” refers to a ((C3-C6)cycloalkyl)-S(═O)₂—O— group in which the cycloalkyl moiety is as defined above. Examples thereof include cyclopropylsulfonyloxy, cyclobutylsulfonyloxy, cyclopentylsulfonyloxy, and cyclohexylsulfonyloxy groups.

In the present invention, unless otherwise specified, the “(C3-C6)halocycloalkylsulfonyloxy” refers to a ((C3-C6)halocycloalkyl)-S(═O)₂—O— group in which the halocycloalkyl moiety is as defined above. Examples thereof include 2,2-difluorocyclopropylsulfonyloxy, 2,2-dichlorocyclopropylsulfonyloxy, 3,3-difluorocyclobutylsulfonyloxy, 3,3-difluorocyclopentylsulfonyloxy, and 4,4-difluorocyclohexylsulfonyloxy groups.

In the present invention, unless otherwise specified, the “(C1-C6)haloalkylamino” refers to a ((C1-C6)haloalkyl)-NH— group in which the haloalkyl moiety is as defined above. Examples thereof include 2-fluoroethylamino, 2,2-difluoroethylamino, 2,2,2-trifluoroethylamino, 2,2,2-trichloroethylamino, pentafluoroethylamino, 3,3,3-trifluoropropylamino, and 1,1,1,3,3,3-hexafluoro-2-propylamino groups.

In the present invention, unless otherwise specified, the “(C1-C6)alkylcarbonylamino” refers to a ((C1-C6)alkyl)-C(═O)—NH— group in which the alkyl moiety is as defined above. Examples thereof include acetylamino, propionylamino, butyrylamino, and isobutyrylamino groups.

In the present invention, unless otherwise specified, the “(C1-C6)haloalkylcarbonylamino” refers to a ((C1-C6)haloalkyl)-C(═O)—NH— group in which the haloalkyl moiety is as defined above. Examples thereof include fluoroacetylamino, difluoroacetylamino, trifluoroacetylamino, chloroacetylamino, trichloroacetylamino, tribromoacetylamino, 3,3,3-trifluoropropionylamino, pentafluoropropionylamino, and 3,3-difluoropropionylamino group.

In the present invention, unless otherwise specified, the “(C1-C6)haloalkoxycarbonylamino” refers to a ((C1-C6)haloalkoxy)-C(═O)—NH— group in which the haloalkoxy moiety is as defined above. Examples thereof include 2-fluoroethoxycarbonylamino, 2,2,2-trifluoroethoxycarbonylamino, 2,2,2-trichloroethoxycarbonylamino, pentafluoroethoxycarbonylamino, 3,3,3-trifluoropropoxycarbonylamino, and heptafluoro-2-propoxycarbonylamino groups.

In the present invention, unless otherwise specified, the “(C1-C6)alkylaminocarbonylamino” refers to a ((C1-C6)alkyl)-NH—C(═O)—NH— group in which the alkyl moiety is as defined above. Examples thereof include methylaminocarbonylamino, ethylaminocarbonylamino, n-propylaminocarbonylamino, isopropylaminocarbonylamino, and tert-butylaminocarbonylamino groups.

In the present invention, unless otherwise specified, the “(C1-C6)haloalkylaminocarbonylamino” refers to a ((C1-C6)haloalkyl)-NH—C(═O)—NH— group in which the haloalkyl moiety is as defined above. Examples thereof include 2-fluoroethylaminocarbonylamino, 2,2,2-trifluoroethylaminocarbonylamino, 2,2,2-trichloroethylaminocarbonylamino, pentafluoroethylaminocarbonylamino, and 1,1,1,3,3,3-hexafluoro-2-propylaminocarbonylamino groups.

In the present invention, unless otherwise specified, the “(C1-C6)alkylsulfinylamino” refers to a ((C1-C6)alkyl)-SO—NH—0 group in which the alkyl moiety is as defined above. Examples thereof include methylsulfinylamino, ethylsulfinylamino, n-propylsulfinylamino, isopropylsulfinylamino, and tert-butylsulfinylamino groups.

In the present invention, unless otherwise specified, the “(C1-C6)haloalkylsulfinylamino” refers to a ((C1-C6)haloalkyl)-S(═O)—NH— group in which the haloalkyl moiety is as defined above. Examples thereof include fluoromethylsulfinylamino, difluoromethylsulfinylamino, trifluoromethylsulfinylamino, chloromethylsulfinylamino, trichloromethylsulfinylamino, 2,2,2-trifluoroethylsulfinylamino, 2,2-difluoroethylsulfinylamino, and 3,3,3-trifluoropropylsulfinylamino groups.

In the present invention, unless otherwise specified, the “(C1-C6)alkylsulfonylamino” refers to a ((C1-C6)alkyl)-S(═O)₂—NH— group in which the alkyl moiety is as defined above. Examples thereof include methylsulfonylamino, ethylsulfonylamino, n-propylsulfonylamino, isopropylsulfonylamino, and tert-butylsulfonylamino groups.

In the present invention, unless otherwise specified, the “(C1-C6)haloalkylsulfonylamino” refers to a ((C1-C6)haloalkyl)-S(═O)₂—NH— group in which the haloalkyl moiety is as defined above. Examples thereof include fluoromethylsulfonylamino, difluoromethylsulfonylamino, trifluoromethylsulfonylamino, chloromethylsulfonylamino, trichloromethylsulfonylamino, 2,2,2-trifluoroethylsulfonylamino, 2,2-difluoroethylsulfonylamino, and 3,3,3-trifluoropropylsulfonylamino groups.

In the present invention, unless otherwise specified, the “tri((C1-C6)alkyl)silyl” refers to a ((C1-C6)alkyl)₃-Si— group in which the alkyl moiety is as defined above, and the three alkyl groups are optionally identical or different from each other. Examples thereof include trimethylsilyl, triethylsilyl, triisopropylsilyl, dimethylisopropylsilyl, and tert-butyldimethylsilyl groups.

In the present invention, unless otherwise specified, the “phenyl (C1-C6)alkoxy” refers to a phenyl-((C1-C6)alkyl)-O— group in which the alkyl moiety is as defined above. Examples thereof include phenylmethoxy, 1-phenylethoxy, 2-phenylethoxy, 1-phenylpropoxy, 2-phenylbutoxy, and 1-phenylpentoxy groups.

In the present invention, unless otherwise specified, the “phenyl (C1-C6)alkylthio” refers to a phenyl-((C1-C6)alkyl)-S— group in which the alkyl moiety is as defined above. Examples thereof include phenylmethylthio, 1-phenylethylthio, 2-phenylethylthio, 1-phenylpropylthio, 2-phenylbutylthio, and 1-phenylpentylthio groups.

In the present invention, unless otherwise specified, the “phenyl (C1-C6)alkylsulfinyl” refers to a phenyl-((C1-C6)alkyl)-S(═O)— group in which the alkyl moiety is as defined above. Examples thereof include phenylmethylsulfinyl, 1-phenylethylsulfinyl, 2-phenylethylsulfinyl, 1-phenylpropylsulfinyl, 2-phenylbutylsulfinyl, and 1-phenylpentylsulfinyl groups.

In the present invention, unless otherwise specified, the “phenyl (C1-C6)alkylsulfonyl” refers to a phenyl-((C1-C6)alkyl)-S(═O)₂— group in which the alkyl moiety is as defined above. Examples thereof include phenylmethylsulfonyl, 1-phenylethylsulfonyl, 2-phenylethylsulfonyl, 1-phenylpropylsulfonyl, 2-phenylbutylsulfonyl, and 1-phenylpentylsulfonyl groups.

In the present invention, unless otherwise specified, the “(C1-C6)alkylthio (C1-C6)alkyl” refers to a ((C1-C6)alkyl)-S—((C1-C6)alkyl)- group in which the alkyl moiety is as defined above. Examples thereof include methylthiomethyl, ethylthiomethyl, n-propylthiomethyl, isopropylthiomethyl, 1-(methylthio)ethyl, 2-(methylthio)ethyl, 2-(ethylthio)ethyl, 1-(n-propylthio)ethyl, 2-(n-propylthio)ethyl, 1-(isopropylthio)ethyl, 2-(isopropylthio)ethyl, 1-(methylthio)propyl, 2-(methylthio)propyl, 3-(methylthio)propyl, 1-(ethylthio)propyl, 2-(ethylthio)propyl, 3-(ethylthio)propyl, 1-(n-propylthio)propyl, 2-(n-propylthio)propyl, 3-(n-propylthio)propyl, 1-(methylthio)butyl, 2-(methylthio)butyl, 3-(methylthio)butyl, 4-(methylthio)butyl, 1-(methylthio)pentyl, 2-(methylthio)pentyl, 3-(methylthio)pentyl, 4-(methylthio)pentyl, 5-(methylthio)pentyl, 2-(n-butylthio)ethyl, 2-(isobutylthio)ethyl, 2-(sec-butylthio)ethyl, 2-(tert-butylthio)ethyl, pentylthiomethyl, and hexylthiomethyl groups.

In the present invention, unless otherwise specified, the “(C1-C6)haloalkylthio (C1-C6)alkyl” refers to a ((C1-C6) haloalkyl)-S—((C1-C6)alkyl)- group in which the haloalkyl moiety and the alkyl moiety are as defined above. Examples thereof include 2-(difluoromethylthio)ethyl, 2-(trifluoromethylthio)ethyl, 2-(2,2-difluoroethylthio)ethyl, 2-(2,2,2-trifluoroethylthio)ethyl, 2-(3,3-difluoropropylthio)ethyl, 2-(3,3,3-trifluoropropylthio)ethyl, 3-(difluoromethylthio)propyl, 3-(trifluoromethylthio)propyl, 3-(2,2-difluoroethylthio)propyl, 3-(2,2,2-trifluoroethylthio)propyl, 3-(3,3-difluoropropylthio)propyl, 3-(3,3,3-trifluoropropylthio)propyl, 4-(trifluoromethylthio)butyl, and 5-(trifluoromethylthio)pentyl groups.

In the present invention, unless otherwise specified, the “(C1-C6)haloalkoxy (C1-C6)alkyl” refers to a ((C1-C6)haloalkyl)-O—((C1-C6)alkyl)- group in which the haloalkoxy moiety and the alkyl moiety are as defined above. Examples thereof include 2-(difluoromethoxy)ethyl, 2-(trifluoromethoxy)ethyl, 2-(2,2-difluoroethoxy)ethyl, 2-(2,2,2-trifluoroethoxy)ethyl, 2-(3,3-difluoropropioxy)ethyl, 2-(3,3,3-trifluoropropioxy)ethyl, 3-(difluoromethoxy)propyl, 3-(trifluoromethoxy)propyl, 3-(2,2-difluoroethoxy)propyl, 3-(2,2,2-trifluoroethoxy)propyl, 3-(3,3-difluoropropioxy)propyl, 3-(3,3,3-trifluoropropioxy)propyl, 4-(trifluoromethoxy)butyl, and 5-(trifluoromethoxy)pentyl groups.

In the present invention, unless otherwise specified, the “cyano (C3-C6)cycloalkyl (C1-C6)alkyl” refers to a (cyano)-((C3-C6)cycloalkyl)-((C1-C6)alkyl group)- group in which the cycloalkyl moiety and the alkyl moiety are as defined above. Examples thereof include (1-cyanocyclopropane-1-yl)methyl, 2-(1-cyanocyclopropane-1-yl)ethyl, (1-cyanocyclobutane-1-yl)methyl, 2-(1-cyanocyclobutane-1-yl)ethyl, (1-cyanocyclopentane-1-yl)methyl, 2-(1-cyanocyclopentane-1-yl)ethyl, (1-cyanocyclohexane-1-yl)methyl, and 2-(1-cyanocyclohexane-1-yl)ethyl groups.

In the present invention, unless otherwise specified, the “hydroxy (C3-C6)cycloalkyl” refers to a (hydroxy)-(C3-C6)cycloalkyl)- group in which the cycloalkyl moiety is as defined above. Examples thereof include 2-(hydroxy)cyclopropyl, 1-(hydroxymethyl)cyclopropyl, 3-(hydroxy)cyclobutyl, 3-(hydroxy)cyclopentyl, 4-(hydroxy)cyclohexyl, 2-(hydroxy)cyclopropyl, 1-(hydroxymethyl)cyclopropyl, 3-(hydroxy)cyclobutyl, 3-(hydroxy)cyclopentyl, and 4-(hydroxy)cyclohexyl groups.

In the present invention, unless otherwise specified, the “(C1-C6)alkylcarbonyloxy (C3-C6)cycloalkyl” refers to a ((C1-C6)alkyl)-C(═O)O— ((C3-C6)cycloalkyl)- group in which the alkyl moiety and the cycloalkyl moiety are as defined above. Examples thereof include 2-(acetoxy)cyclopropyl, 1-(acetoxymethyl)cyclopropyl, 3-(acetoxy)cyclobutyl, 3-(acetoxy)cyclopentyl, 4-(acetoxy)cyclohexyl, 2-(propionyloxy)cyclopropyl, 1-(propionyloxymethyl)cyclopropyl, 3-(propionyloxy)cyclobutyl, 3-(propionyloxy)cyclopentyl, and 4-(propionyloxy)cyclohexyl groups.

In the present invention, unless otherwise specified, the “(C1-C6)alkylsulfonyloxy (C3-C6)cycloalkyl” refers to a ((C1-C6)alkyl)-S(═O)₂—O—((C3-C6)cycloalkyl)- group in which the alkyl moiety is as defined above. Examples thereof include 2-(methylsulfonyloxy)cyclopropyl, 1-(methylsulfonyloxymethyl)cyclopropyl, 3-(methylsulfonyloxy)cyclobutyl, 3-(methylsulfonyloxy)cyclopentyl, 4-(methylsulfonyloxy)cyclohexyl, 2-(ethylsulfonyloxy)cyclopropyl, 1-(ethylsulfonyloxymethyl)cyclopropyl, 3-(ethylsulfonyloxy)cyclobutyl, 3-(ethylsulfonyloxy)cyclopentyl, and 4-(ethylsulfonyloxy)cyclohexyl groups.

In the present invention, unless otherwise specified, the “(C1-C6)alkoxy (C3-C6)cycloalkyl” refers to a ((C1-C6)alkoxy)-((C3-C6)cycloalkyl)- group in which the alkoxy moiety and the cycloalkyl moiety are as defined above. Examples thereof include 1-(methoxy)cyclopropyl, 1-(ethoxy)cyclopropyl, 1-(methoxymethyl)cyclopropyl, 1-(ethoxymethyl)cyclopropyl, 1-(n-propoxy)cyclopropyl, 1-(isopropoxy)cyclopropyl, 1-(methoxy)cyclobutyl, 1-(ethoxy)cyclobutyl, 1-(n-propoxy)cyclobutyl, 1-(isopropoxy)cyclobutyl, 1-(methoxy)cyclopentyl, 1-(ethoxy)cyclopentyl, 1-(methoxy)cyclohexyl, and 1-(ethoxy)cyclohexyl groups.

In the present invention, unless otherwise specified, the “(C1-C6)haloalkoxy (C3-C6)cycloalkyl” refers to a ((C1-C6)haloalkoxy)-((C3-C6)cycloalkyl)- group in which the haloalkoxy moiety and the cycloalkyl moiety are as defined above. Examples thereof include 1-(difluoromethoxy)cyclopropyl, 1-(trifluoromethoxy)cyclopropyl, 1-(2,2-difluoroethoxy)cyclopropyl, 1-(2,2,2-trifluoroethoxy)cyclopropyl, 1-(difluoromethoxymethyl)cyclopropyl, 1-(trifluoromethoxymethyl)cyclopropyl, 1-(2,2-difluoroethoxymethyl)cyclopropyl, 1-(2,2,2-trifluoroethoxymethyl)cyclopropyl, 1-(difluoromethoxy)cyclobutyl, 1-(trifluoromethoxy)cyclobutyl, 1-(2,2-difluoroethoxy)cyclobutyl, 1-(2,2,2-trifluoroethoxy)cyclobutyl, 1-(difluoromethoxy)cyclopentyl, 1-(trifluoromethoxy)cyclopentyl, 1-(2,2-difluoroethoxy)cyclopentyl, 1-(2,2,2-trifluoroethoxy)cyclopentyl, 1-(difluoromethoxy)cyclohexyl, 1-(trifluoromethoxy)cyclohexyl, 1-(2,2-difluoroethoxy)cyclohexyl, and 1-(2,2,2-trifluoroethoxy)cyclohexyl groups.

In the present invention, unless otherwise specified, the “(C1-C6)alkylthio (C3-C6)cycloalkyl” refers to a ((C1-C6)alkyl)-S—((C3-C6)cycloalkyl group)- group in which the alkyl moiety and the cycloalkyl moiety are as defined above. Examples thereof include 1-(methylthio)cyclopropyl, 1-(ethylthio)cyclopropyl, 1-(methylthiomethyl)cyclopropyl, 1-(ethylthiomethyl)cyclopropyl, 1-(n-propylthio)cyclopropyl, 1-(isopropylthio)cyclopropyl, 1-(pentylthio)cyclopropyl, 1-(hexylthio)cyclopropyl, 1-(methylthio)cyclobutyl, 1-(ethylthio)cyclobutyl, 1-(n-propylthio)cyclobutyl, 1-(isopropylthio)cyclobutyl, 1-(pentylthio)cyclobutyl, 1-(hexylthio)cyclobutyl, 1-(methylthio)cyclopentyl, 1-(ethylthio)cyclopentyl, 1-(methylthio)cyclohexyl, and 1-(ethylthio)cyclohexyl groups.

In the present invention, unless otherwise specified, the “(C1-C6)haloalkylthio (C3-C6)cycloalkyl” refers to a ((C1-C6)haloalkyl)-S—((C3-C6)cycloalkyl group)- group in which the haloalkyl moiety and the cycloalkyl moiety are as defined above. Examples thereof include 1-(difluoromethylthio)cyclopropyl, 1-(trifluoromethylthio)cyclopropyl, 1-(2,2-difluoroethylthio)cyclopropyl, 1-(2,2,2-trifluoroethylthio)cyclopropyl, 1-(difluoromethylthiomethyl)cyclopropyl, 1-(trifluoromethylthiomethyl)cyclopropyl, 1-(2,2-difluoroethylthiomethyl)cyclopropyl, 1-(2,2,2-trifluoroethylthiomethyl)cyclopropyl, 1-(difluoromethylthio)cyclobutyl, 1-(trifluoromethylthio)cyclobutyl, 1-(2,2-difluoroethylthio)cyclobutyl, 1-(2,2,2-trifluoroethylthio)cyclobutyl, 1-(difluoromethylthio)cyclopentyl, 1-(trifluoromethylthio)cyclopentyl, 1-(2,2-difluoroethylthio)cyclopentyl, 1-(2,2,2-trifluoroethylthio)cyclopentyl, 1-(difluoromethylthio)cyclohexyl, 1-(trifluoromethylthio)cyclohexyl, 1-(2,2-difluoroethylthio)cyclohexyl, and 1-(2,2,2-trifluoroethylthio)cyclohexyl groups.

In the present invention, unless otherwise specified, the “(C1-C6)alkylsulfinyl (C3-C6)cycloalkyl” refers to a ((C1-C6)alkyl)-S(═O)—((C3-C6)cycloalkyl group)- group in which the alkyl moiety and the cycloalkyl moiety are as defined above. Examples thereof include 1-(methylsulfinyl)cyclopropyl, 1-(ethylsulfinyl)cyclopropyl, 1-(methylsulfinylmethyl)cyclopropyl, 1-(ethylsulfinylmethyl)cyclopropyl, 1-(n-propylsulfinyl)cyclopropyl, 1-(isopropylsulfinyl)cyclopropyl, 1-(pentylsulfinyl)cyclopropyl, 1-(hexylsulfinyl)cyclopropyl, 1-(methylsulfinyl)cyclobutyl, 1-(ethylsulfinyl)cyclobutyl, 1-(n-propylsulfinyl)cyclobutyl, 1-(isopropylsulfinyl)cyclobutyl, 1-(pentylsulfinyl)cyclobutyl, 1-(hexylsulfinyl)cyclobutyl, 1-(methylsulfinyl)cyclopentyl, 1-(ethylsulfinyl)cyclopentyl, 1-(methylsulfinyl)cyclohexyl, and 1-(ethylsulfinyl)cyclohexyl groups.

In the present invention, unless otherwise specified, the “(C1-C6)haloalkylsulfinyl (C3-C6)cycloalkyl” refers to a ((C1-C6)haloalkyl)-S(═O)—((C3-C6)cycloalkyl group)- group in which the haloalkyl moiety and the cycloalkyl moiety are as defined above. Examples thereof include 1-(difluoromethylsulfinyl)cyclopropyl, 1-(trifluoromethylsulfinyl)cyclopropyl, 1-(2,2-difluoroethylsulfinyl)cyclopropyl, 1-(2,2,2-trifluoroethylsulfinyl)cyclopropyl, 1-(difluoromethylsulfinylmethyl)cyclopropyl, 1-(trifluoromethylsulfinylmethyl)cyclopropyl, 1-(2,2-difluoroethylsulfinylmethyl)cyclopropyl, 1-(2,2,2-trifluoroethylsulfinylmethyl)cyclopropyl, 1-(difluoromethylsulfinyl)cyclobutyl, 1-(trifluoromethylsulfinyl)cyclobutyl, 1-(2,2-difluoroethylsulfinyl)cyclobutyl, 1-(2,2,2-trifluoroethylsulfinyl)cyclobutyl, 1-(difluoromethylsulfinyl)cyclopentyl, 1-(trifluoromethylsulfinyl)cyclopentyl, 1-(2,2-difluoroethylsulfinyl)cyclopentyl, 1-(2,2,2-trifluoroethylsulfinyl)cyclopentyl, 1-(difluoromethylsulfinyl)cyclohexyl, 1-(trifluoromethylsulfinyl)cyclohexyl, 1-(2,2-difluoroethylsulfinyl)cyclohexyl, and 1-(2,2,2-trifluoroethylsulfinyl)cyclohexyl groups.

In the present invention, unless otherwise specified, the “(C1-C6)alkylsulfonyl (C3-C6)cycloalkyl” refers to a ((C1-C6)alkyl)-S(═O)₂—((C3-C6)cycloalkyl group)- group in which the alkyl moiety and the cycloalkyl moiety are as defined above. Examples thereof include 1-(methylsulfonyl)cyclopropyl, 1-(ethylsulfonyl)cyclopropyl, 1-(methylsulfonylmethyl)cyclopropyl, 1-(ethylsulfonylmethyl)cyclopropyl, 1-(n-propylsulfonyl)cyclopropyl, 1-(isopropylsulfonyl)cyclopropyl, 1-(pentylsulfonyl)cyclopropyl, 1-(hexylsulfonyl)cyclopropyl, 1-(methylsulfonyl)cyclobutyl, 1-(ethylsulfonyl)cyclobutyl, 1-(n-propylsulfonyl)cyclobutyl, 1-(isopropylsulfonyl)cyclobutyl, 1-(pentylsulfonyl)cyclobutyl, 1-(hexylsulfonyl)cyclobutyl, 1-(methylsulfonyl)cyclopentyl, 1-(ethylsulfonyl)cyclopentyl, 1-(methylsulfonyl)cyclohexyl, and 1-(ethylsulfonyl)cyclohexyl groups.

In the present invention, unless otherwise specified, the “(C1-C6)haloalkylsulfonyl (C3-C6)cycloalkyl” refers to a ((C1-C6)haloalkyl)-S(═O)₂—((C3-C6)cycloalkyl group)- group in which the haloalkyl moiety and the cycloalkyl moiety are as defined above. Examples thereof include 1-(difluoromethylsulfonyl)cyclopropyl, 1-(trifluoromethylsulfonyl)cyclopropyl, 1-(2,2-difluoroethylsulfonyl)cyclopropyl, 1-(2,2,2-trifluoroethylsulfonyl)cyclopropyl, 1-(difluoromethylsulfonylmethyl)cyclopropyl, 1-(trifluoromethylsulfonylmethyl)cyclopropyl, 1-(2,2-difluoroethylsulfonylmethyl)cyclopropyl, 1-(2,2,2-trifluoroethylsulfonylmethyl)cyclopropyl, 1-(difluoromethylsulfonyl)cyclobutyl, 1-(trifluoromethylsulfonyl)cyclobutyl, 1-(2,2-difluoroethylsulfonyl)cyclobutyl, 1-(2,2,2-trifluoroethylsulfonyl)cyclobutyl, 1-(difluoromethylsulfonyl)cyclopentyl, 1-(trifluoromethylsulfonyl)cyclopentyl, 1-(2,2-difluoroethylsulfonyl)cyclopentyl, 1-(2,2,2-trifluoroethylsulfonyl)cyclopentyl, 1-(difluoromethylsulfonyl)cyclohexyl, 1-(trifluoromethylsulfonyl)cyclohexyl, 1-(2,2-difluoroethylsulfonyl)cyclohexyl, and 1-(2,2,2-trifluoroethylsulfonyl)cyclohexyl groups.

In the present invention, unless otherwise specified, the “(C1-C6)alkoxycarbonyl (C3-C6)cycloalkyl” refers to a ((C1-C6)alkyl)-O—C(═O)—((C3-C6)cycloalkyl)- group in which the alkyl moiety and the cycloalkyl moiety are as defined above. Examples thereof include 1-(methoxycarbonyl)cyclopropyl, 1-(ethoxycarbonyl)cyclopropyl, 2-(methoxycarbonyl)cyclopropyl, 2-(ethoxycarbonyl)cyclopropyl, 1-(methoxycarbonyl)cyclobutyl, 1-(ethoxycarbonyl)cyclobutyl, 1-(methoxycarbonyl)cyclopentyl, 1-(ethoxycarbonyl)cyclopentyl, 1-(methoxycarbonyl)cyclohexyl, 1-(ethoxycarbonyl)cyclohexyl, 1-(isopropoxycarbonyl)cyclohexyl, and 1-(tert-butoxycarbonyl)cyclohexyl groups.

In the present invention, unless otherwise specified, the “carboxy (C3-C6)cycloalkyl” refers to a (carboxy)-((C3-C6)cycloalkyl group)- group in which the cycloalkyl moiety is as defined above. Examples thereof include 1-(carboxy)cyclopropyl, 2-(carboxy)cyclopropyl, 1-(carboxy)cyclobutyl, 1-(carboxy)cyclopentyl, and 1-(carboxy)cyclohexyl groups.

In the present invention, unless otherwise specified, the “carbamoyl (C3-C6)cycloalkyl” refers to a (carbamoyl)-((C3-C6)cycloalkyl group)- group in which the cycloalkyl moiety is as defined above. Examples thereof include 1-(carbamoyl)cyclopropyl, 2-(carbamoyl)cyclopropyl, 1-(carbamoyl)cyclobutyl, 1-(carbamoyl)cyclopentyl, and 1-(carbamoyl)cyclohexyl groups.

In the present invention, unless otherwise specified, the “(C1-C6)alkylaminocarbonyl (C3-C6)cycloalkyl” refers to a ((C1-C6)alkyl)-NH—C(═O)—((C3-C6)cycloalkyl)- group in which the alkyl moiety and the cycloalkyl moiety are as defined above. Examples thereof include 1-(methylaminocarbonyl)cyclopropyl, 1-(ethylaminocarbonyl)cyclopropyl, 1-(n-propylaminocarbonyl)cyclopropyl, 1-(isopropylaminocarbonyl)cyclopropyl, 1-(tert-butylaminocarbonyl)cyclopropyl, 1-(methylaminocarbonyl)cyclobutyl, 1-(methylaminocarbonyl)cyclopentyl, and 1-(methylaminocarbonyl)cyclohexyl groups.

In the present invention, unless otherwise specified, the “heterocycloalkyl (C3-C6)cycloalkyl” refers to a (3- to 6-membered heterocycloalkyl)-((C3-C6)cycloalkyl group)- group in which the heterocycloalkyl moiety and the cycloalkyl moiety are as defined above. Examples thereof include 1-(oxetane-2-yl)cyclopropyl, 1-(oxetane-3-yl)cyclopropyl, 1-(tetrahydrofuran-2-yl)cyclopropyl, 1-(tetrahydrofuran-3-yl)cyclopropyl, 1-(tetrahydrothiophene-3-yl)cyclopropyl, 1-(tetrahydropyran-2-yl)cyclopropyl, 1-(tetrahydropyran-3-yl)cyclopropyl, 1-(tetrahydropyran-4-yl)cyclopropyl, 1-(tetrahydrothiopyran-4-yl)cyclopropyl, 1-(morpholine-4-yl)cyclopropyl, 1-(azetidine-3-yl)cyclopropyl, 1-(pyrrolidine-3-yl)cyclopropyl, and 1-(piperidine-4-yl)cyclopropyl groups.

In the present invention, unless otherwise specified, the “(C4-C8)bicycloalkyl” refers to a bicyclic cyclic alkyl group having 4 to 8 carbon atoms. Examples thereof include bicyclo[1.1.0]butane-2-yl, bicyclo[1.1.1]pentane-1-yl, bicyclo [2.1.0]pentane-2-yl, and bicyclo[2.1.0]pentane-5-yl groups.

In the present invention, unless otherwise specified, the “(C1-C6)alkylsulfinyl (C1-C6)alkyl” refers to a ((C1-C6)alkylsulfinyl)-((C1-C6)alkyl)- group in which the alkylsulfinyl moiety and the alkyl moiety are as defined above. Examples thereof include methylsulfinylmethyl, ethylsulfinylmethyl, n-propylsulfinylmethyl, isopropylsulfinylmethyl, 1-(methylsulfinyl)ethyl, 2-(methylsulfinyl)ethyl, 2-(ethylsulfinyl)ethyl, 1-(n-propylsulfinyl)ethyl, 2-(n-propylsulfinyl)ethyl, 1-(isopropylsulfinyl)ethyl, 2-(isopropylsulfinyl)ethyl, 1-(methylsulfinyl)propyl, 2-(methylsulfinyl)propyl, 3-(methylsulfinyl)propyl, 1-(ethylsulfinyl)propyl, 2-(ethylsulfinyl)propyl, 3-(ethylsulfinyl)propyl, 1-(n-propylsulfinyl)propyl, 2-(n-propylsulfinyl)propyl, 3-(n-propylsulfinyl)propyl, 1-(methylsulfinyl)butyl, 2-(methylsulfinyl)butyl, 3-(methylsulfinyl)butyl, 4-(methylsulfinyl)butyl, 1-(methylsulfinyl)pentyl, 2-(methylsulfinyl)pentyl, 3-(methylsulfinyl)pentyl, 4-(methylsulfinyl)pentyl, 5-(methylsulfinyl)pentyl, 2-(n-butylsulfinyl)ethyl, 2-(isobutylsulfinyl)ethyl, 2-(sec-butylsulfinyl)ethyl, 2-(tert-butylsulfinyl)ethyl, pentylsulfinylmethyl, and hexylsulfinylmethyl groups.

In the present invention, unless otherwise specified, the “(C1-C6)alkylsulfonyl (C1-C6)alkyl” refers to a ((C1-C6)alkylsulfonyl)-((C1-C6)alkyl)- group in which the alkylsulfonyl moiety and the alkyl moiety are as defined above. Examples thereof include methylsulfonylmethyl, ethylsulfonylmethyl, n-propylsulfonylmethyl, isopropylsulfonylmethyl, 1-(methylsulfonyl)ethyl, 2-(methylsulfonyl)ethyl, 2-(ethylsulfonyl)ethyl, 1-(n-propylsulfonyl)ethyl, 2-(n-propylsulfonyl)ethyl, 1-(isopropylsulfonyl)ethyl, 2-(isopropylsulfonyl)ethyl, 1-(methylsulfonyl)propyl, 2-(methylsulfonyl)propyl, 3-(methylsulfonyl)propyl, 1-(ethylsulfonyl)propyl, 2-(ethylsulfonyl)propyl, 3-(ethylsulfonyl)propyl, 1-(n-propylsulfonyl)propyl, 2-(n-propylsulfonyl)propyl, 3-(n-propylsulfonyl)propyl, 1-(methylsulfonyl)butyl, 2-(methylsulfonyl)butyl, 3-(methylsulfonyl)butyl, 4-(methylsulfonyl)butyl, 1-(methylsulfonyl)pentyl, 2-(methylsulfonyl)pentyl, 3-(methylsulfonyl)pentyl, 4-(methylsulfonyl)pentyl, 5-(methylsulfonyl)pentyl, 2-(n-butylsulfonyl)ethyl, 2-(isobutylsulfonyl)ethyl, 2-(sec-butylsulfonyl)ethyl, 2-(tert-butylsulfonyl)ethyl, pentylsulfonylmethyl, and hexylsulfonylmethyl groups.

In the present invention, unless otherwise specified, the “(C1-C6)alkoxy (C1-C6)alkoxy (C1-C6)alkyl” refers to a ((C1-C6)alkoxy)-((C1-C6)alkoxy)-((C1-C6)alkyl)- group in which the alkoxy moiety and the alkyl moiety are as defined above. Examples thereof include (2-methoxyethoxy)methyl and (2-ethoxyethoxy)methyl groups.

In the present invention, unless otherwise specified, the “cyano (C1-C6)alkoxy (C1-C6)alkyl” refers to a (cyano)-((C1-C6)alkoxy)-((C1-C6)alkyl)- group in which the alkoxy moiety and the alkyl moiety are as defined above. Examples thereof include a (2-cyanoethoxy)methyl group.

In the present invention, unless otherwise specified, the “(C1-C6)alkylthio (C1-C6)alkoxy (C1-C6)alkyl” refers to a ((C1-C6)alkylthio)-((C1-C6)alkoxy)-((C1-C6)alkyl)- group in which the alkylthio moiety, the alkoxy moiety, and the alkyl moiety are as defined above. Examples thereof include (2-methylthioethoxy)methyl and (2-ethylthioethoxy)methyl groups.

In the present invention, unless otherwise specified, the “(C1-C6)alkylsulfinyl (C1-C6)alkoxy (C1-C6)alkyl” refers to a ((C1-C6)alkylsulfinyl)-((C1-C6)alkoxy)-((C1-C6)alkyl)- group in which the alkylsulfinyl moiety, the alkoxy moiety, and the alkyl moiety are as defined above. Examples thereof include (2-methylsulfinylethoxy)methyl and (2-ethylsulfinylethoxy)methyl groups.

In the present invention, unless otherwise specified, the “(C1-C6)alkylsulfonyl (C1-C6)alkoxy (C1-C6)alkyl” refers to a ((C1-C6)alkylsulfonyl)-((C1-C6)alkoxy)-((C1-C6)alkyl)- group in which the alkylsulfonyl moiety, the alkoxy moiety, and the alkyl moiety are as defined above. Examples thereof include (2-methylsulfonylethoxy)methyl and (2-ethylsulfonylethoxy)methyl groups.

In the present invention, unless otherwise specified, the “(C1-C6)alkylcarbonyloxy (C1-C6)alkyl” refers to a ((C1-C6)alkyl)-C(═O)O—((C1-C6)alkyl)- group in which the alkyl moiety is as defined above. Examples thereof include acetoxymethyl, propionyloxymethyl, isobutyryloxymethyl, and pivaloyloxymethyl groups.

In the present invention, unless otherwise specified, the “(C1-C6)alkoxycarbonyloxy (C1-C6)alkyl” refers to a ((C1-C6)alkyl)-O—C(═O)—O—((C1-C6)alkyl)- group in which the alkyl moiety is as defined above. Examples thereof include methoxycarbonyloxymethyl, ethoxycarbonyloxymethyl, isopropoxycarbonyloxymethyl, tert-butoxycarbonyloxymethyl, and isobutoxycarbonyloxymethyl groups.

In the present invention, unless otherwise specified, the “(C1-C6)alkoxycarbonylamino (C1-C6)alkyl” refers to a ((C1-C6)alkyl)-O—C(═O)—NH—((C1-C6)alkyl)- group in which the alkyl moiety is as defined above. Examples thereof include methoxycarbonylaminomethyl, ethoxycarbonylaminomethyl, isopropoxycarbonylaminomethyl, tert-butoxycarbonylaminomethyl, and isobutoxycarbonylaminomethyl groups.

In the present invention, unless otherwise specified, the “(C2-C6)alkynyl (C3-C6)cycloalkyl” refers to a ((C2-C6)alkynyl)-((C1-C6)cycloalkyl)- group in which the alkynyl moiety and the cycloalkyl moiety are as defined above. Examples thereof include 1-(ethynyl)cyclopropyl, 1-(1-propynyl) cyclopropyl, 1-(2-propynyl)cyclopropyl, 1-(1-butynyl)cyclopropyl, 1-(2-butynyl)cyclopropyl, 1-(3-butynyl)cyclopropyl, and 1-(1-pentynyl)cyclopropyl groups.

In the present invention, unless otherwise specified, the “(C2-C6)alkenyloxycarbonyl” refers to a ((C1-C6)alkenyl)-O—C(═O)— group in which the alkenyl moiety is as defined above. Examples thereof include 1-propenyloxycarbonyl, isopropenyloxycarbonyl, 2-propenyloxycarbonyl, 2-butenyloxycarbonyl, 3-butenyloxycarbonyl, 2-pentenyloxycarbonyl, 3-pentenyloxycarbonyl, 4-pentenyloxycarbonyl, 2-methyl-2-butenyloxycarbonyl, 2,4-pentadienyloxycarbonyl, 2-hexenyloxycarbonyl, 3-hexenyloxycarbonyl, 4-hexenyloxycarbonyl, 5-hexenyloxycarbonyl, and 2,4-hexadienyloxycarbonyl groups.

In the present invention, unless otherwise specified, the “tri((C1-C6)alkyl)silyl (C1-C6)alkyl” refers to a ((C1-C6)alkyl)₃-Si—((C1-C6)alkyl)- group in which the tri(C₁-C₆ alkyl)silyl moiety and the alkyl moiety are as defined above, and three alkyl groups of the ((C1-C6)alkyl)₃ moiety are optionally identical or different from each other. Examples thereof include trimethylsilylmethyl, 1-trimethylsilylethyl, 1-trimethylsilyipropyl, 1-trimethylsilylbutyl, 1-trimethylsilylpentyl, 1-trimethylsilylhexyl, triethylsilylmethyl, 1-triethylsilylethyl, triisopropylsilylmethyl, 1-triisopropylsilylethyl, dimethylisilylmethyl, and tert-butyldimethylsilylmethyl groups.

In the present invention, unless otherwise specified, the “(C1-C6)alkoxymethyl” refers to a ((C1-C6)alkoxy)-methyl group in which the alkoxy moiety is as defined above. Examples thereof include methoxymethyl, ethoxymethyl, n-propoxymethyl, isopropoxymethyl, n-butoxymethyl, isobutoxymethyl, sec-butoxymethyl, tert-butoxymethyl, n-pentoxymethyl, 1-methylbutoxymethyl, 2-methylbutoxymethyl, 3-methylbutoxymethyl, 1-ethylpropoxymethyl, 1,1-dimethylpropoxymethyl, 1,2-dimethylpropoxymethyl, and 2,2-dimethylpropoxymethyl groups.

In the present invention, unless otherwise specified, the “(C1-C12)alkylene” refers to alkylene having 1 to 12 carbon atoms. Examples thereof include methylene, ethylene, n-propylene, n-butylene, n-hexylene, n-heptylene, n-octylene, and n-dodecylene.

In the present invention, unless otherwise specified, the “(C3-C6)cycloalkylene” refers to cycloalkylene having 3 to 6 carbon atoms. Examples thereof include cyclopropylene, cyclobutylene, cyclopentylene, and cyclohexylene.

In the present invention, unless otherwise specified, the “(C1-C6)alkyleneoxy (C1-C12)alkylene” refers to ((C1-C6)alkylene)-O—(C₁-C₁₂ alkylene) in which the alkylene moiety is as defined above. Examples thereof include methyleneoxymethylene (—CH₂—O—CH₂—), methyleneoxyethylene (—CH₂—O—CH₂CH₂—), and ethyleneoxyethylene (—CH₂CH₂—O—CH₂CH₂—).

In the present invention, unless otherwise specified, the “(C2-C6)alkenylene” refers to alkenylene having 2 to 6 carbon atoms. Examples thereof include vinylene, propenylene, and butenylene.

In the present invention, unless otherwise specified, the “(C2-C6)alkynylene” refers to alkynylene having 2 to 6 carbon atoms. Examples thereof include ethynylene, propynylene, butynylene, and pentynylene.

In the present invention, “optionally mono-substituted or poly-substituted with R⁶” means “optionally substituted with one or two or more R⁶”. When the group is substituted with two or more R⁶, the two or more R⁶ are identical to or different from each other, and each represent a halogen atom; a (C1-C6)alkyl; a (C1-C6)haloalkyl; a (C3-C6)cycloalkyl; a (C3-C6)halocycloalkyl; a hydroxy; a (C1-C6)alkoxy; a (C1-C6)haloalkoxy; a (C3-C6)cycloalkoxy; a (C3-C6)halocycloalkoxy; a (C1-C6)alkoxy (C1-C6)alkoxy; a (C1-C6)haloalkoxy (C1-C6)alkoxy; a (C1-C6)haloalkoxy (C1-C6)haloalkoxy; a thiol; a (C1-C6)alkylthio; a (C1-C6)haloalkylthio; a (C2-C6)alkenylthio; a (C2-C6)haloalkenylthio; a (C3-C6)cycloalkylthio; a (C3-C6)halocycloalkylthio; a (C3-C6)cycloalkyl (C1-C6)alkylthio; a (C3-C6)halocycloalkyl (C1-C6)alkylthio; a tri((C1-C6)alkyl)silyl (C1-C6)alkylthio; a (C1-C6)alkylthio (C1-C6)alkoxy; a (C1-C6)haloalkylthio (C1-C6)alkoxy; a (C1-C6)alkylsulfinyl; a (C1-C6)haloalkylsulfinyl; a (C3-C6)cycloalkylsulfinyl; a (C3-C6)halocycloalkylsulfinyl; a (C1-C6)alkylsulfonyl; a (C1-C6)haloalkylsulfonyl; a (C3-C6)cycloalkylsulfonyl; a (C3-C6)halocycloalkylsulfonyl; a formyl; a (C1-C6)alkylcarbonyl; a (C1-C6)haloalkylcarbonyl; a benzoyl (the benzoyl is optionally mono-substituted or poly-substituted with R⁷); a (C1-C6)alkoxycarbonyl; a (C1-C6)haloalkoxycarbonyl; a (C1-C6)alkylaminocarbonyl (the amino moiety is optionally substituted with R¹⁰); a (C1-C6)haloalkylaminocarbonyl (the amino moiety is optionally substituted with R¹⁰); a (C1-C6)alkylaminocarbonylthio (the amino moiety is optionally substituted with R¹⁰); a (C1-C6)haloalkylaminocarbonylthio (the amino moiety is optionally substituted with R¹¹); a formyloxy; a (C1-C6)alkylcarbonyloxy; a (C1-C6)haloalkylcarbonyloxy; a benzoyloxy (the benzoyloxy is optionally mono-substituted or poly-substituted with R⁷); a (C1-C6)alkylsulfinyloxy; a (C1-C6)haloalkylsulfinyloxy; a (C3-C6)cycloalkylsulfinyloxy; a (C3-C6)halocycloalkylsulfinyloxy; a phenylsulfinyloxy (the phenylsulfinyloxy is optionally mono-substituted or poly-substituted with R⁷); a (C1-C6)alkylsulfonyloxy; a (C1-C6)haloalkylsulfonyloxy; a (C3-C6)cycloalkylsulfonyloxy; a (C3-C6)halocycloalkylsulfonyloxy; a phenylsulfonyloxy (the phenyl moiety is optionally mono-substituted or poly-substituted with R⁷); an amino; a (C1-C6)alkylamino (the amino moiety is optionally substituted with R¹⁰); a (C1-C6)haloalkylamino (the amino moiety is optionally substituted with R¹⁰); a (C1-C6)alkylcarbonylamino (the amino moiety is optionally substituted with R¹⁰); a (C1-C6)haloalkylcarbonylamino (the amino moiety is optionally substituted with R¹⁰); a phenylcarbonylamino (the phenyl moiety is optionally mono-substituted or poly-substituted with R⁷, and the amino moiety is optionally substituted with R¹⁰); a (C1-C6)alkoxycarbonylamino (the amino moiety is optionally substituted with R¹⁰); a (C1-C6)haloalkoxycarbonylamino (the amino moiety is optionally substituted with R¹⁰); a (C1-C6)alkylaminocarbonylamino (the amino moiety is optionally substituted with R¹⁰); a (C1-C6)haloalkylaminocarbonylamino (the amino moiety is optionally substituted with R¹⁰); a (C1-C6)alkylsulfinylamino (the amino moiety is optionally substituted with R¹⁰); a (C1-C6)haloalkylsulfinylamino (the amino moiety is optionally substituted with R¹⁰); a phenylsulfinylamino (the phenyl moiety is optionally mono-substituted or poly-substituted with R⁷, and the amino moiety is optionally substituted with R¹⁰); a (C1-C6)alkylsulfonylamino (the amino moiety is optionally substituted with R¹⁰); a (C1-C6)haloalkylsulfonylamino (the amino moiety is optionally substituted with R¹⁰); a phenylsulfonylamino (the phenyl moiety is optionally mono-substituted or poly-substituted with R⁷, and the amino moiety is optionally substituted with R¹⁰); a (C1-C6)alkoxyimino; a tri((C1-C6)alkyl)silyl; a phenyl (the phenyl is optionally mono-substituted or poly-substituted with R⁷); a phenoxy (the phenoxy is optionally mono-substituted or poly-substituted with R⁷); a phenyl (C1-C6)alkoxy (the phenyl moiety is optionally mono-substituted or poly-substituted with R⁷); a phenylthio (the phenylthio is optionally mono-substituted or poly-substituted with R⁷); a phenylsulfinyl (the phenylsulfinyl is optionally mono-substituted or poly-substituted with R⁷); a phenylsulfonyl (the phenylsulfonyl is optionally mono-substituted or poly-substituted with R⁷); a phenyl (C1-C6)alkylthio (the phenyl moiety is optionally mono-substituted or poly-substituted with R⁷); a phenyl (C1-C6)alkylsulfinyl (the phenyl moiety is optionally mono-substituted or poly-substituted with R⁷); a phenyl (C1-C6)alkylsulfonyl (the phenyl moiety is optionally mono-substituted or poly-substituted with R⁷); a —O—N═C(R⁸)(R⁹) group; an adamantyl; an azetidinyl (a nitrogen atom of the azetidinyl is optionally substituted with R¹⁰); a pyrrolidinyl (a nitrogen atom of the pyrrolidinyl is optionally substituted with R¹⁰); a piperidinyl (a nitrogen atom of the piperidinyl is optionally substituted with R¹⁰); a 1,3-dioxolanyl; a 1,3-dioxanyl; a pyrrolyl (the pyrrolyl is optionally mono-substituted or poly-substituted with R⁷); a pyrazolyl (the pyrazolyl is optionally mono-substituted or poly-substituted with R⁷); an imidazolyl (the imidazolyl is optionally mono-substituted or poly-substituted with R⁷); a triazolyl (the triazolyl is optionally mono-substituted or poly-substituted with R⁷); an oxazolyl (the oxazolyl is optionally mono-substituted or poly-substituted with R⁷); an isoxazolyl (the isoxazolyl is optionally mono-substituted or poly-substituted with R⁷); a thiazolyl (the thiazolyl is optionally mono-substituted or poly-substituted with R⁷); an isothiazolyl (the isothiazolyl is optionally mono-substituted or poly-substituted with R⁷); a pyridyl (the pyridyl is optionally mono-substituted or poly-substituted with R⁷, and further a nitrogen atom of the pyridyl is optionally oxidized to form an N-oxide); a pyrimidinyl (the pyrimidinyl is optionally mono-substituted or poly-substituted with R⁷); a pyridyloxy (the pyridyloxy is optionally mono-substituted or poly-substituted with R⁷); a tetrahydrofuranyl (the tetrahydrofuranyl is optionally mono-substituted or poly-substituted with R⁷); a 1,3-dioxoisoindolinyl group (the dioxoisoindolinyl group is optionally mono-substituted or poly-substituted with R⁷); a cyano; a nitro; a carboxy; a thiocyanato; or an aminooxy.

In the present invention, “optionally mono-substituted or poly-substituted with R⁷” means “optionally substituted with one or two or more R⁷”. When the group is substituted with two or more R⁷, the two or more R⁷ are identical to or different from each other, and each represent a halogen atom; a (C1-C6)alkyl; a (C1-C6)haloalkyl; a (C3-C6)cycloalkyl; a (C3-C6)halocycloalkyl; a (C3-C6)cycloalkyl (C1-C6)alkyl; a (C3-C6)halocycloalkyl (C1-C6)alkyl; a (C1-C6)alkoxy; a (C1-C6)haloalkoxy; a (C1-C6)alkylthio; a (C1-C6)haloalkylthio; a (C1-C6)alkylsulfinyl; a (C1-C6)haloalkylsulfinyl; a (C1-C6)alkylsulfonyl; a (C1-C6)haloalkylsulfonyl; a (C1-C6)alkylthio (C1-C6)alkyl; a (C1-C6)haloalkylthio (C1-C6)alkyl; a (C1-C6)alkylsulfonyloxy; a (C1-C6)haloalkylsulfonyloxy; a phenyl (the phenyl is optionally mono-substituted or poly-substituted with a halogen atom, an alkyl, or a haloalkyl); a phenyl (C1-C6)alkyl; a phenyl (C1-C6)alkoxy; a cyano; or a nitro group.

In the present invention, the agriculturally acceptable salt or salt means a salt of any compound of the present invention of formula [I], [II], or [III], which contains a hydroxyl group, a carboxyl group, an amino group, or the like, or a nitrogen atom of a pyridine ring in the structure thereof, with a metal or an organic base, or with a mineral acid or an organic acid. Examples of the metal include an alkali metal such as sodium or potassium, or an alkaline earth metal such as magnesium or calcium. Examples of the organic base include triethylamine or diisopropylamine. Examples of the mineral acid include phosphoric acid, hydrochloric acid, hydrobromic acid, hydroiodic acid, boric acid, or sulfuric acid. Examples of the organic acid include formic acid, acetic acid, lactic acid, ascorbic acid, succinic acid, fumaric acid, maleic acid, oxalic acid, citric acid, benzoic acid, salicylic acid, tartaric acid, methanesulfonic acid, 4-toluenesulfonic acid, and trifluoromethanesulfonic acid.

Next, representative compounds of the compounds included in the 3-alkoxybenzamide derivative of the present invention of formula [I] are shown in Tables 1 to 1062, representative compounds of the compounds included in the hydroxybenzamide derivative of the present invention of formula [II] are shown in Tables 1063 to 1064, and representative compounds of the compounds included in the alkoxybenzoic acid derivative of the present invention of formula [III] are shown in Tables 1065 to 1233. However, the compounds included in the derivatives of the present invention are not limited thereto. In addition, the compound numbers in the tables are referred to in the following description.

The compounds included in the 3-alkoxybenzamide derivative, alkoxybenzoic acid derivative, or hydroxybenzderivative of the present invention may have E- and Z-form geometric isomers depending on the type of the substituent, but the present invention includes a mixture containing these E-, Z-, or E- and Z-forms in an arbitrary ratio. In addition, the compounds included in the present invention may have optical isomers resulting from the presence of one or two or more asymmetric carbon atoms and asymmetric sulfur atoms, but the present invention includes all optically active forms, racemates, or diastereomers.

The following notations in the tables in the present specification represent, for example, corresponding groups as follows.
Me: Methyl;
Et: Ethyl;
n-Pr: n-Propyl;
i-Pr: Isopropyl;
n-Bu: n-Butyl;
t-Bu: tert-Butyl;
n-Pen: n-Pentyl;
t-Pen: tert-Pentyl;
n-Hex: n-Hexyl;
n-Heptyl: n-Heptyl;
n-Octyl: n-Octyl;
n-Nonyl: n-Nonyl;
n-Decyl: n-Decyl;
c-Pr: Cyclopropyl;
c-Pen: Cyclopentyl;
c-Hex: Cyclohexyl;
CHF₂: Difluoromethyl;
CF₃: Trifluoromethyl;
Ph(4-CF₃): 4-Trifluoromethylphenyl;
Ph(2-F-4-CF₃): 2-Fluoro-4-trifluoromethylphenyl;
Ph(3,4,5-Cl₃): 3,4,5-Trichlorophenyl;
c-Pr(2,2-F₂): 2,2-Difluorocyclopropyl;
N(Phth): Phthalimide;
THF: Tetrahydrofuran;

Lengthy table referenced here
US20260363240A1-20261119-T00001
Please refer to the end of the specification for access instructions.

Lengthy table referenced here
US20260363240A1-20261119-T00002
Please refer to the end of the specification for access instructions.

Lengthy table referenced here
US20260363240A1-20261119-T00003
Please refer to the end of the specification for access instructions.

Lengthy table referenced here
US20260363240A1-20261119-T00004
Please refer to the end of the specification for access instructions.

Lengthy table referenced here
US20260363240A1-20261119-T00005
Please refer to the end of the specification for access instructions.

Lengthy table referenced here
US20260363240A1-20261119-T00006
Please refer to the end of the specification for access instructions.

Lengthy table referenced here
US20260363240A1-20261119-T00007
Please refer to the end of the specification for access instructions.

Lengthy table referenced here
US20260363240A1-20261119-T00008
Please refer to the end of the specification for access instructions.

Lengthy table referenced here
US20260363240A1-20261119-T00009
Please refer to the end of the specification for access instructions.

Lengthy table referenced here
US20260363240A1-20261119-T00010
Please refer to the end of the specification for access instructions.

Lengthy table referenced here
US20260363240A1-20261119-T00011
Please refer to the end of the specification for access instructions.

Lengthy table referenced here
US20260363240A1-20261119-T00012
Please refer to the end of the specification for access instructions.

Lengthy table referenced here
US20260363240A1-20261119-T00013
Please refer to the end of the specification for access instructions.

Lengthy table referenced here
US20260363240A1-20261119-T00014
Please refer to the end of the specification for access instructions.

Lengthy table referenced here
US20260363240A1-20261119-T00015
Please refer to the end of the specification for access instructions.

Lengthy table referenced here
US20260363240A1-20261119-T00016
Please refer to the end of the specification for access instructions.

Lengthy table referenced here
US20260363240A1-20261119-T00017
Please refer to the end of the specification for access instructions.

Lengthy table referenced here
US20260363240A1-20261119-T00018
Please refer to the end of the specification for access instructions.

Lengthy table referenced here
US20260363240A1-20261119-T00019
Please refer to the end of the specification for access instructions.

Lengthy table referenced here
US20260363240A1-20261119-T00020
Please refer to the end of the specification for access instructions.

Lengthy table referenced here
US20260363240A1-20261119-T00021
Please refer to the end of the specification for access instructions.

Lengthy table referenced here
US20260363240A1-20261119-T00022
Please refer to the end of the specification for access instructions.

Lengthy table referenced here
US20260363240A1-20261119-T00023
Please refer to the end of the specification for access instructions.

Lengthy table referenced here
US20260363240A1-20261119-T00024
Please refer to the end of the specification for access instructions.

Lengthy table referenced here
US20260363240A1-20261119-T00025
Please refer to the end of the specification for access instructions.

Lengthy table referenced here
US20260363240A1-20261119-T00026
Please refer to the end of the specification for access instructions.

Lengthy table referenced here
US20260363240A1-20261119-T00027
Please refer to the end of the specification for access instructions.

Lengthy table referenced here
US20260363240A1-20261119-T00028
Please refer to the end of the specification for access instructions.

Lengthy table referenced here
US20260363240A1-20261119-T00029
Please refer to the end of the specification for access instructions.

Lengthy table referenced here
US20260363240A1-20261119-T00030
Please refer to the end of the specification for access instructions.

Lengthy table referenced here
US20260363240A1-20261119-T00031
Please refer to the end of the specification for access instructions.

Lengthy table referenced here
US20260363240A1-20261119-T00032
Please refer to the end of the specification for access instructions.

Lengthy table referenced here
US20260363240A1-20261119-T00033
Please refer to the end of the specification for access instructions.

Lengthy table referenced here
US20260363240A1-20261119-T00034
Please refer to the end of the specification for access instructions.

Lengthy table referenced here
US20260363240A1-20261119-T00035
Please refer to the end of the specification for access instructions.

Lengthy table referenced here
US20260363240A1-20261119-T00036
Please refer to the end of the specification for access instructions.

Lengthy table referenced here
US20260363240A1-20261119-T00037
Please refer to the end of the specification for access instructions.

Lengthy table referenced here
US20260363240A1-20261119-T00038
Please refer to the end of the specification for access instructions.

Lengthy table referenced here
US20260363240A1-20261119-T00039
Please refer to the end of the specification for access instructions.

Lengthy table referenced here
US20260363240A1-20261119-T00040
Please refer to the end of the specification for access instructions.

Lengthy table referenced here
US20260363240A1-20261119-T00041
Please refer to the end of the specification for access instructions.

Lengthy table referenced here
US20260363240A1-20261119-T00042
Please refer to the end of the specification for access instructions.

Lengthy table referenced here
US20260363240A1-20261119-T00043
Please refer to the end of the specification for access instructions.

Lengthy table referenced here
US20260363240A1-20261119-T00044
Please refer to the end of the specification for access instructions.

Lengthy table referenced here
US20260363240A1-20261119-T00045
Please refer to the end of the specification for access instructions.

Lengthy table referenced here
US20260363240A1-20261119-T00046
Please refer to the end of the specification for access instructions.

Lengthy table referenced here
US20260363240A1-20261119-T00047
Please refer to the end of the specification for access instructions.

Lengthy table referenced here
US20260363240A1-20261119-T00048
Please refer to the end of the specification for access instructions.

Lengthy table referenced here
US20260363240A1-20261119-T00049
Please refer to the end of the specification for access instructions.

Lengthy table referenced here
US20260363240A1-20261119-T00050
Please refer to the end of the specification for access instructions.

Lengthy table referenced here
US20260363240A1-20261119-T00051
Please refer to the end of the specification for access instructions.

Lengthy table referenced here
US20260363240A1-20261119-T00052
Please refer to the end of the specification for access instructions.

Lengthy table referenced here
US20260363240A1-20261119-T00053
Please refer to the end of the specification for access instructions.

Lengthy table referenced here
US20260363240A1-20261119-T00054
Please refer to the end of the specification for access instructions.

Lengthy table referenced here
US20260363240A1-20261119-T00055
Please refer to the end of the specification for access instructions.

Lengthy table referenced here
US20260363240A1-20261119-T00056
Please refer to the end of the specification for access instructions.

Lengthy table referenced here
US20260363240A1-20261119-T00057
Please refer to the end of the specification for access instructions.

Lengthy table referenced here
US20260363240A1-20261119-T00058
Please refer to the end of the specification for access instructions.

Lengthy table referenced here
US20260363240A1-20261119-T00059
Please refer to the end of the specification for access instructions.

Lengthy table referenced here
US20260363240A1-20261119-T00060
Please refer to the end of the specification for access instructions.

Lengthy table referenced here
US20260363240A1-20261119-T00061
Please refer to the end of the specification for access instructions.

Lengthy table referenced here
US20260363240A1-20261119-T00062
Please refer to the end of the specification for access instructions.

Lengthy table referenced here
US20260363240A1-20261119-T00063
Please refer to the end of the specification for access instructions.

Lengthy table referenced here
US20260363240A1-20261119-T00064
Please refer to the end of the specification for access instructions.

Lengthy table referenced here
US20260363240A1-20261119-T00065
Please refer to the end of the specification for access instructions.

Lengthy table referenced here
US20260363240A1-20261119-T00066
Please refer to the end of the specification for access instructions.

Lengthy table referenced here
US20260363240A1-20261119-T00067
Please refer to the end of the specification for access instructions.

Lengthy table referenced here
US20260363240A1-20261119-T00068
Please refer to the end of the specification for access instructions.

Lengthy table referenced here
US20260363240A1-20261119-T00069
Please refer to the end of the specification for access instructions.

Lengthy table referenced here
US20260363240A1-20261119-T00070
Please refer to the end of the specification for access instructions.

Lengthy table referenced here
US20260363240A1-20261119-T00071
Please refer to the end of the specification for access instructions.

Lengthy table referenced here
US20260363240A1-20261119-T00072
Please refer to the end of the specification for access instructions.

Lengthy table referenced here
US20260363240A1-20261119-T00073
Please refer to the end of the specification for access instructions.

Lengthy table referenced here
US20260363240A1-20261119-T00074
Please refer to the end of the specification for access instructions.

Lengthy table referenced here
US20260363240A1-20261119-T00075
Please refer to the end of the specification for access instructions.

Lengthy table referenced here
US20260363240A1-20261119-T00076
Please refer to the end of the specification for access instructions.

Lengthy table referenced here
US20260363240A1-20261119-T00077
Please refer to the end of the specification for access instructions.

Lengthy table referenced here
US20260363240A1-20261119-T00078
Please refer to the end of the specification for access instructions.

Lengthy table referenced here
US20260363240A1-20261119-T00079
Please refer to the end of the specification for access instructions.

Lengthy table referenced here
US20260363240A1-20261119-T00080
Please refer to the end of the specification for access instructions.

Lengthy table referenced here
US20260363240A1-20261119-T00081
Please refer to the end of the specification for access instructions.

Lengthy table referenced here
US20260363240A1-20261119-T00082
Please refer to the end of the specification for access instructions.

Lengthy table referenced here
US20260363240A1-20261119-T00083
Please refer to the end of the specification for access instructions.

Lengthy table referenced here
US20260363240A1-20261119-T00084
Please refer to the end of the specification for access instructions.

Lengthy table referenced here
US20260363240A1-20261119-T00085
Please refer to the end of the specification for access instructions.

Lengthy table referenced here
US20260363240A1-20261119-T00086
Please refer to the end of the specification for access instructions.

Lengthy table referenced here
US20260363240A1-20261119-T00087
Please refer to the end of the specification for access instructions.

Lengthy table referenced here
US20260363240A1-20261119-T00088
Please refer to the end of the specification for access instructions.

Lengthy table referenced here
US20260363240A1-20261119-T00089
Please refer to the end of the specification for access instructions.

Lengthy table referenced here
US20260363240A1-20261119-T00090
Please refer to the end of the specification for access instructions.

Lengthy table referenced here
US20260363240A1-20261119-T00091
Please refer to the end of the specification for access instructions.

Lengthy table referenced here
US20260363240A1-20261119-T00092
Please refer to the end of the specification for access instructions.

Lengthy table referenced here
US20260363240A1-20261119-T00093
Please refer to the end of the specification for access instructions.

Lengthy table referenced here
US20260363240A1-20261119-T00094
Please refer to the end of the specification for access instructions.

Lengthy table referenced here
US20260363240A1-20261119-T00095
Please refer to the end of the specification for access instructions.

Lengthy table referenced here
US20260363240A1-20261119-T00096
Please refer to the end of the specification for access instructions.

Lengthy table referenced here
US20260363240A1-20261119-T00097
Please refer to the end of the specification for access instructions.

Lengthy table referenced here
US20260363240A1-20261119-T00098
Please refer to the end of the specification for access instructions.

Lengthy table referenced here
US20260363240A1-20261119-T00099
Please refer to the end of the specification for access instructions.

Lengthy table referenced here
US20260363240A1-20261119-T00100
Please refer to the end of the specification for access instructions.

Lengthy table referenced here
US20260363240A1-20261119-T00101
Please refer to the end of the specification for access instructions.

Lengthy table referenced here
US20260363240A1-20261119-T00102
Please refer to the end of the specification for access instructions.

Lengthy table referenced here
US20260363240A1-20261119-T00103
Please refer to the end of the specification for access instructions.

Lengthy table referenced here
US20260363240A1-20261119-T00104
Please refer to the end of the specification for access instructions.

Lengthy table referenced here
US20260363240A1-20261119-T00105
Please refer to the end of the specification for access instructions.

Lengthy table referenced here
US20260363240A1-20261119-T00106
Please refer to the end of the specification for access instructions.

Lengthy table referenced here
US20260363240A1-20261119-T00107
Please refer to the end of the specification for access instructions.

Lengthy table referenced here
US20260363240A1-20261119-T00108
Please refer to the end of the specification for access instructions.

Lengthy table referenced here
US20260363240A1-20261119-T00109
Please refer to the end of the specification for access instructions.

Lengthy table referenced here
US20260363240A1-20261119-T00110
Please refer to the end of the specification for access instructions.

Lengthy table referenced here
US20260363240A1-20261119-T00111
Please refer to the end of the specification for access instructions.

Lengthy table referenced here
US20260363240A1-20261119-T00112
Please refer to the end of the specification for access instructions.

Lengthy table referenced here
US20260363240A1-20261119-T00113
Please refer to the end of the specification for access instructions.

Lengthy table referenced here
US20260363240A1-20261119-T00114
Please refer to the end of the specification for access instructions.

Lengthy table referenced here
US20260363240A1-20261119-T00115
Please refer to the end of the specification for access instructions.

Lengthy table referenced here
US20260363240A1-20261119-T00116
Please refer to the end of the specification for access instructions.

Lengthy table referenced here
US20260363240A1-20261119-T00117
Please refer to the end of the specification for access instructions.

Lengthy table referenced here
US20260363240A1-20261119-T00118
Please refer to the end of the specification for access instructions.

Lengthy table referenced here
US20260363240A1-20261119-T00119
Please refer to the end of the specification for access instructions.

Lengthy table referenced here
US20260363240A1-20261119-T00120
Please refer to the end of the specification for access instructions.

Lengthy table referenced here
US20260363240A1-20261119-T00121
Please refer to the end of the specification for access instructions.

Lengthy table referenced here
US20260363240A1-20261119-T00122
Please refer to the end of the specification for access instructions.

Lengthy table referenced here
US20260363240A1-20261119-T00123
Please refer to the end of the specification for access instructions.

Lengthy table referenced here
US20260363240A1-20261119-T00124
Please refer to the end of the specification for access instructions.

Lengthy table referenced here
US20260363240A1-20261119-T00125
Please refer to the end of the specification for access instructions.

Lengthy table referenced here
US20260363240A1-20261119-T00126
Please refer to the end of the specification for access instructions.

Lengthy table referenced here
US20260363240A1-20261119-T00127
Please refer to the end of the specification for access instructions.

Lengthy table referenced here
US20260363240A1-20261119-T00128
Please refer to the end of the specification for access instructions.

Lengthy table referenced here
US20260363240A1-20261119-T00129
Please refer to the end of the specification for access instructions.

Lengthy table referenced here
US20260363240A1-20261119-T00130
Please refer to the end of the specification for access instructions.

Lengthy table referenced here
US20260363240A1-20261119-T00131
Please refer to the end of the specification for access instructions.

Lengthy table referenced here
US20260363240A1-20261119-T00132
Please refer to the end of the specification for access instructions.

Lengthy table referenced here
US20260363240A1-20261119-T00133
Please refer to the end of the specification for access instructions.

Lengthy table referenced here
US20260363240A1-20261119-T00134
Please refer to the end of the specification for access instructions.

Lengthy table referenced here
US20260363240A1-20261119-T00135
Please refer to the end of the specification for access instructions.

Lengthy table referenced here
US20260363240A1-20261119-T00136
Please refer to the end of the specification for access instructions.

Lengthy table referenced here
US20260363240A1-20261119-T00137
Please refer to the end of the specification for access instructions.

Lengthy table referenced here
US20260363240A1-20261119-T00138
Please refer to the end of the specification for access instructions.

Lengthy table referenced here
US20260363240A1-20261119-T00139
Please refer to the end of the specification for access instructions.

Lengthy table referenced here
US20260363240A1-20261119-T00140
Please refer to the end of the specification for access instructions.

Lengthy table referenced here
US20260363240A1-20261119-T00141
Please refer to the end of the specification for access instructions.

Lengthy table referenced here
US20260363240A1-20261119-T00142
Please refer to the end of the specification for access instructions.

Lengthy table referenced here
US20260363240A1-20261119-T00143
Please refer to the end of the specification for access instructions.

Lengthy table referenced here
US20260363240A1-20261119-T00144
Please refer to the end of the specification for access instructions.

Lengthy table referenced here
US20260363240A1-20261119-T00145
Please refer to the end of the specification for access instructions.

Lengthy table referenced here
US20260363240A1-20261119-T00146
Please refer to the end of the specification for access instructions.

Lengthy table referenced here
US20260363240A1-20261119-T00147
Please refer to the end of the specification for access instructions.

Lengthy table referenced here
US20260363240A1-20261119-T00148
Please refer to the end of the specification for access instructions.

Lengthy table referenced here
US20260363240A1-20261119-T00149
Please refer to the end of the specification for access instructions.

Lengthy table referenced here
US20260363240A1-20261119-T00150
Please refer to the end of the specification for access instructions.

Lengthy table referenced here
US20260363240A1-20261119-T00151
Please refer to the end of the specification for access instructions.

Lengthy table referenced here
US20260363240A1-20261119-T00152
Please refer to the end of the specification for access instructions.

Lengthy table referenced here
US20260363240A1-20261119-T00153
Please refer to the end of the specification for access instructions.

Lengthy table referenced here
US20260363240A1-20261119-T00154
Please refer to the end of the specification for access instructions.

Lengthy table referenced here
US20260363240A1-20261119-T00155
Please refer to the end of the specification for access instructions.

Lengthy table referenced here
US20260363240A1-20261119-T00156
Please refer to the end of the specification for access instructions.

Lengthy table referenced here
US20260363240A1-20261119-T00157
Please refer to the end of the specification for access instructions.

Lengthy table referenced here
US20260363240A1-20261119-T00158
Please refer to the end of the specification for access instructions.

Lengthy table referenced here
US20260363240A1-20261119-T00159
Please refer to the end of the specification for access instructions.

Lengthy table referenced here
US20260363240A1-20261119-T00160
Please refer to the end of the specification for access instructions.

Lengthy table referenced here
US20260363240A1-20261119-T00161
Please refer to the end of the specification for access instructions.

Lengthy table referenced here
US20260363240A1-20261119-T00162
Please refer to the end of the specification for access instructions.

Lengthy table referenced here
US20260363240A1-20261119-T00163
Please refer to the end of the specification for access instructions.

Lengthy table referenced here
US20260363240A1-20261119-T00164
Please refer to the end of the specification for access instructions.

Lengthy table referenced here
US20260363240A1-20261119-T00165
Please refer to the end of the specification for access instructions.

Lengthy table referenced here
US20260363240A1-20261119-T00166
Please refer to the end of the specification for access instructions.

Lengthy table referenced here
US20260363240A1-20261119-T00167
Please refer to the end of the specification for access instructions.

Lengthy table referenced here
US20260363240A1-20261119-T00168
Please refer to the end of the specification for access instructions.

Lengthy table referenced here
US20260363240A1-20261119-T00169
Please refer to the end of the specification for access instructions.

Lengthy table referenced here
US20260363240A1-20261119-T00170
Please refer to the end of the specification for access instructions.

Lengthy table referenced here
US20260363240A1-20261119-T00171
Please refer to the end of the specification for access instructions.

Lengthy table referenced here
US20260363240A1-20261119-T00172
Please refer to the end of the specification for access instructions.

Lengthy table referenced here
US20260363240A1-20261119-T00173
Please refer to the end of the specification for access instructions.

Lengthy table referenced here
US20260363240A1-20261119-T00174
Please refer to the end of the specification for access instructions.

Lengthy table referenced here
US20260363240A1-20261119-T00175
Please refer to the end of the specification for access instructions.

Lengthy table referenced here
US20260363240A1-20261119-T00176
Please refer to the end of the specification for access instructions.

Lengthy table referenced here
US20260363240A1-20261119-T00177
Please refer to the end of the specification for access instructions.

Lengthy table referenced here
US20260363240A1-20261119-T00178
Please refer to the end of the specification for access instructions.

Lengthy table referenced here
US20260363240A1-20261119-T00179
Please refer to the end of the specification for access instructions.

Lengthy table referenced here
US20260363240A1-20261119-T00180
Please refer to the end of the specification for access instructions.

Lengthy table referenced here
US20260363240A1-20261119-T00181
Please refer to the end of the specification for access instructions.

Lengthy table referenced here
US20260363240A1-20261119-T00182
Please refer to the end of the specification for access instructions.

Lengthy table referenced here
US20260363240A1-20261119-T00183
Please refer to the end of the specification for access instructions.

Lengthy table referenced here
US20260363240A1-20261119-T00184
Please refer to the end of the specification for access instructions.

Lengthy table referenced here
US20260363240A1-20261119-T00185
Please refer to the end of the specification for access instructions.

Lengthy table referenced here
US20260363240A1-20261119-T00186
Please refer to the end of the specification for access instructions.

Lengthy table referenced here
US20260363240A1-20261119-T00187
Please refer to the end of the specification for access instructions.

Lengthy table referenced here
US20260363240A1-20261119-T00188
Please refer to the end of the specification for access instructions.

Lengthy table referenced here
US20260363240A1-20261119-T00189
Please refer to the end of the specification for access instructions.

Lengthy table referenced here
US20260363240A1-20261119-T00190
Please refer to the end of the specification for access instructions.

Lengthy table referenced here
US20260363240A1-20261119-T00191
Please refer to the end of the specification for access instructions.

Lengthy table referenced here
US20260363240A1-20261119-T00192
Please refer to the end of the specification for access instructions.

Lengthy table referenced here
US20260363240A1-20261119-T00193
Please refer to the end of the specification for access instructions.

Lengthy table referenced here
US20260363240A1-20261119-T00194
Please refer to the end of the specification for access instructions.

Lengthy table referenced here
US20260363240A1-20261119-T00195
Please refer to the end of the specification for access instructions.

Lengthy table referenced here
US20260363240A1-20261119-T00196
Please refer to the end of the specification for access instructions.

Lengthy table referenced here
US20260363240A1-20261119-T00197
Please refer to the end of the specification for access instructions.

Lengthy table referenced here
US20260363240A1-20261119-T00198
Please refer to the end of the specification for access instructions.

Lengthy table referenced here
US20260363240A1-20261119-T00199
Please refer to the end of the specification for access instructions.

Lengthy table referenced here
US20260363240A1-20261119-T00200
Please refer to the end of the specification for access instructions.

Lengthy table referenced here
US20260363240A1-20261119-T00201
Please refer to the end of the specification for access instructions.

Lengthy table referenced here
US20260363240A1-20261119-T00202
Please refer to the end of the specification for access instructions.

Lengthy table referenced here
US20260363240A1-20261119-T00203
Please refer to the end of the specification for access instructions.

Lengthy table referenced here
US20260363240A1-20261119-T00204
Please refer to the end of the specification for access instructions.

Lengthy table referenced here
US20260363240A1-20261119-T00205
Please refer to the end of the specification for access instructions.

Lengthy table referenced here
US20260363240A1-20261119-T00206
Please refer to the end of the specification for access instructions.

Lengthy table referenced here
US20260363240A1-20261119-T00207
Please refer to the end of the specification for access instructions.

Lengthy table referenced here
US20260363240A1-20261119-T00208
Please refer to the end of the specification for access instructions.

Lengthy table referenced here
US20260363240A1-20261119-T00209
Please refer to the end of the specification for access instructions.

Lengthy table referenced here
US20260363240A1-20261119-T00210
Please refer to the end of the specification for access instructions.

Lengthy table referenced here
US20260363240A1-20261119-T00211
Please refer to the end of the specification for access instructions.

Lengthy table referenced here
US20260363240A1-20261119-T00212
Please refer to the end of the specification for access instructions.

Lengthy table referenced here
US20260363240A1-20261119-T00213
Please refer to the end of the specification for access instructions.

Lengthy table referenced here
US20260363240A1-20261119-T00214
Please refer to the end of the specification for access instructions.

Lengthy table referenced here
US20260363240A1-20261119-T00215
Please refer to the end of the specification for access instructions.

Lengthy table referenced here
US20260363240A1-20261119-T00216
Please refer to the end of the specification for access instructions.

Lengthy table referenced here
US20260363240A1-20261119-T00217
Please refer to the end of the specification for access instructions.

Lengthy table referenced here
US20260363240A1-20261119-T00218
Please refer to the end of the specification for access instructions.

Lengthy table referenced here
US20260363240A1-20261119-T00219
Please refer to the end of the specification for access instructions.

Lengthy table referenced here
US20260363240A1-20261119-T00220
Please refer to the end of the specification for access instructions.

Lengthy table referenced here
US20260363240A1-20261119-T00221
Please refer to the end of the specification for access instructions.

Lengthy table referenced here
US20260363240A1-20261119-T00222
Please refer to the end of the specification for access instructions.

Lengthy table referenced here
US20260363240A1-20261119-T00223
Please refer to the end of the specification for access instructions.

Lengthy table referenced here
US20260363240A1-20261119-T00224
Please refer to the end of the specification for access instructions.

Lengthy table referenced here
US20260363240A1-20261119-T00225
Please refer to the end of the specification for access instructions.

Lengthy table referenced here
US20260363240A1-20261119-T00226
Please refer to the end of the specification for access instructions.

Lengthy table referenced here
US20260363240A1-20261119-T00227
Please refer to the end of the specification for access instructions.

Lengthy table referenced here
US20260363240A1-20261119-T00228
Please refer to the end of the specification for access instructions.

Lengthy table referenced here
US20260363240A1-20261119-T00229
Please refer to the end of the specification for access instructions.

Lengthy table referenced here
US20260363240A1-20261119-T00230
Please refer to the end of the specification for access instructions.

Lengthy table referenced here
US20260363240A1-20261119-T00231
Please refer to the end of the specification for access instructions.

Lengthy table referenced here
US20260363240A1-20261119-T00232
Please refer to the end of the specification for access instructions.

Lengthy table referenced here
US20260363240A1-20261119-T00233
Please refer to the end of the specification for access instructions.

Lengthy table referenced here
US20260363240A1-20261119-T00234
Please refer to the end of the specification for access instructions.

Lengthy table referenced here
US20260363240A1-20261119-T00235
Please refer to the end of the specification for access instructions.

Lengthy table referenced here
US20260363240A1-20261119-T00236
Please refer to the end of the specification for access instructions.

Lengthy table referenced here
US20260363240A1-20261119-T00237
Please refer to the end of the specification for access instructions.

Lengthy table referenced here
US20260363240A1-20261119-T00238
Please refer to the end of the specification for access instructions.

Lengthy table referenced here
US20260363240A1-20261119-T00239
Please refer to the end of the specification for access instructions.

Lengthy table referenced here
US20260363240A1-20261119-T00240
Please refer to the end of the specification for access instructions.

Lengthy table referenced here
US20260363240A1-20261119-T00241
Please refer to the end of the specification for access instructions.

Lengthy table referenced here
US20260363240A1-20261119-T00242
Please refer to the end of the specification for access instructions.

Lengthy table referenced here
US20260363240A1-20261119-T00243
Please refer to the end of the specification for access instructions.

Lengthy table referenced here
US20260363240A1-20261119-T00244
Please refer to the end of the specification for access instructions.

Lengthy table referenced here
US20260363240A1-20261119-T00245
Please refer to the end of the specification for access instructions.

Lengthy table referenced here
US20260363240A1-20261119-T00246
Please refer to the end of the specification for access instructions.

Lengthy table referenced here
US20260363240A1-20261119-T00247
Please refer to the end of the specification for access instructions.

Lengthy table referenced here
US20260363240A1-20261119-T00248
Please refer to the end of the specification for access instructions.

Lengthy table referenced here
US20260363240A1-20261119-T00249
Please refer to the end of the specification for access instructions.

Lengthy table referenced here
US20260363240A1-20261119-T00250
Please refer to the end of the specification for access instructions.

Lengthy table referenced here
US20260363240A1-20261119-T00251
Please refer to the end of the specification for access instructions.

Lengthy table referenced here
US20260363240A1-20261119-T00252
Please refer to the end of the specification for access instructions.

Lengthy table referenced here
US20260363240A1-20261119-T00253
Please refer to the end of the specification for access instructions.

Lengthy table referenced here
US20260363240A1-20261119-T00254
Please refer to the end of the specification for access instructions.

Lengthy table referenced here
US20260363240A1-20261119-T00255
Please refer to the end of the specification for access instructions.

Lengthy table referenced here
US20260363240A1-20261119-T00256
Please refer to the end of the specification for access instructions.

Lengthy table referenced here
US20260363240A1-20261119-T00257
Please refer to the end of the specification for access instructions.

Lengthy table referenced here
US20260363240A1-20261119-T00258
Please refer to the end of the specification for access instructions.

Lengthy table referenced here
US20260363240A1-20261119-T00259
Please refer to the end of the specification for access instructions.

Lengthy table referenced here
US20260363240A1-20261119-T00260
Please refer to the end of the specification for access instructions.

Lengthy table referenced here
US20260363240A1-20261119-T00261
Please refer to the end of the specification for access instructions.

Lengthy table referenced here
US20260363240A1-20261119-T00262
Please refer to the end of the specification for access instructions.

Lengthy table referenced here
US20260363240A1-20261119-T00263
Please refer to the end of the specification for access instructions.

Lengthy table referenced here
US20260363240A1-20261119-T00264
Please refer to the end of the specification for access instructions.

Lengthy table referenced here
US20260363240A1-20261119-T00265
Please refer to the end of the specification for access instructions.

Lengthy table referenced here
US20260363240A1-20261119-T00266
Please refer to the end of the specification for access instructions.

Lengthy table referenced here
US20260363240A1-20261119-T00267
Please refer to the end of the specification for access instructions.

Lengthy table referenced here
US20260363240A1-20261119-T00268
Please refer to the end of the specification for access instructions.

Lengthy table referenced here
US20260363240A1-20261119-T00269
Please refer to the end of the specification for access instructions.

Lengthy table referenced here
US20260363240A1-20261119-T00270
Please refer to the end of the specification for access instructions.

Lengthy table referenced here
US20260363240A1-20261119-T00271
Please refer to the end of the specification for access instructions.

Lengthy table referenced here
US20260363240A1-20261119-T00272
Please refer to the end of the specification for access instructions.

Lengthy table referenced here
US20260363240A1-20261119-T00273
Please refer to the end of the specification for access instructions.

Lengthy table referenced here
US20260363240A1-20261119-T00274
Please refer to the end of the specification for access instructions.

Lengthy table referenced here
US20260363240A1-20261119-T00275
Please refer to the end of the specification for access instructions.

Lengthy table referenced here
US20260363240A1-20261119-T00276
Please refer to the end of the specification for access instructions.

Lengthy table referenced here
US20260363240A1-20261119-T00277
Please refer to the end of the specification for access instructions.

Lengthy table referenced here
US20260363240A1-20261119-T00278
Please refer to the end of the specification for access instructions.

Lengthy table referenced here
US20260363240A1-20261119-T00279
Please refer to the end of the specification for access instructions.

Lengthy table referenced here
US20260363240A1-20261119-T00280
Please refer to the end of the specification for access instructions.

Lengthy table referenced here
US20260363240A1-20261119-T00281
Please refer to the end of the specification for access instructions.

Lengthy table referenced here
US20260363240A1-20261119-T00282
Please refer to the end of the specification for access instructions.

Lengthy table referenced here
US20260363240A1-20261119-T00283
Please refer to the end of the specification for access instructions.

Lengthy table referenced here
US20260363240A1-20261119-T00284
Please refer to the end of the specification for access instructions.

Lengthy table referenced here
US20260363240A1-20261119-T00285
Please refer to the end of the specification for access instructions.

Lengthy table referenced here
US20260363240A1-20261119-T00286
Please refer to the end of the specification for access instructions.

Lengthy table referenced here
US20260363240A1-20261119-T00287
Please refer to the end of the specification for access instructions.

Lengthy table referenced here
US20260363240A1-20261119-T00288
Please refer to the end of the specification for access instructions.

Lengthy table referenced here
US20260363240A1-20261119-T00289
Please refer to the end of the specification for access instructions.

Lengthy table referenced here
US20260363240A1-20261119-T00290
Please refer to the end of the specification for access instructions.

Lengthy table referenced here
US20260363240A1-20261119-T00291
Please refer to the end of the specification for access instructions.

Lengthy table referenced here
US20260363240A1-20261119-T00292
Please refer to the end of the specification for access instructions.

Lengthy table referenced here
US20260363240A1-20261119-T00293
Please refer to the end of the specification for access instructions.

Lengthy table referenced here
US20260363240A1-20261119-T00294
Please refer to the end of the specification for access instructions.

Lengthy table referenced here
US20260363240A1-20261119-T00295
Please refer to the end of the specification for access instructions.

Lengthy table referenced here
US20260363240A1-20261119-T00296
Please refer to the end of the specification for access instructions.

Lengthy table referenced here
US20260363240A1-20261119-T00297
Please refer to the end of the specification for access instructions.

Lengthy table referenced here
US20260363240A1-20261119-T00298
Please refer to the end of the specification for access instructions.

Lengthy table referenced here
US20260363240A1-20261119-T00299
Please refer to the end of the specification for access instructions.

Lengthy table referenced here
US20260363240A1-20261119-T00300
Please refer to the end of the specification for access instructions.

Lengthy table referenced here
US20260363240A1-20261119-T00301
Please refer to the end of the specification for access instructions.

Lengthy table referenced here
US20260363240A1-20261119-T00302
Please refer to the end of the specification for access instructions.

Lengthy table referenced here
US20260363240A1-20261119-T00303
Please refer to the end of the specification for access instructions.

Lengthy table referenced here
US20260363240A1-20261119-T00304
Please refer to the end of the specification for access instructions.

Lengthy table referenced here
US20260363240A1-20261119-T00305
Please refer to the end of the specification for access instructions.

Lengthy table referenced here
US20260363240A1-20261119-T00306
Please refer to the end of the specification for access instructions.

Lengthy table referenced here
US20260363240A1-20261119-T00307
Please refer to the end of the specification for access instructions.

Lengthy table referenced here
US20260363240A1-20261119-T00308
Please refer to the end of the specification for access instructions.

Lengthy table referenced here
US20260363240A1-20261119-T00309
Please refer to the end of the specification for access instructions.

Lengthy table referenced here
US20260363240A1-20261119-T00310
Please refer to the end of the specification for access instructions.

Lengthy table referenced here
US20260363240A1-20261119-T00311
Please refer to the end of the specification for access instructions.

Lengthy table referenced here
US20260363240A1-20261119-T00312
Please refer to the end of the specification for access instructions.

Lengthy table referenced here
US20260363240A1-20261119-T00313
Please refer to the end of the specification for access instructions.

Lengthy table referenced here
US20260363240A1-20261119-T00314
Please refer to the end of the specification for access instructions.

Lengthy table referenced here
US20260363240A1-20261119-T00315
Please refer to the end of the specification for access instructions.

Lengthy table referenced here
US20260363240A1-20261119-T00316
Please refer to the end of the specification for access instructions.

Lengthy table referenced here
US20260363240A1-20261119-T00317
Please refer to the end of the specification for access instructions.

Lengthy table referenced here
US20260363240A1-20261119-T00318
Please refer to the end of the specification for access instructions.

Lengthy table referenced here
US20260363240A1-20261119-T00319
Please refer to the end of the specification for access instructions.

Lengthy table referenced here
US20260363240A1-20261119-T00320
Please refer to the end of the specification for access instructions.

Lengthy table referenced here
US20260363240A1-20261119-T00321
Please refer to the end of the specification for access instructions.

Lengthy table referenced here
US20260363240A1-20261119-T00322
Please refer to the end of the specification for access instructions.

Lengthy table referenced here
US20260363240A1-20261119-T00323
Please refer to the end of the specification for access instructions.

Lengthy table referenced here
US20260363240A1-20261119-T00324
Please refer to the end of the specification for access instructions.

Lengthy table referenced here
US20260363240A1-20261119-T00325
Please refer to the end of the specification for access instructions.

Lengthy table referenced here
US20260363240A1-20261119-T00326
Please refer to the end of the specification for access instructions.

Lengthy table referenced here
US20260363240A1-20261119-T00327
Please refer to the end of the specification for access instructions.

Lengthy table referenced here
US20260363240A1-20261119-T00328
Please refer to the end of the specification for access instructions.

Lengthy table referenced here
US20260363240A1-20261119-T00329
Please refer to the end of the specification for access instructions.

Lengthy table referenced here
US20260363240A1-20261119-T00330
Please refer to the end of the specification for access instructions.

Lengthy table referenced here
US20260363240A1-20261119-T00331
Please refer to the end of the specification for access instructions.

Lengthy table referenced here
US20260363240A1-20261119-T00332
Please refer to the end of the specification for access instructions.

Lengthy table referenced here
US20260363240A1-20261119-T00333
Please refer to the end of the specification for access instructions.

Lengthy table referenced here
US20260363240A1-20261119-T00334
Please refer to the end of the specification for access instructions.

Lengthy table referenced here
US20260363240A1-20261119-T00335
Please refer to the end of the specification for access instructions.

Lengthy table referenced here
US20260363240A1-20261119-T00336
Please refer to the end of the specification for access instructions.

Lengthy table referenced here
US20260363240A1-20261119-T00337
Please refer to the end of the specification for access instructions.

Lengthy table referenced here
US20260363240A1-20261119-T00338
Please refer to the end of the specification for access instructions.

Lengthy table referenced here
US20260363240A1-20261119-T00339
Please refer to the end of the specification for access instructions.

Lengthy table referenced here
US20260363240A1-20261119-T00340
Please refer to the end of the specification for access instructions.

Lengthy table referenced here
US20260363240A1-20261119-T00341
Please refer to the end of the specification for access instructions.

Lengthy table referenced here
US20260363240A1-20261119-T00342
Please refer to the end of the specification for access instructions.

Lengthy table referenced here
US20260363240A1-20261119-T00343
Please refer to the end of the specification for access instructions.

Lengthy table referenced here
US20260363240A1-20261119-T00344
Please refer to the end of the specification for access instructions.

Lengthy table referenced here
US20260363240A1-20261119-T00345
Please refer to the end of the specification for access instructions.

Lengthy table referenced here
US20260363240A1-20261119-T00346
Please refer to the end of the specification for access instructions.

Lengthy table referenced here
US20260363240A1-20261119-T00347
Please refer to the end of the specification for access instructions.

Lengthy table referenced here
US20260363240A1-20261119-T00348
Please refer to the end of the specification for access instructions.

Lengthy table referenced here
US20260363240A1-20261119-T00349
Please refer to the end of the specification for access instructions.

Lengthy table referenced here
US20260363240A1-20261119-T00350
Please refer to the end of the specification for access instructions.

Lengthy table referenced here
US20260363240A1-20261119-T00351
Please refer to the end of the specification for access instructions.

Lengthy table referenced here
US20260363240A1-20261119-T00352
Please refer to the end of the specification for access instructions.

Lengthy table referenced here
US20260363240A1-20261119-T00353
Please refer to the end of the specification for access instructions.

Lengthy table referenced here
US20260363240A1-20261119-T00354
Please refer to the end of the specification for access instructions.

Lengthy table referenced here
US20260363240A1-20261119-T00355
Please refer to the end of the specification for access instructions.

Lengthy table referenced here
US20260363240A1-20261119-T00356
Please refer to the end of the specification for access instructions.

Lengthy table referenced here
US20260363240A1-20261119-T00357
Please refer to the end of the specification for access instructions.

Lengthy table referenced here
US20260363240A1-20261119-T00358
Please refer to the end of the specification for access instructions.

Lengthy table referenced here
US20260363240A1-20261119-T00359
Please refer to the end of the specification for access instructions.

Lengthy table referenced here
US20260363240A1-20261119-T00360
Please refer to the end of the specification for access instructions.

Lengthy table referenced here
US20260363240A1-20261119-T00361
Please refer to the end of the specification for access instructions.

Lengthy table referenced here
US20260363240A1-20261119-T00362
Please refer to the end of the specification for access instructions.

Lengthy table referenced here
US20260363240A1-20261119-T00363
Please refer to the end of the specification for access instructions.

Lengthy table referenced here
US20260363240A1-20261119-T00364
Please refer to the end of the specification for access instructions.

Lengthy table referenced here
US20260363240A1-20261119-T00365
Please refer to the end of the specification for access instructions.

Lengthy table referenced here
US20260363240A1-20261119-T00366
Please refer to the end of the specification for access instructions.

Lengthy table referenced here
US20260363240A1-20261119-T00367
Please refer to the end of the specification for access instructions.

Lengthy table referenced here
US20260363240A1-20261119-T00368
Please refer to the end of the specification for access instructions.

Lengthy table referenced here
US20260363240A1-20261119-T00369
Please refer to the end of the specification for access instructions.

Lengthy table referenced here
US20260363240A1-20261119-T00370
Please refer to the end of the specification for access instructions.

Lengthy table referenced here
US20260363240A1-20261119-T00371
Please refer to the end of the specification for access instructions.

Lengthy table referenced here
US20260363240A1-20261119-T00372
Please refer to the end of the specification for access instructions.

Lengthy table referenced here
US20260363240A1-20261119-T00373
Please refer to the end of the specification for access instructions.

Lengthy table referenced here
US20260363240A1-20261119-T00374
Please refer to the end of the specification for access instructions.

Lengthy table referenced here
US20260363240A1-20261119-T00375
Please refer to the end of the specification for access instructions.

Lengthy table referenced here
US20260363240A1-20261119-T00376
Please refer to the end of the specification for access instructions.

Lengthy table referenced here
US20260363240A1-20261119-T00377
Please refer to the end of the specification for access instructions.

Lengthy table referenced here
US20260363240A1-20261119-T00378
Please refer to the end of the specification for access instructions.

Lengthy table referenced here
US20260363240A1-20261119-T00379
Please refer to the end of the specification for access instructions.

Lengthy table referenced here
US20260363240A1-20261119-T00380
Please refer to the end of the specification for access instructions.

Lengthy table referenced here
US20260363240A1-20261119-T00381
Please refer to the end of the specification for access instructions.

Lengthy table referenced here
US20260363240A1-20261119-T00382
Please refer to the end of the specification for access instructions.

Lengthy table referenced here
US20260363240A1-20261119-T00383
Please refer to the end of the specification for access instructions.

Lengthy table referenced here
US20260363240A1-20261119-T00384
Please refer to the end of the specification for access instructions.

Lengthy table referenced here
US20260363240A1-20261119-T00385
Please refer to the end of the specification for access instructions.

Lengthy table referenced here
US20260363240A1-20261119-T00386
Please refer to the end of the specification for access instructions.

Lengthy table referenced here
US20260363240A1-20261119-T00387
Please refer to the end of the specification for access instructions.

Lengthy table referenced here
US20260363240A1-20261119-T00388
Please refer to the end of the specification for access instructions.

Lengthy table referenced here
US20260363240A1-20261119-T00389
Please refer to the end of the specification for access instructions.

Lengthy table referenced here
US20260363240A1-20261119-T00390
Please refer to the end of the specification for access instructions.

Lengthy table referenced here
US20260363240A1-20261119-T00391
Please refer to the end of the specification for access instructions.

Lengthy table referenced here
US20260363240A1-20261119-T00392
Please refer to the end of the specification for access instructions.

Lengthy table referenced here
US20260363240A1-20261119-T00393
Please refer to the end of the specification for access instructions.

Lengthy table referenced here
US20260363240A1-20261119-T00394
Please refer to the end of the specification for access instructions.

Lengthy table referenced here
US20260363240A1-20261119-T00395
Please refer to the end of the specification for access instructions.

Lengthy table referenced here
US20260363240A1-20261119-T00396
Please refer to the end of the specification for access instructions.

Lengthy table referenced here
US20260363240A1-20261119-T00397
Please refer to the end of the specification for access instructions.

Lengthy table referenced here
US20260363240A1-20261119-T00398
Please refer to the end of the specification for access instructions.

Lengthy table referenced here
US20260363240A1-20261119-T00399
Please refer to the end of the specification for access instructions.

Lengthy table referenced here
US20260363240A1-20261119-T00400
Please refer to the end of the specification for access instructions.

Lengthy table referenced here
US20260363240A1-20261119-T00401
Please refer to the end of the specification for access instructions.

Lengthy table referenced here
US20260363240A1-20261119-T00402
Please refer to the end of the specification for access instructions.

Lengthy table referenced here
US20260363240A1-20261119-T00403
Please refer to the end of the specification for access instructions.

Lengthy table referenced here
US20260363240A1-20261119-T00404
Please refer to the end of the specification for access instructions.

Lengthy table referenced here
US20260363240A1-20261119-T00405
Please refer to the end of the specification for access instructions.

Lengthy table referenced here
US20260363240A1-20261119-T00406
Please refer to the end of the specification for access instructions.

Lengthy table referenced here
US20260363240A1-20261119-T00407
Please refer to the end of the specification for access instructions.

Lengthy table referenced here
US20260363240A1-20261119-T00408
Please refer to the end of the specification for access instructions.

Lengthy table referenced here
US20260363240A1-20261119-T00409
Please refer to the end of the specification for access instructions.

Lengthy table referenced here
US20260363240A1-20261119-T00410
Please refer to the end of the specification for access instructions.

Lengthy table referenced here
US20260363240A1-20261119-T00411
Please refer to the end of the specification for access instructions.

Lengthy table referenced here
US20260363240A1-20261119-T00412
Please refer to the end of the specification for access instructions.

Lengthy table referenced here
US20260363240A1-20261119-T00413
Please refer to the end of the specification for access instructions.

Lengthy table referenced here
US20260363240A1-20261119-T00414
Please refer to the end of the specification for access instructions.

Lengthy table referenced here
US20260363240A1-20261119-T00415
Please refer to the end of the specification for access instructions.

Lengthy table referenced here
US20260363240A1-20261119-T00416
Please refer to the end of the specification for access instructions.

Lengthy table referenced here
US20260363240A1-20261119-T00417
Please refer to the end of the specification for access instructions.

Lengthy table referenced here
US20260363240A1-20261119-T00418
Please refer to the end of the specification for access instructions.

Lengthy table referenced here
US20260363240A1-20261119-T00419
Please refer to the end of the specification for access instructions.

Lengthy table referenced here
US20260363240A1-20261119-T00420
Please refer to the end of the specification for access instructions.

Lengthy table referenced here
US20260363240A1-20261119-T00421
Please refer to the end of the specification for access instructions.

Lengthy table referenced here
US20260363240A1-20261119-T00422
Please refer to the end of the specification for access instructions.

Lengthy table referenced here
US20260363240A1-20261119-T00423
Please refer to the end of the specification for access instructions.

Lengthy table referenced here
US20260363240A1-20261119-T00424
Please refer to the end of the specification for access instructions.

Lengthy table referenced here
US20260363240A1-20261119-T00425
Please refer to the end of the specification for access instructions.

Lengthy table referenced here
US20260363240A1-20261119-T00426
Please refer to the end of the specification for access instructions.

Lengthy table referenced here
US20260363240A1-20261119-T00427
Please refer to the end of the specification for access instructions.

Lengthy table referenced here
US20260363240A1-20261119-T00428
Please refer to the end of the specification for access instructions.

Lengthy table referenced here
US20260363240A1-20261119-T00429
Please refer to the end of the specification for access instructions.

Lengthy table referenced here
US20260363240A1-20261119-T00430
Please refer to the end of the specification for access instructions.

Lengthy table referenced here
US20260363240A1-20261119-T00431
Please refer to the end of the specification for access instructions.

Lengthy table referenced here
US20260363240A1-20261119-T00432
Please refer to the end of the specification for access instructions.

Lengthy table referenced here
US20260363240A1-20261119-T00433
Please refer to the end of the specification for access instructions.

Lengthy table referenced here
US20260363240A1-20261119-T00434
Please refer to the end of the specification for access instructions.

Lengthy table referenced here
US20260363240A1-20261119-T00435
Please refer to the end of the specification for access instructions.

Lengthy table referenced here
US20260363240A1-20261119-T00436
Please refer to the end of the specification for access instructions.

Lengthy table referenced here
US20260363240A1-20261119-T00437
Please refer to the end of the specification for access instructions.

Lengthy table referenced here
US20260363240A1-20261119-T00438
Please refer to the end of the specification for access instructions.

Lengthy table referenced here
US20260363240A1-20261119-T00439
Please refer to the end of the specification for access instructions.

Lengthy table referenced here
US20260363240A1-20261119-T00440
Please refer to the end of the specification for access instructions.

Lengthy table referenced here
US20260363240A1-20261119-T00441
Please refer to the end of the specification for access instructions.

Lengthy table referenced here
US20260363240A1-20261119-T00442
Please refer to the end of the specification for access instructions.

Lengthy table referenced here
US20260363240A1-20261119-T00443
Please refer to the end of the specification for access instructions.

Lengthy table referenced here
US20260363240A1-20261119-T00444
Please refer to the end of the specification for access instructions.

Lengthy table referenced here
US20260363240A1-20261119-T00445
Please refer to the end of the specification for access instructions.

Lengthy table referenced here
US20260363240A1-20261119-T00446
Please refer to the end of the specification for access instructions.

Lengthy table referenced here
US20260363240A1-20261119-T00447
Please refer to the end of the specification for access instructions.

Lengthy table referenced here
US20260363240A1-20261119-T00448
Please refer to the end of the specification for access instructions.

Lengthy table referenced here
US20260363240A1-20261119-T00449
Please refer to the end of the specification for access instructions.

Lengthy table referenced here
US20260363240A1-20261119-T00450
Please refer to the end of the specification for access instructions.

Lengthy table referenced here
US20260363240A1-20261119-T00451
Please refer to the end of the specification for access instructions.

Lengthy table referenced here
US20260363240A1-20261119-T00452
Please refer to the end of the specification for access instructions.

Lengthy table referenced here
US20260363240A1-20261119-T00453
Please refer to the end of the specification for access instructions.

Lengthy table referenced here
US20260363240A1-20261119-T00454
Please refer to the end of the specification for access instructions.

Lengthy table referenced here
US20260363240A1-20261119-T00455
Please refer to the end of the specification for access instructions.

Lengthy table referenced here
US20260363240A1-20261119-T00456
Please refer to the end of the specification for access instructions.

Lengthy table referenced here
US20260363240A1-20261119-T00457
Please refer to the end of the specification for access instructions.

Lengthy table referenced here
US20260363240A1-20261119-T00458
Please refer to the end of the specification for access instructions.

Lengthy table referenced here
US20260363240A1-20261119-T00459
Please refer to the end of the specification for access instructions.

Lengthy table referenced here
US20260363240A1-20261119-T00460
Please refer to the end of the specification for access instructions.

Lengthy table referenced here
US20260363240A1-20261119-T00461
Please refer to the end of the specification for access instructions.

Lengthy table referenced here
US20260363240A1-20261119-T00462
Please refer to the end of the specification for access instructions.

Lengthy table referenced here
US20260363240A1-20261119-T00463
Please refer to the end of the specification for access instructions.

Lengthy table referenced here
US20260363240A1-20261119-T00464
Please refer to the end of the specification for access instructions.

Lengthy table referenced here
US20260363240A1-20261119-T00465
Please refer to the end of the specification for access instructions.

Lengthy table referenced here
US20260363240A1-20261119-T00466
Please refer to the end of the specification for access instructions.

Lengthy table referenced here
US20260363240A1-20261119-T00467
Please refer to the end of the specification for access instructions.

Lengthy table referenced here
US20260363240A1-20261119-T00468
Please refer to the end of the specification for access instructions.

Lengthy table referenced here
US20260363240A1-20261119-T00469
Please refer to the end of the specification for access instructions.

Lengthy table referenced here
US20260363240A1-20261119-T00470
Please refer to the end of the specification for access instructions.

Lengthy table referenced here
US20260363240A1-20261119-T00471
Please refer to the end of the specification for access instructions.

Lengthy table referenced here
US20260363240A1-20261119-T00472
Please refer to the end of the specification for access instructions.

Lengthy table referenced here
US20260363240A1-20261119-T00473
Please refer to the end of the specification for access instructions.

Lengthy table referenced here
US20260363240A1-20261119-T00474
Please refer to the end of the specification for access instructions.

Lengthy table referenced here
US20260363240A1-20261119-T00475
Please refer to the end of the specification for access instructions.

Lengthy table referenced here
US20260363240A1-20261119-T00476
Please refer to the end of the specification for access instructions.

Lengthy table referenced here
US20260363240A1-20261119-T00477
Please refer to the end of the specification for access instructions.

Lengthy table referenced here
US20260363240A1-20261119-T00478
Please refer to the end of the specification for access instructions.

Lengthy table referenced here
US20260363240A1-20261119-T00479
Please refer to the end of the specification for access instructions.

Lengthy table referenced here
US20260363240A1-20261119-T00480
Please refer to the end of the specification for access instructions.

Lengthy table referenced here
US20260363240A1-20261119-T00481
Please refer to the end of the specification for access instructions.

Lengthy table referenced here
US20260363240A1-20261119-T00482
Please refer to the end of the specification for access instructions.

Lengthy table referenced here
US20260363240A1-20261119-T00483
Please refer to the end of the specification for access instructions.

Lengthy table referenced here
US20260363240A1-20261119-T00484
Please refer to the end of the specification for access instructions.

Lengthy table referenced here
US20260363240A1-20261119-T00485
Please refer to the end of the specification for access instructions.

Lengthy table referenced here
US20260363240A1-20261119-T00486
Please refer to the end of the specification for access instructions.

Lengthy table referenced here
US20260363240A1-20261119-T00487
Please refer to the end of the specification for access instructions.

Lengthy table referenced here
US20260363240A1-20261119-T00488
Please refer to the end of the specification for access instructions.

Lengthy table referenced here
US20260363240A1-20261119-T00489
Please refer to the end of the specification for access instructions.

Lengthy table referenced here
US20260363240A1-20261119-T00490
Please refer to the end of the specification for access instructions.

Lengthy table referenced here
US20260363240A1-20261119-T00491
Please refer to the end of the specification for access instructions.

Lengthy table referenced here
US20260363240A1-20261119-T00492
Please refer to the end of the specification for access instructions.

Lengthy table referenced here
US20260363240A1-20261119-T00493
Please refer to the end of the specification for access instructions.

Lengthy table referenced here
US20260363240A1-20261119-T00494
Please refer to the end of the specification for access instructions.

Lengthy table referenced here
US20260363240A1-20261119-T00495
Please refer to the end of the specification for access instructions.

Lengthy table referenced here
US20260363240A1-20261119-T00496
Please refer to the end of the specification for access instructions.

Lengthy table referenced here
US20260363240A1-20261119-T00497
Please refer to the end of the specification for access instructions.

Lengthy table referenced here
US20260363240A1-20261119-T00498
Please refer to the end of the specification for access instructions.

Lengthy table referenced here
US20260363240A1-20261119-T00499
Please refer to the end of the specification for access instructions.

Lengthy table referenced here
US20260363240A1-20261119-T00500
Please refer to the end of the specification for access instructions.

Lengthy table referenced here
US20260363240A1-20261119-T00501
Please refer to the end of the specification for access instructions.

Lengthy table referenced here
US20260363240A1-20261119-T00502
Please refer to the end of the specification for access instructions.

Lengthy table referenced here
US20260363240A1-20261119-T00503
Please refer to the end of the specification for access instructions.

Lengthy table referenced here
US20260363240A1-20261119-T00504
Please refer to the end of the specification for access instructions.

Lengthy table referenced here
US20260363240A1-20261119-T00505
Please refer to the end of the specification for access instructions.

Lengthy table referenced here
US20260363240A1-20261119-T00506
Please refer to the end of the specification for access instructions.

Lengthy table referenced here
US20260363240A1-20261119-T00507
Please refer to the end of the specification for access instructions.

Lengthy table referenced here
US20260363240A1-20261119-T00508
Please refer to the end of the specification for access instructions.

Lengthy table referenced here
US20260363240A1-20261119-T00509
Please refer to the end of the specification for access instructions.

Lengthy table referenced here
US20260363240A1-20261119-T00510
Please refer to the end of the specification for access instructions.

Lengthy table referenced here
US20260363240A1-20261119-T00511
Please refer to the end of the specification for access instructions.

Lengthy table referenced here
US20260363240A1-20261119-T00512
Please refer to the end of the specification for access instructions.

Lengthy table referenced here
US20260363240A1-20261119-T00513
Please refer to the end of the specification for access instructions.

Lengthy table referenced here
US20260363240A1-20261119-T00514
Please refer to the end of the specification for access instructions.

Lengthy table referenced here
US20260363240A1-20261119-T00515
Please refer to the end of the specification for access instructions.

Lengthy table referenced here
US20260363240A1-20261119-T00516
Please refer to the end of the specification for access instructions.

Lengthy table referenced here
US20260363240A1-20261119-T00517
Please refer to the end of the specification for access instructions.

Lengthy table referenced here
US20260363240A1-20261119-T00518
Please refer to the end of the specification for access instructions.

Lengthy table referenced here
US20260363240A1-20261119-T00519
Please refer to the end of the specification for access instructions.

Lengthy table referenced here
US20260363240A1-20261119-T00520
Please refer to the end of the specification for access instructions.

Lengthy table referenced here
US20260363240A1-20261119-T00521
Please refer to the end of the specification for access instructions.

Lengthy table referenced here
US20260363240A1-20261119-T00522
Please refer to the end of the specification for access instructions.

Lengthy table referenced here
US20260363240A1-20261119-T00523
Please refer to the end of the specification for access instructions.

Lengthy table referenced here
US20260363240A1-20261119-T00524
Please refer to the end of the specification for access instructions.

Lengthy table referenced here
US20260363240A1-20261119-T00525
Please refer to the end of the specification for access instructions.

Lengthy table referenced here
US20260363240A1-20261119-T00526
Please refer to the end of the specification for access instructions.

Lengthy table referenced here
US20260363240A1-20261119-T00527
Please refer to the end of the specification for access instructions.

Lengthy table referenced here
US20260363240A1-20261119-T00528
Please refer to the end of the specification for access instructions.

Lengthy table referenced here
US20260363240A1-20261119-T00529
Please refer to the end of the specification for access instructions.

Lengthy table referenced here
US20260363240A1-20261119-T00530
Please refer to the end of the specification for access instructions.

Lengthy table referenced here
US20260363240A1-20261119-T00531
Please refer to the end of the specification for access instructions.

Lengthy table referenced here
US20260363240A1-20261119-T00532
Please refer to the end of the specification for access instructions.

Lengthy table referenced here
US20260363240A1-20261119-T00533
Please refer to the end of the specification for access instructions.

Lengthy table referenced here
US20260363240A1-20261119-T00534
Please refer to the end of the specification for access instructions.

Lengthy table referenced here
US20260363240A1-20261119-T00535
Please refer to the end of the specification for access instructions.

Lengthy table referenced here
US20260363240A1-20261119-T00536
Please refer to the end of the specification for access instructions.

Lengthy table referenced here
US20260363240A1-20261119-T00537
Please refer to the end of the specification for access instructions.

Lengthy table referenced here
US20260363240A1-20261119-T00538
Please refer to the end of the specification for access instructions.

Lengthy table referenced here
US20260363240A1-20261119-T00539
Please refer to the end of the specification for access instructions.

Lengthy table referenced here
US20260363240A1-20261119-T00540
Please refer to the end of the specification for access instructions.

Lengthy table referenced here
US20260363240A1-20261119-T00541
Please refer to the end of the specification for access instructions.

Lengthy table referenced here
US20260363240A1-20261119-T00542
Please refer to the end of the specification for access instructions.

Lengthy table referenced here
US20260363240A1-20261119-T00543
Please refer to the end of the specification for access instructions.

Lengthy table referenced here
US20260363240A1-20261119-T00544
Please refer to the end of the specification for access instructions.

Lengthy table referenced here
US20260363240A1-20261119-T00545
Please refer to the end of the specification for access instructions.

Lengthy table referenced here
US20260363240A1-20261119-T00546
Please refer to the end of the specification for access instructions.

Lengthy table referenced here
US20260363240A1-20261119-T00547
Please refer to the end of the specification for access instructions.

Lengthy table referenced here
US20260363240A1-20261119-T00548
Please refer to the end of the specification for access instructions.

Lengthy table referenced here
US20260363240A1-20261119-T00549
Please refer to the end of the specification for access instructions.

Lengthy table referenced here
US20260363240A1-20261119-T00550
Please refer to the end of the specification for access instructions.

Lengthy table referenced here
US20260363240A1-20261119-T00551
Please refer to the end of the specification for access instructions.

Lengthy table referenced here
US20260363240A1-20261119-T00552
Please refer to the end of the specification for access instructions.

Lengthy table referenced here
US20260363240A1-20261119-T00553
Please refer to the end of the specification for access instructions.

Lengthy table referenced here
US20260363240A1-20261119-T00554
Please refer to the end of the specification for access instructions.

Lengthy table referenced here
US20260363240A1-20261119-T00555
Please refer to the end of the specification for access instructions.

Lengthy table referenced here
US20260363240A1-20261119-T00556
Please refer to the end of the specification for access instructions.

Lengthy table referenced here
US20260363240A1-20261119-T00557
Please refer to the end of the specification for access instructions.

Lengthy table referenced here
US20260363240A1-20261119-T00558
Please refer to the end of the specification for access instructions.

Lengthy table referenced here
US20260363240A1-20261119-T00559
Please refer to the end of the specification for access instructions.

Lengthy table referenced here
US20260363240A1-20261119-T00560
Please refer to the end of the specification for access instructions.

Lengthy table referenced here
US20260363240A1-20261119-T00561
Please refer to the end of the specification for access instructions.

Lengthy table referenced here
US20260363240A1-20261119-T00562
Please refer to the end of the specification for access instructions.

Lengthy table referenced here
US20260363240A1-20261119-T00563
Please refer to the end of the specification for access instructions.

Lengthy table referenced here
US20260363240A1-20261119-T00564
Please refer to the end of the specification for access instructions.

Lengthy table referenced here
US20260363240A1-20261119-T00565
Please refer to the end of the specification for access instructions.

Lengthy table referenced here
US20260363240A1-20261119-T00566
Please refer to the end of the specification for access instructions.

Lengthy table referenced here
US20260363240A1-20261119-T00567
Please refer to the end of the specification for access instructions.

Lengthy table referenced here
US20260363240A1-20261119-T00568
Please refer to the end of the specification for access instructions.

Lengthy table referenced here
US20260363240A1-20261119-T00569
Please refer to the end of the specification for access instructions.

Lengthy table referenced here
US20260363240A1-20261119-T00570
Please refer to the end of the specification for access instructions.

Lengthy table referenced here
US20260363240A1-20261119-T00571
Please refer to the end of the specification for access instructions.

Lengthy table referenced here
US20260363240A1-20261119-T00572
Please refer to the end of the specification for access instructions.

Lengthy table referenced here
US20260363240A1-20261119-T00573
Please refer to the end of the specification for access instructions.

Lengthy table referenced here
US20260363240A1-20261119-T00574
Please refer to the end of the specification for access instructions.

Lengthy table referenced here
US20260363240A1-20261119-T00575
Please refer to the end of the specification for access instructions.

Lengthy table referenced here
US20260363240A1-20261119-T00576
Please refer to the end of the specification for access instructions.

Lengthy table referenced here
US20260363240A1-20261119-T00577
Please refer to the end of the specification for access instructions.

Lengthy table referenced here
US20260363240A1-20261119-T00578
Please refer to the end of the specification for access instructions.

Lengthy table referenced here
US20260363240A1-20261119-T00579
Please refer to the end of the specification for access instructions.

Lengthy table referenced here
US20260363240A1-20261119-T00580
Please refer to the end of the specification for access instructions.

Lengthy table referenced here
US20260363240A1-20261119-T00581
Please refer to the end of the specification for access instructions.

Lengthy table referenced here
US20260363240A1-20261119-T00582
Please refer to the end of the specification for access instructions.

Lengthy table referenced here
US20260363240A1-20261119-T00583
Please refer to the end of the specification for access instructions.

Lengthy table referenced here
US20260363240A1-20261119-T00584
Please refer to the end of the specification for access instructions.

Lengthy table referenced here
US20260363240A1-20261119-T00585
Please refer to the end of the specification for access instructions.

Lengthy table referenced here
US20260363240A1-20261119-T00586
Please refer to the end of the specification for access instructions.

Lengthy table referenced here
US20260363240A1-20261119-T00587
Please refer to the end of the specification for access instructions.

Lengthy table referenced here
US20260363240A1-20261119-T00588
Please refer to the end of the specification for access instructions.

Lengthy table referenced here
US20260363240A1-20261119-T00589
Please refer to the end of the specification for access instructions.

Lengthy table referenced here
US20260363240A1-20261119-T00590
Please refer to the end of the specification for access instructions.

Lengthy table referenced here
US20260363240A1-20261119-T00591
Please refer to the end of the specification for access instructions.

Lengthy table referenced here
US20260363240A1-20261119-T00592
Please refer to the end of the specification for access instructions.

Lengthy table referenced here
US20260363240A1-20261119-T00593
Please refer to the end of the specification for access instructions.

Lengthy table referenced here
US20260363240A1-20261119-T00594
Please refer to the end of the specification for access instructions.

Lengthy table referenced here
US20260363240A1-20261119-T00595
Please refer to the end of the specification for access instructions.

Lengthy table referenced here
US20260363240A1-20261119-T00596
Please refer to the end of the specification for access instructions.

Lengthy table referenced here
US20260363240A1-20261119-T00597
Please refer to the end of the specification for access instructions.

Lengthy table referenced here
US20260363240A1-20261119-T00598
Please refer to the end of the specification for access instructions.

Lengthy table referenced here
US20260363240A1-20261119-T00599
Please refer to the end of the specification for access instructions.

Lengthy table referenced here
US20260363240A1-20261119-T00600
Please refer to the end of the specification for access instructions.

Lengthy table referenced here
US20260363240A1-20261119-T00601
Please refer to the end of the specification for access instructions.

Lengthy table referenced here
US20260363240A1-20261119-T00602
Please refer to the end of the specification for access instructions.

Lengthy table referenced here
US20260363240A1-20261119-T00603
Please refer to the end of the specification for access instructions.

Lengthy table referenced here
US20260363240A1-20261119-T00604
Please refer to the end of the specification for access instructions.

Lengthy table referenced here
US20260363240A1-20261119-T00605
Please refer to the end of the specification for access instructions.

Lengthy table referenced here
US20260363240A1-20261119-T00606
Please refer to the end of the specification for access instructions.

Lengthy table referenced here
US20260363240A1-20261119-T00607
Please refer to the end of the specification for access instructions.

Lengthy table referenced here
US20260363240A1-20261119-T00608
Please refer to the end of the specification for access instructions.

Lengthy table referenced here
US20260363240A1-20261119-T00609
Please refer to the end of the specification for access instructions.

Lengthy table referenced here
US20260363240A1-20261119-T00610
Please refer to the end of the specification for access instructions.

Lengthy table referenced here
US20260363240A1-20261119-T00611
Please refer to the end of the specification for access instructions.

Lengthy table referenced here
US20260363240A1-20261119-T00612
Please refer to the end of the specification for access instructions.

Lengthy table referenced here
US20260363240A1-20261119-T00613
Please refer to the end of the specification for access instructions.

Lengthy table referenced here
US20260363240A1-20261119-T00614
Please refer to the end of the specification for access instructions.

Lengthy table referenced here
US20260363240A1-20261119-T00615
Please refer to the end of the specification for access instructions.

Lengthy table referenced here
US20260363240A1-20261119-T00616
Please refer to the end of the specification for access instructions.

Lengthy table referenced here
US20260363240A1-20261119-T00617
Please refer to the end of the specification for access instructions.

Lengthy table referenced here
US20260363240A1-20261119-T00618
Please refer to the end of the specification for access instructions.

Lengthy table referenced here
US20260363240A1-20261119-T00619
Please refer to the end of the specification for access instructions.

Lengthy table referenced here
US20260363240A1-20261119-T00620
Please refer to the end of the specification for access instructions.

Lengthy table referenced here
US20260363240A1-20261119-T00621
Please refer to the end of the specification for access instructions.

Lengthy table referenced here
US20260363240A1-20261119-T00622
Please refer to the end of the specification for access instructions.

Lengthy table referenced here
US20260363240A1-20261119-T00623
Please refer to the end of the specification for access instructions.

Lengthy table referenced here
US20260363240A1-20261119-T00624
Please refer to the end of the specification for access instructions.

Lengthy table referenced here
US20260363240A1-20261119-T00625
Please refer to the end of the specification for access instructions.

Lengthy table referenced here
US20260363240A1-20261119-T00626
Please refer to the end of the specification for access instructions.

Lengthy table referenced here
US20260363240A1-20261119-T00627
Please refer to the end of the specification for access instructions.

Lengthy table referenced here
US20260363240A1-20261119-T00628
Please refer to the end of the specification for access instructions.

Lengthy table referenced here
US20260363240A1-20261119-T00629
Please refer to the end of the specification for access instructions.

Lengthy table referenced here
US20260363240A1-20261119-T00630
Please refer to the end of the specification for access instructions.

Lengthy table referenced here
US20260363240A1-20261119-T00631
Please refer to the end of the specification for access instructions.

Lengthy table referenced here
US20260363240A1-20261119-T00632
Please refer to the end of the specification for access instructions.

Lengthy table referenced here
US20260363240A1-20261119-T00633
Please refer to the end of the specification for access instructions.

Lengthy table referenced here
US20260363240A1-20261119-T00634
Please refer to the end of the specification for access instructions.

Lengthy table referenced here
US20260363240A1-20261119-T00635
Please refer to the end of the specification for access instructions.

Lengthy table referenced here
US20260363240A1-20261119-T00636
Please refer to the end of the specification for access instructions.

Lengthy table referenced here
US20260363240A1-20261119-T00637
Please refer to the end of the specification for access instructions.

Lengthy table referenced here
US20260363240A1-20261119-T00638
Please refer to the end of the specification for access instructions.

Lengthy table referenced here
US20260363240A1-20261119-T00639
Please refer to the end of the specification for access instructions.

Lengthy table referenced here
US20260363240A1-20261119-T00640
Please refer to the end of the specification for access instructions.

Lengthy table referenced here
US20260363240A1-20261119-T00641
Please refer to the end of the specification for access instructions.

Lengthy table referenced here
US20260363240A1-20261119-T00642
Please refer to the end of the specification for access instructions.

Lengthy table referenced here
US20260363240A1-20261119-T00643
Please refer to the end of the specification for access instructions.

Lengthy table referenced here
US20260363240A1-20261119-T00644
Please refer to the end of the specification for access instructions.

Lengthy table referenced here
US20260363240A1-20261119-T00645
Please refer to the end of the specification for access instructions.

Lengthy table referenced here
US20260363240A1-20261119-T00646
Please refer to the end of the specification for access instructions.

Lengthy table referenced here
US20260363240A1-20261119-T00647
Please refer to the end of the specification for access instructions.

Lengthy table referenced here
US20260363240A1-20261119-T00648
Please refer to the end of the specification for access instructions.

Lengthy table referenced here
US20260363240A1-20261119-T00649
Please refer to the end of the specification for access instructions.

Lengthy table referenced here
US20260363240A1-20261119-T00650
Please refer to the end of the specification for access instructions.

Lengthy table referenced here
US20260363240A1-20261119-T00651
Please refer to the end of the specification for access instructions.

Lengthy table referenced here
US20260363240A1-20261119-T00652
Please refer to the end of the specification for access instructions.

Lengthy table referenced here
US20260363240A1-20261119-T00653
Please refer to the end of the specification for access instructions.

Lengthy table referenced here
US20260363240A1-20261119-T00654
Please refer to the end of the specification for access instructions.

Lengthy table referenced here
US20260363240A1-20261119-T00655
Please refer to the end of the specification for access instructions.

Lengthy table referenced here
US20260363240A1-20261119-T00656
Please refer to the end of the specification for access instructions.

Lengthy table referenced here
US20260363240A1-20261119-T00657
Please refer to the end of the specification for access instructions.

Lengthy table referenced here
US20260363240A1-20261119-T00658
Please refer to the end of the specification for access instructions.

Lengthy table referenced here
US20260363240A1-20261119-T00659
Please refer to the end of the specification for access instructions.

Lengthy table referenced here
US20260363240A1-20261119-T00660
Please refer to the end of the specification for access instructions.

Lengthy table referenced here
US20260363240A1-20261119-T00661
Please refer to the end of the specification for access instructions.

Lengthy table referenced here
US20260363240A1-20261119-T00662
Please refer to the end of the specification for access instructions.

Lengthy table referenced here
US20260363240A1-20261119-T00663
Please refer to the end of the specification for access instructions.

Lengthy table referenced here
US20260363240A1-20261119-T00664
Please refer to the end of the specification for access instructions.

Lengthy table referenced here
US20260363240A1-20261119-T00665
Please refer to the end of the specification for access instructions.

Lengthy table referenced here
US20260363240A1-20261119-T00666
Please refer to the end of the specification for access instructions.

Lengthy table referenced here
US20260363240A1-20261119-T00667
Please refer to the end of the specification for access instructions.

Lengthy table referenced here
US20260363240A1-20261119-T00668
Please refer to the end of the specification for access instructions.

Lengthy table referenced here
US20260363240A1-20261119-T00669
Please refer to the end of the specification for access instructions.

Lengthy table referenced here
US20260363240A1-20261119-T00670
Please refer to the end of the specification for access instructions.

Lengthy table referenced here
US20260363240A1-20261119-T00671
Please refer to the end of the specification for access instructions.

Lengthy table referenced here
US20260363240A1-20261119-T00672
Please refer to the end of the specification for access instructions.

Lengthy table referenced here
US20260363240A1-20261119-T00673
Please refer to the end of the specification for access instructions.

Lengthy table referenced here
US20260363240A1-20261119-T00674
Please refer to the end of the specification for access instructions.

Lengthy table referenced here
US20260363240A1-20261119-T00675
Please refer to the end of the specification for access instructions.

Lengthy table referenced here
US20260363240A1-20261119-T00676
Please refer to the end of the specification for access instructions.

Lengthy table referenced here
US20260363240A1-20261119-T00677
Please refer to the end of the specification for access instructions.

Lengthy table referenced here
US20260363240A1-20261119-T00678
Please refer to the end of the specification for access instructions.

Lengthy table referenced here
US20260363240A1-20261119-T00679
Please refer to the end of the specification for access instructions.

Lengthy table referenced here
US20260363240A1-20261119-T00680
Please refer to the end of the specification for access instructions.

Lengthy table referenced here
US20260363240A1-20261119-T00681
Please refer to the end of the specification for access instructions.

Lengthy table referenced here
US20260363240A1-20261119-T00682
Please refer to the end of the specification for access instructions.

Lengthy table referenced here
US20260363240A1-20261119-T00683
Please refer to the end of the specification for access instructions.

Lengthy table referenced here
US20260363240A1-20261119-T00684
Please refer to the end of the specification for access instructions.

Lengthy table referenced here
US20260363240A1-20261119-T00685
Please refer to the end of the specification for access instructions.

Lengthy table referenced here
US20260363240A1-20261119-T00686
Please refer to the end of the specification for access instructions.

Lengthy table referenced here
US20260363240A1-20261119-T00687
Please refer to the end of the specification for access instructions.

Lengthy table referenced here
US20260363240A1-20261119-T00688
Please refer to the end of the specification for access instructions.

Lengthy table referenced here
US20260363240A1-20261119-T00689
Please refer to the end of the specification for access instructions.

Lengthy table referenced here
US20260363240A1-20261119-T00690
Please refer to the end of the specification for access instructions.

Lengthy table referenced here
US20260363240A1-20261119-T00691
Please refer to the end of the specification for access instructions.

Lengthy table referenced here
US20260363240A1-20261119-T00692
Please refer to the end of the specification for access instructions.

Lengthy table referenced here
US20260363240A1-20261119-T00693
Please refer to the end of the specification for access instructions.

Lengthy table referenced here
US20260363240A1-20261119-T00694
Please refer to the end of the specification for access instructions.

Lengthy table referenced here
US20260363240A1-20261119-T00695
Please refer to the end of the specification for access instructions.

Lengthy table referenced here
US20260363240A1-20261119-T00696
Please refer to the end of the specification for access instructions.

Lengthy table referenced here
US20260363240A1-20261119-T00697
Please refer to the end of the specification for access instructions.

Lengthy table referenced here
US20260363240A1-20261119-T00698
Please refer to the end of the specification for access instructions.

Lengthy table referenced here
US20260363240A1-20261119-T00699
Please refer to the end of the specification for access instructions.

Lengthy table referenced here
US20260363240A1-20261119-T00700
Please refer to the end of the specification for access instructions.

Lengthy table referenced here
US20260363240A1-20261119-T00701
Please refer to the end of the specification for access instructions.

Lengthy table referenced here
US20260363240A1-20261119-T00702
Please refer to the end of the specification for access instructions.

Lengthy table referenced here
US20260363240A1-20261119-T00703
Please refer to the end of the specification for access instructions.

Lengthy table referenced here
US20260363240A1-20261119-T00704
Please refer to the end of the specification for access instructions.

Lengthy table referenced here
US20260363240A1-20261119-T00705
Please refer to the end of the specification for access instructions.

Lengthy table referenced here
US20260363240A1-20261119-T00706
Please refer to the end of the specification for access instructions.

Lengthy table referenced here
US20260363240A1-20261119-T00707
Please refer to the end of the specification for access instructions.

Lengthy table referenced here
US20260363240A1-20261119-T00708
Please refer to the end of the specification for access instructions.

Lengthy table referenced here
US20260363240A1-20261119-T00709
Please refer to the end of the specification for access instructions.

Lengthy table referenced here
US20260363240A1-20261119-T00710
Please refer to the end of the specification for access instructions.

Lengthy table referenced here
US20260363240A1-20261119-T00711
Please refer to the end of the specification for access instructions.

Lengthy table referenced here
US20260363240A1-20261119-T00712
Please refer to the end of the specification for access instructions.

Lengthy table referenced here
US20260363240A1-20261119-T00713
Please refer to the end of the specification for access instructions.

Lengthy table referenced here
US20260363240A1-20261119-T00714
Please refer to the end of the specification for access instructions.

Lengthy table referenced here
US20260363240A1-20261119-T00715
Please refer to the end of the specification for access instructions.

Lengthy table referenced here
US20260363240A1-20261119-T00716
Please refer to the end of the specification for access instructions.

Lengthy table referenced here
US20260363240A1-20261119-T00717
Please refer to the end of the specification for access instructions.

Lengthy table referenced here
US20260363240A1-20261119-T00718
Please refer to the end of the specification for access instructions.

Lengthy table referenced here
US20260363240A1-20261119-T00719
Please refer to the end of the specification for access instructions.

Meanwhile, the compound of the present invention of formula [I] can be produced according to the following production method, but the production method is not limited to these methods. Hereinafter, for example, “the compound represented by general formula [I]”, “the compound of formula [I]”, and “the compound [I]” are synonymous.

### <Production Method 1>

A compound of formula [III-2] among the compounds of the present invention can be produced according to a method based on the reaction scheme exemplified below.

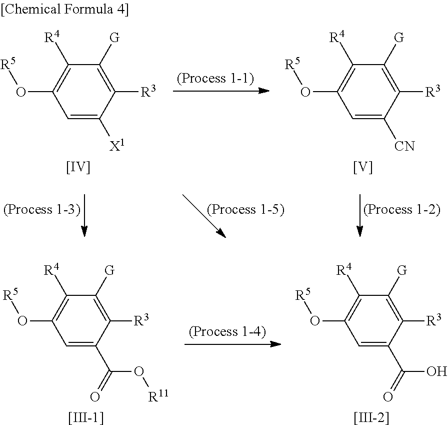

(In the formula, X¹ represents a halogen atom, R¹¹ represents a (C1-C6)alkyl, and G, R³, R⁴, and R⁵ are as defined above.)

That is, the compound of formula [III-2] can be produced by (Process 1-1) converting a compound of formula [IV] into a compound [V] using a cyanide in an appropriate solvent in the presence or absence of an appropriate catalyst, and then (Process 1-2) hydrolyzing the compound of formula [V] in an appropriate solvent in the presence of an appropriate acid or base. In addition, the compound [III-2] can be produced by (Process 1-3) subjecting a compound of formula [IV] to a carbon monoxide insertion reaction in an appropriate solvent in the presence of an appropriate catalyst, an appropriate base, and an appropriate alcohol to obtain a compound of formula [III-1] among the compounds of the present invention, and then (Process 1-4) hydrolyzing the compound of formula [III-1] in an appropriate solvent in the presence of an appropriate acid or base. Furthermore, the compound [III-2] can be produced by (Process 1-5) subjecting a compound of formula [IV] to a carbon monoxide insertion reaction in water or a mixed solvent of water and an appropriate solvent in the presence of an appropriate catalyst and an appropriate base.

### (Process 1-1)

Examples of the cyanide used in the current reaction include sodium cyanide, potassium cyanide, ammonium cyanide, zinc cyanide, copper cyanide, and a mixture thereof. The amount of use of the cyanide may be appropriately selected from the range of usually 1 to 100 mol, and is preferably 1.0 to 5.0 mol per 1 mol of the compound [IV].

Examples of the catalyst used in the current reaction include tetrakis(triphenylphosphine)palladium and [1,1-bis(diphenylphosphino)ferrocene]palladium (II) dichloride dichloromethane adduct. The amount of use of the catalyst may be appropriately selected from the range of usually 0.001 to 0.5 mol, and is preferably 0.01 to 0.2 mol per 1 mol of the compound [IV].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; halogenated hydrocarbons such as dichloromethane, chloroform, and 1,2-dichloroethane; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; alcohols such as methanol, ethanol, and 2-propanol; nitriles such as acetonitrile and propionitrile; esters such as ethyl acetate, butyl acetate, and ethyl propionate; aliphatic hydrocarbons such as pentane, hexane, cyclohexane, and heptane; pyridines such as pyridine and picoline; water, and a mixed solvent thereof. The amount of use of the solvent is 0.1 to 500 liters, and is preferably 0.3 to 50 liters per 1 mol of the compound [IV].

The reaction temperature of the current reaction may be selected from an arbitrary temperature range usually from −30 °C to the reflux temperature in the reaction system, and is preferably in the range of 0 °C to 150 °C.

The reaction time of the current reaction varies depending on the reaction temperature, the reaction substrate, the reaction amount, and the like, but is usually 10 minutes to 72 hours.

After completion of the reaction, the compound [V] can be isolated by conducting an operation of pouring the reaction mixture into water and collecting the precipitated solid by filtration, or subjecting the reaction mixture to extraction with an organic solvent, followed by concentration. The isolated compound [V] also can be further purified by column chromatography, recrystallization, or the like as necessary.

### (Process 1-2)

Examples of the acid that can be used in the current reaction include mineral acids such as hydrochloric acid, hydrobromic acid, and sulfuric acid; carboxylic acids such as acetic acid, propionic acid, and trifluoroacetic acid; and sulfonic acids such as methanesulfonic acid and trifluoromethanesulfonic acid. The amount of use of the acid may be appropriately selected from the range of 1 to moles corresponding to the amount of the solvent, and is preferably 1.0 to 100.0 moles per 1 mole of the compound [V].

Examples of the base that can be used in the current reaction include inorganic bases such as hydroxides of alkali metals, such as lithium hydroxide, sodium hydroxide, and potassium hydroxide; hydroxides of alkaline earth metals, such as calcium hydroxide and magnesium hydroxide; carbonates of alkali metals, such as sodium carbonate and potassium carbonate; and alkali metal bicarbonates such as sodium bicarbonate and potassium bicarbonate. The amount of use of the base may be appropriately selected from the range of 0.1 to 50.0 mol, and is preferably 0.5 to 20.0 mol per 1 mol of the compound [V].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; aprotic polar solvents such as N,N-dimethylacetamide, N-methyl-2-pyrrolidone, dimethyl sulfoxide, sulfolane, and 1,3-dimethyl-2-imidazolidinone; alcohols such as methanol, ethanol, and 2-propanol; ketones such as acetone, methyl ethyl ketone, and cyclohexanone; water, and a mixed solvent thereof. The amount of use of the solvent is 0.1 to 500 liters, and is preferably 0.3 to 30 liters per 1 mol of the compound [V].

The reaction temperature of the current reaction may be selected from an arbitrary temperature range usually from −30 °C to the reflux temperature in the reaction system, and is preferably in the range of 0 °C to 150 °C.

The reaction time of the current reaction varies depending on the reaction temperature, the reaction substrate, the reaction amount, and the like, but is usually 10 minutes to 72 hours.

After completion of the current reaction, the compound [III-2] can be isolated by conducting an operation of pouring the reaction mixture into water to neutralize the mixture and collecting the precipitated solid by filtration, or subjecting the reaction mixture to extraction with an organic solvent, followed by concentration. The isolated compound [III-2] also can be further purified by column chromatography, recrystallization, or the like as necessary.

### (Process 1-3)

As the catalyst that can be used in the current reaction, a transition metal complex or a combination of a transition metal salt and a ligand may be used. Examples of the transition metal complex include tetrakis(triphenylphosphine)palladium, dichloro[1,1′-bis(diphenylphosphino)ferrocene]palladium-dichloromethane adduct, and tris(dibenzylideneacetone)dipalladium(0). Examples of the transition metal salt include palladium chloride, palladium bromide, and palladium acetate. Examples of the ligand include trimethylphosphine, triethylphosphine, tributylphosphine, triphenylphosphine, 1,3-bis(dimethylphosphino)propane, 1,2-bis(diphenylphosphino)ethane, and 1,3-bis(diphenylphosphino)propane. The amount of use of the catalyst is 0.001 to 0.1 mol, and is preferably 0.01 to 0.05 mol per 1 mol of the compound [IV].

Examples of the base that can be used in the current reaction include inorganic bases such as hydroxides of alkali metals, such as lithium hydroxide, sodium hydroxide, and potassium hydroxide; hydroxides of alkaline earth metals, such as calcium hydroxide and magnesium hydroxide; carbonates of alkali metals, such as sodium carbonate and potassium carbonate; and alkali metal bicarbonates such as sodium bicarbonate and potassium bicarbonate; and organic bases such as triethylamine, 4-methylmorpholine, N-ethyldiisopropylamine, 1,8-diazabicyclo[5.4.0]-7-undecene, pyridine, 4-(N,N-dimethylamino)pyridine, and 2,6-lutidine. The amount of use of the base is 0.1 to 10.0 mol, and is preferably 0.1 to 2.0 mol per 1 mol of the compound [IV]. In the case of an organic base, the organic base can also be used as a solvent.

Examples of the alcohol that can be used in the current reaction include methanol, ethanol, and 2-propanol. The amount of use of the alcohols is 0.1 to 500 liters, and is preferably 0.3 to 30 liters per 1 mol of the compound [IV].

The alcohol can also be used as a solvent.

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; alcohols such as methanol, ethanol, and 2-propanol; nitriles such as acetonitrile and propionitrile; ketones such as acetone, methyl ethyl ketone, and cyclohexanone; and a mixed solvent thereof. The amount of use of the solvent is 0.1 to 500 liters, and is preferably 0.3 to 30 liters per 1 mol of the compound [IV].

The reaction temperature of the current reaction may be selected from an arbitrary temperature range usually from −30 °C to the reflux temperature in the reaction system, and is preferably in the range of 0 °C to 150 °C.

The reaction pressure of the current reaction may be selected from an arbitrary pressure range usually from the atmospheric pressure to 50 atm, and is preferably in the range of 1 atm to less than 10 atm.

The reaction time of the current reaction varies depending on the reaction temperature, the reaction pressure, the reaction substrate, the reaction amount, and the like, but is usually 10 minutes to 72 hours.

After completion of the current reaction, the compound [III-1] can be isolated by conducting an operation of pouring the reaction mixture into water to neutralize the mixture and collecting the precipitated solid by filtration, or subjecting the reaction mixture to extraction with an organic solvent, followed by concentration. The isolated compound [III-1] also can be further purified by column chromatography, recrystallization, or the like as necessary.

### (Process 1-4)

The acid, base, and solvent that can be used in the current reaction, the reaction temperature, and the reaction time are the same as those in Process 1-2.

After completion of the current reaction, the compound [III-2] can be isolated by conducting an operation of pouring the reaction mixture into water to neutralize the mixture and collecting the precipitated solid by filtration, or subjecting the reaction mixture to extraction with an organic solvent, followed by concentration. The isolated compound [III-2] also can be further purified by column chromatography, recrystallization, or the like as necessary.

### (Process 1-5)

The base and solvent that can be used in the current reaction, the reaction temperature, and the reaction time are the same as those in Process 1-3.

After completion of the current reaction, the compound [III-2] can be isolated by conducting an operation of pouring the reaction mixture into water to neutralize the mixture and collecting the precipitated solid by filtration, or subjecting the reaction mixture to extraction with an organic solvent, followed by concentration. The isolated compound [III-2] also can be further purified by column chromatography, recrystallization, or the like as necessary.

Furthermore, the compound [III-2] can also be produced by reacting a compound of formula [IV] with an appropriate carbon monoxide equivalent in an appropriate solvent, in the presence of an appropriate acid anhydride, an appropriate base, an appropriate inorganic salt, and an appropriate catalyst, according to the method described in Org. Lett., Vol. 5 (No. 23), page 4269 to 4272 (2003) or Synthesis, Vol. 18, page 3106 to 3110 (2006).

### <Production Method 2>

A compound of formula [I-1] among the compounds of the present invention can be produced using a compound of formula [III-3] according to a method based on the reaction scheme exemplified below.

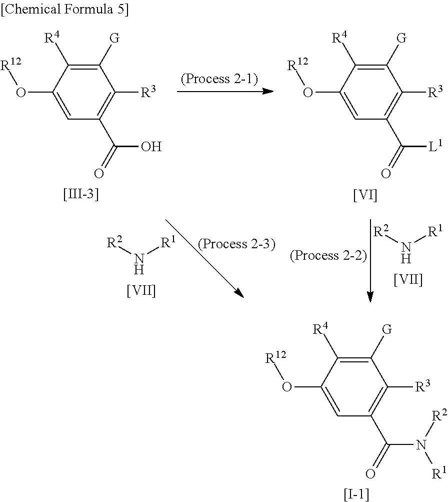

(In the formula, L¹ represents a leaving group such as a halogen atom, OC(═O)OR¹¹, a methanesulfonyloxy, a trifluoromethanesulfonyloxy, a nonafluorobutanesulfonyloxy, a benzenesulfonyloxy, or a p-toluenesulfonyloxy, R¹² represents a hydrogen atom or R⁵, and G, R¹, R², R³, R⁴, R⁵, and R¹¹ are as defined above.)

That is, the compound of formula [I-1] can be produced by (Process 2-1) converting a compound [III-3] into a compound [VI] which is an active derivative such as an acid halide or a mixed acid anhydride in an appropriate solvent, in the presence or absence of an appropriate catalyst, and then (Process 2-2) reacting the compound [VI] with a compound [VII], in an appropriate solvent, in the presence or absence of an appropriate base, in the presence or absence of an appropriate catalyst. Furthermore, the compound of formula [I-1] can also be produced by (Process 2-3) reacting a compound [III-3] and a compound [VII] in an appropriate solvent, in the presence or absence of an appropriate condensing agent and an appropriate additive, in the presence or absence of an appropriate catalyst, in the presence or absence of an appropriate base.

### (Process 2-1)

Examples of the reagent for converting the compound [II-2] into the compound [VI] used in the current reaction include thionyl chloride, oxalyl chloride, phosphoryl chloride, isobutyl chlorocarbonate, methanesulfonyl chloride, trifluoromethanesulfonyl chloride, and p-toluenesulfonyl chloride. The amount of use of the reagent for converting the compound [II-2] into the compound [VI] may be appropriately selected from the range of 0.5 to 30.0 mol, and is preferably 0.5 to 6.0 mol per 1 mol of the compound [III-3].

Examples of the catalyst that can be used in the current reaction include N,N-dimethylformamide. The amount of use of the catalyst may be appropriately selected from the range of 0.01 to 1.0 mol, and is preferably 0.01 to 0.1 mol per 1 mol of the compound [III-3].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; halogenated hydrocarbons such as dichloromethane, chloroform, and 1,2-dichloroethane; nitriles such as acetonitrile and propionitrile; esters such as ethyl acetate, butyl acetate, and ethyl propionate; aliphatic hydrocarbons such as pentane, hexane, cyclohexane, and heptane; and a mixed solvent thereof. The amount of use of the solvent is 0.1 to 100 liters, and is preferably 0.3 to 10 liters per 1 mol of the compound [III-3].

The reaction temperature of the current reaction may be selected from an arbitrary temperature range usually from −30 °C to the reflux temperature in the reaction system, and is preferably in the range of 0 °C to 150 °C.

The reaction time of the current reaction varies depending on the reaction temperature, the reaction substrate, the reaction amount, and the like, but is usually 10 minutes to 24 hours.

After completion of the reaction, the compound [VI] can be isolated by conducting an operation such as concentration.

### (Process 2-2)

The amount of use of the compound [VII] used in the current reaction may be appropriately selected from the range of usually 1 to 5 mol, and is preferably 1.0 to 2.5 mol per 1 mol of the compound [VI].

Examples of the base that can be used in the current reaction include organic bases such as triethylamine, 4-methylmorpholine, N-ethyldiisopropylamine, 1,8-diazabicyclo[5.4.0]-7-undecene, pyridine, 4-(N,N-dimethylamino)pyridine, and 2,6-lutidine; and inorganic bases such as hydroxides of alkali metals, such as lithium hydroxide, sodium hydroxide, and potassium hydroxide; carbonates of alkali metals, such as sodium carbonate and potassium carbonate; hydroxides of alkaline earth metals, such as calcium hydroxide and magnesium hydroxide; and bicarbonates of alkali metals, such as sodium bicarbonate and potassium bicarbonate. The amount of use of the base may be appropriately selected from the range of 1 to 10 mol, and is preferably 1.0 to 5 mol per 1 mol of the compound [VI].

Examples of the solvent that can be used in the current reaction include halogenated hydrocarbons such as dichloromethane, chloroform, and 1,2-dichloroethane; aliphatic hydrocarbons such as pentane, hexane, cyclohexane, and heptane; aromatic hydrocarbons such as benzene, toluene, xylene, and chlorobenzene; esters such as ethyl acetate, butyl acetate, and ethyl propionate; nitriles such as acetonitrile and propionitrile; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; ethers such as diethyl ether, cyclopentyl methyl ether, tetrahydrofuran, 2-methyltetrahydrofuran, and 1,4-dioxane; water, and a mixed solvent thereof. The amount of use of the solvent is 0.1 to 100 liters, and is preferably 0.3 to 50 liters per 1 mol of the compound [VI].

The current reaction can be carried out in the presence of a catalyst as necessary, and examples of the catalyst include tetrabutylammonium bromide, tetrabutylammonium chloride, 15-crown-5, and 18-crown-6. The amount of use of the catalyst may be appropriately selected from the range of 0.00 to 1 mol, and is preferably 0.01 to 0.1 mol per 1 mol of the compound [VI].

The reaction temperature of the current reaction may be selected from an arbitrary temperature range from −20 °C to the reflux temperature in the reaction system, and is preferably in the range of 0 °C to 80 °C.

The reaction time of the current reaction varies depending on the reaction temperature, the reaction substrate, the reaction amount, and the like, but is usually 1 minute to 48 hours.

After completion of the reaction, the compound [I-1] can be isolated by conducting an operation of pouring the reaction mixture into water and collecting the precipitated solid by filtration, or subjecting the reaction mixture to extraction with an organic solvent, followed by concentration. The isolated compound [I-1] also can be further purified by column chromatography, recrystallization, or the like as necessary.

### (Process 2-3)

The amount of use of the compound [VII] used in the current reaction may be appropriately selected from the range of 1 to 3 mol, and is preferably 1.0 to 1.5 mol per 1 mol of the compound [III-3].

Examples of the condensing agent that can be used in the current reaction include 1-ethyl-3-(3-dimethylaminopropyl)carbodiimide hydrochloride, 1-ethyl-3-(3-dimethylaminopropyl)carbodiimide, N,N′-dicyclohexylcarbodiimide, N,N′-diisopropylcarbodiimide, N,N′-carbonyldiimidazole, 4-(4,6-dimethoxy-1,3,5-triazine-2-yl)-4-methylmorpholinium chloride, 1H-benzotriazole-1-yloxytris(dimethylamino)phosphonium hexafluorophosphate, {{[(1-cyano-2-ethoxy-2-oxoethylidene)amino]oxy}-4-morpholinomethylene}dimethylammonium hexafluorophosphate, and O-(7-azabenzotriazole-1-yl)-N,N,N′,N′-tetramethyluronium hexafluorophosphate. The amount of use of the condensing agent may be appropriately selected from the range of 1.0 to 5.0 mol, and is preferably 1.0 to 3.0 mol per 1 mol of the compound [III-3].

When an additive is used in the current reaction, examples of the additive that can be used include 1-hydroxybenzotriazole, N-hydroxysuccinimide, and 1-hydroxy-7-azabenzotriazole. The amount of use of the additive may be appropriately selected from the range of 0.01 to 3.0 mol, and is preferably 1.0 to 1.5 mol per 1 mol of the compound [III-3].

When a base is used in the current reaction, examples of the base that can be used include organic bases such as triethylamine, 4-methylmorpholine, N-ethyldiisopropylamine, 1,8-diazabicyclo[5.4.0]-7-undecene, pyridine, 4-(N,N-dimethylamino)pyridine, and 2,6-lutidine. The amount of use of the base may be appropriately selected from the range of 0.1 to 5.0 mol, and is preferably 0.1 to 3.0 mol per 1 mol of the compound [III-3].

When a catalyst is used in the current reaction, examples of the catalyst that can be used include 4-(N,N-dimethylamino)pyridine. The amount of use of the catalyst may be appropriately selected from the range of 0.001 to 1.0 mol, and is preferably 0.01 to 0.1 mol per 1 mol of the compound [III-3].

Examples of the solvent that can be used in the current reaction include halogenated hydrocarbons such as dichloromethane, chloroform, and 1,2-dichloroethane; aliphatic hydrocarbons such as pentane, hexane, cyclohexane, and heptane; esters such as ethyl acetate, butyl acetate, and ethyl propionate; nitriles such as acetonitrile and propionitrile; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; ethers such as diethyl ether, cyclopentyl methyl ether, tetrahydrofuran, 2-methyltetrahydrofuran, and 1,4-dioxane; and a mixed solvent thereof. The amount of use of the solvent is 0.1 to 100 liters, and is preferably 0.3 to 50 liters per 1 mol of the compound [III-3].

The reaction temperature of the current reaction may be selected from an arbitrary temperature range from −20 °C to the reflux temperature in the reaction system, and is preferably in the range of 0 °C to 80 °C.

The reaction time of the current reaction varies depending on the reaction temperature, the reaction substrate, the reaction amount, and the like, but is usually 1 minute to 96 hours.

After completion of the reaction, the compound [I-1] can be isolated by conducting an operation of pouring the reaction mixture into water and collecting the precipitated solid by filtration, or subjecting the reaction mixture to extraction with an organic solvent, followed by concentration. The isolated compound [I-1] also can be further purified by column chromatography, recrystallization, or the like as necessary.

### <Production Method 3>

A compound of formula [I-2] among the compounds of the present invention can be produced using a compound of formula [II-1] according to a method based on the reaction scheme exemplified below.

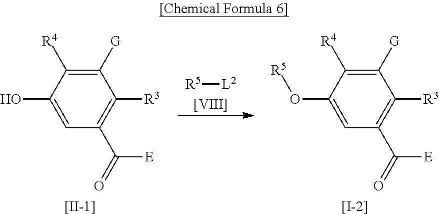

(In the formula, E represents a (C1-C6)alkoxy or a group NR¹R², L² represents a leaving group such as a halogen atom, a methanesulfonyloxy, a trifluoromethanesulfonyloxy, a nonafluorobutanesulfonyloxy, a benzenesulfonyloxy, or a p-toluenesulfonyloxy, and G, R¹, R², R³, R⁴, and R⁵ are as defined above). That is, the compound of the present invention of formula [I-2] can be produced by reacting a compound [II-1] and a compound [VIII] in an appropriate solvent, in the presence of an appropriate base, in the presence or absence of an appropriate catalyst.)

The amount of use of the compound [VIII] used in the current reaction may be appropriately selected from the range of 1.0 to 15.0 mol, and is preferably 1.0 to 6.0 mol per 1 mol of the compound [II-1].

Examples of the base that can be used in the current reaction include inorganic bases such as hydroxides of alkali metals, such as sodium hydroxide and potassium hydroxide; hydroxides of alkaline earth metals, such as calcium hydroxide and magnesium hydroxide; carbonates of alkali metals, such as sodium carbonate and potassium carbonate; and alkali metal bicarbonates such as sodium bicarbonate and potassium bicarbonate; metal hydrides such as sodium hydride and potassium hydride; metal salts of alcohols, such as sodium methoxide, sodium ethoxide, potassium tert-butoxide; and organic bases such as triethylamine, 4-methylmorpholine, N-ethyldiisopropylamine, 1,8-diazabicyclo[5.4.0]-7-undecene, pyridine, 4-(N,N-dimethylamino)pyridine, and 2,6-lutidine. The amount of use of the base may be appropriately selected from the range of 1.0 to 5.0 mol, and is preferably 1.0 to 1.5 mol per 1 mol of the compound [II-1].

Examples of the catalyst that can be used in the current reaction include tetra-n-butylammonium bromide and tetra-n-butylammonium iodide. The amount of use of the catalyst may be appropriately selected from the range of 0.01 to 1.0 mol, and is preferably 0.05 to 0.5 mol per 1 mol of the compound [II-1].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; halogenated hydrocarbons such as dichloromethane, chloroform, and 1,2-dichloroethane; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; alcohols such as methanol, ethanol, and 2-propanol; nitriles such as acetonitrile and propionitrile; esters such as ethyl acetate, butyl acetate, and ethyl propionate; aliphatic hydrocarbons such as pentane, hexane, cyclohexane, and heptane; pyridines such as pyridine and picoline; water, and a mixed solvent thereof. The amount of use of the solvent is 0.1 to 100 liters, and is preferably 0.1 to 15 liters per 1 mol of the compound [II-1].

The reaction temperature of the current reaction may be selected from an arbitrary temperature range usually from −30 °C to the reflux temperature in the reaction system, and is preferably in the range of 0 °C to 150 °C.

The reaction time of the current reaction varies depending on the reaction temperature, the reaction substrate, the reaction amount, and the like, but is usually 10 minutes to 24 hours.

After completion of the reaction, the compound [I-2] can be isolated by conducting an operation of pouring the reaction mixture into water or the like and subjecting the mixture to extraction with an organic solvent, followed by concentration. The isolated compound [I-2] also can be further purified by column chromatography, recrystallization, or the like as necessary.

### <Production Method 4>

A compound of formula [I-2] among the compounds of the present invention can also be produced using a compound of formula [II-1] according to a method based on the reaction scheme exemplified below.

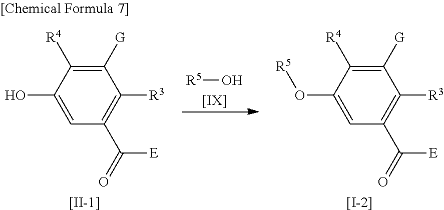

(In the formula, G, R³, R⁴, R⁵, and E are as defined above.)

That is, the compound of the present invention of formula [I-2] can be produced by reacting a compound [II-1] and a compound [IX] in an appropriate solvent, in the presence of a trisubstituted phosphine and an azodicarboxylic acid derivative, or in the presence of phosphorane.

The amount of use of the compound [IX] used in the current reaction may be appropriately selected from the range of 1.0 to 5.0 mol, and is preferably 1.0 to 3.0 mol per 1 mol of the compound [II-1].

Examples of the trisubstituted phosphine that can be used in the current reaction include triphenylphosphine, tributylphosphine, and trimethylphosphine. The amount of use of the trisubstituted phosphine may be appropriately selected from the range of 1.0 to 5.0 mol, and is preferably 1.0 to 3.0 mol per 1 mol of the compound [II-1].

Examples of the azodicarboxylic acid derivative that can be used in the current reaction include diethyl azodicarboxylate, diisopropyl azodicarboxylate, dimethoxyethyl azodicarboxylate, and N,N,N′,N′-tetramethylazodicarboxamide. The amount of use of the azodicarboxylic acid derivative may be appropriately selected from the range of 1.0 to 5.0 mol, and is preferably 1.0 to 3.0 mol per 1 mol of the compound [II-1].

Examples of the phosphorane that can be used in the current reaction include cyanomethylenetrimethylphosphorane and cyanomethylenetributylphosphorane. The amount of use of the phosphorane may be appropriately selected from the range of 1.0 to 5.0 mol, and is preferably 1.0 to 3.0 mol per 1 mol of the compound [II-1].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; halogenated hydrocarbons such as dichloromethane, chloroform, and 1,2-dichloroethane; aliphatic hydrocarbons such as pentane, hexane, cyclohexane, and heptane; ketones such as acetone, methyl ethyl ketone, and cyclohexanone; acetic acid, water, and a mixed solvent thereof. The amount of use of the solvent is 0.1 to 100 liters, and is preferably 0.3 to 15 liters per 1 mol of the compound [II-1].

The reaction temperature of the current reaction may be selected from an arbitrary temperature range usually from −30 °C to the reflux temperature in the reaction system, and is preferably in the range of 0 °C to 150 °C.

The reaction time of the current reaction varies depending on the reaction temperature, the reaction substrate, the reaction amount, and the like, but is usually 10 minutes to 24 hours.

After completion of the reaction, the compound [I-2] can be isolated by conducting an operation of concentrating the reaction mixture as it is, or pouring the reaction mixture into water or the like and subjecting the mixture to extraction with an organic solvent, followed by concentration.

The isolated compound [I-2] also can be further purified by column chromatography, recrystallization, or the like as necessary.

### <Production Method 5>

A compound of formula [I-4] among the compounds of the present invention can be produced using a compound of formula [I-3] according to a method based on the reaction scheme exemplified below.

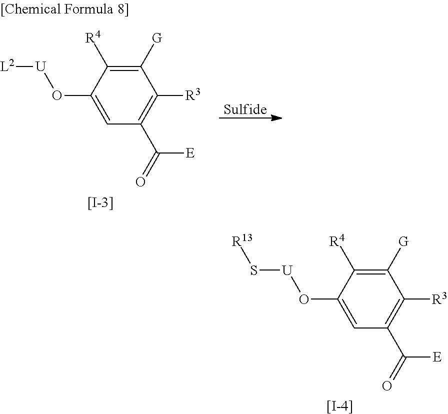

(In the formula, R¹³ represents a hydrogen atom; a cyano; a (C1-C6)alkyl; a (C1-C6)haloalkyl; a (C2-C6)alkenyl; a (C2-C6)haloalkenyl; a (C3-C6)cycloalkyl (C1-C6)alkyl; a (C3-C6)halocycloalkyl (C1-C6)alkyl; a (C3-C6) cycloalkyl; a (C3-C6)halocycloalkyl; a tri (C1-C6 alkyl)silyl (C1-C6)alkyl; or a phenyl, U represents (C1-C12)alkylene; (C3-C6)cycloalkylene; (C2-C6)alkenylene; (C1-C6)alkyleneoxy (C1-C12)alkylene; or (C2-C6)alkynylene, and G, L², R³, R⁴, and E are as defined above.)

That is, the compound of formula [I-4] can be produced by reacting a compound [I-3] and a sulfide in an appropriate solvent, in the presence or absence of an appropriate base, in the presence or absence of an appropriate catalyst.

Examples of the sulfide that can be used in the current reaction include hydrosulfides of alkali metals, such as sodium hydrosulfide and potassium hydrosulfide; thiocyanates such as sodium thiocyanate, potassium thiocyanate, and ammonium thiocyanate; alkyl mercaptans such as methyl mercaptan, ethyl mercaptan, and tert-butyl mercaptan; haloalkyl mercaptans such as 2,2,2-trifluoroethyl mercaptan; cycloalkylalkyl mercaptans such as cyclopropylmethyl mercaptan; and thioureas such as thiourea. The amount of use of the sulfide may be appropriately selected from the range of 1.0 to 10.0 mol, and is preferably 1.0 to 5.0 mol per 1 mol of the compound [I-3].

Examples of the base that can be used in the current reaction include inorganic bases such as hydroxides of alkali metals, such as sodium hydroxide and potassium hydroxide; hydroxides of alkaline earth metals, such as calcium hydroxide and magnesium hydroxide; carbonates of alkali metals, such as sodium carbonate and potassium carbonate; and alkali metal bicarbonates such as sodium bicarbonate and potassium bicarbonate; metal hydrides such as sodium hydride and potassium hydride; metal salts of alcohols, such as sodium methoxide, sodium ethoxide, potassium tert-butoxide; and organic bases such as triethylamine, 4-methylmorpholine, N-ethyldiisopropylamine, 1,8-diazabicyclo[5.4.0]-7-undecene, pyridine, 4-(N,N-dimethylamino)pyridine, and 2,6-lutidine. The amount of use of the base may be appropriately selected from the range of 1.0 to 5.0 mol, and is preferably 1.0 to 2.0 mol per 1 mol of the compound [I-3].

Examples of the catalyst that can be used in the current reaction include sodium iodide and potassium iodide. The amount of use of the catalyst may be appropriately selected from the range of 0.01 to 1.0 mol, and is preferably 0.1 to 0.5 mol per 1 mol of the compound [I-3].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; alcohols such as methanol, ethanol, and 2-propanol; nitriles such as acetonitrile and propionitrile; aliphatic hydrocarbons such as pentane, hexane, cyclohexane, and heptane; pyridines such as pyridine and picoline; and a mixed solvent thereof. The amount of use of the solvent is 0.1 to 100 liters, and is preferably 0.3 to 15 liters per 1 mol of the compound [I-3].

The reaction temperature of the current reaction may be selected from an arbitrary temperature range usually from 0 °C to the reflux temperature in the reaction system, and is preferably in the range of room temperature to 150 °C.

The reaction time of the current reaction varies depending on the reaction temperature, the reaction substrate, the reaction amount, and the like, but is usually 10 minutes to 96 hours.

After completion of the reaction, the compound [I-4] can be isolated by conducting an operation of concentrating the reaction mixture as it is, or pouring the reaction mixture into water or the like and subjecting the mixture to extraction with an organic solvent, followed by concentration.

The isolated compound [I-4] also can be further purified by column chromatography, recrystallization, or the like as necessary.

### <Production Method 6>

A compound of formula [I-6] among the compounds of the present invention can be produced using a compound of formula [I-5] according to a method based on the reaction scheme exemplified below.

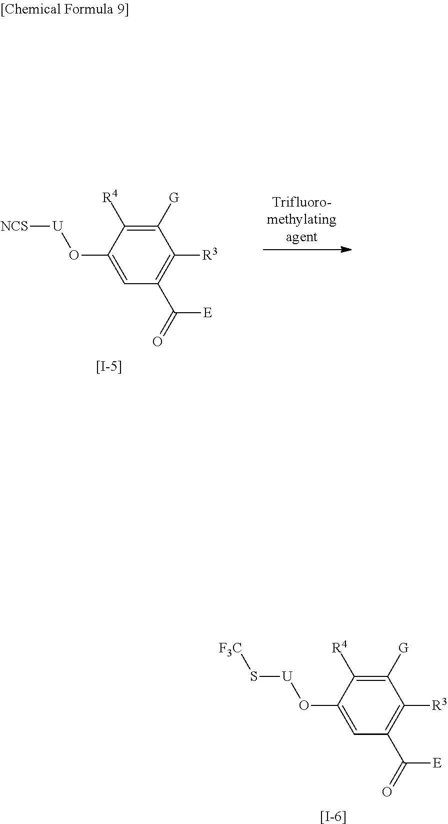

(In the formula, G, R³, R⁴, E, and U are as defined above.)

That is, the compound of formula [I-6] can be produced by reacting a compound [I-5] and a trifluoromethylating agent in an appropriate solvent in the presence of an appropriate catalyst.

Examples of the trifluoromethylating agent used in the current reaction include (trifluoromethyl)trimethylsilane, triethyl(trifluoromethyl)silane, and the trifluoromethylating agent described in WO 2015/199109. The amount of use of the trifluoromethylating agent may be appropriately selected from the range of 1.0 to 10.0 mol, and is preferably 1.0 to 5.0 mol per 1 mol of the compound [I-5].

Examples of the catalyst that can be used in the current reaction include tetra-n-butylammonium fluoride, cesium fluoride, and potassium fluoride. The amount of use of the catalyst may be appropriately selected from the range of 0.01 to 1.0 mol, and is preferably 0.05 to 0.3 mol per 1 mol of the compound [I-5].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; halogenated hydrocarbons such as dichloromethane, chloroform, and 1,2-dichloroethane; nitriles such as acetonitrile and propionitrile; aliphatic hydrocarbons such as pentane, hexane, cyclohexane, and heptane; and a mixed solvent thereof. The amount of use of the solvent is 0.1 to 100 liters, and is preferably 0.3 to 15 liters per 1 mol of the compound [I-5].

The reaction temperature of the current reaction may be selected from an arbitrary temperature range usually from −30 °C to the reflux temperature in the reaction system, and is preferably in the range of 0 °C to room temperature.

The reaction time of the current reaction varies depending on the reaction temperature, the reaction substrate, the reaction amount, and the like, but is usually 10 minutes to 24 hours.

After completion of the reaction, the compound [I-6] can be isolated by conducting an operation of concentrating the reaction mixture as it is, or pouring the reaction mixture into water or the like and subjecting the mixture to extraction with an organic solvent, followed by concentration.

The isolated compound [I-6] also can be further purified by column chromatography, recrystallization, or the like as necessary.

### <Production Method 7>

A compound of formula [I-8] among the compounds of the present invention can be produced using a compound of formula [I-7] according to a method based on the reaction scheme exemplified below.

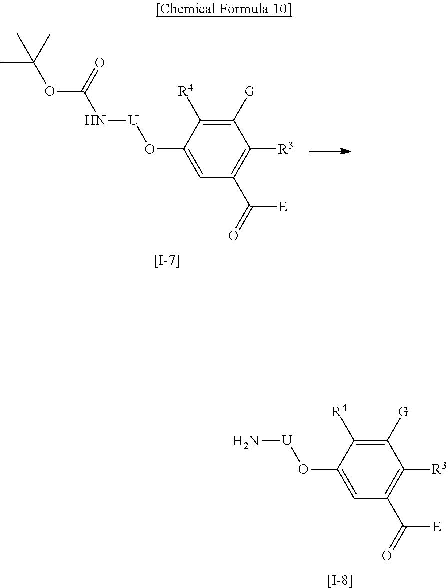

(In the formula, G, R³, R⁴, E, and U are as defined above.)

That is, the compound of formula [I-8] can be produced by reacting a compound [I-7] in an appropriate solvent, in the presence of an appropriate acid, under conditions used for deprotection of a tert-butoxycarbonyl group, for example, according to the method described in GREEN'S PROTECTIVE GROUPS in Organic Synthesis; the 5th edition (John Wiley and Sons, 2014, Peter G. M. Wuts).

### <Production Method 8>

A compound of formula [I-9] among the compounds of the present invention can be produced using a compound of formula [I-8] according to a method based on the reaction scheme exemplified below.

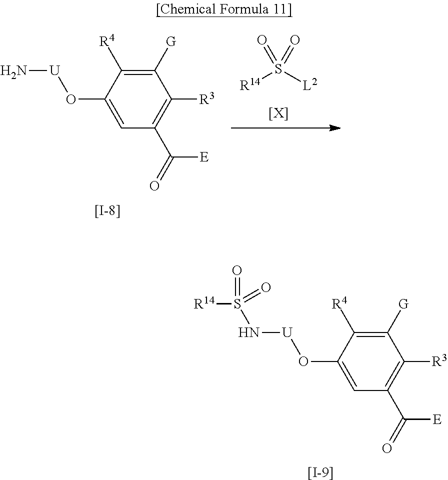

[In the formula, R¹⁴ represents a (C1-C6)alkyl; a (C1-C6)haloalkyl; or a phenyl (the phenyl is optionally mono-substituted or poly-substituted with R⁷), and G, L², R³, R⁴, R⁷, E, and U are as defined above.]

That is, the compound of formula [I-9] can be produced by reacting a compound [I-8] and a compound [X] in an appropriate solvent, in the presence of an appropriate base, in the presence or absence of an appropriate additive.

The amount of use of the compound [X] used in the current reaction may be appropriately selected from the range of 1.0 to 5.0 mol, and is preferably 1.0 to 2.0 mol per 1 mol of the compound [I-8].

Examples of the base that can be used in the current reaction include inorganic bases such as hydroxides of alkali metals, such as lithium hydroxide, sodium hydroxide, and potassium hydroxide; hydroxides of alkaline earth metals, such as calcium hydroxide and magnesium hydroxide; carbonates of alkali metals, such as sodium carbonate and potassium carbonate; and alkali metal bicarbonates such as sodium bicarbonate and potassium bicarbonate; and organic bases such as triethylamine, 4-methylmorpholine, N-ethyldiisopropylamine, 1,8-diazabicyclo[5.4.0]-7-undecene, pyridine, 4-(N,N-dimethylamino)pyridine, and 2,6-lutidine. The amount of use of the base may be appropriately selected from the range of 1.0 to 10.0 mol, and is preferably 1.0 to 3.0 mol per 1 mol of the compound [I-8].

When an additive is used in the current reaction, examples of the additive that can be used include molecular sieve 3A, molecular sieve 4A, and molecular sieve 5A.

The amount of use of the additive may be appropriately selected from the range of 0.01 to 10 g, and is preferably 0.1 to 1.0 g per 1 g of the compound [I-8].

Examples of the solvent that can be used in the current reaction include ethers such as diethyl ether, cyclopentyl methyl ether, tetrahydrofuran, 2-methyltetrahydrofuran, and 1,4-dioxane; halogenated hydrocarbons such as dichloromethane, chloroform, and 1,2-dichloroethane; aromatic hydrocarbons such as benzene, toluene, xylene, and chlorobenzene; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; nitriles such as acetonitrile and propionitrile; aliphatic hydrocarbons such as pentane, hexane, cyclohexane, and heptane; and a mixed solvent thereof. The amount of use of the solvent is 0.1 to 100 liters, and is preferably 0.3 to 30 liters per 1 mol of the compound [I-8].

The reaction temperature of the current reaction may be selected from an arbitrary temperature range usually from −70 °C to the reflux temperature in the reaction system, and is preferably in the range of −20 °C to room temperature.

The reaction time of the current reaction varies depending on the reaction temperature, the reaction substrate, the reaction amount, and the like, but is usually 10 minutes to 24 hours.

After completion of the reaction, the compound [I-9] can be isolated by conducting an operation of filtering off an unnecessary substance, concentrating the resulting substance as necessary, and then subjecting the concentrated resultant to extraction with an organic solvent, or pouring the reaction mixture into water or the like and subjecting the mixture with an organic solvent, followed by concentration. The isolated compound [I-9] also can be further purified by column chromatography, recrystallization, or the like as necessary.

### <Production Method 9>

A compound of formula [I-10] among the compounds of the present invention can be produced using a compound of formula [I-9] according to a method based on the reaction scheme exemplified below.

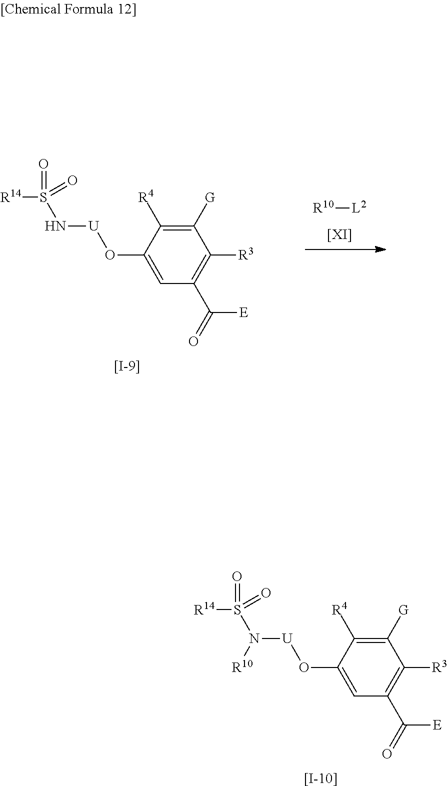

(In the formula, G, L², R³, R⁴, R¹⁰, R¹⁴, E, and U are as defined above.)

That is, the compound of formula [I-10] can be produced by reacting a compound [I-9] with a compound [XI] or di-tert-butyl dicarbonate in an appropriate solvent in the presence of an appropriate base.

The amount of use of the compound [XI] or di-tert-butyl dicarbonate used in the current reaction may be appropriately selected from the range of 1.0 to 5.0 mol, and is preferably 1.0 to 2.0 mol per 1 mol of the compound [I-9].

Examples of the base that can be used in the current reaction include inorganic bases such as hydroxides of alkali metals, such as sodium hydroxide and potassium hydroxide; hydroxides of alkaline earth metals, such as calcium hydroxide and magnesium hydroxide; carbonates of alkali metals, such as sodium carbonate and potassium carbonate; and alkali metal bicarbonates such as sodium bicarbonate and potassium bicarbonate; and organic bases such as triethylamine, 4-methylmorpholine, N-ethyldiisopropylamine, 1,8-diazabicyclo[5.4.0]-7-undecene, pyridine, 4-(N,N-dimethylamino)pyridine, and 2,6-lutidine. The amount of use of the base may be appropriately selected from the range of 1.0 to 10 mol, and is preferably 1.0 to 5.0 mol per 1 mol of the compound [I-9].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; halogenated hydrocarbons such as dichloromethane, chloroform, and 1,2-dichloroethane; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; alcohols such as methanol, ethanol, and 2-propanol; nitriles such as acetonitrile and propionitrile; esters such as ethyl acetate and ethyl propionate; aliphatic hydrocarbons such as pentane, hexane, cyclohexane, and heptane; pyridines such as pyridine and picoline; water, and a mixed solvent thereof. The amount of use of the solvent is 0.1 to 300 liters, and is preferably 0.1 to 100 liters per 1 mol of the compound [I-9].

The reaction temperature of the current reaction may be selected from an arbitrary temperature range usually from −30 °C to the reflux temperature in the reaction system, and is preferably in the range of 0 °C to 150 °C.

The reaction time of the current reaction varies depending on the reaction temperature, the reaction substrate, the reaction amount, and the like, but is usually 10 minutes to 24 hours.

After completion of the reaction, the compound [I-10] can be isolated by conducting an operation of concentrating the reaction mixture as it is, or pouring the reaction mixture into water or the like and subjecting the mixture to extraction with an organic solvent, followed by concentration. The isolated compound [I-10] also can be further purified by column chromatography, recrystallization, or the like as necessary.

### <Production Method 10>

A compound of formula [I-12] among the compounds of the present invention can be produced using a compound of formula [I-11] according to a method based on the reaction scheme exemplified below.

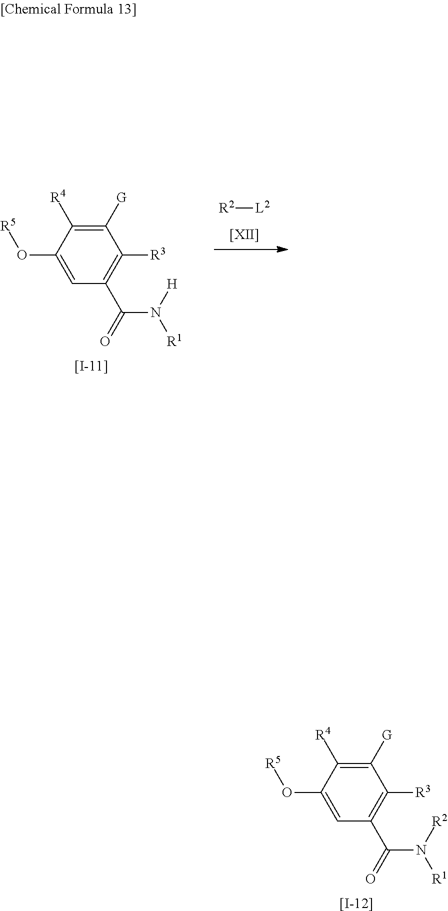

(In the formula, G, L², R¹, R², R³, R⁴, and R⁵ are as defined above.)

That is, the compound of formula [I-12] can be produced by reacting a compound [I-11] and a compound [XII] in an appropriate solvent in the presence of an appropriate base.

The amount of use of the compound [XII] used in the current reaction may be appropriately selected from the range of 1.0 to 30.0 mol, and is preferably 1.0 to 6.0 mol per 1 mol of the compound [I-11].

Examples of the base that can be used in the current reaction include inorganic bases such as hydroxides of alkali metals, such as sodium hydroxide and potassium hydroxide; hydroxides of alkaline earth metals, such as calcium hydroxide and magnesium hydroxide; carbonates of alkali metals, such as sodium carbonate and potassium carbonate; and alkali metal bicarbonates such as sodium bicarbonate and potassium bicarbonate; metal hydrides such as sodium hydride and potassium hydride; and metal salts of alcohols, such as sodium methoxide, sodium ethoxide, and potassium tert-butoxide. The amount of use of the base may be appropriately selected from the range of 1.0 to 10.0 mol, and is preferably 1.0 to 5.0 mol per 1 mol of the compound [I-11].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; halogenated hydrocarbons such as dichloromethane, chloroform, and 1,2-dichloroethane; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; alcohols such as methanol, ethanol, and 2-propanol; nitriles such as acetonitrile and propionitrile; esters such as ethyl acetate, butyl acetate, and ethyl propionate; aliphatic hydrocarbons such as pentane, hexane, cyclohexane, and heptane; pyridines such as pyridine and picoline; water, and a mixed solvent thereof. The amount of use of the solvent is 0.1 to 100 liters, and is preferably 0.1 to 25 liters per 1 mol of the compound [I-11].

The reaction temperature of the current reaction may be selected from an arbitrary temperature range usually from −30 °C to the reflux temperature in the reaction system, and is preferably in the range of −10 °C to 150 °C.

The reaction time of the current reaction varies depending on the reaction temperature, the reaction substrate, the reaction amount, and the like, but is usually 10 minutes to 24 hours.

After completion of the reaction, the compound [I-12] can be isolated by conducting an operation of pouring the reaction mixture into water or the like and subjecting the mixture to extraction with an organic solvent, followed by concentration. The isolated compound [I-12] also can be further purified by column chromatography, recrystallization, or the like as necessary.

### <Production Method 11>

A compound of formula [I-13] among the compounds of the present invention can be produced using a compound of formula [I-1] according to a method based on the reaction scheme exemplified below.

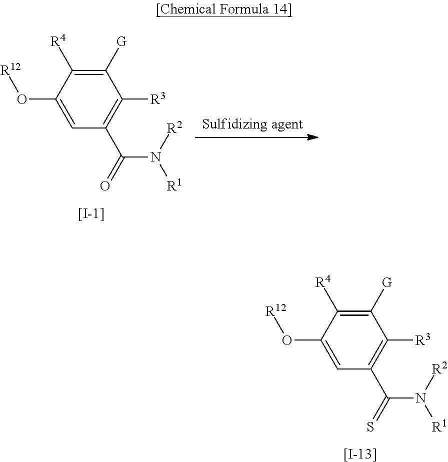

(In the formula, G, R¹, R², R³, R¹, and R¹² are as defined above.)

That is, the compound of formula [I-13] can be produced by reacting a compound [I-1] and an appropriate sulfidizing agent in an appropriate solvent in the presence or absence of an appropriate base.

Examples of the sulfurizing agent that can be used in the current reaction include phosphorus pentasulfide and a Lawesson's reagent (2,4-bis(4-methoxyphenyl)-1,3,2,4-dithiadiphosphetane-2,4-disulfide). The amount of use of the sulfurizing agent may be appropriately selected from the range of 0.5 to 30.0 mol, and is preferably 0.5 to 5.0 mol per 1 mol of the compound [I-1].

Although addition of a base is not necessarily required in the current reaction, when the base is used, examples of the base that can be used include inorganic bases such as carbonates of alkali metals, such as sodium carbonate and potassium carbonate; and organic bases such as triethylamine, 4-methylmorpholine, N-ethyldiisopropylamine, 1,8-diazabicyclo[5.4.0]-7-undecene, pyridine, 4-(N,N-dimethylamino)pyridine, and 2,6-lutidine. The amount of use of the base may be appropriately selected from the range of 0.01 to 10.0 mol, and is preferably 0.1 to 2.0 mol per 1 mol of the compound [I-1].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; halogenated hydrocarbons such as dichloromethane, chloroform, and 1,2-dichloroethane; aliphatic hydrocarbons such as pentane, hexane, cyclohexane, and heptane; water, and a mixed solvent thereof. The amount of use of the solvent is 0.1 to 300 liters, and is preferably 0.3 to 50 liters per 1 mol of the compound [I-1].

The reaction temperature of the current reaction may be selected from an arbitrary temperature range usually from −70 °C to the reflux temperature in the reaction system, and is preferably in the range of 0 °C to 150 °C.

The reaction time of the current reaction varies depending on the reaction temperature, the reaction substrate, the reaction amount, and the like, but is usually 10 minutes to 24 hours.

After completion of the reaction, the compound [I-13] can be isolated by conducting an operation of filtering off an insoluble substance from the reaction mixture and concentrating the resulting substance, or pouring the reaction mixture into water and subjecting the mixture to extraction with an organic solvent, followed by concentration. Alternatively, the compound [I-13] can also be isolated from the reaction mixture by concentrating the solvent. The isolated compound [I-13] also can be further purified by column chromatography, recrystallization, or the like as necessary.

### <Production Method 12>

A compound of formula [II-1] among the compounds of the present invention can be produced using a compound of formula [XIII] according to a method based on the reaction scheme exemplified below.

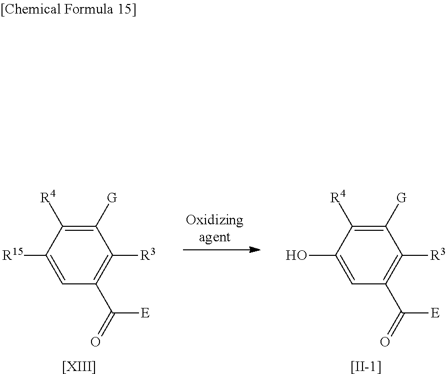

(In the formula, R¹⁵ represents a dihydroxyboryl (—B(OH)₂) or a pinacolato borane-2-yl, and G, R³, R⁴, and E are as defined above.)

That is, the compound of formula [II-1] can be produced by reacting a compound [XIII] with an appropriate oxidizing agent in an appropriate solvent.

Examples of the oxidizing agent that can be used in the current reaction include hydrogen peroxide and 4-methylmorpholine-N-oxide. The amount of use of the oxidizing agent may be appropriately selected from the range of 1.0 to 20.0 mol, and is preferably 1.0 to 6.0 mol per 1 mol of the compound [XIII].

Examples of the solvent that can be used in the current reaction include aromatic hydrocarbons such as benzene, toluene, xylene, and chlorobenzene; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; alcohols such as methanol, ethanol, and 2-propanol; halogenated hydrocarbons such as dichloromethane, chloroform, and 1,2-dichloroethane; aliphatic hydrocarbons such as pentane, hexane, cyclohexane, and heptane; ketones such as acetone, methyl ethyl ketone, and cyclohexanone; acetic acid, water, and a mixed solvent thereof. The amount of use of the solvent is 0.1 to 100 liters, and is preferably 0.3 to 15 liters per 1 mol of the compound [XIII].

The reaction temperature of the current reaction may be selected from an arbitrary temperature range from −30 °C to the reflux temperature in the reaction system, and is preferably in the range of −10 °C to 150 °C.

The reaction time of the current reaction varies depending on the reaction temperature, the reaction substrate, the reaction amount, and the like, but is usually 10 minutes to 24 hours.

After completion of the reaction, the compound [II-1] can be isolated by conducting an operation of pouring the reaction mixture into water or the like and subjecting the mixture to extraction with an organic solvent, followed by concentration. The isolated compound [II-1] also can be further purified by column chromatography, recrystallization, or the like as necessary.

### <Production Method 13>

A compound of formula [I-15] among the compounds of the present invention can be produced according to a method based on the reaction scheme exemplified below.

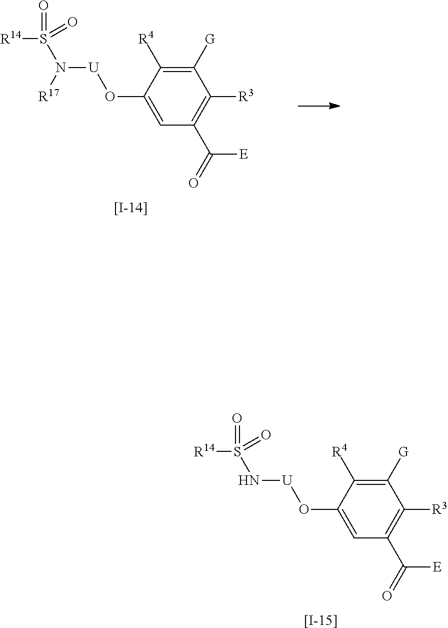

(In the formula, R¹⁷ represents a (C1-C6)alkoxymethyl or a tert-butoxycarbonyl, and E, G, R³, R⁴, R¹⁴ and U are as defined above.)

That is, the compound of formula [I-15] can be produced by reacting a compound [I-14] in an appropriate solvent in the presence of an appropriate acid.

Examples of the acid that can be used in the current reaction include mineral acids such as hydrochloric acid, hydrobromic acid, and sulfuric acid; carboxylic acids such as acetic acid, propionic acid, and trifluoroacetic acid; and sulfonic acids such as methanesulfonic acid and trifluoromethanesulfonic acid. The amount of use of the acid may be appropriately selected from the range of 0.1 to moles corresponding to the amount of the solvent, and is preferably 1.0 to 100.0 moles per 1 mole of the compound [I-14].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; alcohols such as methanol, ethanol, and 2-propanol; halogenated hydrocarbons such as dichloromethane, chloroform, and 1,2-dichloroethane; aliphatic hydrocarbons such as pentane, hexane, cyclohexane, and heptane; ketones such as acetone, methyl ethyl ketone, and cyclohexanone; water, and a mixed solvent thereof. The amount of use of the solvent is 0.1 to 500 liters, and is preferably 0.3 to 30 liters per 1 mol of the compound [I-14].

The reaction temperature of the current reaction may be selected from an arbitrary temperature range usually from −30 °C to the reflux temperature in the reaction system, and is preferably in the range of 0 °C to 150 °C.

The reaction time of the current reaction varies depending on the reaction temperature, the reaction substrate, the reaction amount, and the like, but is usually 10 minutes to 72 hours.

After completion of the current reaction, the compound [I-15] can be isolated by conducting an operation of concentrating the reaction mixture as it is, or pouring the reaction mixture into water or the like and subjecting the mixture to extraction with an organic solvent, followed by concentration. The isolated compound [I-15] also can be further purified by column chromatography, recrystallization, or the like as necessary.

### <Production Method 1 of Intermediate>

A compound of formula [XVI] can be produced using a compound of formula [XIV] according to a method based on the reaction scheme exemplified below.

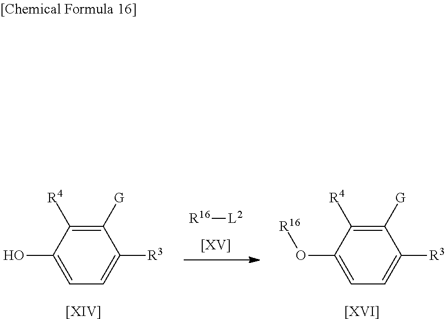

(In the formula, R¹⁶ represents a protecting group such as a (C1-C6)alkylcarbonyl, a (C1-C6)alkoxycarbonyl, a methanesulfonyl, a trifluoromethanesulfonyl, a nonafluorobutanesulfonyl, a benzenesulfonyl, or a p-toluenesulfonyl, and G, L², R³, and R⁴ are as defined above.)

That is, the compound of formula [XVI] can be produced by reacting a compound [XIV] with a compound [XV] in an appropriate solvent, in the presence of an appropriate base, under conditions used for protecting a phenolic hydroxyl group, for example, according to the method described in GREEN'S PROTECTIVE GROUPS in Organic Synthesis; the 5th edition (John Wiley and Sons, 2014, Peter G. M. Wuts).

### <Production Method 2 of Intermediate>

A compound of formula [XVII] can be produced using a compound of formula [XVI] according to a method based on the reaction scheme exemplified below.

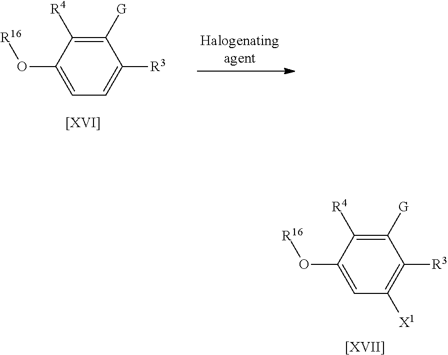

(In the formula, G, X¹, R³, R⁴, and R¹⁶ are as defined above.)

That is, the compound of formula [XVII] can be produced by reacting a compound [XVI] with a halogenating agent in an appropriate solvent.

Examples of the halogenating agent that can be used in the current reaction include chlorine, sulfuryl chloride, N-chlorosuccinimide, bromine, N-bromosuccinimide, 1,3-dibromo-5,5-dimethylhydantoin, iodine, N-iodosuccinimide, 1,3-diiodo-5,5-dimethylhydantoin, potassium iodate, and a mixture thereof. The amount of use of the halogenating agent may be appropriately selected from the range of 0.01 to 10.0 mol, and is preferably 0.02 to 2.0 mol per 1 mol of the compound [XVI].

Examples of the solvent that can be used in the current reaction include halogenated hydrocarbons such as dichloromethane, chloroform, and 1,2-dichloroethane; aliphatic hydrocarbons such as pentane, hexane, cyclohexane, and heptane; nitriles such as acetonitrile and propionitrile; carboxylic acids such as acetic acid, propionic acid, and trifluoroacetic acid; sulfuric acid, water, and a mixed solvent thereof. The amount of use of the solvent is 0.1 to 300 liters, and is preferably 0.3 to 20 liters per 1 mol of the compound [XVI].

The reaction temperature of the current reaction may be selected from an arbitrary temperature range usually from −70 °C to the reflux temperature in the reaction system, and is preferably in the range of −20 °C to 100 °C.

The reaction time of the current reaction varies depending on the reaction temperature, the reaction substrate, the reaction amount, and the like, but is usually 10 minutes to 24 hours.

After completion of the reaction, the compound [XVII] can be isolated by conducting an operation of pouring the reaction mixture into water and collecting the precipitated solid by filtration, or subjecting the reaction mixture to extraction with an organic solvent, followed by concentration. The isolated compound [XVII] also can be further purified by column chromatography, recrystallization, or the like as necessary.

### <Production Method 3 of Intermediate>

A compound of formula [XVIII] can be produced using a compound of formula [XVII] according to a method based on the reaction scheme exemplified below.

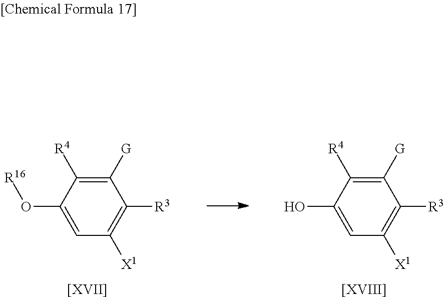

(In the formula, G, X¹, R³, R⁴, and R¹⁶ are as defined above.)

That is, the compound of formula [XVIII] can be produced by hydrolyzing a compound [XVII] in an appropriate solvent in the presence of an appropriate acid or base.

The acid, base, and solvent that can be used in the current reaction, the reaction temperature, and the reaction time are the same as those in Process 1-2 of the production method 1.

After completion of the current reaction, the compound [XVIII] can be isolated by conducting an operation of pouring the reaction mixture into water to neutralize the mixture and collecting the precipitated solid by filtration, or subjecting the reaction mixture to extraction with an organic solvent, followed by concentration. The isolated compound [XVIII] also can be further purified by column chromatography, recrystallization, or the like as necessary.

### <Production Method 4 of Intermediate>

A compound of formula [XIX] can be produced using a compound of formula [XVIII] according to a method based on the reaction scheme exemplified below.

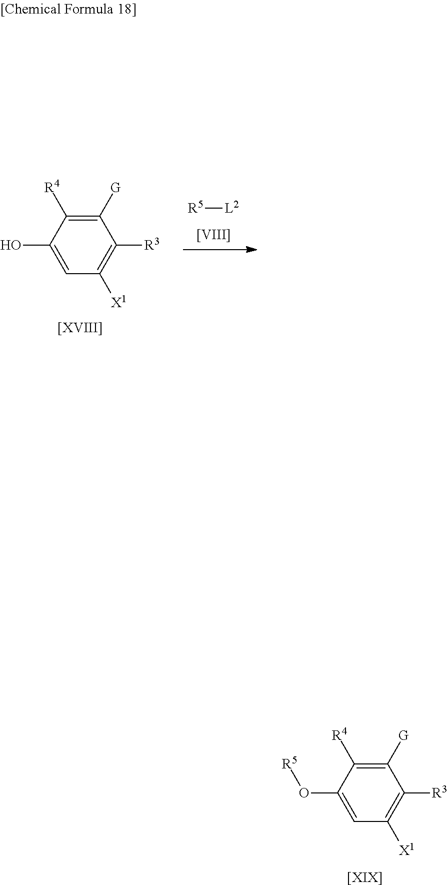

(In the formula, G, L², R³, R⁴, R⁵ and X¹ are as defined above.)

That is, the compound of formula [XIX] can be produced by reacting a compound [XVIII] and a compound [VIII] in an appropriate solvent, in the presence of an appropriate base, in the presence or absence of an appropriate catalyst.

The base and catalyst used in the current reaction, the solvent that can be used in the reaction, the reaction temperature, and the reaction time are the same as those in the production method 3.

After completion of the reaction, the compound [XIX] can be isolated by conducting an operation of pouring the reaction mixture into water and collecting the precipitated solid by filtration, or subjecting the reaction mixture to extraction with an organic solvent, followed by concentration. The isolated compound [XIX] also can be further purified by column chromatography, recrystallization, or the like as necessary.

### <Production Method 5 of Intermediate>

A compound of formula [XIX] can be produced using a compound of formula [XVIII] according to a method based on the reaction scheme exemplified below.

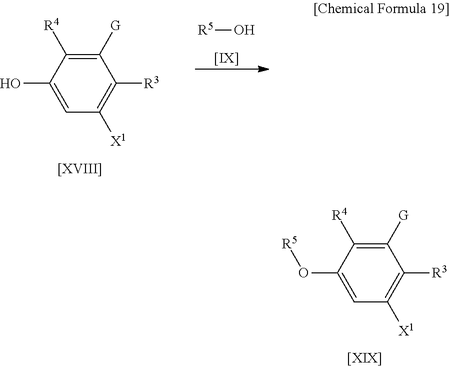

(In the formula, G, R³, R⁴, R⁵, and X¹ are as defined above.)

That is, the compound of formula [XIX] can be produced by reacting a compound [XVIII] and a compound [IX] in an appropriate solvent, in the presence of a trisubstituted phosphine and an azodicarboxylic acid derivative, or in the presence of phosphorane.

The trisubstituted phosphine, azodicarboxylic acid derivative, and phosphorane used in the current reaction, the solvent that can be used in the reaction, the reaction temperature, and the reaction time are the same as those in the production method 4.

After completion of the reaction, the compound [XIX] can be isolated by conducting an operation of pouring the reaction mixture into water and collecting the precipitated solid by filtration, or subjecting the reaction mixture to extraction with an organic solvent, followed by concentration. The isolated compound [XIX] also can be further purified by column chromatography, recrystallization, or the like as necessary.

### <Production Method 6 of Intermediate>

A compound of formula [XXI] can be produced using a compound of formula [XX] according to a method based on the reaction scheme exemplified below.

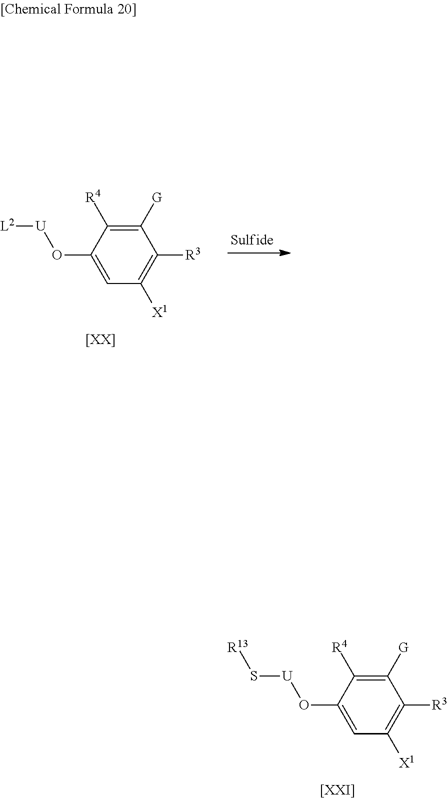

(In the formula, G, L², R³, R⁴, R¹³, U, and X¹ are as defined above.)

That is, the compound of formula [XXI] can be produced by reacting a compound [XX] and a sulfide in an appropriate solvent, in the presence or absence of an appropriate base, in the presence or absence of an appropriate catalyst.

The sulfide and base used in the current reaction, the solvent that can be used in the reaction, the reaction temperature, and the reaction time are the same as those in the production method 5.

After completion of the reaction, the compound [XXI] can be isolated by conducting an operation of pouring the reaction mixture into water and collecting the precipitated solid by filtration, or subjecting the reaction mixture to extraction with an organic solvent, followed by concentration. The isolated compound [XXI] also can be further purified by column chromatography, recrystallization, or the like as necessary.

### <Production Method 7 of Intermediate>

A compound of formula [XXIII] can be produced using a compound of formula [XXII] according to a method based on the reaction scheme exemplified below.

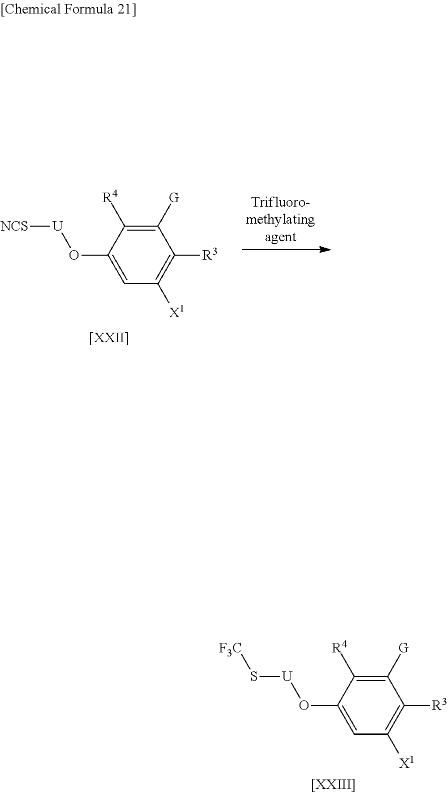

(In the formula, G, R³, R⁴, U, and X¹ are as defined above.)

That is, the compound of formula [XXIII] can be produced by reacting a compound [XXII] and a trifluoromethylating agent in an appropriate solvent in the presence of an appropriate catalyst.

The trifluoromethylating agent and catalyst used in the current reaction, the solvent that can be used in the reaction, the reaction temperature, and the reaction time are the same as those in the production method 6. After completion of the reaction, the compound [XXIII] can be isolated by conducting an operation of pouring the reaction mixture into water and collecting the precipitated solid by filtration, or subjecting the reaction mixture to extraction with an organic solvent, followed by concentration. The isolated compound [XXIII] also can be further purified by column chromatography, recrystallization, or the like as necessary.

### <Production Method 8 of Intermediate>

A compound of formula [XXVI] can be produced using a compound of formula [XVII] according to a method based on the reaction scheme exemplified below.

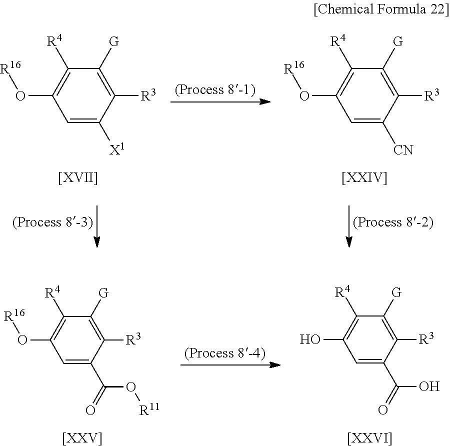

(In the formula, G, R³, R⁴, R¹¹, R¹⁶, and X¹ are as defined above.)

That is, the compound of formula [XXVI] can be produced by (Process 8′-1) converting a compound of formula [XVII] into a compound [XXIV] using a cyanide in an appropriate solvent in the presence or absence of an appropriate catalyst, and then (Process 8′-2) hydrolyzing the compound of formula [XXVI] in an appropriate solvent in the presence of an appropriate acid or base. In addition, the compound [XXVI] can be produced by (Process 8′-3) subjecting a compound of formula [XVII] to a carbon monoxide insertion reaction in an appropriate solvent in the presence of an appropriate catalyst, an appropriate base, and an appropriate alcohol to obtain a compound represented by [XXV] among the compounds of the present invention, and then (Process 8′-4) hydrolyzing the compound of formula [XXV] in an appropriate solvent in the presence of an appropriate acid or base.

The cyanide, catalyst, acid, base, and alcohol used in the current reaction, the solvent that can be used in the reaction, the reaction temperature, and the reaction time are the same as those in the production method 1.

After completion of the reaction, the compound [XXVI] can be isolated by conducting an operation of pouring the reaction mixture into water and collecting the precipitated solid by filtration, or subjecting the reaction mixture to extraction with an organic solvent, followed by concentration. The isolated compound [XXVI] also can be further purified by column chromatography, recrystallization, or the like as necessary.

### <Production Method 9 of Intermediate>

A compound of formula [XXVIII] can be produced using a compound of formula [XXVII] according to a method based on the reaction scheme exemplified below.

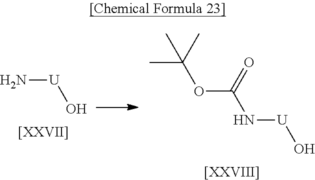

(In the formula, U is as defined above.)

That is, the compound of formula [XXVIII] can be produced by reacting a compound [XXVII] with an appropriate acid anhydride, in an appropriate solvent, in the presence of an appropriate base, under conditions used for protection of an amino group by a tert-butoxycarbonyl group, for example, according to the method described in GREEN'S PROTECTIVE GROUPS in Organic Synthesis; the 5th edition (John Wiley and Sons, 2014, Peter G. M. Wuts).

### <Production Method 10 of Intermediate>

A compound of formula [XXX] can be produced using a compound of formula [XXIX] according to a method based on the reaction scheme exemplified below.

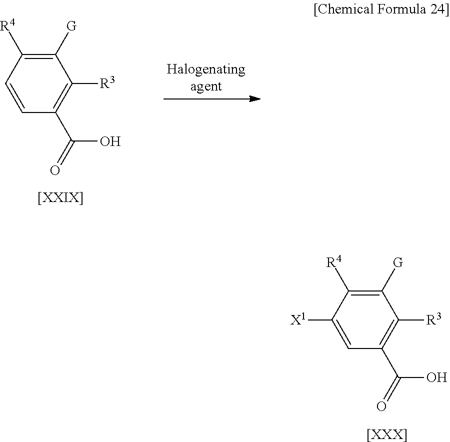

(In the formula, G, R³, R⁴, and X¹ are as defined above.)

That is, the compound of formula [XXX] can be produced by reacting a compound [XXIX] with a halogenating agent in an appropriate solvent.

The halogenating agent used in the current reaction, the solvent that can be used in the reaction, the reaction temperature, and the reaction time are the same as those in the production method 2 of intermediate.

After completion of the reaction, the compound [XXX] can be isolated by conducting an operation of pouring the reaction mixture into water and collecting the precipitated solid by filtration, or subjecting the reaction mixture to extraction with an organic solvent, followed by concentration. The isolated compound [XXX] also can be further purified by column chromatography, recrystallization, or the like as necessary.

### <Production Method 11 of Intermediate>

A compound of formula [XXXII] can be produced using a compound of formula [XXX] according to a method based on the reaction scheme exemplified below.

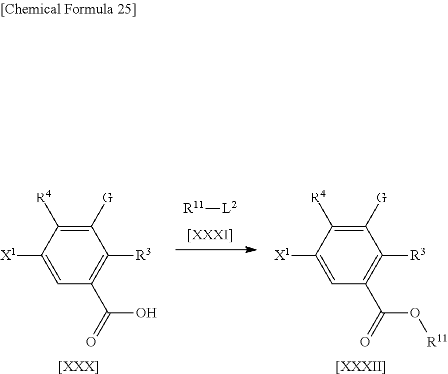

(In the formula, G, L², R³, R⁴, R¹¹, and X¹ are as defined above.)

That is, the compound of formula [XXXII] can be produced by reacting a compound [XXX] and a compound [XXXI] in an appropriate solvent, in the presence of an appropriate base, in the presence or absence of an appropriate catalyst.

The base and catalyst used in the current reaction, the solvent that can be used in the reaction, the reaction temperature, and the reaction time are the same as those in the production method 3.

After completion of the reaction, the compound [XXXII] can be isolated by conducting an operation of pouring the reaction mixture into water and collecting the precipitated solid by filtration, or subjecting the reaction mixture to extraction with an organic solvent, followed by concentration. The isolated compound [XXXII] also can be further purified by column chromatography, recrystallization, or the like as necessary.

### <Production Method 12 of Intermediate>

A compound of formula [XXXIV] can be produced using a compound of formula [XXXII] according to a method based on the reaction scheme exemplified below.

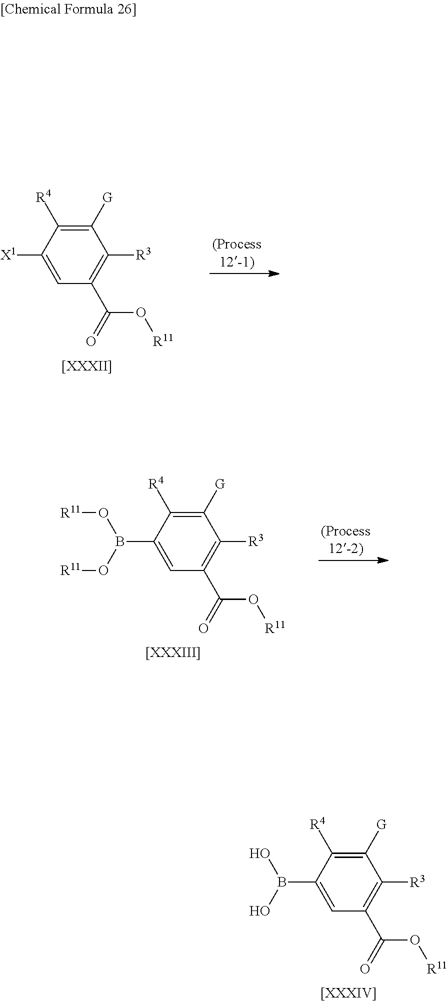

(In the formula, G, R³, R⁴, R¹¹, and X¹ are as defined above.)

That is, the compound of formula [XXXIV] can be produced by (Process 12′-1) reacting a compound [XXXII] with an appropriate organometallic compound in an appropriate solvent, then reacting the resultant with an borate ester to obtain a compound [XXXIII], and (Process 12′-2) reacting the compound [XXXIII] with an acid in an appropriate solvent.

### (Process 12′-1)

Examples of the organometallic compound that can be used in the current reaction include isopropylmagnesium chloride. The amount of use of the organometallic compound may be appropriately selected from the range of 1.0 to 3.0 mol, and is preferably 1.0 to 1.2 mol per 1 mol of the compound [XXXII].

Examples of the borate ester that can be used in the current reaction include trimethyl borate, triethyl borate, triisopropyl borate, and tributyl borate. The amount of use of the borate ester may be appropriately selected from the range of 1.0 to 3.0 mol, and is preferably 1.0 to 1.2 mol per 1 mol of the compound [XXXII].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; aliphatic hydrocarbons such as pentane, hexane, cyclohexane, and heptane; pyridines such as pyridine and picoline; and a mixed solvent thereof. The amount of use of the solvent is 0.1 to 100 liters, and is preferably 0.1 to 5 liters per 1 mol of the compound [XXXII].

The reaction temperature of the current reaction may be selected from an arbitrary range usually from −100 °C to the reflux temperature in the reaction system, and is preferably in the range of −80 °C to room temperature.

The reaction time of the current reaction varies depending on the reaction temperature, the reaction substrate, the reaction amount, and the like, but is usually 5 minutes to 24 hours.

After completion of the reaction, the compound [XXXIII] can be used as it is for the next reaction without isolation.

### (Process 12′-2)

Examples of the acid that can be used in the current reaction include mineral acids such as sulfuric acid and hydrochloric acid. The amount of use of the acid may be appropriately selected from the range of 1.0 to 5.0 mol, and is preferably 0.5 to 2.0 mol per 1 mol of the compound [XXXIII].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; halogenated hydrocarbons such as dichloromethane, chloroform, and 1,2-dichloroethane; alcohols such as methanol, ethanol, and 2-propanol; aliphatic hydrocarbons such as pentane, hexane, cyclohexane, and heptane; ketones such as acetone, methyl ethyl ketone, and cyclohexanone; water, and a mixed solvent thereof. The amount of use of the solvent is 0.1 to 100 liters, and is preferably 0.1 to 5 liters per 1 mol of the compound [XXXIII].

The reaction temperature of the current reaction may be usually selected from an arbitrary range from 0 °C to the reflux temperature in the reaction system, and is preferably in the range of 0 °C to 100 °C.

The reaction time of the current reaction varies depending on the reaction temperature, the reaction substrate, the reaction amount, and the like, but is usually 10 minutes to 24 hours.

After completion of the reaction, the compound [XXXIV] can be isolated by conducting an operation of pouring the reaction mixture into water and collecting the precipitated solid by filtration, or subjecting the reaction mixture to extraction with an organic solvent, followed by concentration. The isolated compound [XXXIV] also can be further purified by column chromatography, recrystallization, or the like as necessary.

### <Production Method 13 of Intermediate>

A compound of formula [XXXVI] can be produced using a compound of formula [XXXV] according to a method based on the reaction scheme exemplified below.

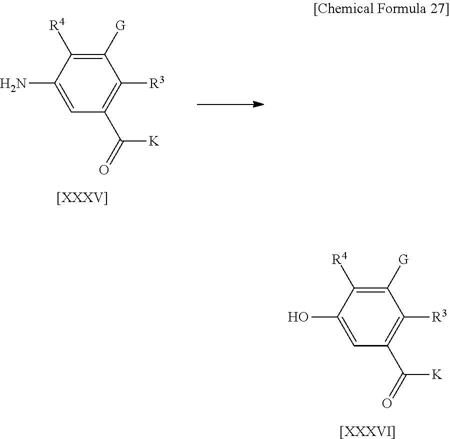

(In the formula, G, R³, R⁴, and K are as defined above.)

That is, the compound of formula [XXXVI] can be produced by reacting a compound [XXXV] with an acid and a nitrous acid derivative in an appropriate solvent, and then reacting the resultant with a metal salt as necessary.

Examples of the acid that can be used in the current reaction include mineral acids such as sulfuric acid and nitric acid, and organic acids such as trifluoroacetic acid and trifluoromethanesulfonic acid. The amount of use of the acid may be appropriately selected from the range of 1 to 20 mol, and is preferably 1.0 to 5.0 mol per 1 mol of the compound [XXXV]. However, when an acid is used as a solvent, the amount of use as the solvent is appropriately selected.

Examples of the nitrous acid derivative that can be used in the current reaction include nitrites such as sodium nitrite and potassium nitrite, and alkyl nitrites such as n-butyl nitrite, isopentyl nitrite, and tert-butyl nitrite. The amount of use of the nitrous acid derivative may be appropriately selected from the range of 1 to 3 mol, and is preferably 1.0 to 1.5 mol per 1 mol of the compound [XXXV].

Examples of the metal salt used as necessary in the current reaction include copper sulfate, copper nitrate, and copper oxide. The amount of use of the metal salt may be appropriately selected from the range of 0 to 2 mol, and is preferably 0 to 1.1 mol per 1 mol of the compound [XXXV].

Examples of the solvent that can be used in the current reaction include ethers such as diethyl ether, cyclopentyl methyl ether, tetrahydrofuran, 2-methyltetrahydrofuran, and 1,4-dioxane; aromatic hydrocarbons such as benzene, toluene, xylene, and chlorobenzene; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; halogenated hydrocarbons such as dichloromethane, chloroform, and 1,2-dichloroethane; alcohols such as methanol, ethanol, and 2-propanol; nitriles such as acetonitrile and propionitrile; esters such as ethyl acetate, butyl acetate, and ethyl propionate; aliphatic hydrocarbons such as pentane, hexane, cyclohexane, and heptane; pyridines such as pyridine and picoline; mineral acids such as acetic acid, hydrochloric acid, sulfuric acid, and nitric acid; organic acids such as trifluoroacetic acid and trifluoromethanesulfonic acid; water, and a mixed solvent thereof. The amount of use of the solvent is 0.1 to 100 liters, and is preferably 0.3 to 10 liters per 1 mol of the compound [XXXV].

The reaction temperature of the current reaction may be selected from an arbitrary range usually from −30 °C to the reflux temperature in the reaction system, and is preferably in the range of 0 °C to 150 °C.

The reaction time of the current reaction varies depending on the reaction temperature, the reaction substrate, the reaction amount, and the like, but is usually 10 minutes to 24 hours.

After completion of the reaction, the compound [XXXVI] can be isolated by conducting an operation of pouring the reaction mixture into water or the like and collecting the precipitated solid by filtration, or subjecting the reaction mixture to extraction with an organic solvent, followed by concentration. The isolated compound [XXXVI]also can be further purified by column chromatography, recrystallization, or the like as necessary. Alternatively, the compound [XXXVI] can be directly used for the next reaction without isolation.

### <Production Method 14 of Intermediate>

A compound of formula [XXXVIII] can be produced using a compound of formula [XXXVII] according to a method based on the reaction scheme exemplified below.

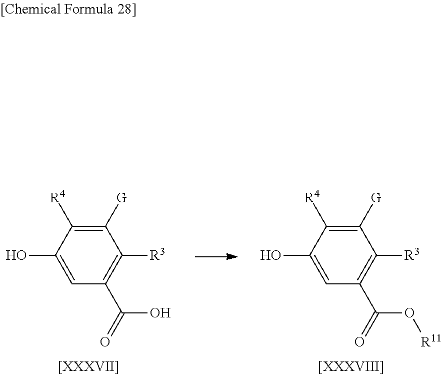

(In the formula, G, R³, R⁴, and R¹¹ are as defined above.)

That is, the compound of formula [XXXVIII] can be produced from a compound [XXXVII] under conditions used for esterification of a carboxy group, for example, according to the method described in GREEN'S PROTECTIVE GROUPS in Organic Synthesis; the 5th edition (John Wiley and Sons, 2014, Peter G. M. Wuts).

The hydroxybenzamide derivative of formula [II] of the present invention is a compound useful as an intermediate in the production of the 3-alkoxybenzamide derivative of formula [I] or agriculturally acceptable salt thereof according to the present invention.

The alkoxybenzoic acid derivative of formula [III] of the present invention or an ester thereof is a compound useful as an intermediate in the production of the 3-alkoxybenzamide derivative of formula [I] or agriculturally acceptable salt thereof according to the present invention.

The agrochemical composition of the present invention contains the 3-alkoxybenzamide derivative of formula [I] or agriculturally acceptable salt thereof according to the present invention as an active ingredient.

The agrochemical composition of the present invention can contain an additive component (carrier) usually used in agrochemical formulations as necessary.

The pest control agent of the present invention contains the 3-alkoxybenzamide derivative of formula [I] or agriculturally acceptable salt thereof according to the present invention as an active ingredient. The pest control agent of the present invention is typically an insecticide, an acaricide, and a nematicide.

The pest control agent of the present invention can contain an additive component (carrier) usually used in an agrochemical formulation as necessary.

Examples of the additive component include a carrier such as a solid carrier or a liquid carrier, a surfactant, a binder or a tackifier, a thickener, a colorant, a spreader, a sticker, an anti-freezing agent, an anti-caking agent, a disintegrant, and a degradation preventing agent. In addition, a preservative, a plant piece, or the like may be used as the additive component as necessary. These additive components may be used alone or in combination of two or more types thereof.

Examples of the solid carrier include mineral carriers such as pyrophyllite clay, kaolin clay, silica clay, talc, diatomaceous earth, zeolite, bentonite, acid clay, activated clay, attapulgus clay, vermiculite, perlite, pumice, white carbon (synthetic silicic acid, synthetic silicate, and the like), and titanium dioxide; plant carriers such as wood powder, cornstalks, walnut shells, fruit cores, chaff, sawdust, bran, soybean flour, powdered cellulose, starch, dextrin, and saccharides; inorganic salt carriers such as calcium carbonate, ammonium sulfate, sodium sulfate, or potassium chloride; polymer carriers such as polyethylene, polypropylene, polyvinyl chloride, polyvinyl acetate, an ethylene-vinyl acetate copolymer, and a urea-aldehyde resin.

Examples of the liquid carrier include monohydric alcohols such as methanol, ethanol, propanol, 2-propanol, butanol, and cyclohexanol; polyhydric alcohols such as ethylene glycol, diethylene glycol, propylene glycol, hexylene glycol, polyethylene glycol, polypropylene glycol, and glycerin; polyhydric alcohol derivatives such as propylene-based glycol ether; ketones such as acetone, methyl ethyl ketone, methyl isobutyl ketone, diisobutyl ketone, cyclohexanone, and isophorone; ethers such as ethyl ether, 1,4-dioxane, cellosolve, dipropyl ether, and tetrahydrofuran; aliphatic hydrocarbons such as normal paraffin, naphthene, isoparaffin, kerosene, and mineral oil; aromatic hydrocarbons such as toluene, C₉-C₁₀ alkylbenzene, xylene, solvent naphtha, alkylnaphthalene, and high-boiling-point aromatic hydrocarbon; halogenated hydrocarbons such as 1,2-dichloroethane, chloroform, and carbon tetrachloride; esters such as ethyl acetate, diisopropyl phthalate, dibutyl phthalate, dioctyl phthalate, and dimethyl adipate; lactones such as γ-butyrolactone; amides such as N,N-dimethylformamide, N,N-diethylformamide, N,N-dimethylacetamide, and N-methyl-2-pyrrolidone; nitriles such as acetonitrile; sulfur compounds such as dimethyl sulfoxide; vegetable oils such as soybean oil, rapeseed oil, cottonseed oil, coconut oil, and castor oil; lower alkyl esters of fatty acids derived from the vegetable oils; and water.

Examples of the surfactant include nonionic surfactants such as sorbitan fatty acid ester, polyoxyethylene sorbitan fatty acid ester, sucrose fatty acid ester, polyoxyethylene fatty acid ester, polyoxyethylene resin acid ester, polyoxyethylene fatty acid diester, polyoxyethylene alkyl ether, polyoxyethylene alkylphenyl ether, polyoxyethylene dialkyl phenyl ether, polyoxyethylene alkyl phenyl ether formalin condensate, polyoxyethylene polyoxypropylene block polymer, alkyl polyoxyethylene polypropylene block polymer ether, polyoxyethylene alkylamine, polyoxyethylene fatty acid amide, polyoxyethylene fatty acid bisphenyl ether, polyalkylene benzylphenyl ether, polyoxyalkylene styrylphenyl ether, acetylene diol, polyoxyalkylene-added acetylene diol, polyoxyethylene ether-type silicone, ester-type silicone, fluorine-based surfactant, polyoxyethylene castor oil, and polyoxyethylene hydrogenated castor oil; anionic surfactants such as alkyl sulfates, polyoxyethylene alkyl ether sulfates, polyoxyethylene alkyl phenyl ether sulfates, polyoxyethylene styryl phenyl ether sulfates, alkylbenzene sulfonates, lignin sulfonates, alkyl sulfosuccinates, naphthalene sulfonates, alkyl naphthalene sulfonates, salts of formalin condensates of naphthalene sulfonates, salts of formalin condensates of alkyl naphthalene sulfonates, fatty acid salts, polycarboxylates, N-methyl-fatty acid sarcosinates, resin acid salts, polyoxyethylene alkyl ether phosphates, and polyoxyethylene alkyl phenyl ether phosphates; cationic surfactants including alkylamine salts such as laurylamine hydrochloride, stearylamine hydrochloride, oleylamine hydrochloride, stearylamine acetate, stearylaminopropylamine acetate, alkyltrimethylammonium chloride, and alkyldimethylbenzalkonium chloride; and amphoteric surfactants including betaine-type surfactants such as dialkyldiaminoethylbetaine and alkyldimethylbenzylbetaine, and amino acid-type surfactants such as dialkylaminoethylglycine and alkyldimethylbenzylglycine.

Examples of the binder and the tackifier include carboxymethyl cellulose and salts thereof, dextrin, water-soluble starch, xanthan gum, guar gum, sucrose, polyvinyl pyrrolidone, gum arabic, polyvinyl alcohol, polyvinyl acetate, sodium polyacrylate, polyethylene glycol, polyethylene oxide, and natural phospholipid (for example, cephalin acid, and lecithin).

Examples of the thickener include water-soluble polymers such as xanthan gum, guar gum, carboxymethyl cellulose, polyvinylpyrrolidone, carboxyvinyl polymers, acrylic polymers, starch derivatives, and polysaccharides; inorganic fine powders such as high-purity bentonite and white carbon; and organic fine powders such as organic bentonite.

Examples of the colorant include inorganic pigments such as iron oxide, titanium oxide, and Prussian blue; and organic dyes such as an alizarin dye, an azo dye, and a metal phthalocyanine dye.

Examples of the spreader include a silicone-based surfactant, cellulose powder, dextrin, processed starch, a polyaminocarboxylic acid chelate compound, crosslinked polyvinyl pyrrolidone, a maleic acid/styrene copolymer, a methacrylic acid copolymer, a half ester of a polymer of a polyhydric alcohol and a dicarboxylic anhydride, a water-soluble salt of polystyrene sulfonic acid, polyoxyethylene alkanediols, polyoxyethylene alkyne diols, and alkyne diols.

Examples of the sticker include various surfactants such as sodium dialkyl sulfosuccinate, polyoxyethylene alkyl ether, polyoxyethylene alkyl phenyl ether, and polyoxyethylene fatty acid ester; paraffin, terpene, polyamide resin, polyacrylate, polyoxyethylene, wax, polyvinyl alkyl ether, alkylphenol formalin condensate, and synthetic resin emulsion.

Examples of the anti-freezing agent include polyhydric alcohols such as ethylene glycol, diethylene glycol, propylene glycol, and glycerin.

Examples of the anti-caking agent include polysaccharides such as starch, alginic acid, mannose, and galactose; polyvinylpyrrolidone, white carbon, ester gum, and petroleum resin.

Examples of the disintegrant include sodium tripolyphosphate, sodium hexametaphosphate, metal stearate, cellulose powder, dextrin, a copolymer of methacrylic acid ester, polyvinylpyrrolidone, a polyaminocarboxylic acid chelate compound, a sulfonated styrene-isobutylene-maleic anhydride copolymer, and a starch-polyacrylonitrile graft copolymer.

Examples of the degradation preventing agent include desiccants such as zeolite, quicklime, and magnesium oxide; antioxidants such as phenol-based, amine-based, sulfur-based, and phosphoric acid-based antioxidants; and ultraviolet absorbers such as salicylic acid-based and benzophenone-based ultraviolet absorbers.

Examples of the preservative include potassium sorbate and 1,2-benzothiazolin-3-one.

Examples of the plant piece include sawdust, coconut shell, corn cob, and tobacco stalk.

On the other hand, when the above-described additive component is contained in the pest control agent of the present invention, the content thereof is usually selected in the range of 5 to 95%, preferably 20 to 90% in the case of a carrier such as a solid carrier or a liquid carrier, usually selected in the range of 0.1% to 30%, preferably 0.5 to 10% in the case of a surfactant, and selected in the range of 0.1 to 30%, preferably 0.5 to 10% in the case of other additives, on a mass basis.

The pest control agent of the present invention is used by being formulated in any formulation such as a powder, a powder granule, a granule, a wettable powder, a water-soluble agent, a water-dispersible granule, a tablet, a jumbo agent, an emulsifiable concentrate, an oil, a liquid, a flowable agent, an emulsion, a microemulsion, a suspoemulsion, a trace spray agent, a microcapsule, a smoking agent, an aerosol agent, a bait agent, or a paste agent.

In actual use of these formulations, they can be used as they are or diluted to a predetermined concentration with a diluent such as water. The application of various formulations containing the compound of the present invention or dilutions thereof can be carried out by an application method that is usually carried out. In other words, the application can be carried out by dispersion (for example, spraying, misting, atomizing, dusting powder, dusting granules, water surface application, and box application), soil application (for example, mixing and irrigation), surface application (for example, application, and powder coating, and coating), seed treatment (for example, smearing, powder coating treatment, and the like), immersion, poison bait, smoking application, and the like. In addition, it is also possible to control the generation and growth of harmful bugs, particularly harmful insects in excrement of domestic animals by feeding the animals with the active ingredient mixed.

The method for controlling a pest of the present invention can be carried out by using the 3-alkoxybenzamide derivative of formula [I] or agriculturally acceptable salt thereof according to the present invention in an amount of an active ingredient by the application method described above.

The blending ratio (mass %) of the active ingredient in the pest control agent of the present invention is appropriately selected as necessary. For example, the blending ratio may be appropriately selected from the range of 0.01 to 20%, preferably 0.05 to 10% in the case of a powder, a powder granule, a fine powder, or the like. The blending ratio may be appropriately selected from the range of 0.1 to 30%, preferably 0.5 to 20% in the case of a granule, or the like. The blending ratio may be appropriately selected from the range of 1 to 70%, preferably 5 to 50% in the case of a wettable powder, a water-dispersible granule, or the like. The blending ratio may be appropriately selected from the range of 1 to 95%, preferably 10 to 80% in the case of a water-soluble agent, a liquid, or the like. The blending ratio may be appropriately selected from the range of 5 to 90%, preferably 10 to 80% in the case of an emulsifiable concentrate, or the like. The blending ratio may be appropriately selected from the range of 1 to 50%, preferably 5 to 30% in the case of an oil, or the like. The blending ratio may be appropriately selected from the range of 5 to 60%, preferably 10 to 50% in the case of a flowable agent, or the like. The blending ratio may be appropriately selected from the range of 5 to 70%, preferably 10 to 60% in the case of an emulsion, a microemulsion, a suspoemulsion, or the like. The blending ratio may be appropriately selected from the range of 1 to 80%, preferably 5 to 50% in the case of a tablet, a bait agent, a paste agent, or the like. The blending ratio may be appropriately selected from the range of 0.1 to 50%, preferably 1 to 30% in the case of a smoking agent or the like. The blending ratio may be appropriately selected from the range of 0.05 to 20%, preferably 0.1 to 10% in the case of an aerosol agent or the like.

These formulations are diluted to an appropriate concentration and dispersed, or directly applied.

When the pest control agent of the present invention is used by being diluted with a diluent, the application is generally performed at an active ingredient concentration of 0.1 to 5,000 ppm. When the formulation is used as it is, the amount of application per unit area is 0.1 to 5,000 g per 1 ha as the active ingredient compound, but the amount of application is not limited thereto.

Although it is needless to say that the pest control agent of the present invention is sufficiently effective even when the compound of the present invention is used alone as an active ingredient, the pest control agent can be mixed with or used in combination with other fertilizers and pesticides, for example, insecticides, acaricides, nematicides, synergists, fungicides, antiviral agents, attractants, herbicides, and plant growth regulators as necessary, and in this case, a more excellent effect may be exhibited.

Next, known insecticides (insecticidal active ingredients), acaricides (acaricidal active ingredients), nematicides (nematicidal active ingredients), and synergist compounds (synergistic active ingredients) which may be mixed or used in combination are exemplified below.

Insecticidal active ingredients, acaricidal active ingredients, nematicidal active ingredients, synergistic active ingredients:
acrinathrin, azadirachtin, azamethiphos, acynonapyr, azinphos-ethyl, azinphos-methyl, acequinocyl, acetamiprid, acetoprole, acephate, azocyclotin, abamectin, afidopyropen, afoxolaner, amidoflumet, amitraz, alanycarb, aldicarb, aldoxycarb, aliethrin [including a d-cis-trans-form and a d-trans-form], isazophos, isamidofos, isocarbophos, isoxathion, isocycloseram, isofenphos-methyl, isoprocarb, epsilon-metofluthrin, epsilon-momfluorothrin, ivermectin, imicyafos, imidacloprid, imiprothrin, indoxacarb, esfenvalerate, ethiofencarb, ethion, ethiprole, ethylene dibromide, etoxazole, etofenprox, ethoprophos, etrimfos, emamectin, emamectin benzoate, endosulfan, empenthrin, oxazosulfyl, oxamyl, oxydemeton-methyl, oxydeprofos, omethoate, Nuclear polyhedrosis virus, cadusafos, kappa-tefluthrin, kappa-bifenthrin, karanjin, cartap, Granulosis virus, carbaryl, carbosulfan, carbofuran, gamma-BHC, xylylcarb, quinalphos, kinoprene, chinomethionat, Entero virus, coumaphos, cryolite, clothianidin, clofentezine, chromafenozide, chlorantraniliprole, chlorethoxyfos, chlordane, chloropicrin, chlorpyrifos, chlorpyrifos-methyl, chlorfenapyr, chlorfenvinphos, chlorfluazuron, chlormephos, chloroprallethrin, Entomopoxi virus, Irido virus, cyazypyr, cyanophos, diafenthiuron, diamidafos, cyantraniliprole, cyetpyrafen, dienochlor, cyenopyrafen, dioxabenzofos, diofenolan, Sigma virus, cyclaniliprole, cycloxaprid, dicrotophos, dichlofenthion, cyclobutrifluram, cycloprothrin, dichlorvos, dicloromezotiaz, dicofol, dicyclanil, disulfoton, dinotefuran, dinobuton, cyhalodiamide, cyhalothrin [including a gamma-form and a lambda-form], cyphenothrin [including a 1R-trans-form], cyfluthrin [including a beta-form], diflubenzuron, cyflumetofen, diflovidazin, cyproflanilide, cyhexatin, cypermethrin [including an alpha-form, a beta-form, a theta-form, and a zeta-form], dimpropyridaz, dimethyl-2,2,2-trichloro-1-hydroxyethylphosphonate (DEP), dimethylvinphos, dimethoate, dimefluthrin, jasmone, cis-jasmone, jasmonic acid, methyl jasmonate, silafluofen, cyromazine, Steinernema carpocapsae, Steinernema kushidai, Steinernema glaseri, spinetoram, spinosad, spirodiclofen, spirotetramat, spiropidion, spiromesifen, suicofuron-sodium, sulfluramid, sulfoxaflor, sulfotep, diazinon, thiacloprid, thiamethoxam, tioxazafen, thiodicarb, thiocyclam, thiosultap, thionazin, thiofanox, thiometon, tyclopyrazoflor, tetrachlorantraniliprole, tetrachlorvinphos, tetradifon, tetraniliprole, tetramethylfluthrin, tetramethrin, tebupirimfos, tebufenozide, tebufenpyrad, tefluthrin, teflubenzuron, demeton-S-methyl, temephos, deltamethrin, terbufos, tralomethrin, transfluthrin, triazamate, triazophos, trichlorfon, Trichoderma asperellum, Trichoderma paecilomyces, Trichoderma harzianum, triflumuron, triflumezopyrim, trimethacarb, tolfenpyrad, naled, nicotine, nicofluprole, nitenpyram, nemadectin, Denso virus, novaluron, noviflumuron, Paecilomyces lilacinus, Barkholderia cepacia, Barkholderia rinojensis, Verticillium lecanii, hydroprene, Pasteuria nishizawae, Pasteuria penetrans, Bacillus amyloliquefaciens, Bacillus firmus, Bacillus sphaericus, Bacillus subtillis, Bacillus thuringiensis, insect toxin produced by Bacillus thuringiensis, Bacillus thuringiensis subsp. Aizawai, Bacillus thuringiensis subsp. Israelensis, Bacillus thuringiensis subsp. Kurstaki, Bacillus thuringiensis subsp. Tenebrionis, Bacillus popilliae, Bacillus licheniformis, vamidothion, parathion, parathion-methyl, halfenprox, halofenozide, bioallethrin, bioallethrin S-cyclopentenyl, bioresmethrin, bis-(2-chloro-1-methyl)ethylether (DCIP), bistrifluron, hydramethylnon, bifenazate, bifenthrin, pyflubumide, piperonyl butoxide, pymetrozine, pyraclofos, pyrafluprole, pyridaphenthion, pyridaben, pyridalyl, pyrifluquinazon, pyriprole, pyriproxyfen, pirimicarb, pyrimidifen, pyriminostrobin, pirimiphos-methyl, pyrethrin, famphur, fipronil, fenazaquin, fenamiphos, fenitrothion, fenoxycarb, fenothiocarb, phenothrin [including a 1R-trans-form], fenobucarb, fenthion, phenthoate, fenvalerate, fenpyroximate, fenbutatin oxide, fenpropathrin, fonofos, sulfuryl fluoride, butocarboxim, butoxycarboxim, buprofezin, furathiocarb, prallethrin, fluacrypyrim, fluazaindolizine, fluazuron, fluensulfone, fluopyram, sodium fluoroacetate, fluxametamide, flucycloxuron, flucythrinate, flusulfamide, fluthrin, fluvalinate [including a tau-form], flupyradifurone, flupyrazofos, flupyrimin, flufiprole, flufenerim, flufenoxystrobin, flufenoxuron, fluhexafon, flubendiamide, flupentiofenox, flumethrin, fluralaner, flurimfen, prothiofos, protrifenbute, flonicamid, propaphos, propargite, prohydrojasmon, profenofos, broflanilide, profluthrin, propetamphos, propoxur, flometoquin, bromopropylate, hexythiazox, hexaflumuron, Pacilimyces tenuipes, Paecilomyces fumosoroceus, Paecilomyces lilacinus, heptafluthrin, heptenophos, permethrin, benclothiaz, benzpyrimoxan, bensultap, benzoximate, bendiocarb, benfuracarb, Pochonia chlamydosporia, Beauveria tenella, Beauveria bassiana, Beauveria brongniartii, phoxim, phosalone, fosthiazate, fosthietan, phosphamidon, phosmet, polynactins, formetanate, phorate, machine oil, malathion, milbemectin, mecarbam, mesulfenfos, methomyl, metaldehyde, metaflumizone, methamidophos, metham, methiocarb, methidathion, methyl isothiocyanate, methyl bromide, methoxychlor, methoxyfenozide, methothrin, metofluthrin, methoprene, metolcarb, mevinphos, meperfluthrin, Monacrosporium phymatophagum, Monacrosporium phymatophagum, monocrotophos, momfluorothrin, Trichoderma harzianum, litlure-A, litlure-B, aluminium phosphide, zinc phosphide, phosphine, lufenuron, rescalure, resmethrin, lepimectin, rotenone, occlusion body of Cytoplasmic polyhedrosis virus, fenbutatin oxide, calcium cyanide, organotins, nicotine-sulfate, (Z)-11-tetradecenyl=acetate, (Z)-11-hexadecenyl, (Z)-11-hexadecenyl=acetate, (Z)-9,12-tetradecadienyl=acetate, (Z)-9-tetradecene-1-ol, (Z,E)-9,11-tetradecadienyl=acetate, (Z,E)-9,12-tetradecadienyl=acetate, 1,1,1-trichloro-2,2-bis(4-chlorophenyl)ethane (DDT), 1,3-dichloropropene, 2,4-dichloro-5-{2-[4-(trifluoromethyl)phenyl]ethoxy}phenyl-2,2,2-trifluoroethyl sulfoxide (Chemical Name, CAS Registry Number: 1472052-11-1), 2,4-dimethyl-5-[6-(trifluoromethylthio)hexyloxy]phenyl-2,2,2-trifluoroethyl sulfoxide (Chemical Name, CAS Registry Number: 1472050-34-2), 2-{2-fluoro-4-methyl-5-[(2,2,2-trifluoroethyl)sulfinyl]phenoxy}-5-(trifluoromethyl)pyridine (Chemical Name, CAS Registry Number: 1448758-62-0), 3-chloro-2-{2-fluoro-4-methyl-5-[(2,2,2-trifluoroethyl)sulfinyl]phenoxy}-5-(trifluoromethyl)pyridine (Chemical Name, CAS Registry Number: 1448761-28-1), 4,6-dinitro-o-cresol (DNOC), 4-fluoro-2-methyl-5-(5,5-dimethylhexyloxy)phenyl-2,2,2-trifluoroethyl sulfoxide (Chemical Name, CAS Registry Number: 1472047-71-4), Bt protein (Cry1Ab, Cry1Ac, Cry1Fa, Cry2Ab, mCry3A, Cry3Ab, Cry3Bb, Cry34/35Abl), methyl eugenol, 4-(p-acetoxyphenyl)-2-butanone, (Z)-10-tetradecenyi=acetate, (E,Z)-4,10-tetradecadienyl=acetate, (Z)-8-dodecenyl=acetate, (Z)-11-tetradecenyl=acetate, (Z)-13-icosene-10-one, 14-methyl-1-octadecene, AKD-1193 (code number), BCS-AA10147 (code number), CL900167 (code number), NA-85 (code number), NI-30 (code number), 0-O-diethyl-O-[4-(dimethylsulfamoyl)phenyl]-phosphorothionate (DSP), 0-ethyl-O-4-(nitrophenyl)phenyiphosphonothioate (EPN), RU15525 (code number), XMC, Z-13-icosene-10-one, ZX18901 (code number), and F4260 (code number).

Next, known fungicides (fungicidal active ingredients) or disease control agent compounds which may be mixed or used in combination are exemplified below.

Fungicidal active ingredients or disease control agent compounds:
Agrobacterim radiobacter, azaconazole, acibenzolar-S-methyl, azoxystrobin, anilazine, amisulbrom, aminopyrifen, ametoctradin, aldimorph, isotianil, isopyrazam, isofetamid, isoflucypram, isoprothiolane, ipconazole, ipflufenoquin, ipfentrifluconazole, iprodione, iprovalicarb, iprobenfos, imazalil, iminoctadine-albesilate, iminoctadine-triacetate, imibenconazole, inpyrfluxam, imprimatin A, imprimatin B, edifenphos, etaconazole, ethaboxam, ethirimol, ethoxyquin, etridiazole, enestroburin, enoxastrobin, epoxiconazole, organic oils, oxadixyl, oxazinylazole, oxathiapiprolin, oxycarboxin, oxine-copper, oxytetracycline, oxpoconazole-fumarate, oxolinic acid, copper dioctanoate, octhilinone, ofurace, orysastrobin, o-phenylphenol, kasugamycin, captafol, carpropamid, carbendazim, carboxin, carvone, Candida oleophila, Candida saitoana, quinoxyfen, quinofumelin, chinomethionat, captan, quinconazole, quintozene, guazatine, cufraneb, coumethoxystrobin, coumoxystrobin, Gliocradium catenulatum, Cryptococcus albidus, kresoxim-methyl, clozylacon, Clonostachys rosea, chlozolinate, chloroinconazide, chlorothalonil, chloroneb, Chaetomium cupreun, Coniothyrium minitans, cyazofamid, diethofencarb, diclocymet, dichlofluanid, dichlobentiazox, diclomezine, dicloran, dichlorophen, dithianon, diniconazole, diniconazole-M, zineb, dinocap, dipymetitrone, diphenylamine, difenoconazole, cyflufenamid, diflumetorim, cyproconazole, cyprodinil, simeconazole, dimethirimol, dimethyl disulfide, dimethomorph, cymoxanil, dimoxystrobin, Pseudozyma flocculosa, Pseudomonas aureofaciens, Pseudomonas chlororaphis, Pseudomonas syringae, Pseudomonas flurorescens, Pseudomonas rhodesiae, ziram, silthiofam, Zucchini yellow mosaic virus, streptomycin, Streptomyces griseoviridis, Streptomyces lygicus, spiroxamine, sedaxane, zoxamide, solatenol, dazomet, Talaromyces flavus, tiadinil, thiabendazole, thiram, thiophanate, thiophanate-methyl, thifluzamide, thiram, tecnazene, tecloftalam, tetraconazole, debacarb, tebuconazole, tebufloquin, terbinafine, dodine, dodemorph, triadimenol, triadimefon, triazoxide, trichlamide, triclopyricarb, Trichoderma asperellum, Trichoderma atroviride, Trichoderma gamsii, Trichoderma stromaticum, Trichoderma paecilomyces, Trichoderma harzianum, Trichoderma viride, Trichoderma virens, Trichoderma polysporum, Trichoderma harzianum rifai, Trichoderma lignorum, tricyclazole, triticonazole, tridemorph, triflumizole, trifloxystrobin, triforine, tolylfluanid, tolclofos-methyl, tolnifanide, tolprocarb, nabam, natamycin, naftifine, nitrapyrin, nitrothal-isopropyl, nuarimol, copper nonyl phenol sulfonate, Paenibacillus polymyxa, Barkholderia cepacia, Bacillus amyloliquefaciens, Bacillus simplex, Bacillus subtilis, Bacillus pumilus, Bacillus licheniformis, harpin protein, Variovorax paradoxus, validamycin, valifenalate, Pantoea agglomerans, picarbutrazox, bixafen, picoxystrobin, Pythium oligandrum, pydiflumetofen, bitertanol, binapacryl, hinokitiol, Erwinia carotovora, Rhizobium vitis, biphenyl, piperalin, hymexazol, pyraoxystrobin, pyraclostrobin, pyraziflumid, pyrazophos, pyrapropoyne, pyrametostrobin, pyriofenone, pyrisoxazole, pyridachiometyl, pyrifenox, pyributicarb, pyribencarb, pyrimethanil, pyroquilon, vinclozolin, ferbam, famoxadone, phenazine oxide, fenamidone, fenaminstrobin, fenarimol, fenoxanil, ferimzone, fenpiclonil, fenpicoxamid, fenpyrazamine, fenbuconazole, fenfuram, fenpropidin, fenpropimorph, fenhexamid, folpet, phthalide, Fusarium oxysporum, bupirimate, fuberidazole, blasticidin-S, furametpyr, furalaxyl, furancarboxylic acid, fluazinam, fluindapyr, fluoxastrobin, fluoxapiprolin, fluopicolide, fluopimomide, fluopyram, fluoroimide, fluxapyroxad, fluquinconazole, furconazole, furconazole-cis, fludioxonil, flusilazole, flusulfamide, flutianil, flutolanil, flutriafol, flufenoxystrobin, flubeneteram, flumetover, flumorph, Phlebiopsis gigantea, proquinazid, prochloraz, procymidone, prothiocarb, prothioconazole, bronopol, propamocarb-hydrochloride, propiconazole, propineb, probenazole, bromuconazole, flometoquin, florylpicoxamid, hexaconazole, benalaxyl, benalaxyl-M, benodanil, benomyl, pefurazoate, penconazole, pencycuron, benzovindiflupyr, benthiazole, benthiavalicarb-isopropyl, penthiopyrad, penflufen, boscalid, fosetyl (including a salt of aluminum, calcium, sodium, or the like), polyoxin, polycarbamate, Bordeaux mixture, mancopper, mancozeb, mandipropamid, mandestrobin, maneb, myclobutanil, mineral oils, mildiomycin, methasulfocarb, metam, metalaxyl, metalaxyl-M, metiram, metyltetraprole, metconazole, metominostrobin, metrafenone, mepanipyrim, mefentrifluconazole, meptyldinocap, mepronil, iodocarb, laminarin, phosphorous acid and salts, copper oxychloride, silver, copper (II) sulfate, cuprous oxide, copper hydroxide, potassium bicarbonate, sodium bicarbonate, sulfur, oxyquinoline sulfate, copper sulfate, (3,4-dichloroisothiazole-5-yl)methyl-4-(tert-butyl) benzoic acid ester (Chemical Name, CAS Registry Number: 1231214-23-5), BAF-045 (code number), BAG-010 (code number), UK-2A (code number), dodecylbenzenesulfonic acid bisethylenediamine copper complex [II] (DBEDC), MIF-1002 (code number), NF-180 (code number), triphenyltin acetate (TPTA), triphenyltin chloride (TPTC), triphenyltin hydroxide (TPTH), and F9650 (code number).

Next, known herbicidal compounds, herbicidal active ingredients, or plant growth regulator compounds which may be mixed or used in combination are exemplified below, but are not limited to these examples.

Herbicidal compounds or herbicidal active ingredients: ioxynil (including lithium salt, sodium salt, a salt with octanoic acid or the like), aclonifen, acrolein, azafenidin, acifluorfen (including a salt with sodium or the like), azimsulfuron, asulam, acetochlor, atrazine, anilofos, amicarbazone, amidosulfuron, amitrole, aminocyclopyrachlor, aminopyralid, amiprofos-methyl, ametryn, Araujia Mosaic Virus, alachlor, Alternaria destruens, alloxydim (including a salt with sodium or the like), ancymidol, isouron, isoxachlortole, isoxaflutole, isoxaben, isodecylalcohol ethoxylate, isoproturon, ipfencarbazone, imazaquin, imazapic (including a salt with amine or the like), imazapyr (including a salt of isopropylamine or the like), imazamethabenz, imazamethabenz-methyl, imazamox, imazethapyr, imazosulfuron, indaziflam, indanofan, eglinazine-ethyl, esprocarb, ethametsulfuron-methyl, ethalfluralin, ethidimuron, ethoxysulfuron, ethoxyfen, ethoxyfen-ethyl, ethofumesate, etobenzanid, epyrifenacil, endothal-disodium, oxadiazon, oxadiargyl, oxaziclomefone, oxasulfuron, oxyfluorfen, oryzalin, Obuda Pepper virus, orthosulfamuron, orbencarb, oleic acid, cafenstrole, caprylic acid, capric acid, carfentrazone-ethyl, karbutilate, carbetamide, quizalofop, quizalofop-ethyl, quizalofop-P-ethyl, quizalofop-P-tefuryl, Xanthomonas campestris, quinoclamine, quinclorac, quinmerac, citric acid, cumyluron, clacyfos, glyphosate (including a salt of sodium, potassium, amine, propylamine, isopropylamine, ammonium, isopropyl ammonium, guanidine, monoethanolamine, choline, BAPMA (N,N-bis-(aminopropyl)methylamine), dimethylamine, or trimesium or the like), glufosinate (including a salt of amine, or sodium or the like), glufosinate-P, glufosinate-P-sodium, clethodim, clodinafop, clodinafop-propargyl, clopyralid (including a monoethanolamine salt), clomazone, chlomethoxyfen, clomeprop, cloransulam-methyl, chloramben, chloridazon, chlorimuron, chlorimuron-ethyl, chlorsulfuron, chlorthal-dimethyl, chlorthiamid, chlorphthalim, chlorflurenol-methyl, chlorpropham, chlorbromuron, chloroxuron, chlorotoluron, ketospiradox (including a salt of sodium, calcium, or ammonia or the like), Colletotrichum orbiculare, Colletotrichum gloeosporioides, Colletotrichum truncatum, Chondrostercum purpureum, saflufenacil, sarmentine, cyanazine, cyanamide, diuron, diethatyl-ethyl, dicamba (including a salt of amine, diethylamine, isopropylamine, diglycolamine, dimethyl ammonium, diolamine, isopropyl ammonium, auramine, potassium, trolamine, BAPMA (N,N-bis-(aminopropyl)methylamine), choline, sodium, or lithium or the like, or an ester such as methyl ester), cycloate, cycloxydim, diclosulam, cyclosulfamuron, cyclopyranil, cyclopyrimorate, dichlobenil, diclofop, diclofop-P-methyl, diclofop-methyl, dichlorprop, dichlorprop-P (including a salt of dimethyl ammonium, potassium, sodium, choline or the like, or an ester such as butotyl ester, 2-ethylhexyl ester, isoctyl ester, or methyl ester), diquat, diquat dibromide, dithiopyr, siduron, dinitramine, cinidon-ethyl, cinosulfuron, dinoseb (including acetate), dinoterb, cyhalofop, cyhalofop-butyl, cypyrafluone, diphenamid, difenzoquat, diflufenican, diflufenzopyr, simazine, dimesulfazet, dimethachlor, dimethametryn, dimethenamid, dimethenamid-P, simetryn, dimepiperate, dimefuron, Pseudomonas fluorescens, cinmethylin, swep, sulcotrione, sulfentrazone, sulfosate, sulfosulfuron, sulfometuron-methyl, sethoxydim, Sclerotinia minor, terbacil, daimuron, thaxtomin A, Tobacco Mild Green Mosaic Tobamovirus, Tobacco Rattle Virus, dalapon, thiazopyr, tiafenacil, thiencarbazone (including sodium salt, methyl ester, or the like), tiocarbazil, thiobencarb, thidiazimin, thidiazuron, thifensulfuron, thifensulfuron-methyl, desmedipham, desmetryne, tetflupyrolimet, thenylchlor, tebutam, tebuthiuron, tepraloxydim, tefuryltrione, terbuthylazine, terbutryn, terbumeton, tembotrione, topramezone, tralkoxydim, triaziflam, triasulfuron, triafamone, tri-allate, trietazine, triclopyr, triclopyr-butotyl, triclopyr-triethylammonium, tritosulfuron, tripyrasulfone, trifludimoxazin, triflusulfuron-methyl, trifluralin, trifloxysulfuron (including a salt of sodium or the like), tribenuron-methyl, tolpyralate, naptalam (including a salt with sodium or the like), naproanilide, napropamide, napropamide-M, nicosulfuron, lactic acid, neburon, norflurazon,
Barkholderia rinojensis, vernolate, paraquat, paraquat dichloride, halauxifen, halauxifen-benzyl, halauxifen-methyl, Burkholderia rinojensis, haloxyfop, haloxyfop-P, haloxyfop-etotyl, haloxyfop-P-methyl, halosafen, halosulfuron-methyl, bixlozone, picloram (including a salt with dichloro ammonium, trolamine or the like), picolinafen, bicyclopyrone, bispyribac-sodium, pinoxaden, bipyrazone, bifenox, piperophos, pyraclonil, pyrasulfotole, pyrazoxyfen, pyrazosulfuron-ethyl, pyrazolynate, bilanafos, pyraflufen, pyraflufen-ethyl, pyridafol, pyrithiobac-sodium, pyridate, pyriftalid, pyributicarb, pyribenzoxim, pyrimisulfan, pyriminobac-methyl, pyroxasulfone, pyroxsulam, Phytophthora palmivora, phenisopham, fenuron, fenoxasulfone, fenoxaprop (including methyl, ethyl, or isopropyl ester), fenoxaprop-P (including methyl, ethyl, or isopropyl ester), fenquinotrione, fenthiaprop-ethyl, fentrazamide, fenpyrazone, phenmedipham, Phoma chenopodicola, Phoma herbarum, Phoma macrostoma, butachlor, butafenacil, butamifos, butylate, Puccinia canaliculata, Puccinia thlaspeos, butenachlor, butralin, butroxydim, flazasulfuron, flamprop (including methyl, ethyl, or isopropyl ester), flamprop-M (including methyl, ethyl, or isopropyl ester), primisulfuron, primisulfuron-methyl, fluazifop-butyl, fluazifop-P, fluazifop-P-butyl, fluazolate, fluometuron, fluoroglycofen-ethyl, flucarbazone-sodium, fluchloralin, flucetosulfuron, fluthiacet-methyl, flupyrsulfuron-methyl (including a salt of sodium, calcium, or ammonia or the like), flufenacet, flufenpyr-ethyl, flupropanate (including sodium salt), flupoxame, flumioxazin, flumiclorac-pentyl, flumetsulam, fluridone, flurtamone, fluroxypyr (including an ester of butometyl, meptyl or the like, or a salt of sodium, calcium, ammonia or the like), flurochloridone, pretilachlor, procarbazone (including a salt with sodium or the like), prodiamine, prosulfuron, prosulfocarb, propaquizafop, propachlor, propazine, propanil, propyzamide, propisochlor, propyrisulfuron, propham, profluazol, prohexadione-calcium, propoxycarbazone, propoxycarbazone-sodium, profoxydim, bromacil, brompyrazon, prometryn, prometon, bromoxynil (including an ester of butyric acid, octanoic acid, or heptanoic acid or the like), bromofenoxim, bromobutide, florasulam, florpyrauxifen, florpyrauxifen-benzyl, hexazinone, pethoxamid, benazolin, benazolin-ethyl, penoxsulam, Pepino Mosaic Virus, heptamaloxyloglucan, beflubutamid, beflubutamid-M, pebulate, pelargonic acid, bencarbazone, benquitrione, benzfendizone, bensulide, bensulfuron, bensulfuron-methyl, benzobicyclon, benzofenap, bentazone, pentanochlor, pendimethalin, pentoxazone, benfluralin, benfuresate, fosamine, fomesafen, foramsulfuron, forchlorfenuron,
mecoprop (including a salt of sodium, potassium, isopropylamine, triethanolamine, dimethylamine, diolamine, trolamine, choline or the like, or an ester such as ethadyl ester, 2-ethylhexyl ester, isoctyl ester, or methyl ester), mecoprop-P-potassium, mesosulfuron (including an ester of methyl or the like), mesotrione, metazachlor, metazosulfuron, methabenzthiazuron, metamitron, metamifop, metam (including a salt of sodium or the like), disodium methanearsonate (DSMA), methiozolin, methyldymuron, metoxuron, metosulam, metsulfuron-methyl, metobromuron, metobenzuron, metolachlor, metribuzin, mepiquat chloride, mefenacet, monosulfuron (including methyl, ethyl, or isopropyl ester), monolinuron, molinate, iodosulfuron, iodosulfulon-methyl-sodium, iofensulfuron, iofensulfuron-sodium, lactofen, lancotrione, linuron, rimsulfuron, lenacil, 2,2,2-trichloroacetic acid (TCA) (including a salt of sodium, calcium, or ammonia or the like), 2,3,6-trichlorobenzoic acid (2,3,6-TBA), 2,4,5-trichlorophenoxyacetic acid (2,4,5-T), 2,4-dichlorophenoxyacetic acid (2,4-D) (including a salt of amine, diethylamine, triethanolamine, isopropylamine, dimethylammonium, diolamine, dodecylammonium, heptylammonium, tetradecylammonium, triethylammonium, tris(2-hydroxypropyl)ammonium, trolamine, choline, sodium, lithium or the like, or an ester such as butotyl ester, 2-butoxypropyl ester, 2-ethylhexyl ester, methyl ester, ethylester, butyl ester, isobutyl ester, octyl ester, pentyl ester, propylester, isoctyl ester, isopropyl ester, meptyl ester, or tefuryl ester), 2,4-dichlorophenoxybutyric acid (2,4-DB) (including a salt of amine, diethylamine, triethanolamine, isopropylamine, dimethylammonium, choline, sodium, lithium or the like, or an ester such as isoctyl ester), 2-amino-3-chloro-1,4-naphthoquinone (ACN), 2-methyl-4-chlorophenoxyacetic acid (MCPA) (including a salt of sodium, dimethylammonium, choline or the like, or an ester such as 2-ethylhexyl ester, isoctyl ester, ethyl ester or the like), 2-methyl-4-chlorophenoxybutyric acid (MCPB) (including sodium salt, ethyl ester, or the like), 4-(2,4-dichlorophenoxy)butyric acid (2,4-DB), 4,6-dinitro-0-cresol (DNOC) (including a salt of amine or sodium or the like), (5S)-3-(3,5-difluorophenyi)-N-[rel-(3R,5R)-5-(trifluoromethylsulfonylcarbamoyl)tetrahydrofuran-3-yl]-5-vinyl-4H-isoxazole-5-carboxamid,
(Chemical Name, CAS Registry Number: 2266183-40-6) (WO 2018/228986 A, WO 2020/114934 A), N4-(2,6-difluorophenyl)-6-(1-fluoro-1-methyl-ethyl)-1,3,5-triazine-2,4-diamine (Chemical Name, CAS Registry Number: 1606999-43-2) (WO 2014/064094 A, WO 2015/162164 A), (5S)-3-(3,5-difluorophenyl)-N-[(3R)-5-(methylsulfonylcarbamoyl)-2,3-dihydrofuran-3-yl]-5-vinyl-4H-isoxazole-5-carboxamid (Chemical Name, CAS Registry Number: 2266190-06-9) (WO 2018/228986 A, WO 2020/114934 A), (5R)-3-(3,5-difluorophenyl)-5-methyl-N-[rel-(3R,5R)-5-(methylsulfonylcarbamoyl)tetrahydrofuran-3-yi]-4H-isoxazole-5-carboxamid (Chemical Name, CAS Registry Number: 2266164-36-5) (WO 2018/228986 A, WO 2020/114934 A), (5R)-3-(3,5-difluorophenyl)-N-[(3R)-5-(methoxycarbamoyl)-2,3-dihydrofuran-3-yl]-5-methyl-4H-isoxazole-5 carboxyamide (Chemical Name, CAS Registry Number: 2266170-31-2) (WO 2018/228986 A, WO 2020/114934 A), 2-[2-(3,4-dimethoxyphenyl)-6-methyl-3-oxo-pyridazine-4-carbonyl]cyclohexane-1,3-dione (Chemical Name, CAS Registry Number: 2138855-12-4) (WO 2017/178582 A, WO 2018/015476 A), 4-hydroxy-1-methyl-3-[4-(trifluoromethyl)-2-pyridyl]imidazolidin-2-one (Chemical Name, CAS Registry Number: 1708087-22-2) (WO 2015/059262 A, WO 2018/015476 A), 6-(1-fluorocyclopentyl)-N4-(2,3,5,6-tetrafluorophenyl)-1,3,5-triazine-2,4-diamine (Chemical Name, CAS Registry Number: 1820807-75-7) (WO 2015/162164 A), 6-(1-fluoro-1-methyl-ethyl)-N4-(2,3,5,6-tetrafluorophenyl)-1,3,5-triazine-2,4-diamine (Chemical Name, CAS Registry Number: 1606999-21-6) (WO 2014/064094 A, WO 2015/162164 A), (5S)-3-(3-fluoro-5-methyl-phenyl)-N-[rel-(3R,5R)-5-(methoxycarbamoyl)tetrahydrofuran-3-yl]-5-vinyl-4H-isoxazole-5-carboxamid (Chemical Name, CAS Registry Number: 2266292-43-5) (WO 2018/228986 A, WO 2020/114934 A), 6-(1-methylcyclobutyl)-N4-(2,3,5,6-tetrafluorophenyl)-1,3,5-triazine-2,4-diamine (Chemical Name, CAS Registry Number: 1607001-97-7) (WO 2014/064094 A, WO 2015/162164 A), AE-F-150944 (code number), F9960 (code number), IR-6396 (code number), MCPA-thioethyl, NC-656 (code number), SYP-298 (code number), SYP-300 (code number), S-ethyldipropylthiocarbamate (EPTC), S-metolachlor, S-9750 (code number), MSMA, and HW-02 (code number).

### Plant Growth Regulators:

1-naphthylacetamide, 1-methylcyclopropene, 1,3-diphenylurea, 2,3,5-triiodobenzoic acid, 2-methyl-4-chlorophenoxybutyric acid (MCPB) [including sodium salt, ethyl ester, or the like], 2-(naphthalene-1-yl)acetamide, 2,6-diisopropylnaphthalene, 3-[(6-chloro-4-phenylquinazoline-2-yl)amino]propane-1-ol, 4-oxo-4-(2-phenylethyl) amino butyric acid (Chemical Name, CAS Registry Number: 1083-55-2), 4-chlorophenoxyacetic acid (4-CPA), 5-aminolevulinic acid hydrochloride, methyl 5-(trifluoromethyl)benzo[b]thiophene-2-carboxylate, aminoethoxyvinylglycine (AVG), n-decanol, aviglycine, ancymidol, abscisic acid, isoprothiolane, inabenfide, indole acetic acid, indole butyric acid, uniconazole, uniconazole-P, Ecolyst, ethychlozate, ethephon, epocholeone, calcium chloride, choline chloride, oxine-sulfate, kinetin, calcium peroxide, carvone, calcium formate, cloxyfonac, cloxyfonac-potassium, cloprop, chlormequat, chlormequat-chloride, chlorpropham, choline, cytokinins, oxydized glutathione, cyanamide, sodium cyanate, cyclanilide, dichlorprop (including a salt of dimethylammonium, potassium, sodium, choline or the like, or an ester such as butotyl ester, 2-ethylhexyl ester, isoctyl ester, or methyl ester), dichlorprop-P (including a salt of sodium, potassium, dimethylammonium or the like, or 2-ethylhexyl ester), diquat, diquat dibromide, dikegulac, gibberellic acid, gibberellin A4, gibberellin A7, dimethipin, sintofen, jasmone, cis-jasmone, jasmonic acid, methyl jasmonate, streptomycin, calcium polysulfide, daminozide, calcium carbonate, thidiazuron, decan-1-ol, triacontanol, triapenthenol, trinexapac-ethyl, tribufos, paclobutrazol, paraffin, bispyribac-sodium, hymexazol, butralin, fluthiacet-methyl, pyraflufen-ethyl, flumetralin, flurprimidol, flurenol, pronitridine, prohydrojasmon, prohexadione-calcium, heptamaloxyloglucan, 6-benzylaminopurine, pendimethalin, forchlorfenuron, formononetin, maleic hydrazide, mepiquat chloride, mefluidide, lipochitooligosaccharides, for example, lipochitooligosaccharides SP104, and calcium sulfate.

Next, known phytotoxicity reducing compounds which may be mixed or used in combination are exemplified.

### Phytotoxicity Reducing Compounds:

isoxadifen, isoxadifen-ethyl, oxabetrinil, octane-1,8-diamine, cloquintocet, cloquintcet-mexyl, dietholate, cyometrinil, dichlormid, dicyclonone, cyprosulfamide, daimuron, 1,8-naphthalic anhydride, fenchlorazole, fenchlorazole-O-ethyl, fenclorim, furilazole, fluxofenim, flurazole, benoxacor, metcamifen, mephenate, mefenpyr, mefenpyr-ethyl, mefenpyr-diethyl, lower alkyl-substituted benzoic acid, 2,2-dichloro-N-(1,3-dioxolan-2-ylmethyl)-N-(2-propenyl)acetamide (PPG-1292), 2-dichloromethyl-2-methyl-1,3-dioxane (MG-191), 3-dichloroacetyl-2,2,5-trimethyl-1,3-oxazolidine (R-29148), 4-dichloroacetyl-1-oxa-4-azaspiro[4.5]decane (AD-67), 4-carboxy-3,4-dihydro-2H-1-benzopyran-4-acetic acid (CL-304415, code number), MON4660 (code number), metcamifen, N1,N2-diallyl-N2-dichloroacetylglycinamide (DKA-24, code number), 1-bromo-4-[(chloromethyl)sulfonyl]benzene (CSB), 2-propenyl-1-oxa-4-azaspiro[4,5]decane-4-carbodithioate (MG-838, code number), 3-(dichloroacetyl)-2,2-dimethyl-1,3-oxazolidine (R-28725, code number), R-29148 (code number), and 1-(dichloroacetyl)azepane (TI-35, code number).

Next, known biological pesticides which may be mixed or used in combination are exemplified.

### Biological Pesticides:

Haplothrips brevitubus, Franklinothrips vespiformis, Diglyphus isaea, Encarsia formosa, Amblyseius cucumeris, Pseudaphycus malinus, Amblyseius womersleyi, Aphidius colemani, Eretmocerus eremicus, Aphidoletes aphidimyza, Amblyseius swirskii, Orius strigicollis, Phytoseiulus persimilis, Amblyseius degenerans, Phytoseiulus persimilis, Orius sauteri, Dacnusa sibirica, Amblyseius californicus, Chrysoperla nipponensis, and Anicetus beneficus.

Next, known agricultural materials which may be mixed or used in combination will be exemplified.

### Agricultural Materials:

ethylene, hypochlorous acid solution (limited to those obtained by electrolysis of hydrochloric acid or aqueous potassium chloride solution), sodium bicarbonate, vinegar, humic substances, humic acid, fulvic acid, seaweed extracts, polysaccharides, amino acid, microbial materials, functional components derived from animals and plants, microbial metabolites, microbial activation materials, soil stickers, soil permeability control materials, soil water retention materials, and the like, as well as bio-stimulants.

Next, known agricultural fertilizer components which may be mixed or used in combination are exemplified.

Examples of the fertilizer include an inorganic fertilizer and an organic fertilizer. Specific examples thereof include ammonium chloride, ammonium sulfate, ammonium nitrate, ammonium dihydrogen phosphate, ammonium urea nitrate, urea, calcium cyanamide, potassium nitrate, superphosphate of lime, double superphosphate of lime, potassium dihydrogen phosphate, potassium chloride, potassium sulfate, potassium carbonate, potassium silicate, oil cake, fish meal, rice bran, bat guano, and fermented chicken manure.

The pest control agent of the present invention configured as described above exhibits an excellent control effect on pests such as Orthoptera pests, Thysanoptera pests, Hemiptera pests, Coleoptera pests, Diptera pests, Lepidoptera pests, Hymenoptera pests, Collembola pests, Thysanura pests, Blattodea pests, Psocoptera pests, Mallophaga pests, Phthiraptera pests, plant parasitic mites, plant parasitic nematodes, plant parasitic mollusks, other harmful animals, unpleasant animals, hygiene pests, and parasites. Examples of such pests include the following species.

Examples of the Orthoptera pests include Tettigoniidae pests such as Ruspolia lineosa; Gryllidae pests such as Teleogryllus emma and Truljalia hibinonis; Gryllotalpidae pests such as Gryllotalpa orientalis; Acrididae pests such as Oxya hyla intricate, Locusta migratoria, Melanoplus sanguinipes, Melanoplus differentialis, and Melanoplus femurrubrum; Pyrgomorphidae pests such as Atractomorpha lata; Eneopteridae pests such as Euscyrtus japonicus; and Tridactylidae pests such as Xya japonicus.

Examples of the Thysanoptera pests include Thripidae pests such as Frankliniella intonsa, Frankliniella occidentalis, Scirtothrips dorsalis, Thrips palmi, Thrips tabaci, Thrips setosus, Heliothrips haemorrhoidalis, and Stenchaetothrips biformis; and Phlaeothripidae pests such as Ponticulothrips diospyrosi, Liothrips wasabiae, and Haplothrips aculeatus.

Examples of the Hemiptera pests include Cicadidae pests such as Mogannia minuta; Aphrophoridae pests such as Aphrophora intermedia and Mahanarva fimbriolata; Membracidae pests such as Machaerotypus sibiricus; Cicadellidae pests such as Arboridia apicalis, Empoasca onukii, Nephotettix cincticeps, Nephotettix malayanus, Nephotettix virescens, Nephotettix nigropictus, Recilia dorsalis, Amrasca biguttula, Idioscopus nitidulus, Idioscopus clypealis, Amritodus atkinsoni, Empoasca fabae, and Dalbulus maidis; Cixiidae pests such as Pentastiridius apicalis; Delphacidae pests such as Laodelphax striatellus, Nilaparvata lugens, Sogatella furcifera, and Peregrinus maidis; Meenoplidae pests such as Nisia nervosa; Derbidae pests such as Kamendaka saccharivora; Achilidae pests such as Achilus flammeus; Ricaniidae pests such as Orosanga japonicus; Flatidae pests such as Mimophantia maritima; Psyllidae pests such as Cacopsylla pyrisuga and Diaphorina citri; Calophyidae pests such as Calophya mangiferae; Phylloxeridae pests such as Daktulosphaira vitifoliae; Adelgidae pests such as Adelges laricis and Adelges tsugae; Aphididae pests such as Acyrthosiphon pisum, Aphis gossypii, Aphis spiraecola, Lipaphis erysimi, Brevicoryne brassicae, Myzus persicae, Schizaphis graminum, Rhopalosiphum padi, Toxoptera aurautii, Aulacorthum solani, Macrosiphum euphorbiae, Nasonovia ribisnigri, Sitobion avenae, and Aphis glycines; Aleyrodidae pests such as Aleurocanthus camelliae, Aleurocanthus spiniferus, Bemisia tabaci, Bemisia argentifolii, and Trialeurodes vaporariorum; Monophlebidae pests such as Drosicha corpulenta and Icerya purchasi; Pseudococcidae pests such as Dysmicoccus brevipes, Planococcus citri, and Pseudococcus comstocki; Coccidae pests such as Ceroplastes ceriferus and Ceroplastes rubens; Aclerdidae pests such as Aclerda takahashii; Diaspididae pests such as Aonidiella aurantii, Diaspidiotus perniciosus, Pseudaulacaspis pentagoa, and Unaspis yanonensis; Miridae pests such as Lygus lineolaris, Trigonotylus caelestialium, Apolygus lucorum, Nesidiocoris tenuis, and Halticus bractatus; Tingidae pests such as Stephanitis pyrioides and Stephanitis nashi; Pentatomidae pests such as Eurydema rugosa, Eysarcoris lewisi, Eysarcoris aeneus, Lagynotomus elongatus, Nezara viridula, Plautia crossota, Nezara antennata, Eushistus heros, Piezodorus guildini, Tibraca limbativentris, and Dichelops furcatus; Plataspidae pests such as Megacopta cribraria; Urostylididae pests such as Urochela luteovoria; Lygaeidae pests such as Cavelerius saccharivorus; Malcidae pests such as Malcus japonicus; Pyrrhocoridae pests such as Dysdercus cingulatus; Alydidae pests such as Leptocorisa acuta and Leptocorisa chinensis; Coreidae pests such as Anacanthocoris striicornis; Rhopalidae pests such as Rhopalus maculatus; Cydnidae pests such as Scaptocoris castanea; and Cimicidae pests such as Cimex lectularis.

Examples of the Coleoptera pests include Scarabaeidae pests such as Anomara cuprea, Anomara rufocuprea, Popillia japonica, Oxycetonia jucunda, Anomala geniculata, Oryctes rhinoceros, Heptophylla picea, and Phyllophaga cuyabana; Elateridae pests such as Agriotes ogurae, Agriotes lineatus, Agriotes obscurus, Melanotus okinawensis, and Melanotus fortnumi; Dermestidae pests such as Anthrenus verbasci; Bostrychidae pest such as Heterobostrychus hamatipennis; Anobiidae pests such as Stegobium paniceum; Ptinidae pests such as Pitinus clavipes; Trogossitidae pests such as Tenebroides mauritanicus; Cleridae pests such as Necrobia rufipes; Nitidulidae pests such as Carpophilus hemipterus and Meligethes aeneus; Silvanidae pests such as Ahasverus advena; Laemophloeidae pests such as Cryptolestes ferrugineus; Coccinellidae pests such as Epilachna varivestis and Henosepilachna vigintioctopunctata; Tenebrionidae pests such as Tenebrio molitor and Tribolium castaneum; Meloidae pest such as Epicauta gorhami; Cerambycidae pests such as Anoplophora glabripennis, Xylotrechus pyrrhoderus, Monochamus alternatus, and Dectes texanus; Bruchinae pests such as Callosobruchus chinensis; Chrysomelidae pests such as Leptinotarsa decemlineata, Diabrotica virgifera, Diabrotica barberi, Diabrotica undecimpunctata howardi, Aulacophora femoralis, Phaedon brassicae, Cassida nebulosa, Oulema oryzae, Epilachna varivestis, Phyllotreta striolata, Demotina fasciculata, Psylliodes chrysocephala, Cerotoma trifurcate, Colaspis brunnea, Colaspis crinnicornis, Odontota horni, Chaetocnema pulicaria, and Diabrotica balteata; Brentidae pests such as Cylas formicarius; Curculionidae pests such as Hypera postica, Listroderes costirostris, Euscepes postfasciatus, Curculio sikkimensis, Anthonomus grandis, and Sternechus subsignatus; Erirhinidae pests such as Echinocnemus bipunctatus, Lissorhoptrus oryzophilus, and Oryzophagus oryzae; Rhynchophoridae pests such as Sitophilus zeamais, Sphenophrus venatus, and Sphenophorus levis; Scolytidae pests such as Tomicus piniperda; Platypodidae pests such as Crossotarsus niponicus; and Lyctinae pests such as Lyctus brunneus.

Examples of the Diptera pests include Tipulidae pests such as Tipula aino; Bibionidae pests such as Plecia nearctica; Mycetophilidae pests such as Exechia shiitakevora; Sciaridae pests such as Pnyxia scabiei and Bradysia agrestis; Cecidomyiidae pests such as Asphondylia yushimai, Mayetiola destructor, and Dasineura oxycoccana; Culicidae pests such as Aedes aegypti and Culex pipiens pallens; Simuliidae pests such as Simulium takahashii; Chironomidae pests such as Chironomus oryzae; Tabanidae pests such as Chrysops suavis and Tabanus trigonus; Syrphidae pests such as Eumerus strigatus; Tephritidae pests such as Bactrocera dorsalis, Euphranta japonica, and Ceratitis capitata; Agromyzidae pests such as Liriomyza trifolii, Liriomyza sativae, Agromyza oryzae, Liriomyza bryoniae, Chromatomyia horticola, and Liriomyza chinensis; Chloropidae pests such as Meromyza nigriventris; Drosophilidae pests such as Drosophila suzukii and Drosophila melanogaster; Ephydridae pests such as Hydrellia griseola; Hippoboscidae pests such as Hippobosca equina; Scathophagidae pests such as Parallelpmma sasakawae; Anthomyiidae pests such as Delia antiqua and Delia platura; Fanniidae pests such as Fannia canicularis; Muscidae pest such as Musca domestica and Stomoxys calcitrans; Sarcophagidae pests such as Sarcophaga peregrina; Gasterophilidae pests such as Gasterophilus intestinalis; Hypodermatidae pests such as Hypoderma lineatum; and Oestridae pests such as Oestrus ovis.

Examples of the Lepidoptera pests include Hepialidae pests such as Endoclita excrescens; Heliozelidae pests such as Antispila ampelopsia; Cossidae pests such as Zeuzera leuconotum and Cossus insularis; Tortricidae pests such as Archips fuscocupreanus, Adoxophyes orana fasciata, Grapholita molesta, Homona magnanima, Leguminivora glycinivorella, Cydia pomonella, and Lobesia botrana; Tortricidae pests such as Eupoecilia ambiguella; Psychidae pests such as Bambalina sp. and Eumeta minuscula; Tineidae pests such as Nemapogon granella and Tinea translucens; Bucculatricidae pest such as Bucculatrix pyrivorella; Tischeriidae pests such as Lyonetia clerkella and Lyonetiaprunifoliella malinella; Gracillariidae pests such as Caloptilia theivora and Phyllonorycter ringoniella; Phyllocnistidae pests such as Phyllocnistis citrella; Acrolepiidae pests such as Acrolepiopsis sapporensis; Plutellidae pests such as Plutella xylostella; Yponomeutidae pests such as Yponomeuta orientalis; Argyresthidae pests such as Argyresthia conjugella; Sesiidae pests such as Nokona regalis and Synanthedin hector; Gelechiidae pests such as Phthorimaea operculella, Sitotroga cerealella, Pectinophora gossypiella, and Tuta absoluta; Carposinidae pests such as Carposina sasakii; Zygaenidae pest such as Illiberis pruni; Limacodidae pests such as Monema flavescens; Crambidae pests such as Ancylolomia japonica, Chilo suppressalis, Cnaphalocrocis medinalis, Ostrinia furnacalis, Hellulla undalis, Conogethes punctiferlis, Diaphania indica, Parapediasia teterrella, and Ostrinia nubilalis; Diatraea saccharalis, Pyralidae pests such as Cadra cautella and Galleria mellonella; Pterophoridae pests such as Nippoptilia vitis; Papilionidae pests such as Papilio xuthus; Pieridae pests such as Pieris rapae; Hesperiidae pests such as Parnara guttata; Geometridae pests such as Ascotis selenaria; Lasiocampidae pests such as Dendrolimus spectabilis and Malacosoma neustrium testaceum; Sphingidae pests such as Agrius convolvuli; Lymantriinae pests such as Arna pseudoconspersa, Orygia recens approximans, and Lymantria dispar; Arctiidae pests such as Hyphantria cunea; and Noctuidae pests such as Agrotis ipsilon, Agrotis segetum, Autographa nigrisigna, Helicoverpa armigera, Helicoverpa zea, Heliothis virescens, Spodoptera exigua, Spodoptera litura, Chrysodeix includens, Spodoptera frugiperda, and Nephelodes minians.

Examples of the Hymenoptera pests include Argidae pests such as Arge pagana; Tenthredinidae pests such as Apethymus kuri and Athalia rosae ruficornis; Cynipidae pests such as Dryocosmus kuriphilus; Vespidae pests such as Vespa simillima xanthoptera; Formicidae pests such as Solenopsis invicta and Linepithema humile; and Megachilidae pests such as Megachile nipponica.

Examples of the Collembola pests include Sminthuridae pests such as Bourletiella hortensis.

Examples of the Thysanura pests include Lepismatidae pests such as Lepisma saccharina and Ctenolepisma villosa.

Examples of the Blattodea pests include Blattidae pests such as Periplaneta americana; Blattellidae pests such as Blattella germanica; Termitidae pests such as Odontotermes formosanus; Kalotermitidae pests such as Incisitermes minor and Cryptotermes domesticus; and Rhinotermitidae pests such as Coptotermes formosanus and Reticulitermes speratus.

Examples of the Psocoptera pests include Trogiomorpha pests such as Trogium pulsatorium; and Troctomorpha pests such as Liposcelis corrodens.

Examples of the Dermaptera pests include Labiduridae pests such as Labodura riparia.

Examples of the Mallophaga pests include Menoponidae pests such as Lipeurus caponis; and Trichodectidae pests such as Damalinia bovis.

Examples of the Phthiraptera pests include Haematopinidae pests such as Haematopinus suis; Pediculidae pests such as Pediculus humanus; Linognathidae pests such as Linognathus setosus; and Pthiridae pests such as Pthirus pubis.

Examples of the Acari pests include Eupodidae pests such as Penthaleus major; Tarsonemidae pests such as Phytonemus pallidus and Polyphagotarsonemus latus; Pyemotidae pests such as Siteroptes sp.; Tenuipalpidae pests such as Brevipalpus lewisi; Tuckerellidae pests such as Tuckerella pavoniformis; Tetranychidae pests such as Eotetranychus boreus, Panonychus citri, Panonychus ulmi, Tetranychus urticae, and Tetranychus kanzawai; Nalepellidae pests such as Trisetacus pini; Eriophyidae pests such as Aculops pelekassi, Epitrimerus pyri, Phyllocoptruta oleivora, and Aculops lycopersici; Diptilomiopidae pests such as Diptacus crenatae; Acaridae pests such as Aleuroglyphus ovatus, Tyrophagus putrescentiae, and Rhizoglyphus robini; Varroidae pests such as Varroa jacobsoni; Dermanyssidae pests such as Dermanyssus gallinae; Macronyssidae pests such as Ornithonyssus sylviarum; Ixodidae pests such as Boophilus microplus, Rhipicephalus sanguineus, and Haemaphysalis longicornis; and Sarcoptidae pests such as Sarcoptes scabiei.

Examples of the plant parasitic nematodes include those belonging to the family Longidoridae such as Xiphinema index; those belonging to the family Trichodoridae such as Paratrichodorus minor; those belonging to the family Rhabditidae such as Rhabditella sp.; those belonging to the family Tylenchidae such as Aglenchus sp.; those belonging to the family Tylodoridae such as Cephalenchus sp.; those belonging to the family Anguinidae such as Nothotylenchus acris and Ditylenchus destructor; those belonging to the family Hoplolaimidae such as Rotylenchulus reniformis and Helicotylenchus dihystera; those belonging to the family Paratylenchidae such as Paratylenchus curvitatus; those belonging to the family Meloidogynidae such as Meloidogyne incognita, Meloidogyne hapla, Meloidogyne javanica, Meloidogyne chitwoodi, and Meloidogyne fallax; those belonging to the family Heteroderidae such as Globodera rostochiensis, Globodera pallida, Heterodera glycines, and Heterodera Schachtii; those belonging to the family Telotylenchidae such as Tylenchorhynchus claytoni; those belonging to the family Psilenchidae such as Psilenchus sp.; those belonging to the family Criconematidae such as Criconemoides sp.; those belonging to the family Tylenchulidae such as Tylenchulus semipenetrans; those belonging to the family Sphaeronematinae such as Sphaeronema camelliae; those belonging to the family Pratylenchidae such as Radopholus citrophilus, Radopholus similis, Nacobbus aberrans, Pratylenchus penetrans, Pratylenchus coffeae, Pratylenchus zeae, and Pratylenchus brachyurus; those belonging to the family Iotonchiidae such as Iotonchium ungulatum; those belonging to the family Aphelenchus avenae such as Aphelenchus avenae; those belonging to the family Aphelenchoididae such as Aphelenchoides besseyi and Aphelenchoides fragariae; and those belonging to the family Parasitaphelenchidae such as Bursaphelenchus xylophilus.

Examples of the plant parasitic mollusks include those belonging to the family Pilinidae such as Pomacea canaliculata; those belonging to the family Veronicellidae such as Leavicaulis alte; those belonging to the family Achatinidae such as Achatina fulica; those belonging to the family Philomycidae such as Meghimatium bilineatum; those belonging to the family Succineidae such as Succinealauta; those belonging to the family Discidae such as Discus pauper; those belonging to the family Zonitidae such as Zonitoides yessoensis; those belonging to the family Limacidae such as Limax flavus, Lehmannia valentiana, and Deroceras reticulatum; those belonging to the family Helicarionidae such as Parakaliella harimensis; and those belonging to the family Bradybaenidae such as Acusta despecta sieboldiana and Bradybaena similaris.

Examples of the other pests such as harmful animals, unpleasant animals, hygiene pests, pests harmful to domestic animals, and parasites include Procambarus clarkii of the family Cambaridae of the order Decapoda, Porcellio scaber of the family Porcellionidae of the order Isopoda, Armadillidium vulgare of the family Armadillidiidae, Chilopoda pests such as Thereuonema tuberculata of the family Scutigeridae of the order Scutigeromorpha, and Scolopendra subspinipes of the order Scolopendromorpha, Diplopoda pests such as Oxidus gracilis of the family Paradoxosomatidae of the order Polydesmida, Theridiidae hasseltii of the family Theridiidae of the order Araneae, Cheiracanthium japonicum of the family Clubionidae of the order Araneae, Androctonus crassicauda of the order Scorpiones, endoparasitic nematodes such as Ascaris lumbricoides, Syphacia sp., and Wuchereria bancrofti, and endoparasitic platyhelminthes such as Distomum sp., Paragonimus westermanii, Metagonimus yokokawai, Schistosoma japonicum, Taenia solium, Taeniarhynchus saginatus, Echinococcus sp., and Diphyllobothrium latum.

The pest control agent of the present invention also exhibits a control effect on the pests and the like exemplified above which have acquired resistance to existing pest control agents. In addition, the pest control agent of the present invention can also be used for plants which have acquired characteristics such as pest resistance, disease resistance, and herbicide resistance by genetic recombination, artificial crossing, and the like.

The “plant imparted with resistance by breeding methods or gene recombination techniques” of the present invention includes not only plants imparted with resistance by classical variety crossing and resistance imparted by gene recombination techniques, but also plants imparted with resistance by new plant breeding techniques (NBTs) in which a molecular biological technique is combined with a conventional crossing technique. The new plant breeding techniques (NBTs) are described in a book “Understanding new plant breeding techniques” (International Academic Publishing Co., Ltd., written by Ryo Ohsawa and Hiroshi Ezura), a review article “Genome Editing Tools in Plants” (Genes 2017, 8, 399, written by Tapan Kumar Mohanta, Tufail Bashir, Abeer Hashem, Elsayed Fathi Abd_Allah, and Hanhong Bae), and the like.

Next, the production method, formulation method, and use of the compound of the present invention will be described in detail in the following Examples, but the present invention is not limited to these Examples at all. The melting point, which is a physical property value of the compound of the present invention, was measured with an MP-500V micro melting point measuring apparatus manufactured by Yanaco Technical Science. The refractive index was measured using an Abbe refractometer manufactured by ATAGO Co., Ltd. The ¹H NMR spectrum was measured using JNM-LA400 (400 MHz), JNM-LA300 (300 MHz), or JNM-ECS300 (300 MHz) manufactured by JEOL Ltd. with tetramethylsilane (TMS) as an internal standard. The high resolution mass spectrometry spectrum (HEMS) was measured using a Q Exactive Focus Hybrid Quadrupole-Orbitrap LC-MS/MS System (ESI, positive mode) manufactured by Thermo Fisher Scientific Inc.

The production example of a production intermediate of the compound of the present invention is also described.

### EXAMPLES

### Example 1

### Production of (R)-2-chloro-4-fluoro-5-{2-[(1,1,1-trifluoro-N-methylmethyl)sulfonamido]propoxy}-N-(2,2,2-trifluoroethyl) benzamide (compound of present invention No.: C-0191)

### (1) Production of (R)-2-(tert-butoxycarbonylamino)-1-propanol

To a mixture of 5.00 g (66.6 mmol) of (R)-2-amino-1-propanol, 7.40 g (73.1 mmol) of triethylamine, and 70 mL of tetrahydrofuran, 14.53 g (66.6 mmol) of di-tert-butyl dicarbonate was added under ice cooling, and the mixture was stirred at room temperature for 15 hours. Ethyl acetate was added thereto, and the organic layer was washed with 2N hydrochloric acid and brine, and dried over anhydrous magnesium sulfate. The solvent was distilled off in vacuo to obtain 13.24 g (yield: quantitative) of a crude target product. This was directly used in the next process without purification.

¹H-NMR data (400 MHz, CDCl₃/TMS δ (ppm)): 1.15 (3H, d), 1.45 (9H, s), 0.72 (1H, brs), 3.51 (1H, dd), 3.64 (1H, dd), 3.77 (1H, brs), 4.65 (1H, brs)

### (2) Production of (R)-(2-aminopropoxy)-2-chloro-4-fluoro-N-(2,2,2-trifluoroethyl) benzamide (compound of present invention No.: A-0368)

To a mixture of 3.00 g (11.0 mmol) of 2-chloro-4-fluoro-5-hydroxy-N-(2,2,2-trifluoroethyl) benzamide, 2.52 g (14.4 mmol) of (R)-2-(tert-butoxycarbonylamino)-1-propanol, 3.78 g (14.4 mmol) of triphenylphosphine, and 70 mL of tetrahydrofuran, 2.91 g (14.4 mmol) of diisopropyl azodicarboxylate was added under ice cooling, and the mixture was stirred at room temperature for 16 hours. Further, 1.89 g (7.21 mmol) of triphenylphosphine and 1.46 g (7.22 mmol) of diisopropyl azodicarboxylate were added thereto at room temperature, and the mixture was stirred at room temperature for 24 hours. The solvent was distilled off in vacuo, and the residue was purified by silica gel column chromatography (developing solvent, n-hexane:ethyl acetate=3:1) to obtain (R)-tert-butyl-(1-{4-chloro-2-fluoro-5-[(2,2,2-trifluoroethyl)carbamoyl]phenoxy}propan-2-yl)carbamate. To a mixture of the obtained (R)-tert-butyl-(1-{4-chloro-2-fluoro-5-[(2,2,2-trifluoroethyl)carbamoyl]phenoxy}propan-2-yl)carbamate and 30 mL of dichloromethane, 14 mL of trifluoroacetic acid was added at room temperature, and the mixture was stirred for 19 hours. Further, 2N hydrochloric acid was added thereto, and the aqueous layer was washed with isopropyl ether. Then, an aqueous sodium hydroxide solution was added to the obtained aqueous layer. The mixture was subjected to extraction with ethyl acetate, and the obtained organic layer was dried over anhydrous sodium sulfate. The solvent was distilled off in vacuo to obtain 3.50 g (yield: 96%) of a crude target product. This was directly used in the next process without purification.

¹H-NMR data (400 MHz, CDCl₃/TMS δ (ppm)): 1.16 (3H, d), 3.34-3.41 (1H, m), 3.73 (1H, dd), 3.94 (1H, dd), 4.08-4.17 (2H, m), 6.97 (1H, brs), 7.22 (1H, d), 7.40 (1H, d)

### (3) Production of (R)-2-chloro-4-fluoro-N-(2,2,2-trifluoroethyl)-5-{2-[(trifluoromethyl)sulfonamido]propoxy}benzamide (compound of present invention No.: C-0005)

To a mixture of 3.30 g (10.0 mmol) of crude (R)-(2-aminopropoxy)-2-chloro-4-fluoro-N-(2,2,2-trifluoroethyl)benzamide, 1.52 g (15.0 mmol) of triethylamine, 1.50 g of molecular sieve (4A), 50 mL of tetrahydrofuran, and 50 mL of dichloromethane, 2.54 g (15.1 mmol) of trifluoromethanesulfonyl chloride was added under ice cooling, and the mixture was stirred at room temperature for 17 hours. Chloroform was added to thereto, and the organic layer was washed with 2N hydrochloric acid and brine, and dried over anhydrous sodium sulfate. The solvent was distilled off in vacuo, and the residue was purified by silica gel column chromatography (developing solvent, n-hexane:ethyl acetate=3:1) to obtain 2.34 g (yield: 51%) of a target product.

¹H-NMR data (400 MHz, DMSO-d6/TMS δ (ppm)): 1.26 (3H, d), 3.88 (1H, m), 3.96-4.14 (4H, m), 7.28 (1H, d), 7.55 (1H, d), 9.12 (1H, t), 9.62 (1H, brs)

### (4) Production of (R)-2-chloro-4-fluoro-5-{2-[(1,1,1-trifluoro-N-methylmethyl)sulfonamido]propoxy}-N-(2,2,2-trifluoroethyl)benzamide (compound of present invention No.: C-0191)

To a mixture of 1.18 g (2.56 mmol) of (R)-2-chloro-4-fluoro-N-(2,2,2-trifluoroethyl)-5-{2-[(trifluoromethyl)sulfonamido]propoxy}benzamide and 0.46 g (3.3 mmol) of potassium carbonate in 30 mL of acetonitrile, 0.47 g (3.3 mmol) of methyl iodide was added at room temperature, and the mixture was stirred for 23 hours. The mixture was filtered, and the solvent was distilled off in vacuo. The residue was purified by silica gel column chromatography (developing solvent, n-hexane:ethyl acetate=3:1) to obtain 0.81 g (yield: 67%) of a target product.

¹H-NMR data (300 MHz, CDCl₃/TMS δ (ppm)): 1.40 (3H, d), 3.05 (3H, s), 4.05-4.21 (4H, m), 4.40-4.51 (1H, m), 6.81 (1H, brs), 7.20 (1H, d), 7.43 (1H, d)

### Example 2

### Production of (S)-2-chloro-4-fluoro-5-{2-[(1,1,1-trifluoro-N-methylmethyl)sulfonamido]propoxy}-N-(2,2,2-trifluoroethyl)benzamide (compound of present invention No.: C-0192)

### (1) Production of (S)-(2-aminopropoxy)-2-chloro-4-fluoro-N-(2,2,2-trifluoroethyl)benzamide (compound of present invention No.: A-0369)

To a mixture of 3.00 g (11.0 mmol) of 2-chloro-4-fluoro-5-hydroxy-N-(2,2,2-trifluoroethyl)benzamide, 2.13 g (12.2 mmol) of (S)-2-(tert-butoxycarbonylamino)-1-propanol, 3.19 g (12.5 mmol) of triphenylphosphine, and 60 mL of tetrahydrofuran, 2.46 g (12.17 mmol) of diisopropyl azodicarboxylate was added under ice cooling, and the mixture was stirred at room temperature for 13 hours. Further, 3.19 g (12.2 mmol) of triphenylphosphine and 2.46 g (12.2 mmol) of diisopropyl azodicarboxylate were added thereto at room temperature, and the mixture was stirred at room temperature for 8 hours. The solvent was distilled off in vacuo, and the residue was purified by silica gel column chromatography (developing solvent, n-hexane:ethyl acetate=3:1) to obtain (S)-tert-butyl-(1-{4-chloro-2-fluoro-5-[(2,2,2-trifluoroethyl)carbamoyl]phenoxy}propan-2-yl)carbamate. To a mixture of the obtained (S)-tert-butyl-(1-{4-chloro-2-fluoro-5-[(2,2,2-trifluoroethyl)carbamoyl]phenoxy}propan-2-yl)carbamate and 31 mL of dichloromethane, 14 mL of trifluoroacetic acid was added at room temperature, and the mixture was stirred for 1.5 hours. The solvent was distilled off in vacuo, 2N hydrochloric acid was added to the residue, and the aqueous layer was washed with isopropyl ether. Then, an aqueous sodium hydroxide solution was added to the obtained aqueous layer. The mixture was subjected to extraction with ethyl acetate, and the obtained organic layer was dried over anhydrous sodium sulfate. The solvent was distilled off in vacuo to obtain 3.90 g (yield: quantitative) of a crude target product. This was directly used in the next process without purification.

¹H-NMR data (400 MHz, CDCl₃/TMS δ (ppm)): 1.16 (3H, d), 3.34-3.39 (1H, m), 3.71 (1H, dd), 3.93 (1H, dd), 4.12 (2H, m), 7.14 (1H, brs), 7.15 (1H, d), 7.36 (1H, d)

### (2) Production of (S)-2-chloro-4-fluoro-N-(2,2,2-trifluoroethyl)-5-{2-[(trifluoromethyl)sulfonamido]propoxy}benzamide (compound of present invention No.: C-0006)

To a mixture of 1.50 g (4.56 mmol) of crude (S)-(2-aminopropoxy)-2-chloro-4-fluoro-N-(2,2,2-trifluoroethyl)benzamide, 0.69 g (6.8 mmol) of triethylamine, 0.75 g of molecular sieve (4A), 45 mL of tetrahydrofuran, and 45 mL of dichloromethane, 1.15 g (6.82 mmol) of trifluoromethanesulfonyl chloride was added under ice cooling, and the mixture was stirred at room temperature for 17 hours. The mixture was filtered, ethyl acetate was added thereto, and the organic layer was washed with a saturated aqueous ammonium chloride solution and dried over anhydrous sodium sulfate. The solvent was distilled off in vacuo, and the residue was purified by silica gel column chromatography (developing solvent, n-hexane:ethyl acetate=3:1) to obtain 1.07 g (yield: 51%) of a target product.

¹H-NMR data (400 MHz, DMSO-d6/TMS δ (ppm)): 1.26 (3H, d), 3.88 (1H, brs), 3.96-4.15 (4H, m), 7.29 (1H, d), 7.55 (1H, d), 9.12 (1H, t), 9.62 (1H, brs)

### (3) Production of (S)-2-chloro-4-fluoro-5-{2-[(1,1,1-trifluoro-N-methylmethyl)sulfonamido]propoxy}-N-(2,2,2-trifluoroethyl)benzamide (compound of present invention No.: C-0192)

To a mixture of 0.86 g (1.9 mmol) of (S)-2-chloro-4-fluoro-N-(2,2,2-trifluoroethyl)-5-{2-[(trifluoromethyl)sulfonamido]propoxy}benzamide and 0.34 g (2.5 mmol) of potassium carbonate in 20 mL of acetonitrile, 0.34 g (2.4 mmol) of methyl iodide was added at room temperature, and the mixture was stirred for 23 hours. The mixture was filtered, and the solvent was distilled off in vacuo. The residue was purified by silica gel column chromatography (developing solvent, n-hexane:ethyl acetate=3:1) to obtain 0.86 g (yield: 97%) of a target product.

¹H-NMR data (300 MHz, CDCl₃/TMS δ (ppm)): 1.41 (3H, d), 3.05 (3H, s), 4.03-4.22 (4H, m), 4.40-4.51 (1H, m), 6.80 (1H, brs), 7.20 (1H, d), 7.44 (1H, d)

### Example 3

### Production of 2-chloro-4-fluoro-5-{2-methyl-3-[(trifluoromethyl)thio]propoxy}-N-(2,2,2-trifluoroethyl)benzamide (compound of present invention No.: B-0009)

### (1) Production of 2-chloro-5-(3-chloro-2-methylpropoxy)-4-fluoro-N-(2,2,2-trifluoroethyl)benzamide (compound of present invention No.: A-0037)

To a mixture of 3.00 g (11.0 mmol) of 2-chloro-4-fluoro-5-hydroxy-N-(2,2,2-trifluoroethyl)benzamide and 100 mL of N,N-dimethylformamide, 1.99 g (14.4 mmol) of potassium carbonate, 0.36 g (1.1 mmol) of tetrabutylammonium bromide, and 3.79 g (22.1 mmol) of 1-bromo-3-chloro-2-methylpropane were added at room temperature, and the mixture was stirred at 70 °C for 7 hours. The mixture was allowed to cool at room temperature and then filtered, and ethyl acetate and hexane were added thereto. The mixture was washed with water, and dried over anhydrous sodium sulfate. The solvent was distilled off in vacuo, and the residue was purified by silica gel column chromatography (developing solvent, n-hexane:ethyl acetate=5:1) to obtain 3.71 g (purity: about 50%, yield: 46%) of a target product.

¹H-NMR data (400 MHz, CDCl₃/TMS δ (ppm)): 1.15 (3H, d), 2.32-2.48 (1H, m), 3.68 (2H, ddd), 3.98-4.19 (4H, m), 6.79 (1H, brs), 7.16 (1H, d), 7.44 (1H, d)

### (2) Production of 2-chloro-4-fluoro-5-(2-methyl-3-thiocyanatopropoxy)-N-(2,2,2-trifluoroethyl)benzamide (compound of present invention No.: A-0758)

To a mixture of 3.71 g (purity: about 50%, about 5.12 mmol) of 2-chloro-5-(3-chloro-2-methylpropoxy)-4-fluoro-N-(2,2,2-trifluoroethyl)benzamide and 60 mL of ethanol, 1.99 g (20.5 mmol) of potassium thiocyanate and 0.17 g (1.0 mmol) of potassium iodide were added at room temperature, and the mixture was stirred under reflux for 3 days. The mixture was allowed to cool at room temperature and then filtered, and the solvent was distilled off in vacuo. The residue was purified by silica gel column chromatography (developing solvent, n-hexane:ethyl acetate=3:1) to obtain 0.84 g (yield: 43%) of a target product.

¹H-NMR data (300 MHz, CDCl₃/TMS δ (ppm)): 1.24 (3H, d), 2.40-2.55 (1H, m), 3.06 (1H, dd), 3.23 (1H, dd), 3.99 (1H, dd), 4.07-4.18 (3H, m), 6.83 (1H, brs), 7.17 (1H, d), 7.42 (1H, d)

### (3) Production of 2-chloro-4-fluoro-5-{2-methyl-3-[(trifluoromethyl)thio]propoxy}-N-(2,2,2-trifluoroethyl)benzamide (compound of present invention No.: B-0009)

To a mixture of 0.76 g (2.0 mmol) of 2-chloro-4-fluoro-5-(2-methyl-3-thiocyanatopropoxy)-N-(2,2,2-trifluoroethyl)benzamide, 0.70 g (4.9 mmol) of (trifluoromethyl)trimethylsilane, and 20 mL of tetrahydrofuran, 0.2 mL (0.2 mmol) of tetrabutylammonium fluoride (about 1 mol/L tetrahydrofuran solution) was added under ice cooling, and the mixture was stirred at 0 °C for 4 hours. The solvent was distilled off in vacuo, ethyl acetate was added to the residue. The mixture was washed with a saturated aqueous sodium bicarbonate solution, and dried over anhydrous sodium sulfate. The solvent was distilled off in vacuo, and the residue was purified by silica gel column chromatography (developing solvent, n-hexane:ethyl acetate=5:1) to obtain 0.47 g (yield: 56%) of a target product.

¹H-NMR data (300 MHz, CDCl₃/TMS δ (ppm)): 1.18 (3H, d), 2.27-2.40 (1H, m), 2.94 (1H, dd), 3.13 (1H, dd), 3.95 (1H, dd), 4.06 (1H, dd), 4.14 (2H, m), 6.81 (1H, brs), 7.17 (1H, d), 7.42 (1H, d)

### Example 4

### Production of N-acetyl-2-chloro-4-fluoro-N-(2,2,2-trifluoroethyl)-5-({5-[(trifluoromethyl)thio]pentyl}oxy)benzamide (compound of present invention No.: A-6814)

To a mixture of 0.30 g (0.68 mmol) of 2-chloro-4-fluoro-N-(2,2,2-trifluoroethyl)-5-({5-[(trifluoromethyl)thio]pentyl}oxy)benzamide in 12 mL of N,N-dimethylformamide, 0.038 g (purity: 55%, 0.87 mmol) of sodium hydride was added at room temperature, and the mixture was stirred at room temperature for 30 minutes. Thereafter, 0.069 g (0.88 mmol) of acetyl chloride was added under ice cooling, and the mixture was further stirred at room temperature for 1.5 hours. Ethyl acetate and hexane were added thereto, and the mixture was washed with a saturated aqueous sodium bicarbonate solution and brine, and dried over anhydrous magnesium sulfate. The solvent was distilled off in vacuo, and the residue was purified by silica gel column chromatography (developing solvent, n-hexane:ethyl acetate=5:1) to obtain 0.22 g (yield: 67%) of a target product.

¹H-NMR data (300 MHz, CDCl₃/TMS δ (ppm)): 1.56-1.66 (2H, m), 1.74-1.91 (4H, m), 2.37 (3H, s), 2.91 (2H, t), 4.04 (2H, t), 4.40 (2H, brd), 7.02 (1H, d), 7.18 (1H, d)

### Example 5

### Production of 2-chloro-N-(cyanomethyl)-4-fluoro-N-(2,2,2-trifluoroethyl)-5-({5-[(trifluoromethyl)thio]pentyl}oxy)benzamide (compound of present invention No.: A-6692)

To a mixture of 0.40 g (0.91 mmol) of 2-chloro-4-fluoro-N-(2,2,2-trifluoroethyl)-5-((5-((trifluoromethyl)thio)pentyl)oxy)benzamide in 20 mL of N,N-dimethylformamide, 0.124 g (purity: 55%, 2.84 mmol) of sodium hydride was added at room temperature, and the mixture was stirred at room temperature for 30 minutes.

Thereafter, 0.55 g (4.6 mmol) of bromoacetonitrile was added thereto at room temperature, and the mixture was further stirred at room temperature for 3 hours. Ethyl acetate and hexane were added thereto, and the mixture was washed with a saturated aqueous sodium bicarbonate solution and brine, and dried over anhydrous magnesium sulfate. The solvent was distilled off in vacuo, and the residue was purified by silica gel column chromatography (developing solvent, n-hexane:ethyl acetate=5:1) to obtain 0.18 g (yield: 41%) of a target product.

¹H-NMR data (300 MHz, CDCl₃/TMS δ (ppm)): 1.60-1.66 (2H, m), 1.72-1.88 (4H, m), 2.91 (2H, t), 3.86-4.91 (6H, m), 6.89-6.97 (1H, m), 7.15-7.26 (1H, m)

### Example 6

### Production of 4-fluoro-2-methyl-N-(2,2,2-trifluoroethyl)-5-({5-[(trifluoromethyl)thio]pentyl}oxy)benzamide (compound of present invention No.: A-4712)

### (1) Production of 2-fluoro-4-methylphenyl methanesulfonate

To a mixture of 6.03 g (47.8 mmol) of 2-fluoro-4-methylphenol, 5.31 g (52.5 mmol) of triethylamine, and 70 mL of dichloromethane, 6.02 g (52.6 mmol) of methanesulfonyl chloride was added under ice cooling, and the mixture was stirred at room temperature for 5 hours. Then, 2N hydrochloric acid was added thereto, and the mixture was subjected to extraction with chloroform. The obtained organic layer was washed with brine, and dried over anhydrous magnesium sulfate. The solvent was distilled off in vacuo, and the residue was purified by silica gel column chromatography (developing solvent, n-hexane:ethyl acetate=5:1) to obtain 9.76 g (yield: quantitative) of a target product.

¹H-NMR data (400 MHz, CDCl₃/TMS δ (ppm)) 2.36 (3H, s), 3.19 (3H, s), 6.97 (1H, d), 7.02 (1H, d), 7.25 (1H, t)

### (2) Production of 2-fluoro-5-iodo-4-methylphenyl methanesulfonate

A mixture of 2.18 g (8.59 mmol) of powdery iodine, 1.00 g (4.67 mmol) of potassium iodate, and 90 mL of a 90% aqueous sulfuric acid solution was stirred at 50 °C for 1 hour. Further, a mixture of 4.00 g (19.6 mmol) of 2-fluoro-4-methylphenyl methanesulfonate and 10 mL of 90% sulfuric acid was added dropwise at 0 °C. The mixture was stirred for 1.5 hours at 0 °C. The mixture was added dropwise to ice water, and this was subjected to extraction with toluene. The obtained organic layer was washed with an aqueous sodium thiosulfate solution, a saturated aqueous sodium bicarbonate solution, and brine, and dried over anhydrous magnesium sulfate. The solid obtained by distilling off the solvent in vacuo was washed with a mixed solution of hexane and isopropyl ether, and then filtered, followed by drying to obtain 6.14 g (yield: 95%) of a target product.

¹H-NMR data (400 MHz, CDCl₃/TMS δ (ppm)): 2.43 (3H, s), 3.22 (3H, s), 7.12 (1H, d), 7.78 (1H, d)

### (3) Production of 2-fluoro-5-iodo-4-methylphenol

To a mixture of 8.05 g (24.4 mmol) of 2-fluoro-5-iodo-4-methylphenyl methanesulfonate and 30 mL of water, 2.93 g (73.3 mmol) of sodium hydroxide was added at room temperature, and the mixture was stirred under reflux for 3 hours. Then, 2N hydrochloric acid was added thereto, and the mixture was subjected to extraction with ethyl acetate. The obtained organic layer was washed with brine, and dried over anhydrous magnesium sulfate. The solvent was distilled off in vacuo, and the residue was purified by silica gel column chromatography (developing solvent, n-hexane:ethyl acetate=7:1) to obtain 6.18 g (yield: quantitative) of a target product.

¹H-NMR data (400 MHz, CDCl₃/TMS δ (ppm)): 2.33 (3H, s), 5.47 (1H, brs), 6.96 (1H, d), 7.44 (1H, d)

### (4) Production of 1-[(5-bromopentyl)oxy]-2-fluoro-5-iodo-4-methylbenzene

To a solution of 5.00 g (19.8 mmol) of 2-fluoro-5-iodo-4-methylphenol, 22.8 g (99.2 mmol) of 1,5-dibromopentane, and 250 mL of acetonitrile, 3.56 g (25.8 mmol) of potassium carbonate and 0.64 g (1.99 mmol) of tetrabutylammonium bromide were added at room temperature, and the mixture was stirred under reflux for 2 hours. The mixture was allowed to cool at room temperature and then filtered, and the solvent was distilled off in vacuo (0.1 mmHg, 65 °C). The residue was purified by silica gel column chromatography (developing solvent, n-hexane:ethyl acetate=20:1) to obtain 7.17 g (yield: 90%) of a target product.

¹H-NMR data (300 MHz, CDCl₃/TMS δ (ppm)): 1.60-1.68 (2H, m), 1.78-1.88 (2H, m), 1.89-1.99 (2H, m), 2.34 (3H, s), 3.44 (2H, t), 3.99 (2H, t), 6.96 (1H, d), 7.34 (1H, d)

### (5) Production of 1-fluoro-4-iodo-5-methyl-2-((5-thiocyanatopentyl)oxy)benzene

To a mixed solution of 7.07 g (17.6 mmol) of 1-((5-bromopentyl)oxy)-2-fluoro-5-iodo-4-methylbenzene and 80 mL of ethanol, 3.47 g (35.7 mmol) of potassium thiocyanate was added at room temperature, and the mixture was stirred under reflux for 4 hours. The mixture was allowed to cool at room temperature and then filtered, and the solvent was distilled off in vacuo. Ethyl acetate was added to the residue, and this was washed with brine and dried over anhydrous magnesium sulfate. The solvent was distilled off in vacuo, and the residue was purified by silica gel column chromatography (developing solvent, n-hexane:ethyl acetate=9:1) to obtain 6.10 g (yield: 91%) of a target product.

¹H-NMR data (300 MHz, CDCl₃/TMS δ (ppm)): 1.60-1.70 (2H, m), 1.80-1.97 (4H, m), 2.34 (3H, s), 2.98 (2H, t), 4.00 (2H, t), 6.96 (1H, d), 7.34 (1H, d)

### (6) Production of [5-(2-fluoro-5-iodo-4-methylphenoxy)pentyl](trifluoromethyl)sulfane

To a mixture of 5.99 g (15.8 mmol) of 1-fluoro-4-iodo-5-methyl-2-[(5-thiocyanatopentyl)oxy]benzene, 8.98 g (63.2 mmol) of (trifluoromethyl)trimethylsilane, and 80 mL of tetrahydrofuran, 1.6 mL (1.6 mmol) of tetrabutylammonium fluoride (about 1 mol/L tetrahydrofuran solution) was added under ice cooling, and the mixture was stirred for 2.5 hours while the same temperature was maintained. The solvent was distilled off in vacuo, ethyl acetate was added to the residue, and this was washed with brine and dried over anhydrous magnesium sulfate. The solvent was distilled off in vacuo, and the residue was purified by silica gel column chromatography (developing solvent, n-hexane:ethyl acetate=15:1) to obtain 6.44 g (yield: 97%) of a target product.

¹H-NMR data (300 MHz, CDCl₃/TMS δ (ppm)): 1.54-1.65 (2H, m), 1.73-1.87 (4H, m), 2.34 (3H, s), 2.91 (2H, t), 3.98 (2H, t), 6.96 (1H, d), 7.33 (1H, d)

### (7) Production of 4-fluoro-2-methyl-5-({5-[(trifluoromethyl)thio]pentyl}oxy)benzoic acid (compound of present invention No.: F-0965)

A mixed solution of 1.93 g (28.4 mmol) of sodium formate, 1.93 g (18.9 mmol) of acetic anhydride, 2.45 g (19.0 mmol) of N,N-diisopropylethylamine, and 17 mL of N,N-dimethylformamide was stirred at room temperature for 1 hour under a nitrogen stream.

The resulting mixed solution was added to a mixed solution of 2.00 g (4.74 mmol) of (5-(2-fluoro-5-iodo-4-methylphenoxy)pentyl) (trifluoromethyl)sulfane, 0.22 g (0.24 mmol) of tris(dibenzylideneacetone)dipalladium, 0.60 g (14 mmol) of lithium chloride, and 20 mL of N,N-dimethylformamide at room temperature, and the mixture was stirred at 80 °C for 3 hours. After the mixture was allowed to cool to room temperature, a mixed solution obtained by stirring 1.93 g (28.4 mmol) of sodium formate, 1.93 g (18.9 mmol) of acetic anhydride, 2.45 g (19.0 mmol) of N,N-diisopropylethylamine, and 17 mL of N,N-dimethylformamide at room temperature for 0.5 hours under a nitrogen stream was added thereto. The mixture was stirred at 80 °C for 1 hour. After the mixture was allowed to cool to room temperature, ethyl acetate was added thereto. The mixture was washed with 2N hydrochloric acid and brine, and dried over anhydrous magnesium sulfate. The solvent was distilled off in vacuo to obtain 2.12 g of a crude target product.

¹H-NMR data (300 MHz, CDCl₃/TMS δ (ppm)) 1.57-1.66 (2H, m), 1.74-1.91 (4H, m), 2.57 (3H, s), 2.92 (2H, t), 4.06 (2H, t), 7.10 (1H, d), 7.67 (1H, d)

### (8) Production of 4-fluoro-2-methyl-N-(2,2,2-trifluoroethyl)-5-({5-[(trifluoromethyl)thio]pentyl}oxy)benzamide (compound of present invention No.: A-4712)

To a mixture of 2.12 g of crude 4-fluoro-2-methyl-5-({5-[(trifluoromethyl)thio]pentyl}oxy)benzoic acid, 1.09 g (5.69 mmol) of 1-ethyl-3-(3-dimethylaminopropyl)carbodiimide hydrochloride, 0.87 g (5.7 mmol) of 1-hydroxybenzotriazole monohydrate, 0.94 g (9.5 mmol) of 2,2,2-trifluoroethylamine, 15 mL of tetrahydrofuran, and 15 mL of dichloromethane, 0.96 g (9.5 mmol) of triethylamine was added under ice cooling, and the mixture was stirred at room temperature for 20 hours. The solvent was distilled off from the reaction mixture in vacuo, and ethyl acetate was added to the residue. The organic layer was washed with a saturated aqueous sodium bicarbonate solution and brine, and dried over anhydrous magnesium sulfate. The solvent was distilled off in vacuo, and the residue was purified by silica gel column chromatography (developing solvent, n-hexane:ethyl acetate=5:1) to obtain 0.97 g (yield: 49%, 2 processes) of a target product.

¹H-NMR data (300 MHz, CDCl₃/TMS δ (ppm)): 1.56-1.66 (2H, m), 1.73-1.89 (4H, m), 2.36 (3H, s), 2.92 (2H, t), 4.02 (2H, t), 4.04-4.15 (2H, m), 5.95 (1H, brs), 6.95 (1H, d), 7.00 (1H, d)

### Example 7

### Production of 2-chloro-4-fluoro-N-(2,2,2-trifluoroethyl)-5-({5-[(trifluoromethyl)thio]pentyl}oxy)benzamide (compound of present invention No.: A-4689)

### (1) Production of 4-chloro-2-fluorophenyl methanesulfonate

To a mixture of 40.0 g (273 mmol) of 4-chloro-2-fluorophenol, 30.4 g (300 mmol) of triethylamine, and 400 mL of dichloromethane, 34.4 g (300 mmol) of methanesulfonyl chloride was added under ice cooling, and the mixture was stirred at room temperature for 19 hours. Then, 2N hydrochloric acid was added thereto, and the mixture was subjected to extraction with chloroform. The obtained organic layer was washed with brine, and dried over anhydrous magnesium sulfate. The solvent was distilled off in vacuo to obtain 64.47 g of a crude target product.

¹H-NMR data (300 MHz, CDCl₃/TMS δ (ppm)): 7.16-7.21 (1H, m), 7.26 (1H, dd), 7.33 (1H, t)

### (2) Production of 4-chloro-2-fluoro-5-iodophenyl methanesulfonate

A mixture of 30.48 g (120.1 mmol) of powdery iodine, 14.02 g (65.5 mmol) of potassium iodate, and 1,200 mL of a 90% aqueous sulfuric acid solution was stirred at 50 °C for 1 hour. Further, a mixture of 64.47 g of crude 4-chloro-2-fluorophenyl methanesulfonate and 50 mL of 90% sulfuric acid was added dropwise at 0 °C. After the dropwise addition, the mixture was stirred for 3 hours at 0 °C. The mixture was added dropwise to ice water, and this was subjected to extraction with toluene. The obtained organic layer was washed with a saturated aqueous sodium bicarbonate solution and brine, and dried over anhydrous magnesium sulfate. The solid obtained by distilling off the solvent in vacuo was washed with a mixed solution of hexane and isopropyl ether, and then filtered, followed by drying to obtain 67.05 g (yield: 70%, 2 processes) of a target product.

¹H-NMR data (400 MHz, CDCl₃/TMS δ (ppm)) 3.25 (3H, s), 7.37 (1H, d), 7.84 (1H, d)

### (3) Production of 4-chloro-5-cyano-2-fluorophenyl methanesulfonate

To 15.0 g (42.8 mmol) of 4-chloro-2-fluoro-5 iodophenyl methanesulfonate, 5.02 g (42.8 mmol) of zinc cyanide, and 250 mL of N,N-dimethylformamide, 4.50 g (3.89 mmol) of tetrakis(triphenylphosphine)palladium was added at room temperature, and the mixture was stirred at 80 °C for 4.5 hours. After the mixture was allowed to cool at room temperature, ethyl acetate and hexane were added. The mixture was washed with a saturated aqueous sodium bicarbonate solution and brine, and then dried over anhydrous magnesium sulfate. The solvent was distilled off in vacuo, and the residue was purified by silica gel column chromatography (developing solvent, n-hexane:ethyl acetate=8:1) to obtain 9.02 g (yield: 84%) of a target product.

¹H-NMR data (400 MHz, CDCl₃/TMS δ (ppm)): 3.31 (3H, s), 7.44 (1H, d), 7.73 (1H, d)

### (4) Production of 2-chloro-4-fluoro-5-hydroxybenzoic acid

A mixed solution of 21.80 g (87.33 mmol) of 4-chloro-5-cyano-2-fluorophenyl methanesulfonate and 320 mL of a 60% aqueous sulfuric acid solution was stirred at 120 °C for 27 hours. The mixed solution was allowed to cool at room temperature, and then added to ice water. This was subjected to extraction with toluene, and the obtained organic layer was dried over anhydrous magnesium sulfate. The solid obtained by distilling off the solvent in vacuo was washed with a chloroform solution, and then filtered, followed by drying to obtain 10.39 g (yield: 62%) of a target product.

¹H-NMR data (400 MHz, DMSO-d6/TMS δ (ppm)): 7.42 (1H, d), 7.44 (1H, d)

### (5) Production of 2-chloro-4-fluoro-5-hydroxy-N-(2,2,2-trifluoroethyl)benzamide (compound of present invention No.: E-0006)

To a mixture of 9.46 g (49.6 mmol) of 2-chloro-4-fluoro-5-hydroxybenzoic acid, 11.42 g (59.57 mmol) of 1-ethyl-3-(3-dimethylaminopropyl)carbodiimide hydrochloride, 9.11 g (59.5 mmol) of 1-hydroxybenzotriazole monohydrate, 9.84 g (99.3 mmol) of 2,2,2-trifluoroethylamine, and 500 mL of N,N-dimethylformamide, 10.05 g (99.32 mmol) of triethylamine was added under ice cooling, and the mixture was stirred at room temperature for 14 hours. Ethyl acetate was added thereto, and the mixture was washed with 2N hydrochloric acid, and dried over anhydrous magnesium sulfate. The solvent was distilled off in vacuo, and the residue was purified by silica gel column chromatography (developing solvent, n-hexane:ethyl acetate=1:3) to obtain 12.55 g (yield: 93%) of a target product.

¹H-NMR data (400 MHz, DMSO/TMS δ (ppm)): 4.00-4.09 (2H, m), 6.99 (1H, d), 7.44 (1H, d), 9.09 (1H, t), 10.48 (1H, s)

### (6) Production of 5-[(5-bromopentyl)oxy]-2-chloro-4-fluoro-N-(2,2,2-trifluoroethyl)benzamide (compound of present invention No.: A-0030)

To a solution of 1.09 g (4.01 mmol) of 2-chloro-4-fluoro-5-hydroxy-N-(2,2,2-trifluoroethyl)benzamide, 4.99 g (21.7 mmol) of 1,5-dibromopentane, and 50 mL of N,N-dimethylformamide, 0.78 g (5.6 mmol) of potassium carbonate and 0.14 g (0.43 mmol) of tetrabutylammonium bromide were added at room temperature, and the mixture was stirred at 100 °C for 2.5 hours. The mixture was allowed to cool to room temperature and then filtered, and ethyl acetate was added thereto. The mixture was washed with water, and dried over anhydrous magnesium sulfate. The solvent was distilled off in vacuo, and the residue was purified by silica gel column chromatography (developing solvent, n-hexane:ethyl acetate=5:1) to obtain 0.87 g (yield: 52%) of a target product.

¹H-NMR data (400 MHz, CDCl₃/TMS δ (ppm)): 1.60-1.67 (2H, m), 1.83-1.98 (4H, m), 3.44 (2H, t), 4.06 (2H, t), 4.09-4.17 (2H, m), 6.79 (1H, brs), 7.15 (1H, d), 7.41 (1H, d)

### (7) Production of 2-chloro-4-fluoro-5-[(5-thiocyanatopentyl)oxy]-N-(2,2,2-trifluoroethyl)benzamide (compound of present invention No.: A-0752)

To a mixed solution of 0.79 g (1.9 mmol) of 5-[(5-bromopentyl)oxy]-2-chloro-4-fluoro-N-(2,2,2-trifluoroethyl)benzamide and 20 mL of ethanol, 0.37 g (3.8 mmol) of potassium thiocyanate was added at room temperature, and the mixture was stirred under reflux for 4 hours. The mixture was allowed to cool at room temperature and then filtered, and the solvent was distilled off in vacuo. Ethyl acetate was added to the residue, and this was washed with brine and dried over anhydrous magnesium sulfate. The solvent was distilled off in vacuo, and the residue was purified by silica gel column chromatography (developing solvent, n-hexane:ethyl acetate=5:1) to obtain 0.66 g (yield: 88%) of a target product.

¹H-NMR data (300 MHz, CDCl₃/TMS δ (ppm)): 1.61-1.71 (2H, m), 1.84-1.97 (4H, m), 2.99 (2H, t), 4.06-4.19 (4H, m), 6.79 (1H, brs), 7.17 (1H, d), 7.42 (1H, d)

### (8) Production of 2-chloro-4-fluoro-N-(2,2,2-trifluoroethyl)-5-({5-[(trifluoromethyl)thio]pentyl}oxy)benzamide (compound of present invention No.: A-4689)

To a mixture of 0.58 g (1.5 mmol) of 2-chloro-4-fluoro-5-[(5-thiocyanatopentyl)oxy]-N-(2,2,2-trifluoroethyl)benzamide, 0.88 g (6.2 mmol) of (trifluoromethyl)trimethylsilane, and 10 mL of tetrahydrofuran, 0.16 mL (0.16 mmol) of tetrabutylammonium fluoride (about 1 mol/L tetrahydrofuran solution) was added under ice cooling, and the mixture was stirred for 3 hours. The solvent was distilled off in vacuo, and the residue was purified by silica gel column chromatography (developing solvent, n-hexane:ethyl acetate=7:1) to obtain 0.60 g (yield: 93%) of a target product.

¹H-NMR data (400 MHz, CDCl₃/TMS δ (ppm)) 1.57-1.64 (2H, m), 1.75-1.89 (4H, m), 2.91 (2H, t), 4.06 (2H, t), 4.09-4.18 (2H, m), 6.77 (1H, brs), 7.16 (1H, d), 7.43 (1H, d)

### Example 8

### Production of 2-chloro-4-fluoro-N-(2,2,2,-trifluoroethyl)-5-({5-[(trifluoromethyl)thio]pentyl}oxy)thiobenzamide (compound of present invention No.: A-5172)

A mixture of 0.16 g (0.36 mmol) of 2-chloro-4-fluoro-N-(2,2,2-trifluoroethyl)-5-({5-[(trifluoromethyl)thio]pentyl}oxy)benzamide, 0.15 g (0.37 mmol) of a Lawesson's reagent, and 15 mL of toluene was heated reflux for 15 hours. After the mixture was cooled at room temperature, the precipitated solid was removed by filtration, and the filtrate was concentrated in vacuo. The residue was purified by silica gel column chromatography (developing solvent, n-hexane:ethyl acetate=4:1) to obtain 0.15 g (yield: 90%) of a target product.

¹H-NMR data (400 MHz, CDCl₃/TMS δ (ppm)): 1.54-1.64 (2H, m), 1.74-1.89 (4H, m), 2.91 (2H, t), 4.05 (2H, t), 4.55-4.63 (2H, m), 7.12 (1H, d), 7.25 (1H, d), 7.52 (1H, brs)

### Example 9

### Production of ethyl 4-fluoro-5-hydroxy-2-methylbenzoate

### (1) Production of 5-bromo-4-fluoro-2-methylbenzoic acid

To a mixture of 20.00 g (129.8 mmol) of 4-fluoro-2-methylbenzoic acid and 100 mL of concentrated sulfuric acid, 24.24 g (136.2 mmol) of N-bromosuccinimide was added at 0 °C or lower, and the mixture was stirred at room temperature overnight. The mixture was poured into ice water, and the precipitated solid was filtered and washed with hexane and water. The obtained solid was dissolved in ethyl acetate, washed with water and brine, and dried over anhydrous magnesium sulfate. The solvent was distilled off in vacuo to obtain 30.30 g of a crude target product (including regioisomers). This was directly used in the next process without purification.

¹H-NMR data (400 MHz, CDCl₃/TMS δ (ppm)) 2.51 (3H, t), 7.37 (1H, d), 8.07 (1H, d), 13.23 (1H, brs)

### (2) Production of ethyl 5-bromo-4-fluoro-2-methylbenzoate

To a mixture of 30.30 g of crude 5-bromo-4-fluoro-2-methylbenzoic acid, 19.76 g (0.143 mol) of potassium carbonate, and 200 mL of N,N-dimethylformamide, 22.25 g (0.143 mol) of ethyl iodide was added under ice cooling. The mixture was stirred at room temperature overnight, and ethyl acetate and hexane were added thereto. The mixture was washed with water and brine, and dried over anhydrous magnesium sulfate. The solvent was distilled off in vacuo and purified by recrystallization with hexane to obtain 16.97 g (yield: 49%, 2 processes) of a target product.

¹H-NMR data (400 MHz, CDCl₃/TMS δ (ppm)): 1.40 (3H, t), 2.57 (3H, s), 4.35 (2H, q), 7.00 (1H, d), 8.14 (1H, d)

### (3) Production of [5-(ethoxycarbonyl)-2-fluoro-4-methylphenyl]boronic acid

To a mixture of 2.0 g (7.7 mmol) of ethyl 5-bromo-4-fluoro-2-methylbenzoate and 20 mL of tert-butyl methyl ether, 17.01 mL (1.0 M THF solution, 17.01 mmol) of isopropylmagnesium chloride was added at −60 °C. After the mixture was stirred at room temperature for 1 hour, 2.16 g (11.5 mmol) of triisopropyl borate was added thereto at −60 °C, and the mixture was stirred at room temperature overnight. A 15% aqueous sulfuric acid solution was added under ice cooling, and this was further stirred at room temperature for 4 hours. Ethyl acetate was added thereto, and the mixture was washed with water and brine, and dried over anhydrous magnesium sulfate. The solvent was distilled off in vacuo, and the obtained solid was washed with hexane to obtain 1.23 g (yield: 71%) of a target product.

¹H-NMR data (400 MHz, CDCl₃/TMS δ (ppm)) 1.40 (3H, t), 2.63 (3H, s), 4.36 (2H, q), 5.29 (2H, d), 6.93 (1H, d), 8.40 (1H, d)

### (4) Production of ethyl 4-fluoro-5-hydroxy-2-methylbenzoate

To a mixture of 4.03 g (17.8 mmol) of [5-(ethoxycarbonyl)-2-fluoro-4-methylphenyl]boronic acid and 70 mL of toluene, 19.61 mL (50% aqueous solution, about 4.8 mol/L, 94 mmol) of 4-methylmorpholine-N-oxide was added at room temperature, and the mixture was stirred under reflux for 8 hours. After the mixture was allowed to cool at room temperature, 2N hydrochloric acid and brine were added thereto. The obtained organic layer was dried over anhydrous magnesium sulfate. The solvent was distilled off in vacuo, and the residue was purified by silica gel column chromatography (developing solvent, n-hexane:ethyl acetate=5:1) to obtain 2.89 g (yield: 82%) of a target product.

¹H-NMR data (400 MHz, CDCl₃/TMS δ (ppm)): 1.38 (3H, t), 2.52 (3H, s), 4.33 (2H, q), 5.13 (1H, brs), 6.94 (1H, d), 7.62 (1H, d)

### Example 10

### Production of N-acetyl-2-chloro-4-fluoro-N-(2,2,2-trifluoroethyl)-5-{2-[(trifluoromethyl)sulfonamido]propoxy}benzamide (compound of present invention No.: C-4214)

### (1) Production of tert-butyl(1-{4-chloro-2-fluoro-5-[(2,2,2-trifluoroethyl)carbamoyl]phenoxy}propan-2-yl) [(trifluoromethyl)sulfonyl]carbamate (compound of present invention No.: C-9422)

To a mixture of 0.60 g (1.3 mmol) of 2-chloro-4-fluoro-N-(2,2,2-trifluoroethyl)-5-{2-[(trifluoromethyl)sulfonamido]propoxy}benzamide (compound of present invention No.: C-0004) produced by a similar method to that of Example 2 (1) and (2), 0.16 g (1.6 mmol) of triethylamine, 15.9 mg (0.130 mmol) of 4-dimethylaminopyridine, and 4 mL of tetrahydrofuran, 0.28 g (1.3 mmol) of di-tert-butyl dicarbonate was added at room temperature, and the mixture was stirred at room temperature for 24 hours. An aqueous citric acid solution was added to the mixture, and the mixture was subjected extraction with ethyl acetate. The obtained organic layer was washed with water and brine, and dried over anhydrous magnesium sulfate. The solvent was distilled off in vacuo, and the residue was purified by silica gel column chromatography (developing solvent, n-hexane:ethyl acetate=2:1) to obtain 0.45 g (yield: 62%) of a target product.

¹H-NMR data (400 MHz, CDCl₃/TMS δ (ppm)): 1.50 (9H, s), 1.53 (3H, d), 4.08-4.22 (3H, m), 4.48 (1H, t), 4.71 (1H, m), 6.74 (1H, brs), 7.18 (1H, d), 7.43 (1H, d)

### (2) Production of tert-butyl(1-{5-[acetyl(2,2,2-trifluoroethyl)carbamoyl]-4-chloro-2-fluorophenoxy}propan-2-yl) [(trifluoromethyl)sulfonyl]carbamate (compound of present invention No.: C-10702)

To a mixture of 0.44 g (0.78 mmol) of tert-butyl(1-{4-chloro-2-fluoro-5-[(2,2,2-trifluoroethyl)carbamoyl]phenoxy}propan-2-yl) [(trifluoromethyl)sulfonyl]carbamate and 10 mL of tetrahydrofuran, 44.5 mg (purity: 55%, 1.02 mmol) of sodium hydride was added at room temperature, and the mixture was stirred at room temperature for 10 minutes. Then, 0.08 g (1 mmol) of acetyl chloride was added thereto at room temperature, and the mixture was stirred at room temperature for 2 hours. An aqueous citric acid solution was added to the mixture, and the mixture was subjected extraction with ethyl acetate. The obtained organic layer was washed with water and brine, and dried over anhydrous magnesium sulfate. The solvent was distilled off in vacuo, and the residue was purified by silica gel column chromatography (developing solvent, n-hexane:ethyl acetate=3:1) to obtain 0.36 g (yield: 76%) of a target product.

¹H-NMR data (400 MHz, CDCl₃/TMS δ (ppm)): 1.50 (9H, s), 1.53 (3H, d), 2.37 (3H, s), 4.17 (1H, m), 4.31-4.49 (3H, m), 4.70 (1H, m), 7.04 (1H, d), 7.19 (1H, d)

### (3) Production of N-acetyl-2-chloro-4-fluoro-N-(2,2,2-trifluoroethyl)-5-{2-[(trifluoromethyl)sulfonamido]propoxy}benzamide (compound of present invention No.: C-4214)

To a mixture of 0.30 g (0.50 mmol) of tert-butyl(1-{5-[acetyl(2,2,2-trifluoroethyl)carbamoyl]-4-chloro-2-fluorophenoxy}propan-2-yl) [(trifluoromethyl)sulfonyl]carbamate and 6 mL of chloroform, 3 mL of trifluoroacetic acid was added at room temperature, and the mixture was stirred at room temperature for 4 hours. The solvent was distilled off in vacuo, and the residue was purified by silica gel column chromatography (developing solvent, n-hexane:ethyl acetate=3:1) to obtain 0.19 g (yield: 76%) of a target product.

¹H-NMR data (400 MHz, CDCl₃/TMS δ (ppm)): 1.48 (3H, d), 2.39 (3H, s), 3.99 (3H, m), 4.40 (2H, q), 5.28 (1H, d), 7.03 (1H, d), 7.22 (1H, d)

### Example 11

### Production of (S)-2-chloro-N-cyclopropyl-4-fluoro-5-(2-{[1,1,1-trifluoro-N-(methoxymethyl)methyl]sulfonamido}propoxy)benzamide (compound of present invention No.: C-0664)

### (1) Production of 4-chloro-2-fluorophenyl methyl carbonate

To a mixture of 50.00 g (341.2 mmol) of 4-chloro-2-fluorophenol, 15.01 g (375.3 mmol) of sodium hydroxide, and 340 mL of water, 38.69 g (409.5 mmol) of methyl chloroformate was added in an ice bath, and the mixture was stirred at room temperature for 2 hours. Water was added to the mixture, and the mixture was subjected to extraction with hexane. The obtained organic layer was washed with brine, and dried over anhydrous magnesium sulfate. The solvent was distilled off in vacuo to obtain 69.40 g (yield: 99%) of a target product.

¹H-NMR data (400 MHz, CDCl₃/TMS δ (ppm)) 3.94 (3H, s), 7.12-7.20 (2H, m), 7.22 (1H, dd)

### (2) Production of 5-bromo-4-chloro-2-fluorophenyl methyl carbonate

To a mixture of 34.13 g (166.8 mmol) of 4-chloro-2-fluorophenyl methyl carbonate and 140 mL of concentrated sulfuric acid, 26.24 g (91.77 mmol) of 1,3-dibromo-5,5-dimethylhydantoin was added little by little in an ice bath, and the mixture was stirred in an ice bath for 2 hours. The mixture was poured into ice, followed by extraction with dichloromethane. The obtained organic layer was washed with a saturated aqueous sodium bicarbonate solution, an aqueous sodium thiosulfate solution, and brine, and dried over anhydrous magnesium sulfate. The solvent was distilled off in vacuo to obtain an oil, and n-pentane was added to the oil to obtain crystals. The crystals were collected by filtration to obtain 34.06 g (yield: 72%) of a target product.

¹H-NMR data (300 MHz, CDCl₃/TMS δ (ppm)): 3.94 (3H, s), 7.34 (1H, d), 7.52 (1H, d)

### (3) Production of 5-bromo-4-chloro-2-fluorophenol

To a mixture of 22.00 g (77.61 mmol) of 5-bromo-4-chloro-2-fluorophenyl methyl carbonate and 220 mL of methanol, 6.53 g (purity: 85.5%, 99.5 mmol) of potassium hydroxide was added in an ice bath, and the mixture was stirred at room temperature for 1 hour. The solvent was distilled off in vacuo, 1N hydrochloric acid was added to the residue, and the mixture was subjected to extraction with dichloromethane. The obtained organic layer was washed with water and brine, and dried over anhydrous magnesium sulfate. The solvent was distilled off in vacuo to obtain 17.08 g (yield: 98%) of a target product.

¹H-NMR data (300 MHz, CDCl₃/TMS δ (ppm)): 5.36 (1H, brs), 7.22 (1H, d), 7.29 (1H, d)

### (4) Production of (S)-tert-butyl-[1-(5-bromo-4-chloro-2-fluorophenoxy)propan-2-yl]carbamate

To a mixture of 17.08 g (75.76 mmol) of 5-bromo-4-chloro-2-fluorophenol, 15.93 g (90.91 mmol) of (S)-2-(tert-butoxycarbonylamino)-1-propanol, 23.85 g (90.93 mmol) of triphenylphosphine, and 230 mL of tetrahydrofuran, 18.38 g (90.90 mmol) of diisopropyl azodicarboxylate was added under ice cooling, and the mixture was stirred at room temperature for 15 hours. The solvent was distilled off in vacuo, and the residue was purified by silica gel column chromatography (developing solvent, n-hexane:ethyl acetate=4:1). The mixed solution of n-hexane and ethyl acetate after purification was washed with a 0.5 M aqueous sodium hydroxide solution. The solvent was distilled off in vacuo to obtain 24.32 g (yield: 84%) of a target product.

¹H-NMR data (300 MHz, CDCl₃/TMS δ (ppm)): 1.30 (3H, d), 1.45 (9H, s), 3.90-4.10 (3H, m), 4.72 (1H, brs), 7.20 (1H, d), 7.21 (1H, d)

### (5) Production of methyl (S)-5-[2-(tert-butoxycarbonylamino)propoxy]-2-chloro-4-fluorobenzoate (compound of present invention No.: G-0432)

A mixture of 11.70 g (30.58 mmol) of (S)-tert-butyl-[l-(5-bromo-4-chloro-2-fluorophenoxy)propan-2-yl]carbamate, 0.25 g (0.31 mmol) of [1,1′-bis(diphenylphosphino)ferrocene]palladium (II) dichloride dichloromethane adduct, 6.19 g (61.2 mmol) of triethylamine, and 50 mL of methanol was heated with stirring at 100 °C for 8.5 hours under a carbon monoxide atmosphere (9.0 kgf/cm²). After the mixture was allowed to cool at room temperature, ethyl acetate was added thereto. Then, insoluble substances were removed by filtration, and the solvent was distilled off in vacuo. The residue was purified by silica gel column chromatography (developing solvent, n-hexane:ethyl acetate=4:1) to obtain 9.91 g (yield: 90%) of a target product.

¹H-NMR data (300 MHz, CDCl₃/TMS δ (ppm)): 1.31 (3H, d), 1.45 (9H, s), 3.92 (3H, s), 3.98-4.13 (3H, m), 4.75 (1H, brs), 7.19 (1H, d), 7.50 (1H, d)

### (6) Production of methyl (S)-5-(2-aminopropoxy)-2-chloro-4-fluorobenzoate hydrochloride (compound of present invention No.: G-0383)

To a mixture of 19.15 g (52.93 mmol) of methyl (S)-5-[2-(tert-butoxycarbonylamino)propoxy]-2-chloro-4-fluorobenzoate and 80 mL of 1,4-dioxane, 80 mL of a 1,4-dioxane solution of 4N hydrogen chloride was added at room temperature, and the mixture was stirred at room temperature for 6 hours. The solvent was distilled off in vacuo to obtain 15.10 g (yield: 96%) of a target product.

¹H-NMR data (300 MHz, DMSO-d6/TMS δ (ppm)): 1.31 (3H, d), 3.61 (1H, m), 3.87 (3H, s), 4.18-4.30 (2H, m), 7.66 (1H, d), 7.67 (1H, d), 8.38 (3H, brs)

### (7) Production of methyl (S)-2-chloro-4-fluoro-5-{2-[(trifluoromethyl)sulfonamido]propoxy}benzoate (compound of present invention No.: G-0518)

To a mixture of 15.10 g (50.65 mmol) of methyl (S)-5-(2-aminopropoxy)-2-chloro-4-fluorobenzoate hydrochloride, 15.38 g (152.0 mmol) of triethylamine, 6.34 g of molecular sieve (4A), 200 mL of tetrahydrofuran, and 200 mL of dichloromethane, 11.10 g (65.87 mmol) of trifluoromethanesulfonyl chloride was added under ice cooling, and the mixture was stirred at room temperature for 24 hours. The mixture was filtered, the solvent was distilled off in vacuo, and then ethyl acetate was added to the residue. The organic layer was washed with 1N hydrochloric acid, water, and brine, and dried over anhydrous magnesium sulfate. The solvent was distilled off in vacuo, and the residue was purified by silica gel column chromatography (developing solvent, n-hexane:ethyl acetate=3:1) to obtain 13.84 g (yield: 69%) of a target product.

¹H-NMR data (300 MHz, CDCl₃/TMS δ (ppm)): 1.49 (3H, d), 3.93 (3H, s), 4.02 (3H, m) 5.42 (1H, brs), 7.23 (1H, s), 7.49 (1H, d)

### (8) Production of (S)-2-chloro-4-fluoro-5-{2-[(trifluoromethyl)sulfonamido]propoxy}benzoic acid (compound of present invention No.: F-0511)

To a mixture of 13.84 g (35.15 mmol) of methyl (S)-2-chloro-4-fluoro-5-{2-[(trifluoromethyl)sulfonamido]propoxy}benzoate, 30 mL of tetrahydrofuran, 150 mL of ethanol, and 40 mL of water, 4.42 g (105 mmol) of lithium hydroxide monohydrate was added at room temperature, and the mixture was stirred at room temperature for 15 hours. The solvent was distilled off in vacuo, 1N hydrochloric acid was added to the residue, and the mixture was subjected to extraction with ethyl acetate. The obtained organic layer was washed with water and brine, and dried over anhydrous magnesium sulfate. The solvent was distilled off in vacuo to obtain 12.07 g (yield: 90%) of a target product.

¹H-NMR data (300 MHz, DMSO-d6/TMS δ (ppm)): 1.25 (3H, d), 3.87 (1H, m), 3.99 (1H, m), 4.16 (1H, dd), 7.57 (1H, d), 7.59 (1H, d), 9.65 (1H, brs)

### (9) Production of (S)-2-chloro-N-cyclopropyl-4-fluoro-5-{2-[(trifluoromethyl)sulfonamido]propoxy}benzamide (compound of present invention No.: C-0130)

To a mixture of 5.00 g (13.2 mmol) of (S)-2-chloro-4-fluoro-5-{2-[(trifluoromethyl)sulfonamido]propoxy}benzoic acid, 27.6 mg (0.378 mmol) of N,N-dimethylformamide, and 60 mL of dichloromethane, 3.34 g (26.3 mmol) of oxalyl chloride was added at room temperature, and the mixture was stirred for 1 hour. The mixture was concentrated in vacuo, and the residue was dissolved in 20 mL of dichloromethane. The obtained mixture was added dropwise to a mixture of 1.50 g (26.3 mmol) of cyclopropylamine, 2.67 g (26.4 mmol) of triethylamine, 60 mL of dichloromethane, and 60 mL of tetrahydrofuran under ice cooling, followed by stirring at room temperature for 18 hours. The solvent was distilled off in vacuo, 1N hydrochloric acid was added to the residue, and the mixture was subjected to extraction with ethyl acetate. The obtained organic layer was washed with water and brine, and dried over anhydrous magnesium sulfate. The solvent was distilled off in vacuo, and the residue was purified by silica gel column chromatography (developing solvent, n-hexane:ethyl acetate=1:1) to obtain 4.59 g (yield: 83%) of a target product.

¹H-NMR data (300 MHz, DMSO-d6/TMS δ (ppm)): 0.53 (2H, m), 0.69 (2H, m), 1.25 (3H, d), 2.79 (1H, m), 3.87 (1H, m), 3.97 (1H, dd), 4.11 (1H, dd), 7.26 (1H, d), 7.49 (1H, d), 8.45 (1H, brs), 9.63 (1H, brs)

### (10) Production of (S)-2-chloro-N-cyclopropyl-4-fluoro-5-(2-{[1,1,1-trifluoro-N-(methoxymethyl)methyl]sulfonamido}propoxy)benzamide (compound of present invention No.: C-0664)

To a mixture of 1.50 g (3.58 mmol) of (S)-2-chloro-N-cyclopropyl-4-fluoro-5-{2-[(trifluoromethyl)sulfonamido]propoxy}benzamide, 0.54 g (5.3 mmol) of triethylamine, and 30 mL of acetonitrile, 0.43 g (5.3 mmol) of chloromethyl methyl ether was added dropwise under ice cooling, and the mixture was stirred at room temperature for 15 hours. Water was added thereto, and the mixture was subjected to extraction with ethyl acetate. The obtained organic layer was washed with water and brine, and dried over anhydrous magnesium sulfate. The solvent was distilled off in vacuo, and the residue was purified by silica gel column chromatography (developing solvent, n-hexane:ethyl acetate=1:1) to obtain 1.60 g (yield: 97%) of a target product.

¹H-NMR data (300 MHz, CDCl₃/TMS δ (ppm)): 0.66 (2H, m), 0.90 (2H, m), 1.46 (3H, d), 2.92 (1H, m), 3.38 (3H, s), 4.10 (1H, dd), 4.24 (1H, dd), 4.37-4.48 (1H, m), 4.80-4.91 (2H, m), 6.52 (1H, brs), 7.13 (1H, d), 7.40 (1H, d)

### Example 12

### Production of (S)-2-chloro-N-(1-cyanocyclopropyl)-4-fluoro-5-(2-{[1,1,1-trifluoro-N-(methoxymethyl)methyl]sulfonamido}propoxy)benzamide (compound of present invention No.: C-3076)

### (1) Production of (S)-2-chloro-N-(1-cyanocyclopropyl)-4-fluoro-5-{2-[(trifluoromethyl)sulfonamido]propoxy}benzamide (compound of present invention No.: C-2980)

To a mixture of 0.14 g (0.37 mmol) of (S)-2-chloro-4-fluoro-5-{2-[(trifluoromethyl)sulfonamido]propoxy}benzoic acid produced in Example 11 (8), 2.7 mg (0.037 mmol) of N,N-dimethylformamide, and 10 mL of dichloromethane, 0.10 g (0.79 mmol) of oxalyl chloride was added at room temperature, and the mixture was stirred for 1 hour. The mixture was concentrated in vacuo, and the residue was dissolved in 3 mL of dichloromethane. The obtained mixture was added dropwise to a mixture of 0.13 g (1.1 mmol) of 1-cyanocyclopropylamine hydrochloride, 0.22 g (2.2 mmol) of triethylamine, and 10 mL of dichloromethane under ice cooling, followed by stirring at room temperature for 18 hours. Then, 1N hydrochloric acid was added thereto, and the mixture was subjected to extraction with dichloromethane. The obtained organic layer was washed with brine, and dried over anhydrous magnesium sulfate. The solvent was distilled off in vacuo, and the residue was purified by silica gel column chromatography (developing solvent, n-hexane:ethyl acetate=1:1) to obtain 0.14 g (yield: 86%) of a target product.

¹H-NMR data (300 MHz, CDCl₃/TMS δ (ppm)): 1.39 (2H, m), 1.46 (3H, d), 1.67 (2H, m), 4.06-4.15 (3H, m), 5.77 (1H, brs), 7.14 (1H, brs), 7.16 (1H, d), 7.43 (1H, d)

### (2) Production of (S)-2-chloro-N-(1-cyanocyclopropyl)-4-fluoro-5-(2-{[1,1,1-trifluoro-N-(methoxymethyl)methyl]sulfonamido}propoxy)benzamide (compound of present invention No.: C-3076)

To a mixture of 0.20 g (0.45 mmol) of (S)-2-chloro-N-(1-cyanocyclopropyl)-4-fluoro-5-{2-[(trifluoromethyl)sulfonamido]propoxy}benzamide, 0.12 g (0.93 mmol) of N,N-diisopropylethylamine, and 5 mL of acetonitrile, 0.07 g (0.9 mmol) of chloromethyl methyl ether was added dropwise under ice cooling, and the mixture was stirred at room temperature for 15 hours. Water was added thereto, and the mixture was subjected to extraction with ethyl acetate. The obtained organic layer was washed with brine, and dried over anhydrous magnesium sulfate. The solvent was distilled off in vacuo, and the residue was purified by silica gel column chromatography (developing solvent, n-hexane:ethyl acetate=1:1) to obtain 0.19 g (yield: 86%) of a target product.

¹H-NMR data (300 MHz, CDCl₃/TMS δ (ppm)): 1.39 (2H, m), 1.49 (3H, d), 1.68 (2H, m), 3.38 (3H, s), 4.11 (1H, dd), 4.25 (1H, dd), 4.44 (1H, m), 4.86 (2H, m), 7.04 (1H, brs), 7.16 (1H, d), 7.47 (1H, d) [0366]

### Example 13

### Production of (S)-2-chloro-N-(1-cyanocyclopropyl)-3-methyl-5-{2-[(trifluoromethyl)sulfonamido]propoxy)benzamide

### (1) Production of methyl 2-chloro-5-hydroxy-3-methylbenzoate

To a mixture of 3.5 g (18 mmol) of methyl 5-amino-2-chloro-3-methylbenzoate produced by the method described in WO 2020/117877, 50 mL of concentrated sulfuric acid, and 50 mL of water, 1.5 g (22 mmol) of sodium nitrite was added under ice cooling, and the mixture was stirred at room temperature for 2 hours and then stirred under reflux for 3 hours. The reaction mixture was cooled to room temperature, then ice water was added thereto, and the mixture was subjected to extraction with ethyl acetate. The organic layer was washed with water, and dried over anhydrous magnesium sulfate. The solvent was distilled off in vacuo to obtain a crude mixture containing a target product and 2-chloro-5-hydroxy-3-methylbenzoic acid. The obtained crude mixture containing a target product and 2-chloro-5-hydroxy-3-methylbenzoic acid was dissolved in 100 mL of methanol. Then, 3.7 g (38 mmol) of concentrated sulfuric acid was added thereto at room temperature, and the mixture was stirred under reflux for 8 hours. The solvent was distilled off in vacuo, water was added to the residue, and the mixture was subjected to extraction with ethyl acetate. The organic layer was washed with water, and dried over anhydrous magnesium sulfate. The solvent was distilled off in vacuo, and the residue was purified by silica gel column chromatography (developing solvent, n-hexane:ethyl acetate=2:1) to obtain 1.8 g (yield: 50%) of a target product.

¹H-NMR data (300 MHz, CDCl₃/TMS δ (ppm)) 2.36 (3H, s), 3.92 (3H, s), 5.50 (1H, brs), 6.88 (1H, d), 7.07 (1H, d)

### (2) Production of methyl (S)-5-[2-(tert-butoxycarbonylamino)propoxy]-2-chloro-3-methylbenzoate (compound of present invention No.: H-0001)

To a mixture of 1.8 g (9.0 mmol) of methyl 2-chloro-5-hydroxy-3-methylbenzoate, 1.9 g (11 mmol) of (S)-2-(tert-butoxycarbonylamino)-1-propanol, 2.8 g (11 mmol) of triphenylphosphine, and 100 mL of tetrahydrofuran, 2.2 g (11 mmol) of diisopropyl azodicarboxylate was added under ice cooling, and the mixture was stirred at room temperature for 15 hours. The solvent was distilled off in vacuo, and the residue was purified by silica gel column chromatography (developing solvent, n-hexane:ethyl acetate=2:1) to obtain 1.3 g (yield: 40%) of a target product.

¹H-NMR data (300 MHz, CDCl₃/TMS δ (ppm)): 1.27 (3H, d), 1.45 (9H, s), 2.39 (3H, s), 3.90 (2H, d), 3.92 (3H, s), 4.05 (1H, m), 4.71 (1H, brs), 6.94 (1H, d), 7.10 (1H, d)

### (3) Production of methyl (S)-5-(2-aminopropoxy)-2-chloro-3-methylbenzoate hydrochloride (compound of present invention No.: H-0002)

To a mixture of 1.3 g (3.6 mmol) of methyl (S)-5-[2-(tert-butoxycarbonylamino)propoxy]-2-chloro-3-methylbenzoate and 10 mL of 1,4-dioxane, 3.7 g of a 1,4-dioxane solution of 4N hydrogen chloride was added at room temperature, and the mixture was stirred at room temperature for 16 hours. The solvent was distilled off in vacuo, and the obtained white crystals were washed with ethyl acetate to obtain 880 mg (yield: 82%) of a target product.

¹H-NMR data (300 MHz, DMSO-d6/TMS δ (ppm)): 1.27 (3H, d), 2.36 (3H, s), 3.57 (1H, m), 3.86 (3H, s), 4.02 (1H, dd), 4.15 (1H, dd), 7.19 (1H, d), 7.22 (1H, d), 8.18 (3H, brs)

### (4) Production of methyl (S)-2-chloro-3-methyl-5-{2-[(trifluoromethyl)sulfonamido]propoxy}benzoate (compound of present invention No.: H-0003)

To a mixture of 880 mg (2.99 mmol) of methyl (S)-5-(2-aminopropoxy)-2-chloro-3-methylbenzoate hydrochloride, 908 mg (8.97 mmol) of triethylamine, 100 mg of molecular sieve (4A), 30 mL of tetrahydrofuran, and 60 mL of dichloromethane, 756 mg (4.49 mmol) of trifluoromethanesulfonyl chloride was added under ice cooling, and the mixture was stirred at room temperature for 17 hours. The mixture was filtered, a saturated aqueous sodium bicarbonate solution was added thereto, and the mixture was subjected to extraction with ethyl acetate. The organic layer was washed with 1N hydrochloric acid, and dried over anhydrous magnesium sulfate. The solvent was distilled off in vacuo, and the residue was purified by silica gel column chromatography (developing solvent, n-hexane:ethyl acetate=3:1) to obtain 390 mg (yield: 33%) of a target product.

¹H-NMR data (300 MHz, CDCl₃/TMS δ (ppm)): 1.46 (3H, d), 2.37 (3H, s), 3.94 (3H, s), 3.95 (1H, m), 4.06 (2H, m), 5.14 (1H, brs), 6.94 (1H, d), 7.10 (1H, d)

### (5) Production of (S)-2-chloro-3-methyl-5-{2-[(trifluoromethyl)sulfonamido]propoxy}benzoic acid (compound of present invention No.: H-0004)

To a mixture of 390 mg (1.00 mmol) of methyl (S)-2-chloro-3-methyl-5-{2-[(trifluoromethyl)sulfonamido]propoxy}benzoate, 20 mL of methanol, and 20 mL of water, 126 mg (3.00 mmol) of lithium hydroxide monohydrate was added at room temperature, and the mixture was stirred at room temperature for 15 hours. Then, 1N hydrochloric acid was added thereto, and the mixture was subjected to extraction with ethyl acetate. The obtained organic layer was washed with water, and dried over anhydrous magnesium sulfate. The solvent was distilled off in vacuo to obtain 370 mg (yield: 98%) of a target product.

¹H-NMR data (300 MHz, DMSO-d6/TMS δ (ppm)): 1.23 (3H, d), 2.33 (3H, s), 3.83 (1H, m), 3.85 (1H, dd), 3.99 (1H, dd), 7.06 (1H, d), 7.10 (1H, d)

### (6) Production of (S)-2-chloro-N-(1-cyanocyclopropyl)-3-methyl-5-{2-[(trifluoromethyl)sulfonamido]propoxy) benzamide (compound of present invention No.: H-0005)

To a mixture of 430 mg (1.14 mmol) of (S)-2-chloro-3-methyl-5-{2-[(trifluoromethyl)sulfonamido]propoxy}benzoic acid, 8.2 mg (0.11 mmol) of N,N-dimethylformamide, and 30 mL of dichloromethane, 436 mg (3.44 mmol) of oxalyl chloride was added at room temperature, and the mixture was stirred for 3 hours. The mixture was concentrated in vacuo, and the residue was dissolved in 10 mL of dichloromethane. The obtained mixture was added dropwise to a mixture of 407 mg (3.43 mmol) of 1-cyanocyclopropylamine hydrochloride, 695 mg (6.87 mmol) of triethylamine, and 30 mL of dichloromethane under ice cooling, followed by stirring at room temperature for 18 hours. Then, 1N hydrochloric acid was added thereto, and the mixture was subjected to extraction with ethyl acetate. The obtained organic layer was washed with a saturated aqueous sodium bicarbonate solution, and dried over anhydrous magnesium sulfate. The solvent was distilled off in vacuo, and the residue was purified by silica gel column chromatography (developing solvent, n-hexane:ethyl acetate=3:2) to obtain 280 mg (yield: 56%) of a target product.

¹H-NMR data (300 MHz, CDCl₃/TMS δ (ppm)): 1.39 (5H, m), 1.63 (2H, dd), 2.34 (3H, s), 3.98 (3H, m), 5.78 (1H, brs), 6.87 (1H, d), 6.93 (1H, d), 6.97 (1H, brs)

### (7) Production of (S)-2-chloro-N-(1-cyanocyclopropyl)-3-methyl-5-(2-{[1,1,1-trifluoro-N-(methoxymethyl)methyl]sulfonamido}propoxy)benzamide (compound of present invention No.: H-0006)

To a mixture of 150 mg (0.341 mmol) of (S)-2-chloro-N-(1-cyanocyclopropyl)-3-methyl-5-{2-[(trifluoromethyl)sulfonamido]propoxy}benzamide, 138 mg (1.36 mmol) of triethylamine, and 30 mL of acetonitrile, 54.9 mg (0.682 mmol) of chloromethyl methyl ether was added dropwise at room temperature, and the mixture was stirred at room temperature for 15 hours. Water was added thereto, and the mixture was subjected to extraction with ethyl acetate. The obtained organic layer was dried over anhydrous magnesium sulfate. The solvent was distilled off in vacuo, and the residue was purified by silica gel column chromatography (developing solvent, n-hexane:ethyl acetate=1:1) to obtain 110.1 mg (yield: 67%) of a target product.

¹H-NMR data (300 MHz, CDCl₃/TMS δ (ppm)): 1.43 (5H, m), 1.67 (2H, dd), 2.37 (3H, s), 3.37 (3H, s), 4.01 (1H, m), 4.14 (1H, m), 4.40 (1H, m), 4.86 (2H, m), 6.67 (1H, brs), 6.90 (1H, d), 7.01 (1H, d) [0374]

Physical property values of the compound [I] of the present invention synthesized in accordance with the above-described Examples, including the values in the above-described Examples, are shown in the following Tables 1234 to 1244, physical property values of the compound [II] of the present invention are similarly shown in Tables 1245 to 1246, and physical property values of the compound [III] of the present invention are similarly shown in Tables 1247 to 1253.

The compound numbers and symbols in the tables are the same as described above.

TABLE 1234

| | Physical property value | |
|---|---|---|
| Compound Number | Melting point (°C) | Refractive index (n_{D}²⁰) |
| A-0008 | 91-92 | |
| A-0015 | 64-66 | |
| A-0028 | 107-110 | |
| A-0030 | 86-88 | |
| A-0031 | 110-113 | |
| A-0035 | 78-79 | |
| A-0037 | 82-85 | |
| A-0049 | 88-90 | |
| A-0052 | 71-72 | |
| A-0056 | 90-93 | |
| A-0059 | 79-80 | |
| A-0060 | 47-48 | |
| A-0063 | 55-56 | |
| A-0064 | 48-50 | |
| A-0105 | 129-130 | |
| A-0106 | 92-95 | |
| A-0170 | 82-84 | |
| A-0179 | | 1.4858 |
| A-0243 | 117-120 | |
| A-0273 | | 1.4781 |
| A-0367 | 100-103 | |
| A-0368 | 75-78 | |
| A-0369 | | 1.4932 |
| A-0370 | 68-71 | |
| A-0371 | | 1.4941 |
| A-0373 | 153-155 | |
| A-0405 | 63-66 | |
| A-0419 | 111-113 | |
| A-0447 | | 1.4881 |
| A-0474 | 116-119 | |
| A-0475 | | 1.4862 |
| A-0479 | | 1.4862 |
| A-0480 | | 1.4852 |
| A-0507 | 119-121 | |
| A-0512 | 142-144 | |
| A-0538 | 107-109 | |
| A-0598 | 129-130 | |
| A-0724 | 187-190 | |
| A-0726 | 154-156 | |
| A-0751 | 107-108 | |
| A-0752 | 85-87 | |
| A-0753 | 85-87 | |
| A-0809 | 82-83 | |

TABLE 1235

| | Physical property value | |
|---|---|---|
| Compound Number | Melting point (°C) | Refractive index (n_{D}²⁰) |
| A-0942 | 66-68 | |
| A-1310 | 122-124 | |
| A-1477 | 73-75 | |
| A-1483 | 90-93 | |
| A-1523 | 100-101 | |
| A-1529 | 62-63 | |
| A-1960 | 145-146 | |
| A-3459 | 97-99 | |
| A-3506 | 161-162 | |
| A-3821 | 152-153 | |
| A-3828 | 131-134 | |
| A-4665 | 64-67 | |
| A-4687 | 96-98 | |
| A-4688 | 74-75 | |
| A-4689 | 88-89 | |
| A-4690 | 81-83 | |
| A-4691 | 81-84 | |
| A-4693 | 103-106 | |
| A-4710 | 91-94 | |
| A-4712 | 88-89 | |
| A-4715 | 76-77 | |
| A-4735 | 94-95 | |
| A-4758 | 88-89 | |
| A-4781 | 75-78 | |
| A-4850 | 56-58 | |
| A-4969 | | 1.5141 |
| A-5011 | 82-83 | |
| A-5172 | 97-100 | |
| A-5797 | 180-183 | |
| A-5977 | 85-86 | |
| A-6161 | 41-43 | |
| A-6482 | | 1.4748 |
| A-6497 | | 1.4718 |
| A-6542 | | 1.4709 |
| A-6572 | | 1.4723 |
| A-6602 | | 1.4641 |
| A-6632 | | 1.4540 |
| A-6662 | | 1.4801 |
| A-6692 | | 1.4820 |
| A-6708 | | 1.4911 |
| A-6709 | | 1.4804 |
| A-6724 | | 1.4809 |
| A-6754 | | 1.4711 |

TABLE 1236

| | Physical property value | |
|---|---|---|
| Compound Number | Melting point (°C) | Refractive index (n_{D}²⁰) |
| A-6784 | | 1.4730 |
| A-6814 | | 1.4810 |
| A-6844 | | 1.4760 |
| A-6859 | | 1.4714 |
| A-6889 | | 1.4751 |
| A-6934 | 63-64 | |
| A-7024 | | 1.4698 |
| A-7054 | | 1.4739 |

TABLE 1237

| Compound | | |
|---|---|---|
| Number | Physical property value [¹H-NMR data TMS δ (ppm)] | |
| A-0061 | 300 MHz, CDCl₃ | 4.02-4.23 (2H, m), 6.75 (1H, brs), 7.35 (1H, d), 7.73 (1H, d) |
| A-0155 | 300 MHz, CDCl₃ | 1.44 (3H, d), 3.33 (1H, q), 3.99-4.06 (2H, m), 4.08-4.20 (2H, m), 6.78 (1H, |
| | | brs), 7.18 (1H, d), 7.45 (1H, d) |
| A-0156 | 300 MHz, CDCl₃ | 1.43 (3H, d), 2.35 (3H, s), 3.88-4.03 (2H, m), 4.08-4.20 (3H, m), 6.85 (1H, |
| | | brs), 7.18 (1H, d), 7.51 (1H, d) |
| A-0372 | 300 MHz, CDCl₃ | 1.17 (3H, d), 1.72 (2H, brs), 3.33-3.45 (1H, m), 3.73 (1H, t), 3.93 (1H, dd), |
| | | 4.05-4.19 (2H, m), 7.00 (1H, brs), 7.16 (1H, d), 7.38 (1H, d) |
| A-0414 | 300 MHz, CDCl₃ | 1.25 (3H, d), 1.43 (9H, s), 3.95-4.19 (3H, m), 4.69 (1H, brs), 4.96 (2H, q), |
| | | 6.83 (1H, brs), 7.15 (1H, d), 7.44 (1H, d) |
| A-0417 | 300 MHz, CDCl₃ | 0.99 (3H, t), 1.20-1.32 (2H, m), 1.44 (9H, s), 3.75-3.92 (1H, m), 4.09-4.20 |
| | | (2H, m), 4.71 (1H, brs), 4.98 (2H, q), 6.83 (1H, brs), 7.17 (1H, d), 7.46 (1H, |
| | | d) |
| A-0418 | 400 MHz, CDCl₃ | 0.98 (6H, d), 1.44 (9H, s), 1.94-2.10 (1H, m), 3.32-3.78 (1H, m), 4.10-4.23 |
| | | (2H, m), 4.73 (1H, brs), 4.97 (2H, q), 6.83 (1H, brs), 7.17 (1H, d), 7.44 (1H, |
| | | d) |
| A-0491 | 400 MHz, CDCl₃ | 0.93 (3H, t), 1.55-1.83 (2H, m), 3.17-3.39 (2H, m), 4.04-4.24 (2H, m), 4.27- |
| | | 4.39 (1H, m), 5.12 (1H, brs), 6.81 (1H, brs), 7.14 (1H, d), 7.42 (1H, d), 7.76 |
| | | (2H, d), 7.98 (2H, d) |
| A-0758 | 300 MHz, CDCl₃ | 1.24 (3H, d), 2.40-2.55 (1H, m), 3.06 (1H, dd), 3.23 (1H, dd), 3.99 (1H, dd), |
| | | 4.07-4.18 (3H, m), 6.83 (1H, brs), 7.17 (1H, d), 7.42 (1H, d) |
| A-0799 | 300 MHz, CDCl₃ | 2.18-2.31 (2H, m), 2.36 (3H, s), 3.75 (2H, t), 4.11 (2H, t), 4.07-4.22 (2H, m), |
| | | 5.94 (1H, brs), 6.95 (1H, d), 7.30 (1H, d) |
| A-3167 | 300 MHz, CDCl₃ | 0.61-0.67 (4H, m), 0.75-0.79 (2H, m), 0.86-0.92 (2H, m), 1.72 (2H, brs), |
| | | 2.91 (1H, m), 3.92 (2H, s), 6.51 (1H, brs), 7.12 (1H, d), 7.38 (1H, d) |
| A-3168 | 300 MHz, CDCl₃ | 0.62-0.67 (2H, m), 0.83-0.97 (6H, m), 1.45 (9H, s), 2.91 (1H, m), 4.05 (2H, |
| | | s), 5.15 (1H, brs), 6.42 (1H, brs), 7.11 (1H, d), 7.33 (1H, d) |

TABLE 1238

| Compound | | |
|---|---|---|
| Number | Physical property value [¹H-NMR data TMS δ (ppm)] | |
| A-3822 | 300 MHz, | 0.62-0.68 (2H, m), 0.86-0.93 (2H, m), 1.88 (1H, brs), 2.28 (1H, m), 2.91 |
| | CDCl₃ | (1H, m), 3.80 (2H, d), 4.09-4.21 (4H, m), 6.53 (1H, brs), 7.12 (1H, d), 7.44 |
| | | (1H, d) |
| A-3825 | 300 MHz, | 0.62-0.68 (2H, m), 0.86-0.93 (2H, m), 1.45 (9H, s), 2.92 (1H, m), 3.77 (2H, |
| | CDCl₃ | m), 3.99 (1H, dd), 4.06-4.19 (4H, m), 6.57 (1H, brs), 7.12 (1H, d), 7.42 (1H, |
| | | d) |
| A-3829 | 300 MHz, | 0.62-0.68 (2H, m), 0.86-0.93 (2H, m), 2.55 (1H, m), 2.89 (1H, m), 3.56-3.62 |
| | CDCl₃ | (2H, m), 3.71-3.81 (2H, m), 4.18-4.26 (2H, m), 6.58 (1H, brs), 7.11 (1H, d), |
| | | 7.39 (1H, d) |
| A-3129 | 300 MHz, | 0.62-0.67 (2H, m), 0.86-0.93 (2H, m), 1.40 (3H, d), 2.39 (1H, m), |
| | CDCl₃ | 2.92 (1H, m), 3.62-3.73 (2H, m), 3.99-4.03 (2H, m), 6.48 (1H, brs), |
| | | 7.11 (1H, d), 7.42 (1H, d) |
| A-3169 | 300 MHz, | 0.62-0.68 (2H, m), 0.86-0.93 (2H, m), 0.98-1.03 (2H, m), 1.24-1.29 |
| | CDCl₃ | (2H, m), 2.91 (1H, m), 4.04 (2H, s), 6.02 (1H, brs), 6.51 (1H, brs), 7.14 (1H, |
| | | d), 7.35 (1H, d) |
| A-3820 | 300 MHz, | 0.62-0.67 (2H, m), 0.85-0.92 (2H, m), 1.10 (3H, d), 2.50 (1H, m), |
| | CDCl₃ | 2.91 (1H, m), 3.70 (1H, dd), 3.84 (1H, dd), 3.92-4.00 (2H, m), 6.46 |
| | | (1H, brs), 7.03 (1H, d), 7.32 (1H, d), 7.70-7.74 (2H, m), 7.80-7.84 (2H, m) |
| A-7142 | 300 MHz, | 0.97-1.02 (4H, m), 1.23-1.27 (2H, m), 1.56-1.60 (2H, m), 4.06 (2H, |
| | DMSO-d₆ | s), 7.29 (1H, d), 7.57 (1H, d), 9.36 (1H, brs), 10.16 (1H, brs) |

TABLE 1239

Physical property value

| | | |
|---|---|---|
| | | Refractive index |
| Compound Number | Melting point (°C) | (n_{D}²⁰) |

| | |
|---|---|
| B-0009 | 1.4759 |

TABLE 1240

Physical property value

| | | Refractive index |
|---|---|---|
| Compound Number | Melting point (°C) | (n_{D}²⁰) |
| C-0001 | | 1.4715 |
| C-0002 | | 1.4705 |
| C-0004 | 120-123 | |
| C-0005 | 119-122 | |
| C-0006 | 112-115 | |
| C-0010 | 129-132 | |
| C-0011 | 160-162 | |
| C-0035 | 129-130 | |
| C-0066 | 127-128 | |
| C-0068 | | 1.4962 |
| C-0128 | 92-95 | |
| C-0129 | 120-123 | |
| C-0130 | | 1.5023 |
| C-0188 | | 1.4692 |
| C-0190 | | 1.4699 |
| C-0192 | | 1.4691 |
| C-0196 | | 1.4731 |
| C-0197 | | 1.4731 |
| C-0221 | | 1.4619 |
| C-0314 | | 1.5051 |
| C-0315 | | 1.5009 |
| C-0316 | | 1.5039 |
| C-0448 | | 1.3495 |
| C-0590 | | 1.4699 |
| C-0664 | | 1.5002 |
| C-0698 | | 1.4749 |
| C-0806 | 67-70 | |
| C-0880 | 82-85 | |
| C-0988 | 108-111 | |
| C-1023 | 123-125 | |
| C-1349 | | 1.4760 |
| C-1367 | | 1.4744 |
| C-1403 | | 1.3779 |
| C-1439 | | 1.4764 |
| C-1457 | | 1.4732 |
| C-1475 | | 1.4720 |
| C-1511 | | 1.4695 |
| C-2980 | 54-57 | |
| C-3076 | | 1.4963 |
| C-4162 | 119-121 | |
| C-4214 | | 1.4694 |
| C-9400 | 112-114 | |
| C-9422 | 109-110 | |

TABLE 1241

Physical property value

| | | Refractive index |
|---|---|---|
| Compound Number | Melting point (°C) | (n_{D}²⁰) |
| C-9432 | 118-120 | |
| C-9464 | 51-54 | |
| C-9496 | | 1.5176 |
| C-9528 | 163-165 | |
| C-9688 | | 1.4945 |
| C-9752 | | 1.4948 |
| C-9784 | | 1.4919 |
| C-9944 | 66-69 | |
| C-11248 | | 1.4974 |
| C-11256 | 117-120 | |
| C-11264 | | 1.4880 |
| C-11272 | | 1.4892 |
| C-11408 | 56-59 | |
| C-11416 | 109-111 | |
| C-11512 | | 1.5022 |
| C-11528 | 135-138 | |
| C-11536 | 173-176 | |
| C-11568 | 132-133 | |
| C-11656 | 72-75 | |
| C-11664 | 90-93 | |
| C-11680 | | 1.5005 |
| C-11688 | | 1.5033 |
| C-11704 | | 1.4877 |
| C-11712 | | 1.5010 |
| C-11720 | 106-107 | |
| C-11728 | 143-146 | |
| C-11752 | 82-85 | |
| C-11776 | 110-113 | |
| C-11784 | | 1.5327 |
| C-11792 | | 1.5266 |
| C-11800 | 180-183 | |
| C-11808 | 96-99 | |
| C-11816 | 164-166 | |
| C-11824 | 141-142 | |
| C-11840 | 90-93 | |
| C-13648 | | 1.4891 |
| C-3550 | | 1.5011 |
| C-8914 | | 1.4874 |
| C-8950 | | 1.4889 |
| C-9334 | | 1.5080 |
| C-9338 | 59-62 | |
| C-9848 | | 1.4952 |
| C-11392 | 109-112 | |
| C-11584 | 57-60 | |

TABLE 1242

| Compound Number | Physical property value [¹H-NMR data TMS δ (ppm)] | |
|---|---|---|
| C-0191 | 300 MHz, CDCl₃ | 1.40 (3H, d), 3.05 (3H, s), 4.05-4.21 (4H, m), 4.40-4.51 (1H, m), 6.81 (1H, |
| | | brs), 7.20 (1H, d), 7.43 (1H, d) |
| C-0374 | 300 MHz, CDCl₃ | 1.27 (3H, t), 1.45 (3H, d), 3.38-3.63 (2H, m), 3.98-4.24 (4H, m), 4.28-4.44 |
| | | (1H, m), 6.80 (1H, brs), 7.20 (1H, d), 7.43 (1H, d) |
| C-1022 | 400 MHz, CDCl₃ | 0.95 (3H, s), 1.58-1.76 (2H, m), 2.48 (3H, s), 2.83-3.04 (3H, m), 3.09 (3H, |
| | | s), 3.46-3.72 (2H, m), 4.68 (1H, brs), 7.42 (1H, d), 7.48 (1H, d) |
| C-9560 | 300 MHz, CDCl₃ | 0.63-0.68 (2H, m), 0.86-0.93 (2H, m), 1.44 (3H, d), 2.10 (3H, s), 2.91 (1H, |
| | | m), 4.05 (1H, dd), 4.28-4.42 (4H, m), 6.42 (1H, brs), 7.12 (1H, d), 7.25 (1H, |
| | | d) |
| C-9592 | 300 MHz, CDCl₃ | 0.63-0.68 (2H, m), 0.87-0.93 (2H, m), 1.48 (3H, d), 2.91 (1H, m), 3.63 (3H, |
| | | s), 4.12 (1H, dd), 4.28 (2H, s), 4.32 (1H, m), 4.43 (1H, m), 6.46 (1H, brs), |
| | | 7.13 (1H, d), 7.32 (1H, d) |
| C-9656 | 300 MHz, CDCl₃ | 0.63-0.68 (2H, m), 0.87-0.93 (2H, m), 1.48 (3H, d), 2.05 (3H, s), 2.92 (1H, |
| | | m), 4.15 (2H, m), 4.45 (1H, m), 5.40 (2H, m), 6.53 (1H, brs), 7.15 (1H, d), |
| | | 7.40 (1H, d) |
| C-9720 | 300 MHz, CDCl₃ | 0.62-0.67 (2H, m), 0.86-0.93 (2H, m), 1.16 (9H, s), 1.48 (3H, d), 2.92 (1H, |
| | | m), 4.11-4.20 (2H, m), 4.44-4.50 (1H, m), 5.56 (2H, m), 6.52 (1H, brs), 7.14 |
| | | (1H, d), 7.38 (1H, d) |
| C-10702 | 300 MHz, CDCl₃ | 1.50 (9H, s), 1.53 (3H, d), 2.37 (3H, s), 4.19 (1H, m), 4.31-4.49 (3H, m), |
| | | 4.70 (1H, m), 7.04 (1H, d), 7.19 (1H, d) |
| C-11312 | 300 MHz, CDCl₃ | 0.73-0.77 (2H, m), 0.86-0.90 (2H, m), 1.46 (3H, d), 1.49 (3H, s), 4.01-4.14 |
| | | (3H, m), 5.73 (1H, brs), 6.64 (1H, brs), 7.13 (1H, d), 7.34 (1H, d) |
| C-11400 | 300 MHz, CDCl₃ | 1.20-1.26 (2H, m), 1.37-1.42 (2H, m), 1.47 (3H, d), 2.22 (1H, s), 5.54 (1H, |
| | | brs), 6.94 (1H, brs), 7.15 (1H, d), 7.46 (1H, d) |
| C-11520 | 300 MHz, CDCl₃ | 1.46 (3H, d), 2.14-2.33 (2H, m), 2.43-2.53 (2H, m), 2.84-2.93 (2H, m), 4.03- |
| | | 4.15 (3H, m), 5.60 (1H, brs), 7.12 (1H, brs), 7.17 (1H, d), 7.48 (1H, d) |
| C-11552 | 300 MHz, DMSO-d6 | 1.26 (3H, d), 1.76 (3H, s), 3.80 (1H, dd), 3.88 (1H, m), 3.95-4.05 (2H, m), |
| | | 4.10-4.15 (2H, m), 4.42 (1H, dd), 4.58 (1H, m), 7.36 (1H, d), 7.54 (1H, d), |
| | | 9.08 (1H, brs), 9.64 (1H, brs) |

TABLE 1243

| Compound Number | Physical property value [¹H-NMR data TMS δ (ppm)] | |
|---|---|---|
| C-11560 | 300 MHz, DMSO-d6 | 1.27 (3H, d), 2.99 (3H, s), 3.19 (1H, ddd), 3.76 (1H, m), 3.85-4.01 (3H, m), |
| | | 4.09-4.66 (3H, m), 7.32 (1H, d), 7.54 (1H, d), 8.84 (1H, brs), 9.65 (1H, brs) |
| C-11624 | 300 MHz, CDCl₃ | 1.46 (3H, d), 2.20 (6H, s), 3.99-4.16 (3H, m), 5.59 (1H, brs), 6.34 (1H, brs), |
| | | 7.14 (1H, d), 7.43 (1H, d) |
| C-11672 | 300 MHz, CDCl₃ | 1.47 (3H, d), 2.11-2.21 (2H, m), 2.71-2.82 (4H, m), 2.96-3.09 (2H, m), 4.03- |
| | | 4.16 (3H, m), 5.50 (1H, brs), 6.68 (1H, brs), 7.19 (1H, d), 7.49 (1H, d) |
| C-11696 | 300 MHz, CDCl₃ | 0.32 (1H, m), 0.41 (1H, m), 0.48-0.62 (2H, m), 0.92 (1H, m), 1.32 (3H, d), |
| | | 1.45 (3H, d), 3.59 (1H, m), 4.01-4.12 (3H, m), 5.93 (1H, brs), 6.38 (1H, brs), |
| | | 7.15 (1H, d), 7.35 (1H, d) |
| C-11744 | 300 MHz, CDCl₃ | 1.46 (3H, d), 1.82 (6H, s), 4.05-4.16 (3H, m), 5.64 (1H, brs), 6.77 (1H, brs), |
| | | 7.16 (1H, d), 7.46 (1H, d) |
| C-11760 | 300 MHz, CDCl₃ | 1.46 (3H, d), 3.91 (3H, s), 4.04-4.16 (3H, m), 5.85 (1H, brs), 7.17 (1H, d), |
| | | 7.34 (1H, d), 9.08 (1H, brs) |

TABLE 1244

Physical property value

| | | Refractive index |
|---|---|---|
| Compound Number | Melting point (°C) | (n_{D}²⁰) |
| D-0006 | 130-132 | |
| D-0125 | | 1.5130 |
| D-0130 | | 1.5077 |
| D-0592 | 69-70 | |
| D-0664 | | 1.5043 |
| D-2977 | 65-68 | |
| D-2980 | 195-198 | |
| D-2992 | 203-206 | |
| D-3076 | | 1.5029 |
| D-4423 | | 1.4248 |
| D-4495 | | 1.4657 |

TABLE 1245

Physical property value

| | | Refractive index |
|---|---|---|
| Compound Number | Melting point (°C) | (n_{D}²⁰) |
| E-0002 | 127-130 | |
| E-0006 | 124-126 | |
| E-0007 | 101-102 | |
| E-0011 | 108-109 | |
| E-0012 | 136-138 | |

TABLE 1246

| Compound Number | Physical property value [¹H-NMR data TMS δ (ppm)] | |
|---|---|---|
| E-0010 | 400 MHz, DMSO-d6 | 2.21 (3H, s), 4.04 (2H, q), 6.95 (1H, d), 7.05 (1H, d), 8.86 (1H, brs), 9.88 |
| | | (1H, brs) |
| E-0030 | 300 MHz, DMSO-d6 | 0.46-0.52 (2H, m), 0.64-0.71 (2H, m), 2.78 (1H, m), 6.93 (1H, d), 7.38 (1H, |
| | | d), 8.40 (1H, brs), 10.39 (1H, brs) |

TABLE 1247

Physical property value

| | | Refractive index |
|---|---|---|
| Compound Number | Melting point (°C) | (n_{D}²⁰) |
| F-0423 | 160-161 | |
| F-0510 | 133-136 | |
| F-1861 | 103-104 | |

TABLE 1248

| Compound Number | Physical property value [¹H-NMR data TMS δ (ppm)] | |
|---|---|---|
| F-0165 | 400 MHz, CDCl₃ | 1.58-1.64 (2H, m), 1.73-1.90 (4H, m), 2.83 (2H, t), 4.06 (2H, t), 6.81 (1H, t), |
| | | 7.20 (1H, d), 7.62 (1H, d) |
| F-0171 | 400 MHz, CDCl₃ | 1.57-1.65 (2H, m), 1.75-1.90 (4H, m), 2.92 (2H, t), 4.06 (2H, t), 7.20 (1H, d), |
| | | 7.62 (1H, d) |
| F-0509 | 300 MHz, CDCl₃ | 1.50 (3H, d), 4.04-4.20 (3H, m), 5.32 (1H, brs), 7.28 (1H, d), 7.65 (1H, d) |
| F-0511 | 300 MHz, CDCl₃ | 1.50 (3H, d), 4.04-4.20 (3H, m), 5.34 (1H, brs), 7.28 (1H, d), 7.64 (1H, d) |
| F-0965 | 400 MHz, CDCl₃ | 1.57-1.66 (2H, m), 1.73-1.91 (4H, m), 2.57 (3H, s), 2.92 (2H, t), 4.06 (2H, t), |
| | | 7.10 (1H, d), 7.67 (1H, d) |
| F-1303 | 400 MHz, DMSO-d6 | 1.26 (3H, d), 2.46 (3H, s), 3.83-3.92 (1H, m), 3.96 (1H, t), 4.10 (1H, dd), |
| | | 7.21 (1H, d), 7.57 (1H, d), 9.61(1H, brs), 12.93 (1H, brs) |
| F-1626 | 300 MHz, DMSO-d6 | 3.53 (2H, t), 4.10 (2H, t), 7.12 (1H, dd), 7.30 (1H, d), 7.46 (1H, d) |
| F-1644 | 300 MHz, DMSO-d6 | 1.23 (3H, d), 3.82-3.94 (2H, m), 4.04 (1H, m), 7.11 (1H, dd), 7.30 (1H, d), |
| | | 7.46 (1H, d) |
| F-1645 | 300 MHz, DMSO-d6 | 1.34 (3H, d), 3.44 (1H, dd), 3.62 (1H, d), 4.57 (1H, m), 5.42 (1H, s), 7.03 |
| | | (1H, d), 7.41 (1H, d), 7.52 (1H, d) |
| F-1773 | 400 MHz, CDCl₃ | 1.56-1.63 (2H, m), 1.72-1.83 (4H, m), 2.57 (3H, s), 2.84 (2H, t), 3.99 (2H, t), |
| | | 6.81 (1H, t), 7.01 (1H, dd), 7.17 (1H, d), 7.67 (1H, d) |
| F-1779 | 400 MHz, CDCl₃ | 1.57-1.64 (2H, m), 1.75-1.86 (4H, m), 2.58 (3H, s), 2.92 (2H, t), 3.99 (2H, t), |
| | | 7.01 (1H, dd), 7.17 (1H, d), 7.58 (1H, d), 11.71 (1H, brs) |
| F-1810 | 400 MHz, CDCl₃ | 1.48 (3H, d), 2.58 (3H, s), 3.97 (1H, m), 4.11 (2H, m), 5.23 (1H, brs), 7.02 |
| | | (1H, dd), 7.21 (1H, d), 7.56 (1H, dd) |

TABLE 1249

| Compound Number | Physical property value [1H-NMR data TMS δ (ppm)] | |
|---|---|---|
| F-1923 | 300 MHz, DMSO-d6 | 1.52 (3H, d), 4.14 (3H, m), 5.77 (1H, brs), 7.38 (1H, s), 7.83 (1H, s), 8.00 |
| | | (1H, s) |
| F-1926 | 300 MHz, DMSO-d6 | 1.48 (3H, d), 4.00 (1H, m), 4.13 (2H, m), 5.50 (1H, brs), 7.11 (1H, dd), 7.29 |
| | | (1H, d), 7.56 (1H, d) |
| F-0074 | 300 MHz, DMSO-d6 | 1.00 (4H, s), 4.09 (2H, s), 7.54 (1H, d), 7.51 (1H, d) |
| F-0387 | 300 MHz, DMSO-d6 | 0.84-1.02 (4H, m), 1.38 (3H, d), 2.67 (1H, m), 4.22-4.43 (3H, m), |
| | | 7.57 (1H, d), 7.63 (1H, d) |

TABLE 1250

Physical property value

| | | |
|---|---|---|
| | | Refractive index |
| Compound Number | Melting point (°C) | (n_{D}²⁰) |

| | |
|---|---|
| G-0430 | 1.5010 |
| G-1893 | 1.4862 |
| G-3809 | 1.4978 |
| G-4392 | 1.5055 |
| G-4480 | 1.5072 |

TABLE 1251

| Compound Number | Physical property value [¹H-NMR data TMS δ (ppm)] | |
|---|---|---|
| G-0036 | 300 MHz, CDCl₃ | 1.15 (3H, d), 2.39 (1H, m), 3.65 (1H, dd), 3.71 (1H, dd), 3.92 (3H, s), 3.96- |
| | | 4.05 (2H, m), 7.18 (1H, d), 7.48 (1H, d) |
| G-0104 | 300 MHz, CDCl₃ | 2.31 (3H, s), 3.92 (3H, s), 4.64 (2H, s), 7.34 (1H, d), 7.42 (1H, d) |
| G-0378 | 300 MHz, DMSO-d6 | 1.09 (3H, d), 3.18 (2H, brs), 3.23 (1H, m), 3.86 (3H, s), 3.88-3.96 (2H, m), |
| | | 7.59 (1H, d), 7.63(1H, d) |
| G-0381 | 300 MHz, DMSO-d6 | 1.28 (3H, d), 3.61 (1H, m), 3.87 (3H, s), 4.16 (1H, dd), 4.25 (1H, dd), 7.65 |
| | | (1H, d), 7.68(1H, d), 8.14 (3H, brs) |
| G-0382 | 300 MHz, CDCl₃ | 1.32 (3H, d), 3.52 (1H, m), 3.91 (3H, s), 3.97-4.12 (2H, m), 6.03 (2H, brs), |
| | | 7.25 (1H, d), 7.55 (1H, d) |
| G-0383 | 300 MHz, DMSO-d6 | 1.29 (3H, d), 3.63 (1H, m), 3.87 (3H, s), 4.17 (1H, dd), 4.27 (1H, dd), 7.66 |
| | | (1H, d), 7.68(1H, d), 8.25 (3H, brs) |
| G-0393 | 300 MHz, CDCl₃ | 0.31-0.41 (2H, m), 0.44-0.56 (2H, m), 1.23 (3H, d), 2.17 (1H, m), 3.26 (1H, |
| | | m), 3.92 (3H, s), 3.94-4.01 (3H, m), 7.19 (1H, d), 7.50 (1H, d) |
| G-0431 | 300 MHz, CDCl₃ | 1.31 (3H, d), 1.45 (9H, s), 3.92 (3H, s), 3.96-4.09 (3H, m), 4.75 (1H, brs), |
| | | 7.19 (1H, d), 7.50 (1H, d) |
| G-0432 | 300 MHz, CDCl₃ | 1.29 (3H, d), 1.43 (9H, s), 3.91 (3H, s), 3.96-4.09 (3H, m), 4.72 (1H, brs), |
| | | 7.18 (1H, d), 7.49 (1H, d) |
| G-0516 | 300 MHz, CDCl₃ | 1.49 (3H, d), 3.93 (3H, s), 4.02-4.18 (3H, m), 5.28 (1H, brs), 7.24 (1H, d), |
| | | 7.49 (1H, d) |
| G-0517 | 300 MHz, CDCl₃ | 1.49 (3H, d), 3.93 (3H, s), 4.02-4.17 (3H, m), 5.49 (1H, brs), 7.23 (1H, d), |
| | | 7.49 (1H, d) |
| G-0518 | 300 MHz, CDCl₃ | 1.49 (3H, d), 3.94 (3H, s), 4.02-4.18 (3H, m), 5.24 (1H, brs), 7.24 (1H, d), |
| | | 7.49 (1H, d) |

TABLE 1252

| Compound Number | Physical property value [¹H-NMR data TMS δ (ppm)] | |
|---|---|---|
| G-1641 | 300 MHz, DMSO-d6 | 3.20 (2H, t), 3.86 (3H, s), 4.23 (2H, t), 7.21 (1H, dd), 7.38 (1H, d), 7.52 (1H, |
| | | d), 8.20 (3H, brs) |
| G-1652 | 300 MHz, DMSO-d6 | 1.28 (3H, d), 3.60 (1H, m), 3.87 (3H, s), 4.03 (1H, dd), 4.19 (1H, dd), 7.22 |
| | | (1H, dd), 7.40 (1H, d), 7.53 (1H, d), 8.23 (1H, brs) |
| G-1656 | 300 MHz, DMSO-d6 | 1.27 (3H, d), 3.01 (1H, dd), 3.12 (1H, dd), 3.86 (3H, s), 4.72 (1H, s), 7.24 |
| | | (1H, d), 7.40 (1H, d), 7.51 (1H, d), 8.17 (1H, brs) |
| G-1668 | 300 MHz, CDCl₃ | 1.45 (9H, s), 3.54 (2H, m), 3.93 (3H, s), 4.21 (2H, t), 4.93 (1H brs), 6.96 |
| | | (1H, dd), 7.32 (1H, d), 7.35(1H, d) |
| G-1674 | 300 MHz, CDCl₃ | 1.29 (3H, d), 1.45 (9H, s), 3.93 (3H, s), 3.94-4.11 (3H, m), 4.71 (1H, brs), |
| | | 6.98 (1H, dd), 7.34 (1H, d), 7.35 (1H, d) |
| G-1677 | 300 MHz, CDCl₃ | 1.27 (3H, d), 1.44 (9H, s) 3.24 (1H, m), 3.46 (1H, m), 3.927 (3H, s), 4.49 |
| | | (1H, m), 4.89 (1H, brs), 6.97 (1H, dd), 7.34 (d, 1H), 7.35 (1H, d,) |
| G-1703 | 300 MHz, CDCl₃ | 3.70 (2H, t), 3.93 (1H, s), 4.12 (2H, t), 5.40 (1H, s), 6.96 (1H, dd), 7.33 (1H, |
| | | d), 7.36 (1H, d) |
| G-1721 | 300 MHz, CDCl₃ | 1.46 (3H, d), 3.94 (3H, s), 3.96 (1H, m), 4.05-4.13 (2H, m), 5.24 (1H, brs), |
| | | 6.97 (1H, dd), 7.34 (1H, d), 7.37 (1H, d) |
| G-1722 | 300 MHz, CDCl₃ | 1.32 (3H, d), 3.42 (1H, dd), 3.60 (1H, dd), 4.55 (1H, m), 5.35 (1H, brs), 6.97 |
| | | (1H, d), 7.35 (1H, d), 7.37 (1H, d) |
| G-1855 | 300 MHz, DMSO-d6 | 1.29 (3H, d), 2.44 (3H, s), 3.58 (1H, m), 3.83 (3H, s), 4.00 (1H, dd), 4.14 |
| | | (1H, dd), 7.13 (1H, dd), 7.28 (1H, d), 7.41 (1H, d), 8.17 (3H, brs) |
| G-1877 | 300 MHz, CDCl₃ | 1.29 (3H, d), 1.45 (9H, s), 2.52 (3H, s), 3.88 (3H, s), 3.93 (2H, m), 4.04 (1H, |
| | | m), 4.76 (1H, brs), 6.96 (1H, dd), 7.14 (1H, d), 7.44(1H, d) |
| G-1924 | 300 MHz, CDCl₃ | 1.46 (3H, d), 2.53 (3H, s), 3.90 (3H, s), 3.95 (1H, m), 4.07 (1H, m), 5.25 |
| | | (1H, brs), 6.95 (1H, dd), 7.18 (1H, d), 7.43 (1H, d) |

TABLE 1253

| Compound Number | Physical property value [1H-NMR data TMS δ (ppm)] | |
|---|---|---|
| G-2040 | 300 MHz, DMSO-d6 | 1.30 (3H, d), 3.63, (1H, m), 3.91 (3H, s), 4.15 (1H, dd), 4.34 (1H, dd), 7.63 |
| | | (1H, m), 7.81 (2H, m), 8.22 (3H, brs) |
| G-2043 | 300 MHZ, CDCl₃ | 1.31 (3H, d), 1.45 (9H, s), 3.93 (3H, s), 4.01 (2H, d), 4.05 (1H, m), |
| | | 4.72 (1H, brs), 7.26 (1H, m), 7.73 (1H, m), 7.90 (1H, m) |
| G-2046 | 300 MHz, CDCl₃ | 1.49 (3H, d), 3.94 (3H, s), 4.11 (3H, m), 5.18 (1H, brs), 7.34 (1H, m), 7.73 |
| | | (1H, m), 7.95 (1H, m) |
| G-2052 | 300 MHz, DMSO-d6 | 1.28 (3H, d), 3.62 (1H, m), 3.87 (3H, s), 4.04 (1H, dd), 4.22 (1H, dd), 7.34 |
| | | (1H, dd), 7.50 (2H. m), 8.10 (3H, brs) |
| G-2055 | 300 MHz, CDCl₃ | 1.28 (3H, d), 1.45 (9H, s), 3.93 (3H, s), 3.96 (2H, d), 4.07 (1H, m), |
| | | 4.67 (1H, brs), 7.07 (1H, dd), 7.23 (1H, d), 7.44 (1H, d) |
| G-2058 | 300 MHz, CDCl₃ | 1.47 (3H, d), 3.94 (3H, s), 3.98 (1H, m), 4.11 (2H, m), 5.17 (1H, brs), 7.07 |
| | | (1H, dd), 7.28 (1H, d), 7.44 (1H, d) |
| G-3697 | 300 MHz, CDCl₃ | 1.19 (3H, s), 1.39 (3H, t), 2.04 (2H, brs), 2.52 (3H, s), 3.40 (1H, m), |
| | | 3.77 (1H, dd), 3.96 (1H, dd), 4.35 (2H, q), 6.94 (1H, d), 7.55 (1H, |
| | | d) |
| G-3749 | 300 MHz, CDCl₃ | 1.31 (3H, d), 1.39 (3H, t), 1.45 (9H, s), 2.52 (3H, s), 3.91-4.21 (3H, |
| | | m), 4.35 (2H, q), 4.81 (1H, brs), 6.94 (1H, d), 7.56 (1H, d) |
| G-3835 | 300 MHz, CDCl₃ | 1.42 (3H, t), 1.47 (3H, d), 2.53 (3H, s), 4.02-4.15 (3H, m), 4.35 |
| | | (2H, q), 5.55 (1H, brs), 6.96 (1H, d), 7.54 (1H, d) |
| G-0074 | 300 MHz, DMSO-d6 | 0.93-0.98 (2H, m), 1.09-1.15 (2H, m), 3.86 (3H, s), 4.25 (2H, s), |
| | | 7.63 (1H, s), 7.67 (1H, s), 8.69 (3H, brs) |
| G-0075 | 300 MHz, CDCl₃ | 0.88-0.99 (4H, m), 1.43 (9H, s), 3.92 (3H, s), 4.05 (2H, s), 5.18 (1H, brs), |
| | | 7.19 (1H, d), 7.47 (1H, d) |
| G-0076 | 300 MHz, CDCl₃ | 1.01-1.05 (2H, m), 1.26-1.30 (2H, m), 3.93 (3H, s), 4.05 (2H, s), |
| | | 5.77 (1H, brs), 7.23 (1H, d), 7.46 (1H, d) |
| G-0106 | 300 MHz, CDCl₃ | 2.98 (1H, brs), 3.94 (3H, s), 4.24 (1H, dd), 4.33 (1H, dd), 4.43 (1H, m), 7.23 |
| | | (1H, d), 7.54 (1H, d) |
| G-0107 | 300 MHz, CDCl₃ | 3.77 (2H, brs), 3.94 (3H, s), 4.27 (2H, s), 7.25 (1H, d), 7.59 (1H, d) |
| G-0108 | 300 MHz, CDCl₃ | 3.94 (3H, s), 5.05 (2H, s), 7.23 (1H, d), 7.61 (1H, d), 8.79 (1H, brs) |
| G-0394 | 300 MHz, CDCl₃ | 0.84-0.94 (2H, m), 1.01 (1H, m), 1.15 (1H, m), 1.50 (3H, d), 2.63 |
| | | (1H, m), 3.94 (3H, s), 4.07 (1H, dd), 4.28 (1H, dd), 4.48 (1H, m), 7.22 (1H, |
| | | d), 7.48 (1H, d) |
| G-0770 | 300 MHz, CDCl₃ | 1.12 (3H, d), 2.57 (1H, m), 3.72 (1H, dd), 3.84 (1H, dd), 3.91 (3H, |
| | | s), 3.94-4.01 (2H, m), 7.11 (1H, d), 7.43 (1H, d), 7.70-7.73 (2H, m), |
| | | 7.82-7.85 (2H, m) |

Next, formulation examples of the pest control agent of the present invention using the 3-alkoxybenzamide derivative or agriculturally acceptable salt thereof according to the present invention produced as described above will be specifically described. However, the type and blending ratio of the compound and the additive are not limited thereto, and can be changed in a wide range. In the following description, “part” means part by mass.

### [Formulation Example 1] Emulsifiable Concentrate

Each compound listed in Tables 1 to 1062 and Tables 1234 to 1244: 10 parts
Cyclohexanone: 30 parts
Polyoxyethylene alkyl aryl ether: 11 parts
Calcium alkylbenzenesulfonate: 4 parts
Methylnaphthalene: 45 parts

The above components were uniformly dissolved to obtain an emulsifiable concentrate.

### [Formulation Example 2] Wettable Powder

Each compound listed in Tables 1 to 1062 and Tables 1234 to 1244: 10 parts
Naphthalenesulfonic acid formalin condensate sodium salt: 0.5 parts
Polyoxyethylene alkyl aryl ether: 0.5 parts
Diatomaceous earth: 24 parts
Clay: 65 parts

The above components were uniformly mixed and pulverized to obtain a wettable powder.

### [Formulation Example 3] Powder

Each compound listed in Tables 1 to 1062 and Tables 1234 to 1244: 2 parts
Diatomaceous earth: 5 parts
Clay: 93 parts

The above components were uniformly mixed and pulverized to obtain a powder.

### [Formulation Example 4] Granule

Each compound listed in Tables 1 to 1062 and Tables 1234 to 1244: 5 parts
Sodium salt of lauryl alcohol sulfate: 2 parts
Sodium lignin sulfonate: 5 parts
Carboxymethyl cellulose: 2 parts
Clay: 86 parts

The above components were uniformly mixed and pulverized. An amount corresponding to 20 parts of water was added to the mixture. The mixture was kneaded and processed into a granular shape of 14 to 32 mesh using an extrusion granulator, followed by drying to obtain a granule.

### [Formulation Example 5] Flowable Agent

Each compound listed in Tables 1 to 1062 and Tables 1234 to 1244: 20 parts
Polyoxyethylene styrenated phenyl ether sulfate: 4 parts
Ethylene glycol: 7 parts
Silicone AF-118N (manufactured by Asahi Kasei Corporation): 0.02 parts
Water: 68.98 parts

The above components were mixed for 30 minutes with a high-speed stirrer, and pulverized with a wet pulverizer to obtain a flowable agent.

### [Formulation Example 6] Water-Dispersible Granule

Each compound listed in Tables 1 to 1062 and Tables 1234 to 1244: 10 parts
Sodium lignin sulfonate: 5 parts
Polyoxyethylene alkyl aryl ether: 1 part
Sodium polycarboxylate: 3 parts
White carbon: 5 parts
Pregelatinized starch: 1 part
Calcium carbonate: 65 parts
Water: 10 parts

The above components were mixed, kneaded, and pressed to obtain a granule. The obtained granule was dried in a fluidized bed drier to obtain a water-dispersible granule.

Next, effects of the pest control agent of the present invention will be described with reference to test examples.

### [Test Example 1] Test for Insecticidal Activity Against Plutella xylostella

The wettable powder prepared in accordance with Formulation Example 2 was diluted with water to a concentration of 500 ppm as an active ingredient. Cabbage leaves were immersed in the chemical solution, air-dried, and then placed in a plastic cup. Ten second instar larvae of Plutella xylostella were released into the cup, and the cup was capped. Thereafter, the cup was placed in a room at constant temperature of 25 °C. The number of dead insects was examined at 6 days after the treatment, and the mortality rate was caluculated by the calculation formula of Equation 1. The number of replicate is one in each test.

$$Mortality rate ( \% ) = ( 1 - \frac{The number of surviving insects}{The number of insects provided for the test} ) * 100 \qquad [ Equation 1 ]$$

The compound numbers of the compounds showing a mortality rate of 90% or more in this test are listed below.
A-0052, A-0056, A-0059, A-0060, A-0061, A-0063, A-0243, A-0405, A-0447, A-0475, A-0480, A-0491, A-0598, A-1310, A-3829, A-4710, A-4715, A-4969, A-6572, A-6692, A-6724, A-6754, A-6889, B-0009, C-0001, C-0002, C-0004, C-0006, C-0010, C-0011, C-0068, C-0128, C-0130, C-0188, C-0190, C-0192, C-0196, C-0197, C-0314, C-0316, C-0374, C-0448, C-0590, C-0664, C-0698, C-0806, C-0880, C-0988, C-1023, C-11248, C-11256, C-11264, C-11272, C-11312, C-11392, C-11400, C-11512, C-11520, C-11528, C-11536, C-11568, C-11624, C-11664, C-11680, C-11696, C-11712, C-11720, C-11744, C-11752, C-11784, C-11792, C-11800, C-1349, C-1367, C-1403, C-1439, C-1457, C-1475, C-1511, C-2980, C-3076, C-3550, C-4162, C-4214, C-8914, C-8950, C-9334, C-9338, C-9400, C-9422, C-9432, C-9464, C-9496, C-9528, C-9560, C-9592, C-9656, C-9688, C-9720, C-9752, C-9784, C-9848, C-9944, D-0006, D-0125, D-0130, D-0592, D-0664, D-2980, D-3076, D-4495

### [Test Example 2] Test for Insecticidal Activity Against Helicoverpa armigera

The wettable powder prepared in accordance with Formulation Example 2 was diluted with water to a concentration of 500 ppm as an active ingredient. Cabbage leaves were immersed in the chemical solution, air-dried, and then placed in a plastic cup. Five hatching larvae of Helicoverpa armigera were released into the cup, and the cup was capped. Thereafter, the cup was placed in a room at constant temperature of 25 °C. The number of dead insects was examined at 6 days after the treatment, and the mortality rate was caluculated by the calculation formula of Equation 1. The number of replicate is two in each test.

The compound numbers of the compounds showing a mortality rate of 90% or more in this test are listed below.
A-0059, A-0063, A-0243, A-0405, A-0491, A-1913, A-3459, A-3506, A-3829, A-4969, B-0009, C-0002, C-0004, C-0006, C-0068, C-0128, C-0130, C-0190, C-0314, C-0316, C-0374, C-0448, C-0590, C-0664, C-0698, C-0880, C-0988, C-1349, C-1403, C-1439, C-1457, C-1475, C-1511, C-4162, C-4214, C-9400, C-9422, C-9432, C-9464, C-9496, C-9656, C-9688, C-9720, C-9752, C-9784, C-9944, C-11256, C-11264, C-11272, C-11312, C-11400, C-11512, C-11520, C-11528, C-11568, C-11624, C-11664, C-11680, C-11688, C-11696, C-11712, C-11720, C-11752, C-11784, C-11792, D-0006, D-0125, D-0130, D-0592, D-0664, D-2992, D-3076, D-4495

### [Test Example 3] Test for Insecticidal Activity Against Nilaparvata lugens

The wettable powder prepared in accordance with Formulation Example 2 was diluted with water to a concentration of 500 ppm as an active ingredient. The rice sprouts were immersed in the chemical solution, and placed in a plastic cup. Ten second instar larvae of Nilaparvata lugens were released into the plastic cup, and the cup was capped. Thereafter, the cup was placed in a room at constant temperature of 25 °C. The number of dead insects was examined at 6 days after the treatment, and the mortality rate was caluculated by the calculation formula of Equation 1. The number of replicate is one in each test.

The compound numbers of the compounds showing a mortality rate of 90% or more in this test are listed below.
A-0015, A-0049, A-0052, A-0056, A-0059, A-0060, A-0063, A-0064, A-0179, A-0370, A-0447, A-0474, A-0479, A-0752, A-0809, A-1483, A-1529, A-4688, A-4690, A-4710, A-4850, A-4969, A-6161, A-6482, A-6572, A-6602, A-6632, A-6662, A-6708, A-6709, A-6754, A-6814, A-6889, A-7024, B-0009, C-0001, C-0004, C-0006, C-0035, C-0066, C-0068, C-0128, C-0129, C-0130, C-0190, C-0191, C-0192, C-0314, C-0315, C-0316, C-0374, C-0448, C-0590, C-0664, C-0698, C-0806, C-0880, C-0988, C-11248, C-11256, C-11264, C-11272, C-11312, C-11392, C-11400, C-11408, C-11512, C-11520, C-11528, C-11536, C-11552, C-11584, C-11624, C-11656, C-11664, C-11680, C-11688, C-11696, C-11704, C-11712, C-11720, C-11728, C-11744, C-11752, C-11760, C-11776, C-11792, C-11808, C-11816, C-11824, C-11840, C-1349, C-13648, C-1367, C-1403, C-1439, C-1457, C-1475, C-1511, C-2980, C-3076, C-3550, C-4162, C-4214, C-8914, C-8950, C-9334, C-9338, C-9400, C-9422, C-9432, C-9464, C-9496, C-9528, C-9560, C-9592, C-9656, C-9688, C-9720, C-9752, C-9784, C-9848, C-9944, D-0006, D-0125, D-0130, D-0592, D-0664, D-2980, D-2992, D-3076, D-4495, G-3809, G-4480

### [Test Example 4] Test for Control Effect Against Aphis gossypii

The wettable powder prepared in accordance with Formulation Example 2 was diluted with water to a concentration of 500 ppm as an active ingredient. A cucumber seedling previously inoculated with Aphis gossypii nymphs was immersed in the chemical solution, and air-dried. The treated cucumber seedling was placed in a room at constant temperature of 25 °C. The number of surviving insects was counted at 3 days after the treatment, and the mortality rate was caluculated by the calculation formula of Equation 1. The number of replicate is one in each test.

The compound numbers of the compounds showing a mortality rate of 90% or more in this test are listed below.
A-0052, A-0059, A-0060, A-0063, A-0064, A-0179, A-0405, A-0447, A-0474, A-3829, A-6572, A-6934, B-0009, C-0001, C-0004, C-0006, C-0066, C-0068, C-0128, C-0129, C-0130, C-0190, C-0191, C-0192, C-0314, C-0315, C-0316, C-0374, C-0448, C-0590, C-0664, C-0698, C-0806, C-0880, C-0988, C-1023, C-11248, C-11256, C-11264, C-11272, C-11312, C-11392, C-11400, C-11408, C-11512, C-11520, C-11528, C-11536, C-11568, C-11584, C-11624, C-11656, C-11664, C-11680, C-11688, C-11696, C-11704, C-11712, C-11720, C-11728, C-11744, C-11752, C-11760, C-11776, C-11784, C-11808, C-11816, C-11824, C-11840, C-1349, C-13648, C-1367, C-1403, C-1439, C-1457, C-1475, C-1511, C-2980, C-3076, C-3550, C-4162, C-4214, C-8914, C-9334, C-9338, C-9400, C-9422, C-9432, C-9464, C-9496, C-9528, C-9560, C-9592, C-9656, C-9688, C-9720, C-9752, C-9784, C-9848, C-9944, D-0006, D-0130, D-0592, D-0664, D-2980, D-2992, D-3076, D-4423, D-4495

### [Test Example 5] Test for control effect against Tetranychus urticae

The wettable powder prepared in accordance with Formulation Example 2 was diluted with water to a concentration of 500 ppm as an active ingredient. A soybean seedling previously inoculated with 35 female adults of Tetranychus urticae was immersed in the chemical solution, and air-dried. The treated soybean seedling was placed in a room at constant temperature of 25 °C The number of surviving female adults was examined at 13 days after the treatment, and the controlling value was determined by the calculation formula of Equation 2. The number of replicate is one in each test.

$$Controlling value = ( 1 - \frac{\begin{matrix}The number of surviving \\ female adults on treated seedling\end{matrix}}{\begin{matrix}The number of surviving \\ female adults on untreated seedling\end{matrix}} ) * 100 \qquad [ Equation 2 ]$$

The compound numbers of the compounds showing a controlling value of 90 or more in this test are listed below.
A-0008, A-0015, A-0030, A-0037, A-0049, A-0052, A-0056, A-0059, A-0060, A-0063, A-0064, A-0170, A-0179, A-0243, A-0273, A-0405, A-0447, A-0474, A-0475, A-0479, A-0480, A-0491, A-0507, A-0538, A-0598, A-0753, A-0758, A-0942, A-1310, A-1477, A-1483, A-1529, A-1960, A-3828, A-3829, A-4665, A-4687, A-4688, A-4689, A-4691, A-4693, A-4710, A-4712, A-4715, A-4735, A-4758, A-4850, A-4969, A-5011, A-5172, A-5977, A-6161, A-6482, A-6497, A-6572, A-6662, A-6692, A-6708, A-6754, A-6814, A-6844, A-6859, A-6889, A-6934, B-0009, C-0001, C-0002, C-0004, C-0005, C-0006, C-0035, C-0066, C-0068, C-0128, C-0129, C-0130, C-0188, C-0190, C-0191, C-0192, C-0196, C-0221, C-0314, C-0315, C-0316, C-0374, C-0448, C-0590, C-0664, C-0698, C-0806, C-0880, C-0988, C-1022, C-1349, C-1367, C-1403, C-1439, C-1457, C-1475, C-1511, C-2980, C-3076, C-4162, C-4214, C-9400, C-9422, C-9432, C-9464, C-9496, C-9656, C-9688, C-9720, C-9752, C-9784, C-9944, C-11248, C-11256, C-11264, C-11272, C-11312, C-11400, C-11512, C-11520, C-11528, C-11568, C-11624, C-11656, C-11664, C-11680, C-11688, C-11696, C-11704, C-11712, C-11720, C-11728, C-11744, C-11752, C-11760, C-11776, C-11784, C-11792, C-11816, C-11824, D-0006, D-0125, D-0130, D-0592, D-0664, D-2977, D-2980, D-2992, D-3076, D-4495

### INDUSTRIAL APPLICABILITY

The present invention provides a novel compound having excellent insecticidal activity and a production intermediate thereof, is useful in the field of pesticides and the field of agriculture, and has industrial applicability.

LENGTHY TABLES
The patent application contains a lengthy table section. A copy of the table is available in electronic form from the USPTO web site (https://seqdata.uspto.gov/docdetail?docId=US20260363240A1). An electronic copy of the table will also be available from the USPTO upon request and payment of the fee set forth in 37 CFR 1.19(b)(3).

1-14. (canceled)

15. A pest control agent comprising a 3-alkoxybenzamide derivative of formula [I] or an agriculturally acceptable salt thereof as an active ingredient, 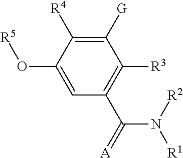
wherein in the formula [I],
A represents an oxygen atom; or a sulfur atom,
G represents a hydrogen atom,
R¹ represents fluoromethyl; difluoromethyl; trifluoromethyl; chloromethyl; dichloromethyl; trichloromethyl; bromomethyl; dibromomethyl; tribromomethyl; iodomethyl; chlorodifluoromethyl; dichlorofluoromethyl; 1-fluoroethyl; 2-fluoroethyl; 1,1-difluoroethyl; 2,2-difluoroethyl; 2,2,2-trifluoroethyl; 1,1,2,2-tetrafluoroethyl; pentafluoroethyl; 1-chloroethyl; 1,1-dichloroethyl; 2,2-dichloroethyl; 2,2,2-trichloroethyl; 1,1,2,2-tetrachloroethyl; pentachloroethyl; 1-bromoethyl; 2,2,2-tribromoethyl; 1-iodoethyl; 2-iodoethyl; 2-chloro-2,2-difluoroethyl; 2,2-dichloro-2-fluoroethyl; 1-fluoropropyl; 2-fluoropropyl; 3-fluoropropyl; 1,1-difluoropropyl; 2,2-difluoropropyl; 3,3-difluoropropyl; 3,3,3-trifluoropropyl; 2,2,3,3,3-pentafluoropropyl; 1,1,2,3,3,3-hexafluoropropyl; heptafluoropropyl; 1-fluoro-2-propyl; 2-fluoro-2-propyl; 1,1-difluoro-2-propyl; 1,2-difluoro-2-propyl; 1,3-difluoro-2-propyl; 1,2,3-trifluoro-2-propyl; 1,1,3,3-tetrafluoro-2-propyl; 1,1,1,3,3,3-hexafluoro-2-propyl; heptafluoro-2-propyl; 1-chloropropyl; 2-chloropropyl; 3-chloropropyl; 1,1-dichloropropyl; 2,2-dichloropropyl; 3,3-dichloropropyl; 3,3,3-trichloropropyl; 2,2,3,3,3-pentachloropropyl; heptachloropropyl; 1-chloro-2-propyl; 2-chloro-2-propyl; 1,1-dichloro-2-propyl; 1,2-dichloro-2-propyl; 1,3-dichloro-2-propyl; 1,2,3-trichloro-2-propyl; 1,1,3,3-tetrachloro-2-propyl; 1,1,1,3,3,3-hexachloro-2-propyl; heptachloro-2-propyl; 1-bromopropyl; 2-bromopropyl; 3-bromopropyl; 1-bromo-2-propyl; 2-bromo-2-propyl; 1-iodopropyl 2-iodopropyl; 3-iodopropyl; 1-iodo-2-propyl; 2-iodo-2-propyl; 1-fluorobutyl; 2-fluorobutyl; 3-fluorobutyl; 4-fluorobutyl; 4,4-difluorobutyl; 4,4,4-trifluorobutyl; 4,4,4-trifluoro-3-methylbutyl; 3,3,4,4,4-pentafluorobutyl; 2,2,3,4,4,4-hexafluorobutyl; 2,2,3,3,4,4,4-heptafluorobutyl; nonafluorobutyl; 1,1,1-trifluoro-2-butyl; 4,4,4-trifluoro-2-butyl 3,3,4,4,4-pentafluoro-2-butyl; nonafluoro-2-butyl; 1,1,1,3,3,3-hexafluoro-2-(trifluoromethyl)-2-propyl; 1-chlorobutyl; 2-chlorobutyl; 3-chlorobutyl; 4-chlorobutyl; 4,4-dichlorobutyl; 4,4,4-trichlorobutyl; nonachlorobutyl; 1,1,1-trichloro-2-butyl; 4,4,4-trichloro-2-butyl; nonachloro-2-butyl; 1-bromobutyl; 2-bromobutyl; 3-bromobutyl; 4-bromobutyl; 1-iodobutyl; 2-iodobutyl; 3-iodobutyl; 4-iodobutyl; 4-chloro-1,1,2,2,3,3,4,4-octafluorobutyl; 4-bromo-1,1,2,2,3,3,4,4-octafluorobutyl; 1-fluoropentyl; 2-fluoropentyl; 3-fluoropentyl; 4-fluoropentyl; 5-fluoropentyl; 5,5,5-trifluoropentyl; 4,4,5,5,5-pentafluoropentyl; 3,3,4,4,5,5,5-heptafluoropentyl; 2,2,3,3,4,4,5,5-octafluoropentyl; 2,2,3,3,4,4,5,5,5-nonafluoropentyl; undecafluoropentyl; 1-chloropentyl; 2-chloropentyl; 3-chloropentyl; 4-chloropentyl; 5-chloropentyl; 5,5,5-trichloropentyl; 4,4,5,5,5-pentachloropentyl; 3,3,4,4,5,5,5-heptachloropentyl; 2,2,3,3,4,4,5,5,5-nonachloropentyl; undecachloropentyl; 1-bromopentyl; 2-bromopentyl; 3-bromopentyl; 4-bromopentyl; 5-bromopentyl; 5-iodopentyl; 1-fluorohexyl; 2-fluorohexyl; 3-fluorohexyl; 4-fluorohexyl; 5-fluorohexyl; 6-fluorohexyl; 6,6,6-trifluorohexyl; 5,5,6,6,6-pentafluorohexyl; 4,4,5,5,6,6,6-heptafluorohexyl; 3,3,4,4,5,5,6,6,6-nonafluorohexyl; 2,2,3,3,4,4,5,5,6,6,6-undecafluorohexyl; 2,2,3,3,4,4,5,5,6,6-decafluorohexyl; tridecafluorohexyl; 1-chlorohexyl; 2-chlorohexyl; 3-chlorohexyl; 4-chlorohexyl; 5-chlorohexyl; 6-chlorohexyl; 5-bromohexyl; 6-bromohexyl; 5-iodohexyl; 6-iodohexyl; a C₃-C₆ cycloalkyl; a C3-C₆ halocycloalkyl; a C₂-C₆ alkenyl; a C₂-C₆ alkynyl; a C₁-C₆ alkoxy; a C₁-C₆ haloalkoxy; a C₁-C₆ alkoxy C₁-C₆ alkyl; a C₃-C₆ cycloalkyl C₁-C₆ alkyl; a cyano C₁-C₆ alkyl; a cyano C₃-C₆ cycloalkyl; a cyano C₃-C₆ cycloalkyl C₁-C₆ alkyl; a cyano heterocycloalkyl; a C₂-C₆ alkynyl C₃-C₆ cycloalkyl; a C₁-C₆ alkoxycarbonyl C₃-C₆ cycloalkyl; a carboxy C₃-C₆ cycloalkyl; a carbamoyl C₃-C₆ cycloalkyl; a C₄-C₈ bicycloalkyl; a phenyl C₁-C₆ alkyl, wherein the phenyl moiety is optionally mono-substituted or poly-substituted with R⁷; or a heteroaryl C₁-C₃ alkyl, wherein the heteroaryl moiety is optionally mono-substituted or poly-substituted with R⁷;
R² represents a hydrogen atom; a C₁-C₆ haloalkyl; a C₂-C₆ alkynyl; a cyano C₁-C₆ alkyl; a hydroxy C₁-C₆ alkyl; a C₁-C₆ alkoxy C₁-C₆ alkyl; a C₁-C₆ alkylcarbonyl; a C₁-C₆ alkoxycarbonyl; a C₁-C₆ haloalkoxycarbonyl; a C₁-C₆ alkoxycarbonyl C₁-C₆ alkyl; an aminocarbonyl C₁-C₆ alkyl; a C₁-C₆ alkylaminocarbonyl C₁-C₆ alkyl, wherein the amino moiety is optionally substituted with R¹⁰; a C₁-C₆ alkylcarbonyloxy C₁-C₆ alkyl; or a C₁-C₆ alkoxycarbonyloxy C₁-C₆ alkyl or
R¹ and R², together with the nitrogen atom to which R¹ and R² are attached form a 3- to 8-membered heterocyclic ring or a 3- to 8-membered heterocyclic ring having 1 to 4 heteroatoms selected from an oxygen atom, a sulfur atom, and a nitrogen atom, wherein the formed heterocyclic ring is optionally substituted with a halogen atom, a cyano, a nitro, a C₁-C₆ alkyl, a C₁-C₆ alkoxy, a C₁-C₆ haloalkyl, or an oxo,
R³ represents a halogen atom; a C₁-C₆ alkyl; a C₁-C₆ haloalkyl; or a C₁-C₆ haloalkoxy,
R⁴ represents a hydrogen atom; a halogen atom; or a C₁-C₆ alkyl,
R⁵ represents a C₂-C₁₂ alkyl, wherein the C₂-C₁₂ alkyl is optionally mono-substituted or poly-substituted with R⁶; or a C₂-C₆ alkenyl, wherein the C₂-C₆ alkenyl is optionally mono-substituted or poly-substituted with R⁶,
R⁶ represents a halogen atom; a C₁-C₆ alkyl; a C₁-C₆ haloalkyl; a C₃-C₆ cycloalkyl; a hydroxy; a thiol; a C₁-C₆ alkylthio; a C₁-C₆ haloalkylthio; a C₁-C₆ alkylsulfinyl; a C₁-C₆ haloalkylsulfinyl; a C₁-C₆ alkylsulfonyl; a C₁-C₆ haloalkylsulfonyl; a C₁-C₆ alkylcarbonyl; an amino; a C₁-C₆ alkylcarbonylamino, wherein the amino moiety is optionally substituted with R¹⁰; a C₁-C₆ haloalkylcarbonylamino, wherein the amino moiety is optionally substituted with R¹⁰; a C₁-C₆ alkoxycarbonylamino, wherein the amino moiety is optionally substituted with R¹⁰; a C₁-C₆ haloalkoxycarbonylamino, wherein the amino moiety is optionally substituted with R¹⁰; a C₁-C₆ haloalkylsulfinylamino, wherein the amino moiety is optionally substituted with R¹⁰; a C₁-C₆ haloalkylsulfonylamino, wherein the amino moiety is optionally substituted with R¹⁰; a phenylsulfonylamino, wherein the phenyl moiety is optionally mono-substituted or poly-substituted with R⁷, and the amino moiety is optionally substituted with R¹⁰; a phenyl, wherein the phenyl is optionally mono-substituted or poly-substituted with R⁷; an azetidinyl, wherein a nitrogen atom of the azetidinyl is optionally substituted with R¹⁰; a 1,3-dioxolanyl; a pyrazolyl, wherein the pyrazolyl is optionally mono-substituted or poly-substituted with R⁷; a triazolyl, wherein the triazolyl is optionally mono-substituted or poly-substituted with R⁷; a 1,3-dioxoisoindolinyl, wherein the 1,3-dioxoisoindolinyl is optionally mono-substituted or poly-substituted with R⁷; or a thiocyanato,
R⁷ represents a halogen atom; a C₁-C₆ alkyl; a C₁-C₆ haloalkyl; or a cyano, and
R¹⁰ represents a C₁-C₆ alkyl; a C₁-C₆ haloalkyl; a C₃-C₆ cycloalkyl; a C₁-C₆ alkoxy C₁-C₆ alkyl; a C₁-C₆ alkoxy C₁-C₆ alkoxy C₁-C₆ alkyl; a C₁-C₆ alkylthio C₁-C₆ alkyl; a C₁-C₆ alkylsulfonyl C₁-C₆ alkyl; a cyano C₁-C₆ alkyl; a C₁-C₆ alkylcarbonyl; a C₁-C₆ alkoxycarbonyl; a C₁-C₆ haloalkylsulfonyl; a C₁-C₆ alkylcarbonyl C₁-C₆ alkyl; a C₁-C₆ alkoxycarbonyl C₁-C₆ alkyl;
a C₁-C₆ alkylcarbonyloxy C₁-C₆ alkyl; a C₁-C₆ alkoxycarbonyloxy C₁-C₆ alkyl; or a 5-methyl-1,3-dioxole-2-one-4-ylmethyl.

16. The pest control agent according to claim 15, wherein the pest control agent is an insecticide, a nematicide, and an acaricide.

17. A process for controlling a pest, comprising a step of applying an effective amount of a 3-alkoxybenzamide derivative of formula [I] or an agriculturally acceptable salt thereof to an agricultural or horticultural crop or a place where the agricultural or horticultural crop is to be grown or being grown, 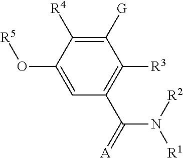
wherein in the formula [I],
A represents an oxygen atom; or a sulfur atom,
G represents a hydrogen atom,
R¹ represents fluoromethyl; difluoromethyl; trifluoromethyl; chloromethyl; dichloromethyl; trichloromethyl; bromomethyl; dibromomethyl; tribromomethyl; iodomethyl; chlorodifluoromethyl; dichlorofluoromethyl; 1-fluoroethyl; 2-fluoroethyl; 1,1-difluoroethyl; 2,2-difluoroethyl; 2,2,2-trifluoroethyl; 1,1,2,2-tetrafluoroethyl; pentafluoroethyl; 1-chloroethyl; 1,1-dichloroethyl; 2,2-dichloroethyl; 2,2,2-trichloroethyl; 1,1,2,2-tetrachloroethyl; pentachloroethyl; 1-bromoethyl; 2,2,2-tribromoethyl; 1-iodoethyl; 2-iodoethyl; 2-chloro-2,2-difluoroethyl; 2,2-dichloro-2-fluoroethyl; 1-fluoropropyl; 2-fluoropropyl; 3-fluoropropyl; 1,1-difluoropropyl; 2,2-difluoropropyl; 3,3-difluoropropyl; 3,3,3-trifluoropropyl; 2,2,3,3,3-pentafluoropropyl; 1,1,2,3,3,3-hexafluoropropyl; heptafluoropropyl; 1-fluoro-2-propyl; 2-fluoro-2-propyl; 1,1-difluoro-2-propyl; 1,2-difluoro-2-propyl; 1,3-difluoro-2-propyl; 1,2,3-trifluoro-2-propyl; 1,1,3,3-tetrafluoro-2-propyl; 1,1,1,3,3,3-hexafluoro-2-propyl; heptafluoro-2-propyl; 1-chloropropyl; 2-chloropropyl; 3-chloropropyl; 1,1-dichloropropyl; 2,2-dichloropropyl; 3,3-dichloropropyl; 3,3,3-trichloropropyl; 2,2,3,3,3-pentachloropropyl; heptachloropropyl; 1-chloro-2-propyl; 2-chloro-2-propyl; 1,1-dichloro-2-propyl; 1,2-dichloro-2-propyl; 1,3-dichloro-2-propyl; 1,2,3-trichloro-2-propyl; 1,1,3,3-tetrachloro-2-propyl; 1,1,1,3,3,3-hexachloro-2-propyl; heptachloro-2-propyl; 1-bromopropyl; 2-bromopropyl; 3-bromopropyl; 1-bromo-2-propyl; 2-bromo-2-propyl; 1-iodopropyl 2-iodopropyl; 3-iodopropyl; 1-iodo-2-propyl; 2-iodo-2-propyl; 1-fluorobutyl; 2-fluorobutyl; 3-fluorobutyl; 4-fluorobutyl; 4,4-difluorobutyl; 4,4,4-trifluorobutyl; 4,4,4-trifluoro-3-methylbutyl; 3,3,4,4,4-pentafluorobutyl; 2,2,3,4,4,4-hexafluorobutyl; 2,2,3,3,4,4,4-heptafluorobutyl; nonafluorobutyl; 1,1,1-trifluoro-2-butyl; 4,4,4-trifluoro-2-butyl 3,3,4,4,4-pentafluoro-2-butyl; nonafluoro-2-butyl; 1,1,1,3,3,3-hexafluoro-2-(trifluoromethyl)-2-propyl; 1-chlorobutyl; 2-chlorobutyl; 3-chlorobutyl; 4-chlorobutyl; 4,4-dichlorobutyl; 4,4,4-trichlorobutyl; nonachlorobutyl; 1,1,1-trichloro-2-butyl; 4,4,4-trichloro-2-butyl; nonachloro-2-butyl; 1-bromobutyl; 2-bromobutyl; 3-bromobutyl; 4-bromobutyl; 1-iodobutyl; 2-iodobutyl; 3-iodobutyl; 4-iodobutyl; 4-chloro-1,1,2,2,3,3,4,4-octafluorobutyl; 4-bromo-1,1,2,2,3,3,4,4-octafluorobutyl; 1-fluoropentyl; 2-fluoropentyl; 3-fluoropentyl; 4-fluoropentyl; 5-fluoropentyl; 5,5,5-trifluoropentyl; 4,4,5,5,5-pentafluoropentyl; 3,3,4,4,5,5,5-heptafluoropentyl; 2,2,3,3,4,4,5,5-octafluoropentyl; 2,2,3,3,4,4,5,5,5-nonafluoropentyl; undecafluoropentyl; 1-chloropentyl; 2-chloropentyl; 3-chloropentyl; 4-chloropentyl; 5-chloropentyl; 5,5,5-trichloropentyl; 4,4,5,5,5-pentachloropentyl; 3,3,4,4,5,5,5-heptachloropentyl; 2,2,3,3,4,4,5,5,5-nonachloropentyl; undecachloropentyl; 1-bromopentyl; 2-bromopentyl; 3-bromopentyl; 4-bromopentyl; 5-bromopentyl; 5-iodopentyl; 1-fluorohexyl; 2-fluorohexyl; 3-fluorohexyl; 4-fluorohexyl; 5-fluorohexyl; 6-fluorohexyl; 6,6,6-trifluorohexyl; 5,5,6,6,6-pentafluorohexyl; 4,4,5,5,6,6,6-heptafluorohexyl; 3,3,4,4,5,5,6,6,6-nonafluorohexyl; 2,2,3,3,4,4,5,5,6,6,6-undecafluorohexyl; 2,2,3,3,4,4,5,5,6,6-decafluorohexyl; tridecafluorohexyl; 1-chlorohexyl; 2-chlorohexyl; 3-chlorohexyl; 4-chlorohexyl; 5-chlorohexyl; 6-chlorohexyl; 5-bromohexyl; 6-bromohexyl; 5-iodohexyl; 6-iodohexyl; a C₃-C₆ cycloalkyl; a C3-C₆ halocycloalkyl; a C₂-C₆ alkenyl; a C₂-C₆ alkynyl; a C₁-C₆ alkoxy; a C₁-C₆ haloalkoxy; a C₁-C₆ alkoxy C₁-C₆ alkyl; a C₃-C₆ cycloalkyl C₁-C₆ alkyl; a cyano C₁-C₆ alkyl; a cyano C₃-C₆ cycloalkyl; a cyano C₃-C₆ cycloalkyl C₁-C₆ alkyl; a cyano heterocycloalkyl; a C2-C6 alkynyl C₃-C₆ cycloalkyl; a C₁-C₆ alkoxycarbonyl C₃-C₆ cycloalkyl; a carboxy C₃-C₆ cycloalkyl; a carbamoyl C₃-C₆ cycloalkyl; a C₄-C₈ bicycloalkyl; a phenyl C₁-C₆ alkyl, wherein the phenyl moiety is optionally mono-substituted or poly-substituted with R⁷; or a heteroaryl C₁-C₃ alkyl, wherein the heteroaryl moiety is optionally mono-substituted or poly-substituted with R⁷;
R² represents a hydrogen atom; a C₁-C₆ haloalkyl; a C₂-C₆ alkynyl; a cyano C₁-C₆ alkyl; a hydroxy C₁-C₆ alkyl; a C₁-C₆ alkoxy C₁-C₆ alkyl; a C₁-C₆ alkylcarbonyl; a C₁-C₆ alkoxycarbonyl; a C₁-C₆ haloalkoxycarbonyl; a C₁-C₆ alkoxycarbonyl C₁-C₆ alkyl; an aminocarbonyl C₁-C₆ alkyl; a C₁-C₆ alkylaminocarbonyl C₁-C₆ alkyl, wherein the amino moiety is optionally substituted with R¹⁰; a C₁-C₆ alkylcarbonyloxy C₁-C₆ alkyl; or a C₁-C₆ alkoxycarbonyloxy C₁-C₆ alkyl or
R¹ and R², together with the nitrogen atom to which R¹ and R² are attached form a 3- to 8-membered heterocyclic ring or a 3- to 8-membered heterocyclic ring having 1 to 4 heteroatoms selected from an oxygen atom, a sulfur atom, and a nitrogen atom, wherein the formed heterocyclic ring is optionally substituted with a halogen atom, a cyano, a nitro, a C₁-C₆ alkyl, a C₁-C₆ alkoxy, a C₁-C₆ haloalkyl, or an oxo,
R³ represents a halogen atom; a C₁-C₆ alkyl; a C₁-C₆ haloalkyl; or a C₁-C₆ haloalkoxy,
R⁴ represents a hydrogen atom; a halogen atom; or a C₁-C₆ alkyl,
R⁵ represents a C₂-C₁₂ alkyl, wherein the C₂-C₁₂ alkyl is optionally mono-substituted or poly-substituted with R⁶; or a C₂-C₆ alkenyl, wherein the C₂-C₆ alkenyl is optionally mono-substituted or poly-substituted with R⁶,
R⁶ represents a halogen atom; a C₁-C₆ alkyl; a C₁-C₆ haloalkyl; a C₃-C₆ cycloalkyl; a hydroxy; a thiol; a C₁-C₆ alkylthio; a C₁-C₆ haloalkylthio; a C₁-C₆ alkylsulfinyl; a C₁-C₆ haloalkylsulfinyl; a C₁-C₆ alkylsulfonyl; a C₁-C₆ haloalkylsulfonyl; a C₁-C₆ alkylcarbonyl; an amino; a C₁-C₆ alkylcarbonylamino, wherein the amino moiety is optionally substituted with R¹⁰; a C₁-C₆ haloalkylcarbonylamino, wherein the amino moiety is optionally substituted with R¹⁰; a C₁-C₆ alkoxycarbonylamino, wherein the amino moiety is optionally substituted with R¹⁰; a C₁-C₆ haloalkoxycarbonylamino, wherein the amino moiety is optionally substituted with R¹⁰; a C₁-C₆ haloalkylsulfinylamino, wherein the amino moiety is optionally substituted with R¹⁰; a C₁-C₆ haloalkylsulfonylamino, wherein the amino moiety is optionally substituted with R¹⁰; a phenylsulfonylamino, wherein the phenyl moiety is optionally mono-substituted or poly-substituted with R⁷, and the amino moiety is optionally substituted with R¹⁰; a phenyl, wherein the phenyl is optionally mono-substituted or poly-substituted with R⁷; an azetidinyl, wherein a nitrogen atom of the azetidinyl is optionally substituted with R¹⁰; a 1,3-dioxolanyl; a pyrazolyl, wherein the pyrazolyl is optionally mono-substituted or poly-substituted with R⁷; a triazolyl, wherein the triazolyl is optionally mono-substituted or poly-substituted with R⁷; a 1,3-dioxoisoindolinyl, wherein the 1,3-dioxoisoindolinyl is optionally mono-substituted or poly-substituted with R⁷; or a thiocyanato.

18. A process for controlling a pest, comprising a step of applying an effective amount of an agrochemical composition comprising a 3-alkoxybenzamide derivative of formula [I] or an agriculturally acceptable salt thereof as an active ingredient to an agricultural or horticultural crop or a place where the agricultural or horticultural crop is to be grown or being grown, in a single or divided dose, 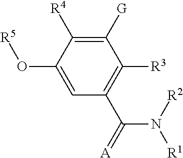
wherein in the formula [I],
A represents an oxygen atom; or a sulfur atom,
G represents a hydrogen atom,
R¹ represents fluoromethyl; difluoromethyl; trifluoromethyl; chloromethyl; dichloromethyl; trichloromethyl; bromomethyl; dibromomethyl; tribromomethyl; iodomethyl; chlorodifluoromethyl; dichlorofluoromethyl; 1-fluoroethyl; 2-fluoroethyl; 1,1-difluoroethyl; 2,2-difluoroethyl; 2,2,2-trifluoroethyl; 1,1,2,2-tetrafluoroethyl; pentafluoroethyl; 1-chloroethyl; 1,1-dichloroethyl; 2,2-dichloroethyl; 2,2,2-trichloroethyl; 1,1,2,2-tetrachloroethyl; pentachloroethyl; 1-bromoethyl; 2,2,2-tribromoethyl; 1-iodoethyl; 2-iodoethyl; 2-chloro-2,2-difluoroethyl; 2,2-dichloro-2-fluoroethyl; 1-fluoropropyl; 2-fluoropropyl; 3-fluoropropyl; 1,1-difluoropropyl; 2,2-difluoropropyl; 3,3-difluoropropyl; 3,3,3-trifluoropropyl; 2,2,3,3,3-pentafluoropropyl; 1,1,2,3,3,3-hexafluoropropyl; heptafluoropropyl; 1-fluoro-2-propyl; 2-fluoro-2-propyl; 1,1-difluoro-2-propyl; 1,2-difluoro-2-propyl; 1,3-difluoro-2-propyl; 1,2,3-trifluoro-2-propyl; 1,1,3,3-tetrafluoro-2-propyl; 1,1,1,3,3,3-hexafluoro-2-propyl; heptafluoro-2-propyl; 1-chloropropyl; 2-chloropropyl; 3-chloropropyl; 1,1-dichloropropyl; 2,2-dichloropropyl; 3,3-dichloropropyl; 3,3,3-trichloropropyl; 2,2,3,3,3-pentachloropropyl; heptachloropropyl; 1-chloro-2-propyl; 2-chloro-2-propyl; 1,1-dichloro-2-propyl; 1,2-dichloro-2-propyl; 1,3-dichloro-2-propyl; 1,2,3-trichloro-2-propyl; 1,1,3,3-tetrachloro-2-propyl; 1,1,1,3,3,3-hexachloro-2-propyl; heptachloro-2-propyl; 1-bromopropyl; 2-bromopropyl; 3-bromopropyl; 1-bromo-2-propyl; 2-bromo-2-propyl; 1-iodopropyl 2-iodopropyl; 3-iodopropyl; 1-iodo-2-propyl; 2-iodo-2-propyl; 1-fluorobutyl; 2-fluorobutyl; 3-fluorobutyl; 4-fluorobutyl; 4,4-difluorobutyl; 4,4,4-trifluorobutyl; 4,4,4-trifluoro-3-methylbutyl; 3,3,4,4,4-pentafluorobutyl; 2,2,3,4,4,4-hexafluorobutyl; 2,2,3,3,4,4,4-heptafluorobutyl; nonafluorobutyl; 1,1,1-trifluoro-2-butyl; 4,4,4-trifluoro-2-butyl 3,3,4,4,4-pentafluoro-2-butyl; nonafluoro-2-butyl; 1,1,1,3,3,3-hexafluoro-2-(trifluoromethyl)-2-propyl; 1-chlorobutyl; 2-chlorobutyl; 3-chlorobutyl; 4-chlorobutyl; 4,4-dichlorobutyl; 4,4,4-trichlorobutyl; nonachlorobutyl; 1,1,1-trichloro-2-butyl; 4,4,4-trichloro-2-butyl; nonachloro-2-butyl; 1-bromobutyl; 2-bromobutyl; 3-bromobutyl; 4-bromobutyl; 1-iodobutyl; 2-iodobutyl; 3-iodobutyl; 4-iodobutyl; 4-chloro-1,1,2,2,3,3,4,4-octafluorobutyl; 4-bromo-1,1,2,2,3,3,4,4-octafluorobutyl; 1-fluoropentyl; 2-fluoropentyl; 3-fluoropentyl; 4-fluoropentyl; 5-fluoropentyl; 5,5,5-trifluoropentyl; 4,4,5,5,5-pentafluoropentyl; 3,3,4,4,5,5,5-heptafluoropentyl; 2,2,3,3,4,4,5,5-octafluoropentyl; 2,2,3,3,4,4,5,5,5-nonafluoropentyl; undecafluoropentyl; 1-chloropentyl; 2-chloropentyl; 3-chloropentyl; 4-chloropentyl; 5-chloropentyl; 5,5,5-trichloropentyl; 4,4,5,5,5-pentachloropentyl; 3,3,4,4,5,5,5-heptachloropentyl; 2,2,3,3,4,4,5,5,5-nonachloropentyl; undecachloropentyl; 1-bromopentyl; 2-bromopentyl; 3-bromopentyl; 4-bromopentyl; 5-bromopentyl; 5-iodopentyl; 1-fluorohexyl; 2-fluorohexyl; 3-fluorohexyl; 4-fluorohexyl; 5-fluorohexyl; 6-fluorohexyl; 6,6,6-trifluorohexyl; 5,5,6,6,6-pentafluorohexyl; 4,4,5,5,6,6,6-heptafluorohexyl; 3,3,4,4,5,5,6,6,6-nonafluorohexyl; 2,2,3,3,4,4,5,5,6,6,6-undecafluorohexyl; 2,2,3,3,4,4,5,5,6,6-decafluorohexyl; tridecafluorohexyl; 1-chlorohexyl; 2-chlorohexyl; 3-chlorohexyl; 4-chlorohexyl; 5-chlorohexyl; 6-chlorohexyl; 5-bromohexyl; 6-bromohexyl; 5-iodohexyl; 6-iodohexyl; a C₃-C₆ cycloalkyl; a C₃-C₆ halocycloalkyl; a C₂-C₆ alkenyl; a C₂-C₆ alkynyl; a C₁-C₆ alkoxy; a C₁-C₆ haloalkoxy; a C₁-C₆ alkoxy C₁-C₆ alkyl; a C₃-C₆ cycloalkyl C₁-C₆ alkyl; a cyano C₁-C₆ alkyl; a cyano C₃-C₆ cycloalkyl; a cyano C₃-C₆ cycloalkyl C₁-C₆ alkyl; a cyano heterocycloalkyl; a C₂-C₆ alkynyl C₃-C₆ cycloalkyl; a C₁-C₆ alkoxycarbonyl C₃-C₆ cycloalkyl; a carboxy C₃-C₆ cycloalkyl; a carbamoyl C₃-C₆ cycloalkyl; a C₄-C₈ bicycloalkyl; a phenyl C₁-C₆ alkyl, wherein the phenyl moiety is optionally mono-substituted or poly-substituted with R⁷; or a heteroaryl C₁-C₃ alkyl, wherein the heteroaryl moiety is optionally mono-substituted or poly-substituted with R⁷;
R² represents a hydrogen atom; a C₁-C₆ haloalkyl; a C₂-C₆ alkynyl; a cyano C₁-C₆ alkyl; a hydroxy C₁-C₆ alkyl; a C₁-C₆ alkoxy C₁-C₆ alkyl; a C₁-C₆ alkylcarbonyl; a C₁-C₆ alkoxycarbonyl; a C₁-C₆ haloalkoxycarbonyl; a C₁-C₆ alkoxycarbonyl C₁-C₆ alkyl; an aminocarbonyl C₁-C₆ alkyl; a C₁-C₆ alkylaminocarbonyl C₁-C₆ alkyl, wherein the amino moiety is optionally substituted with R¹⁰; a C₁-C₆ alkylcarbonyloxy C₁-C₆ alkyl; or a C₁-C₆ alkoxycarbonyloxy C₁-C₆ alkyl or
R¹ and R², together with the nitrogen atom to which R¹ and R² are attached form a 3- to 8-membered heterocyclic ring or a 3- to 8-membered heterocyclic ring having 1 to 4 heteroatoms selected from an oxygen atom, a sulfur atom, and a nitrogen atom, wherein the formed heterocyclic ring is optionally substituted with a halogen atom, a cyano, a nitro, a C₁-C₆ alkyl, a C₁-C₆ alkoxy, a C₁-C₆ haloalkyl, or an oxo,
R³ represents a halogen atom; a C₁-C₆ alkyl; a C₁-C₆ haloalkyl; or a C₁-C₆ haloalkoxy,
R⁴ represents a hydrogen atom; a halogen atom; or a C₁-C₆ alkyl,
R⁵ represents a C₂-C₁₂ alkyl, wherein the C₂-C₁₂ alkyl is optionally mono-substituted or poly-substituted with R⁶; or a C₂-C₆ alkenyl, wherein the C₂-C₆ alkenyl is optionally mono-substituted or poly-substituted with R⁶,
R⁶ represents a halogen atom; a C₁-C₆ alkyl; a C₁-C₆ haloalkyl; a C₃-C₆ cycloalkyl; a hydroxy; a thiol; a C₁-C₆ alkylthio; a C₁-C₆ haloalkylthio; a C₁-C₆ alkylsulfinyl; a C₁-C₆ haloalkylsulfinyl; a C₁-C₆ alkylsulfonyl; a C₁-C₆ haloalkylsulfonyl; a C₁-C₆ alkylcarbonyl; an amino; a C₁-C₆ alkylcarbonylamino, wherein the amino moiety is optionally substituted with R¹⁰; a C₁-C₆ haloalkylcarbonylamino, wherein the amino moiety is optionally substituted with R¹⁰; a C₁-C₆ alkoxycarbonylamino, wherein the amino moiety is optionally substituted with R¹⁰; a C₁-C₆ haloalkoxycarbonylamino, wherein the amino moiety is optionally substituted with R¹⁰; a C₁-C₆ haloalkylsulfinylamino, wherein the amino moiety is optionally substituted with R¹⁰; a C₁-C₆ haloalkylsulfonylamino, wherein the amino moiety is optionally substituted with R¹⁰; a phenylsulfonylamino, wherein the phenyl moiety is optionally mono-substituted or poly-substituted with R⁷, and the amino moiety is optionally substituted with R¹⁰; a phenyl, wherein the phenyl is optionally mono-substituted or poly-substituted with R⁷; an azetidinyl, wherein a nitrogen atom of the azetidinyl is optionally substituted with R¹⁰; a 1,3-dioxolanyl; a pyrazolyl, wherein the pyrazolyl is optionally mono-substituted or poly-substituted with R⁷; a triazolyl, wherein the triazolyl is optionally mono-substituted or poly-substituted with R⁷; a 1,3-dioxoisoindolinyl, wherein the 1,3-dioxoisoindolinyl is optionally mono-substituted or poly-substituted with R⁷; or a thiocyanato.

19. The process for controlling a pest according to claim 17, wherein a place where a pest control agent is applied is a paddy field, a dry field, a lawn, an orchard, a greenhouse, a raising seeding facility, and a plant factory.

20. The process for controlling a pest according to claim 17, wherein the 3-alkoxybenzamide derivative of formula [I] or the agriculturally acceptable salt thereof is used as an insecticide, a nematicide, and an acaricide.

21. A process for using a pest control agent, characterized in that, in the process, the pest control agent according to claim 15 is applied for controlling a pest harmful to an agricultural or horticultural crop.

22. The process for controlling a pest according to claim 18, wherein a place where a pest control agent is applied is a paddy field, a dry field, a lawn, an orchard, a greenhouse, a raising seeding facility, and a plant factory.

23. The process for controlling a pest according to claim 18, wherein the 3-alkoxybenzamide derivative of formula [I] or the agriculturally acceptable salt thereof is used as an insecticide, a nematicide, and an acaricide.

24. The process for controlling a pest according to claim 19, wherein the 3-alkoxybenzamide derivative of formula [I] or the agriculturally acceptable salt thereof is used as an insecticide, a nematicide, and an acaricide.

25. The process for controlling a pest according to claim 22, wherein the 3-alkoxybenzamide derivative of formula [I] or the agriculturally acceptable salt thereof is used as an insecticide, a nematicide, and an acaricide.
